(19) United States
(12) Patent Application Publication
(10) Pub. No.: US 2023/0374519 A1
(43) Pub. Date: Nov. 23, 2023

(54) COMPOUNDS AND METHODS FOR MODULATING PMP22

(71) Applicant: Ionis Pharmaceuticals, Inc., Carlsbad, CA (US)

(72) Inventors: Eric E. Swayze, Encinitas, CA (US); Holly Kordasiewicz, San Diego, CA (US); Punit P. Seth, Carlsbad, CA (US); Hien Thuy Zhao, San Diego, CA (US); Michael T. Migawa, Carlsbad, CA (US); Ruben E. Valas, Oceanside, CA (US); Thazha P. Prakash, Carlsbad, CA (US)

(73) Assignee: Ionis Pharmaceuticals, Inc., Carlsbad, CA (US)

(21) Appl. No.: 18/001,848

(22) PCT Filed: Jun. 18, 2021
(86) PCT No.: PCT/US2021/038126

§ 371 Date: Dec. 14, 2022

Related U.S. Application Data
(60) Provisional application No. 63/041,474, filed on Jun. 19, 2020.
(60) Provisional application No. 63/041,643, filed on Jun. 19, 2020.

(51) Int. Cl. C12N 15/113; A61K 31/7125; A61P 25/28

(52) U.S. Cl. CPC C12N 15/1138; A61K 31/7125; A61P 25/28; C12N 2310/11; C12N 2310/14; C12N 2310/323; C12N 2310/322; C12N 2310/321; C12N 2310/3233; C12N 2310/3181; C12N 2310/315; C12N 2310/31; C12N 2310/3525; C12N 2310/3341; C12N 2310/3515; C12N 2310/351; C12N 2310/3125; C12N 2310/346; C12N 2320/35

(57) ABSTRACT

Provided are compounds, methods, and pharmaceutical compositions for reducing the amount or activity of PMP22 RNA in a cell or animal, and in certain instances reducing the amount of PMP22 protein in a cell or animal. Such compounds, methods, and pharmaceutical compositions are useful to ameliorate at least one symptom or hallmark of a neurodegenerative disease. Such symptoms and hallmarks include demyelination, progressive axonal damage and/or loss, weakness and wasting of foot and lower leg muscles, foot deformities, and weakness and atrophy in the hands. Such neurodegenerative diseases include Charcot-Marie-Tooth disease.

### SEQUENCE LISTING

The present application is being filed along with a Sequence Listing in electronic format. The Sequence Listing is provided as a file entitled BIOL0390WOSEQ_ST25.txt, created on Jun. 18, 2021 which is 252 KB in size. The information in the electronic format of the sequence listing is incorporated herein by reference in its entirety.

### FIELD

Provided are compounds, methods, and pharmaceutical compositions for reducing the amount or activity of PMP22 RNA in a cell or subject, and in certain instances reducing the amount of PMP22 protein in a cell or subject. Such compounds, methods, and pharmaceutical compositions are useful to ameliorate at least one symptom or hallmark of a neurodegenerative disease. Such symptoms and hallmarks include demyelination, progressive axonal damage and/or loss, weakness and wasting of foot and lower leg muscles, foot deformities, and weakness and atrophy in the hands. Such neurodegenerative diseases include Charcot-Marie-Tooth disease, Charcot-Marie-Tooth disease type 1A, Charcot-Marie-Tooth disease type 1E, and Dejerine Sottas Syndrome.

### BACKGROUND

Charcot-Marie-Tooth disease (CMT) is one of the most common inherited neurological disorders, affecting approximately 1 in 2,500 people in the United States. CMT, also known as hereditary motor and sensory neuropathy (HMSN) or peroneal muscular atrophy, comprises a group of disorders that affect peripheral nerves. Charcot-Marie-Tooth disease type 1A (CMT1A) is an inherited neurodegenerative disease caused by duplication of the PMP22 gene. It is the most common inherited peripheral neuropathy and is characterized by progressive distal motor weakness. Symptoms are caused by progressive demyelination of peripheral neurons, followed by axonal dysfunction and/or degeneration (Krajewski, et. al, “Neurological dysfunction and axonal degeneration in Charcot-Marie-Tooth disease type 1A”, Brain, 2000, 123(Pt. 7):1516-1527). Symptoms include weakness and wasting of foot and lower leg muscles, foot deformities, and weakness and atrophy in the hands. Additionally, myelin deficits can be detected by electrophysiology, and often appear years before symptom onset (Kim, et al., “Comparison between Clinical Disabilities and Electrophysiological Values in Charcot-Marie-Tooth 1A Patients with PMP22 Duplication”, J. Clin. Neuro., 2012, 8(2):139-145). Charcot-Marie-Tooth disease type 1E (CMT1E) and Dejerine-Sottas Syndrome are inherited neurodegenerative diseases caused by mutations in the PMP22 gene. Symptoms include impaired motor development, distal muscle weakness, foot deformities, and a loss of deep tendon reflex (Li, et al., “The PMP22 Gene and Its Related Diseases”, Mol. Neurobiol., 2013, 47(2): 673-698).

Currently there is a lack of acceptable options for treating neurodegenerative diseases such as CMT disease, CMT1A, CMT1E, and Dejerine-Sottas Syndrome. It is therefore an object herein to provide compounds, methods, and pharmaceutical compositions for the treatment of such diseases.

### SUMMARY OF THE INVENTION

Provided herein are compounds, methods and pharmaceutical compositions for reducing the amount or activity of PMP22 RNA, and in certain embodiments reducing the amount of PMP22 protein in a cell or subject. In certain embodiments, the subject has a neurodegenerative disease. In certain embodiments, the subject has Charcot-Marie-Tooth disease. In certain embodiments, the subject has Charcot-Marie-Tooth disease type 1A (CMT1A). In certain embodiments, the subject has Charcot-Marie-Tooth disease type 1E (CMT1E). In certain embodiments, the subject has Dejerine-Sottas Syndrome. In certain embodiments, compounds useful for reducing expression of PMP22 RNA are oligomeric compounds. In certain embodiments, compounds useful for reducing expression of PMP22 RNA are modified oligonucleotides. In certain embodiments, compounds useful for reducing expression of PMP22 RNA are modified oligonucleotides attached to a conjugate group.

Also provided are methods useful for ameliorating at least one symptom or hallmark of a neurodegenerative disease. In certain embodiments, the neurodegenerative disease is Charcot-Marie-Tooth disease. In certain embodiments, the neurodegenerative disease is CMT1A. In certain embodiments, the neurodegenerative disease is CMT1E. In certain embodiments, the neurodegenerative disease is Dejerine-Sottas Syndrome. In certain embodiments, the symptom or hallmark includes demyelination, progressive axonal damage and/or loss, weakness and wasting of foot and lower leg muscles, foot deformities, and weakness and atrophy in the hands.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/of” unless stated otherwise. Furthermore, the use of the term “including” as well as other forms, such as “includes” and “included,” is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank, ENSEMBL, and NCBI reference sequence records, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-$3-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′-OH group of a furanosyl sugar moiety. A “2′-MOE sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the p-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′-OH group of a furanosyl sugar moiety. A “2′-O-methyl sugar moiety” or “2′-OMe sugar moiety” means a sugar moiety with a 2′-OCH₃ group in place of the 2′-OH group of a furanosyl sugar moiety. Unless otherwise indicated, a 2′-OMe sugar moiety is in the $-D-ribosyl configuration.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity. An antisense compound comprises an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide. Sense oligonucleotides include but are not limited to sense RNAi oligonucleotides.

As used herein, “antisense agent” means an antisense compound and optionally one or more additional features, such as a sense compound.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is demyelination, progressive axonal damage and/or loss, weakness and wasting of foot and lower leg muscles, foot deformities, and weakness and atrophy in the hands.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms of the first ring thereby forming a bicyclic structure. In certain embodiments, the first ring of the bicyclic sugar moiety is a furanosyl moiety. In certain embodiments, the furanosyl sugar moiety is a ribosyl moiety. In certain embodiments, the bicyclic sugar moiety does not comprise a furanosyl moiety.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or portion thereof, means that the oligonucleotide, or a portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety. As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-susbstituted nucleoside. In certain embodiments, a deoxy region supports RNase H activity. In certain embodiments, a deoxy region is the gap or internal region of a gapmer.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” The internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of function or structure, including loss of motor function and death of neurons. In certain embodiments, the neurodegenerative disease is a peripheral neuropathy. In certain embodiments, the neurodegenerative disease is Charcot-Marie-Tooth disease. In certain embodiments, the neurodegenerative disease is CMT1A. In certain embodiments, the neurodegenerative disease is CMT1E. In certain embodiments, the disease is Dejerine-Sottas Syndrome.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound or a fragment of a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject.

In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means a therapeutic agent in a form outside the body that is converted to a different form within a subject or cells thereof. Typically, conversion of a prodrug within the subject is facilitated by the action of an enzymes (e.g., endogenous or viral enzyme) or chemicals present in cells or tissues and/or by physiologic conditions.

As used herein, “reducing the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to the antisense compound of double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “self-complementary” in reference to an oligonucleotide means an oligonucleotide that at least partially hybridizes to itself.

As used herein, “standard in vivo assay” means the assay described in any of Example 3 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate intemucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) $-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom or hallmark of a disease.

### CERTAIN EMBODIMENTS

The present disclosure provides the following non-limiting numbered embodiments:
Embodiment 1. An oligomeric compound, comprising
   a modified oligonucleotide and a conjugate group, wherein the modified oligonucleotide consists of 12 to 50 linked nucleosides wherein the nucleobase sequence of the modified oligonucleotide is at least 90% complementary to an equal length portion of a PMP22 RNA, and wherein the modified oligonucleotide comprises at least one modification selected from a modified sugar, a sugar surrogate, and a modified internucleoside linkage.
Embodiment 2. An oligomeric compound comprising a modified oligonucleotide and a conjugate group, wherein the modified oligonucleotide consists of 12 to 50 linked nucleosides and having a nucleobase sequence comprising at least 12, 13, 14, 15, or 16 nucleobases of any of SEQ ID NOs: 18-321.
Embodiment 3. The oligomeric compound of any of embodiments 1-2, wherein the modified oligonucleotide has a nucleobase sequence that is at least 80%, 85%, 90%, 95%, or 100% complementary to any of the nucleobase sequences of SEQ ID NOs: 1-8 when measured across the entire nucleobase sequence of the modified oligonucleotide.
Embodiment 4. The oligomeric compound of any of embodiments 1-3, wherein the modified oligonucleotide comprises at least one modified nucleoside.
Embodiment 5. The oligomeric compound of embodiment 4, wherein the modified oligonucleotide comprises at least one modified nucleoside comprising a modified sugar moiety.
Embodiment 6. The oligomeric compound of embodiment 5, wherein the modified oligonucleotide comprises at least one modified nucleoside comprising a bicyclic sugar moiety.
Embodiment 7. The oligomeric compound of embodiment 6, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 8. The oligomeric compound of any of embodiments 5-7, wherein the modified oligonucleotide comprises at least one modified nucleoside comprising a non-bicyclic modified sugar moiety.
Embodiment 9. The oligomeric compound of embodiment 8, wherein the modified oligonucleotide comprises at least one modified nucleoside comprising a non-bicyclic modified sugar moiety comprising a 2′-MOE modified sugar moiety or 2′-OMe modified sugar moiety.
Embodiment 10. The oligomeric compound of any of embodiments 5-9, wherein the modified oligonucleotide comprises at least one modified nucleoside comprising a sugar surrogate.
Embodiment 11. The oligomeric compound of embodiment 10, wherein the modified oligonucleotide comprises at least one modified nucleoside comprising a sugar surrogate selected from morpholino and PNA.
Embodiment 12. The oligomeric compound of any of embodiments 1-11, wherein the modified oligonucleotide has a sugar motif comprising:
   a 5′-region consisting of 1-5 linked 5′-region nucleosides;
   a central region consisting of 6-10 linked central region nucleosides; and
   a 3′-region consisting of 1-5 linked 3′-region nucleosides; wherein
   each of the 5′-region nucleosides and each of the 3′-region nucleosides comprises a modified sugar moiety and
   each of the central region nucleosides comprises a 2′-β-D-deoxyribosyl sugar moiety.
Embodiment 13. The oligomeric compound of embodiment 12, wherein the modified oligonucleotide has
   a 5′-region consisting of 3 linked 5′-region nucleosides;
   a central region consisting of 10 linked central region nucleosides; and
   a 3′-region consisting of 3 linked 3′-region nucleosides; wherein
   each of the 5′-region nucleosides and each of the 3′-region nucleosides comprises a cEt sugar moiety and each of the central region nucleosides comprises a 2′-β-D-deoxyribosyl sugar moiety.
Embodiment 14. The oligomeric compound of any of embodiments 1-13, wherein the modified oligonucleotide comprises at least one modified internucleoside linkage.
Embodiment 15. The oligomeric compound of embodiment 14, wherein each internucleoside linkage of the modified oligonucleotide is a modified internucleoside linkage.
Embodiment 16. The oligomeric compound of embodiment 14 or 15 wherein at least one internucleoside linkage is a phosphorothioate internucleoside linkage.
Embodiment 17. The oligomeric compound of any one of embodiments 14, 15, or 16 wherein at least one internucleoside linkage is a methoxypropyl phosphonate internucleoside linkage.
Embodiment 18. The oligomeric compound of any of embodiment 14, 16, or 17 wherein the modified oligonucleotide comprises at least one phosphodiester internucleoside linkage.
Embodiment 19. The oligomeric compound of any of embodiments 14, 16, 17, or 18, wherein each internucleoside linkage is independently selected from a phosphodiester internucleoside linkage, a phosphorothioate intemucleoside linkage, or a methoxypropyl phosphonate internucleoside linkage.
Embodiment 20. The oligomeric compound of any of embodiments 1-19, wherein the modified oligonucleotide comprises a modified nucleobase.
Embodiment 21. The oligomeric compound of embodiment 20, wherein the modified nucleobase is a 5-methyl cytosine.
Embodiment 22. The oligomeric compound of any one of embodiments 1-21, wherein the modified oligonucleotide consists of 12-30, 12-22, 12-20,14-18, 14-20, 15-17, 15-25, 16-20, 18-22 or 18-20 linked nucleosides.
Embodiment 23. The oligomeric compound of any one of embodiments 1-22, wherein the modified oligonucleotide consists of 16 linked nucleosides.
Embodiment 24. The oligomeric compound of any one of embodiments 1-23, consisting of the modified oligonucleotide and the conjugate group.
Embodiment 25. The oligomeric compound of any one of embodiments 1-24, wherein the conjugate group comprises a conjugate moiety and a conjugate linker.
Embodiment 26. The oligomeric compound of embodiment 25, wherein the conjugate moiety is a lipophilic group.
Embodiment 27. The oligomeric compound of embodiment 25, wherein the conjugate moiety is selected from a C22 alkyl, C20 alkyl, C16 alkyl, C10 alkyl, C21 alkyl, C19 alkyl. C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.
Embodiment 28. The oligomeric compound of embodiment 25, wherein the conjugate moiety is a 6-palmitamidohexyl conjugate moiety.
Embodiment 29. The oligomeric compound of any of embodiments 25-28, wherein the conjugate linker is a phosphodiester linker.
Embodiment 30. The oligomeric compound of any one of embodiments 1-29, wherein the conjugate group has the following structure:

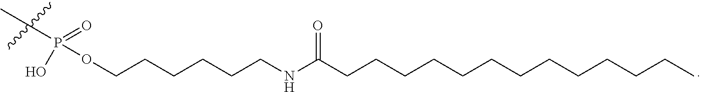 [SMILES: *P(=O)(O)OCCCCCCNC(=O)CCCCCCCCCCCCC]

Embodiment 31. The oligomeric compound of any one of embodiments 25-28, wherein the conjugate linker consists of a single bond.
Embodiment 32. The oligomeric compound of embodiments 25-28, wherein the conjugate linker is cleavable.
Embodiment 33. The oligomeric compound of embodiments 25-28, wherein the conjugate linker comprises 1-3 linker-nucleosides.
Embodiment 34. The oligomeric compound of any of embodiments 25-33, wherein the conjugate group is attached to the modified oligonucleotide at the 5′-end of the modified oligonucleotide.
Embodiment 35. The oligomeric compound of any of embodiments 25-33, wherein the conjugate group is attached to the modified oligonucleotide at the 3′-end of the modified oligonucleotide.
Embodiment 36. The oligomeric compound of any of embodiments 1-35, comprising a terminal group.
Embodiment 37. The oligomeric compound of any of embodiments 1-36 wherein the oligomeric compound is a singled-stranded oligomeric compound.
Embodiment 38. The oligomeric compound of any one of embodiments 1-32 or 34-37, wherein the oligomeric compound does not comprise linker-nucleosides.
Embodiment 39. An oligomeric duplex comprising an oligomeric compound of any one of embodiments 1-23, 25-36, or 38.
Embodiment 40. An oligomeric compound according to the following chemical structure:

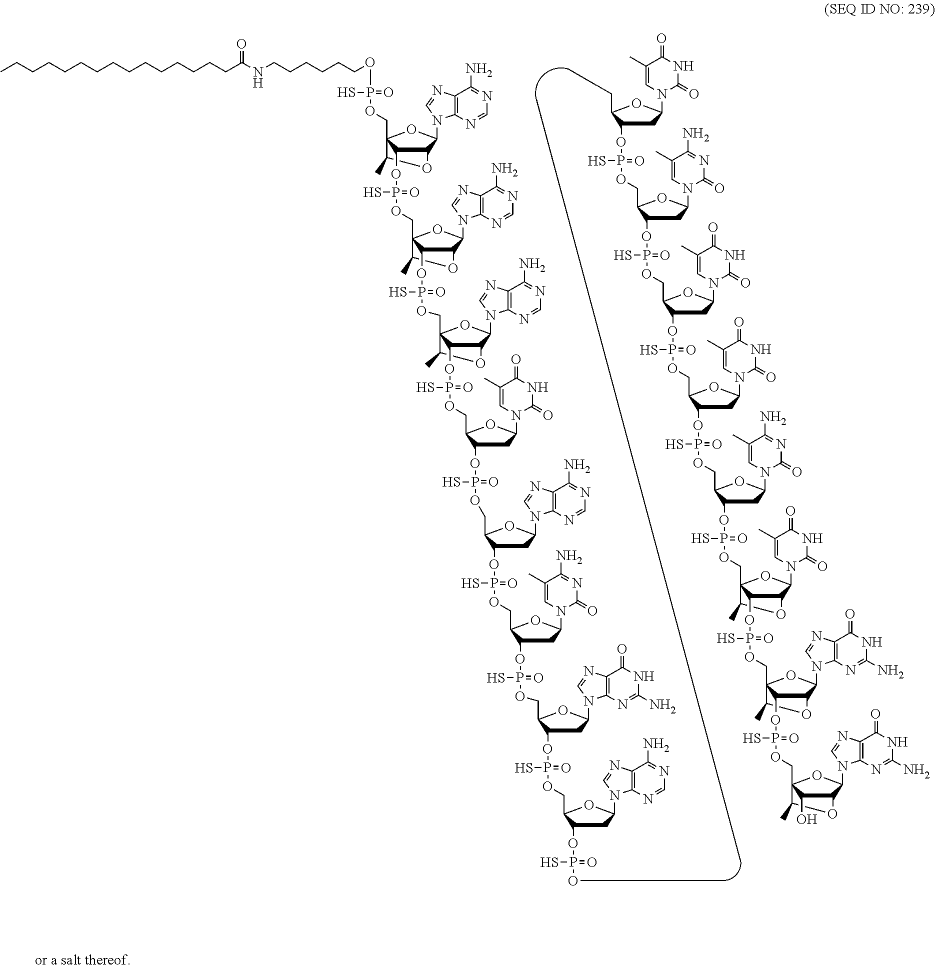

Embodiment 41. The oligomeric compound of embodiment 39, which is the sodium salt or the potassium salt.
Embodiment 42. An oligomeric compound according to the following chemical structure:

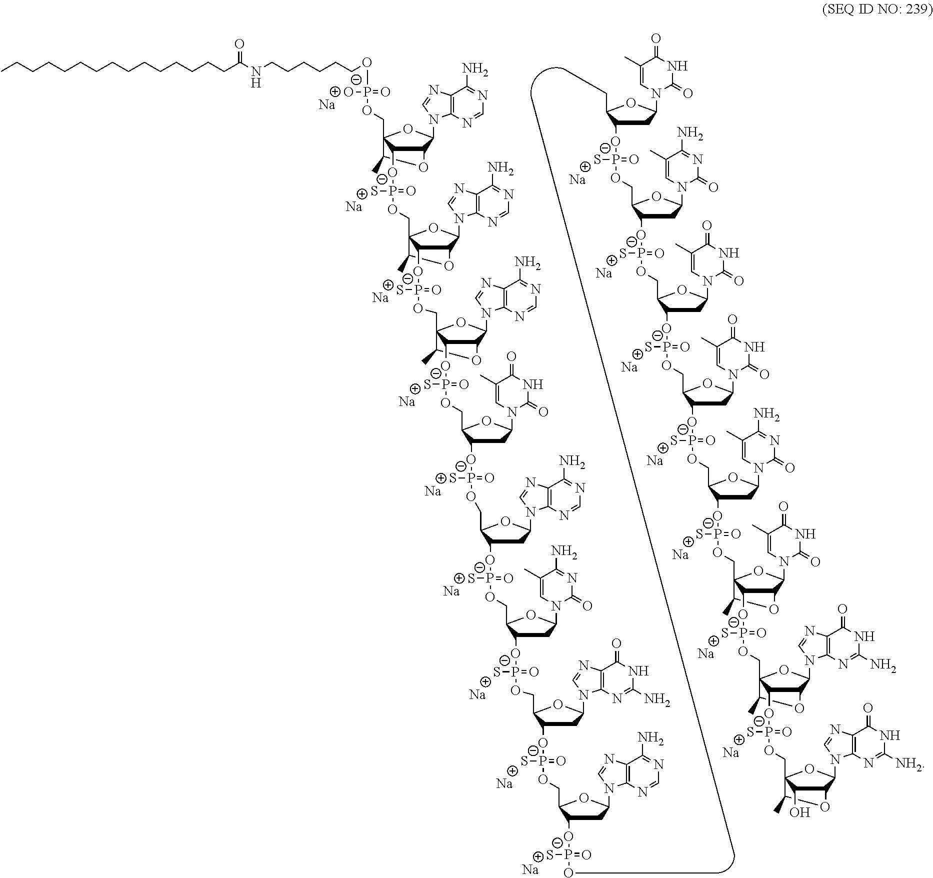

Embodiment 43. An oligomeric comprising a modified oligonucleotide and conjugate group according to the following chemical notation: (6-palmitamidohexyl) Aks Aks Aks Tds Ads ^{m}Cds Gds Ads Tds ^{m}Cds Tds Tds ^{m}Cds Tks Gks Gk (SEQ ID NO:239), wherein:
   A=an adenine nucleobase,
   mC=a 5-methyl cytosine nucleobase,
   G=a guanine nucleobase,
   T=a thymine nucleobase,
   k=a cEt sugar moiety,
   d=a 2′-β-D-deoxyribosyl sugar moiety, and
   s=a phosphorothioate intemucleoside linkage.
Embodiment 44. An antisense compound comprising or consisting of an oligomeric compound of any of embodiments 1-36 or an oligomeric duplex of embodiment 37.
Embodiment 45. A pharmaceutical composition comprising an oligomeric compound of any of embodiments 1-38 or 40-44 or an oligomeric duplex of embodiment 39 or 44 and a pharmaceutically acceptable carrier or diluent.
Embodiment 46. The pharmaceutical composition of embodiment 45, wherein the pharmaceutically acceptable diluent is phosphate buffered saline.
Embodiment 47. The pharmaceutical composition of embodiment 45, wherein the pharmaceutical composition consists essentially of the modified oligonucleotide and phosphate buffered saline.
Embodiment 48. A method comprising administering to an animal a pharmaceutical composition of any of embodiments 45-47.
Embodiment 49. A method of treating a disease associated with PMP22 comprising administering to an individual having or at risk for developing a disease associated with PMP22 a therapeutically effective amount of a pharmaceutical composition according to any of embodiments 45-47; and thereby treating the disease associated with PMP22.
Embodiment 50. The method of embodiment 49, wherein the PMP22-associated disease is Dejerine-Sottas Syndrome.
Embodiment 51. The method of embodiment 49, wherein the PMP22-associated disease is Charcot-Marie-Tooth disease.
Embodiment 52. The method of embodiment 51, wherein the Charcot-Marie-Tooth disease is CMT1A.
Embodiment 53. The method of embodiment 51, wherein the Charcot-Marie-Tooth disease is CMT1E.
Embodiment 54. The method of any of embodiments 49-53, wherein at least one symptom or hallmark of the PMP22-associated disease is ameliorated.
Embodiment 55. The method of embodiment 54, wherein the symptom or hallmark is demyelination, progressive axonal damage and/or loss, weakness and wasting of foot and lower leg muscles, foot deformities, and weakness and atrophy in the hands.
Embodiment 56. An oligomeric compound, comprising a modified oligonucleotide consisting of 12 to 50 linked nucleosides wherein the nucleobase sequence of the modified oligonucleotide is at least 90% complementary to an equal length portion of a PMP22 RNA, and wherein the modified oligonucleotide comprises at least one modification selected from a modified sugar, a sugar surrogate, and a modified internucleoside linkage.
Embodiment 57. An oligomeric compound comprising a modified oligonucleotide consisting of 12 to 50 linked nucleosides and having a nucleobase sequence comprising at least 12, 13, 14, 15, or 16 nucleobases of any of SEQ ID NOs: 19, 193-197, 199-205, 207-218, 220-226, or 238-239.
Embodiment 58. The oligomeric compound of embodiment 56 or 57, wherein the modified oligonucleotide has a nucleobase sequence that is at least 80%, 85%, 90%, 95%, or 100% complementary to any of the nucleobase sequences of SEQ ID NO: 1-8 when measured across the entire nucleobase sequence of the modified oligonucleotide.
Embodiment 59. The oligomeric compound of any one of embodiments 56-58, wherein the modified oligonucleotide comprises at least one modified nucleoside.
Embodiment 60. The oligomeric compound of embodiment 59, wherein the modified oligonucleotide comprises at least one modified nucleoside comprising a modified sugar moiety.
Embodiment 61. The oligomeric compound of embodiment 60, wherein the modified oligonucleotide comprises at least one modified nucleoside comprising a bicyclic sugar moiety.
Embodiment 62. The oligomeric compound of embodiment 61, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 63. The oligomeric compound of any one of embodiments 59-62, wherein the modified oligonucleotide comprises at least one modified nucleoside comprising a non-bicyclic modified sugar moiety.
Embodiment 64. The oligomeric compound of embodiment 63, wherein the modified oligonucleotide comprises at least one modified nucleoside comprising a non-bicyclic modified sugar moiety comprising a 2′-MOE modified sugar or 2′-OMe modified sugar.
Embodiment 65. The oligomeric compound of any one of embodiments 59-64, wherein the modified oligonucleotide comprises at least one modified nucleoside comprising a sugar surrogate.
Embodiment 66. The oligomeric compound of embodiment 65, wherein the modified oligonucleotide comprises at least one modified nucleoside comprising a sugar surrogate selected from morpholino and PNA.
Embodiment 67. The oligomeric compound of any one of embodiments 56-66, wherein the modified oligonucleotide has a sugar motif comprising:
   a 5′-region consisting of 1-5 linked 5′-region nucleosides;
   a central region consisting of 6-10 linked central region nucleosides; and
   a 3′-region consisting of 1-5 linked 3′-region nucleosides; wherein
   each of the 5′-region nucleosides and each of the 3′-region nucleosides comprises a modified sugar moiety and each of the central region nucleosides comprises a 2′-β-D-deoxyribosyl sugar moiety.
Embodiment 68. The oligomeric compound of embodiment 67, wherein the modified oligonucleotide has
   a 5′-region consisting of 3 linked 5′-region nucleosides;
   a central region consisting of 10 linked central region nucleosides; and
   a 3′-region consisting of 3 linked 3′-region nucleosides; wherein
   each of the 5′-region nucleosides and each of the 3′-region nucleosides comprises a cEt sugar moiety and each of the central region nucleosides comprises a 2′-β-D-deoxyribosyl sugar moiety.
Embodiment 69. The oligomeric compound of any one of embodiments 56-68, wherein the modified oligonucleotide comprises at least one modified internucleoside linkage.
Embodiment 70. The oligomeric compound of embodiment 69, wherein each internucleoside linkage of the modified oligonucleotide is a modified internucleoside linkage.
Embodiment 71. The oligomeric compound of embodiment 69 or 70 wherein at least one internucleoside linkage is a phosphorothioate internucleoside linkage.
Embodiment 72. The oligomeric compound of embodiment 69 or 70 wherein the modified oligonucleotide comprises at least one phosphodiester internucleoside linkage.
Embodiment 73. The oligomeric compound of any one of embodiments 69, 71, or 72, wherein each internucleoside linkage is independently selected from a phosphodiester intemucleoside linkage or a phosphorothioate internucleoside linkage.
Embodiment 74. The oligomeric compound of any one of embodiments 56-73, wherein the modified oligonucleotide comprises a modified nucleobase.
Embodiment 75. The oligomeric compound of embodiment 74, wherein the modified nucleobase is a 5-methyl cytosine.
Embodiment 76. The oligomeric compound of any one of embodiments 56-75, wherein the modified oligonucleotide consists of 12-30, 12-22, 12-20,14-18, 14-20, 15-17, 15-25, 16-20, 18-22 or 18-20 linked nucleosides.
Embodiment 77. The oligomeric compound of any one of embodiments 56-76, wherein the modified oligonucleotide consists of 16 linked nucleosides.
Embodiment 78. The oligomeric compound of any one of embodiments 56-77, consisting of the modified oligonucleotide.
Embodiment 79. The oligomeric compound of any one of embodiments 56-77, further comprising a conjugate group.
Embodiment 80. The oligomeric compound of embodiment 79, wherein the conjugate group comprises a conjugate moiety and a conjugate linker.
Embodiment 81. The oligomeric compound of embodiment 80, wherein the conjugate linker consists of a single bond.
Embodiment 82. The oligomeric compound of embodiments 80-81, wherein the conjugate linker is cleavable.
Embodiment 83. The oligomeric compound of embodiments 80 or 82, wherein the conjugate linker comprises 1-3 linker-nucleosides.
Embodiment 84. The oligomeric compound of any one of embodiments 80-83, wherein the conjugate group is attached to the modified oligonucleotide at the 5′-end of the modified oligonucleotide.
Embodiment 85. The oligomeric compound of any one of embodiments 80-83, wherein the conjugate group is attached to the modified oligonucleotide at the 3′-end of the modified oligonucleotide.
Embodiment 86. The oligomeric compound of any one of embodiments 56-85, further comprising a terminal group.
Embodiment 87. The oligomeric compound of any one of embodiments 56-86 wherein the oligomeric compound is a singled-stranded oligomeric compound.
Embodiment 88. The oligomeric compound of any one of embodiments 56-82 or 84-87, wherein the oligomeric compound does not comprise linker-nucleosides.
Embodiment 89. An oligomeric duplex comprising an oligomeric compound of any one of embodiments 56-78, 80-86, or 88.
Embodiment 90. An antisense compound comprising or consisting of an oligomeric compound of any one of embodiments 56-88 or an oligomeric duplex of embodiment 89.
Embodiment 91. A pharmaceutical composition comprising an oligomeric compound of any one of embodiments 56-88 or an oligomeric duplex of embodiment 89 and a pharmaceutically acceptable carrier or diluent.
Embodiment 92. The pharmaceutical composition of embodiment 91, wherein the pharmaceutically acceptable diluent is phosphate buffered saline.
Embodiment 93. The pharmaceutical composition of embodiment 92, wherein the pharmaceutical composition consists essentially of the modified oligonucleotide and phosphate buffered saline.
Embodiment 94. A method comprising administering to an animal a pharmaceutical composition of any one of embodiments 91-93.
Embodiment 95. A method of treating a disease associated with PMP22 comprising administering to an individual having or at risk for developing a disease associated with PMP22 a therapeutically effective amount of a pharmaceutical composition according to any one of embodiments 91-93; and thereby treating the disease associated with PMP22.
Embodiment 96. The method of embodiment 95, wherein the PMP22-associated disease is Dejerine-Sottas Syndrome.
Embodiment 97. An oligomeric compound comprising a modified oligonucleotide consisting of 12 to 50 linked nucleosides, wherein the nucleobase sequence of the modified oligonucleotide is at least 90% complementary to an equal length portion of a PMP22 nucleic acid, and wherein the modified oligonucleotides comprises at least one modification selected from a modified sugar moiety and a modified internucleoside linkage.
Embodiment 98. The oligomeric compound of embodiment 97, wherein the PMP22 nucleic acid has the nucleobase sequence of any of SEQ ID NOs: 1-8.
Embodiment 99. The oligomeric compound of embodiment 97 or embodiment 98, wherein the nucleobase sequence of the modified oligonucleotide is at least 95% or is 100% complementary to an equal length portion of the PMP22 nucleic acid.
Embodiment 100. An oligomeric compound, wherein the oligomeric compound comprises a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, or 16, contiguous nucleobases of any of the nucleobase sequences of any of SEQ ID NOs: 18-321.
Embodiment 101. An oligomeric compound, wherein the oligomeric compound comprises a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of any of the nucleobase sequences of any of SEQ ID NOs: 322-632.
Embodiment 102. The oligomeric compound of embodiment 100, wherein the nucleobase sequence of the modified oligonucleotide comprises the nucleobase sequence of any of SIDs 18-321.
Embodiment 103. The oligomeric compound of embodiment 101, wherein the nucleobase sequence of the modified oligonucleotide comprises the nucleobase sequence of any of SIDs 322-632.
Embodiment 104. The oligomeric compound of embodiment 100, wherein the nucleobase sequence of the modified oligonucleotide consists of the nucleobase sequence of any of SIDs 18-321.
Embodiment 105. The oligomeric compound of embodiment 101, wherein the nucleobase sequence of the modified oligonucleotide consists of nucleobase sequence of any of SIDs 322-632.
Embodiment 106. The oligomeric compound of any of embodiments 101-105, wherein the nucleobase sequence of the modified oligonucleotide is at least 90%, at least 95%, or 100% complementary to an equal length portion of a PMP22 nucleic acid, wherein the PMP22 nucleic acid has the nucleobase sequence of any of SEQ ID NOs: 1-8.
Embodiment 107. The oligomeric compound of any of embodiments 97-106, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.
Embodiment 108. The oligomeric compound of any of embodiments 97-107, wherein at least one nucleoside of the modified oligonucleotide comprises a modified sugar moiety.
Embodiment 109. The oligomeric compound of embodiment 108, wherein the modified sugar moiety comprises a bicyclic sugar moiety.
Embodiment 110. The oligomeric compound of embodiment 109, wherein the bicyclic sugar moiety comprises a 2′-4′ bridge, wherein the 2′-4′ bridge is selected from —O—CH₂—; and —O—CH(CH₃)—.
Embodiment 111. The oligomeric compound of embodiment 108, wherein the modified sugar moiety comprises a non-bicyclic modified sugar moiety.
Embodiment 112. The oligomeric compound of embodiment 111, wherein the non-bicyclic modified sugar moiety is a 2′-MOE sugar moiety, a 2′-OMe sugar moiety, or a 2′-F sugar moiety.
Embodiment 113. The oligomeric compound of any of embodiments 97-112, wherein at least one nucleoside of the modified oligonucleotide comprises a sugar surrogate.
Embodiment 114. The oligomeric compound of embodiment 113, wherein the sugar surrogate is selected from morpholino and PNA.
Embodiment 115. The oligomeric compound of any of embodiments 97-114, wherein the modified oligonucleotide comprises at least one modified internucleoside linkage.
Embodiment 116. The oligomeric compound of embodiment 115, wherein at least one modified internucleoside linkage is a phosphorothioate internucleoside linkage.
Embodiment 117. The oligomeric compound of embodiment 115, wherein at least one modified internucleoside linkage is a methoxy propyl internucleoside linkage.
Embodiment 118. The oligomeric compound of embodiment 115, wherein each internucleoside linkage is a modified internucleoside linkage.
Embodiment 119. The oligomeric compound of embodiment 119, wherein each internucleoside linkage is a phosphorothioate internucleoside linkage.
Embodiment 120. The oligomeric compound of any of embodiments 97-116, wherein each internucleoside linkage of the modified oligonucleotide is independently selected from a phosphodiester internucleoside linkage and a phosphorothioate internucleoside linkage.
Embodiment 121. The oligomeric compound of any of embodiments 97-116, wherein each internucleoside linkage of the modified oligonucleotide is independently selected from a phosphodiester internucleoside linkage, a phosphorothioate internucleoside linkage and a mesyl phosphoramidate internucleoside linkage.
Embodiment 122. The oligomeric compound of any of embodiments 97-116, wherein each internucleoside linkage of the modified oligonucleotide is independently selected from a phosphodiester internucleoside linkage, a phosphorothioate internucleoside linkage, and a methoxy propyl internucleoside linkage.
Embodiment 123. The oligomeric compound of any of embodiments 97-116, wherein each internucleoside linkage of the modified oligonucleotide is independently selected from a phosphorothioate internucleoside linkage and a methoxy propyl internucleoside linkage.
Embodiment 124. The oligomeric compound of any of embodiments 97-116, wherein the modified oligonucleotide has a backbone motif selected from sssssssssssssss, ssssqssssssssss, sssssqsssssssss, or ssssssqssssssss wherein “s” is a phosphorothioate internucleoside linkage and “q” is a methoxypropyl internucleoside linkage.
Embodiment 125. The oligomeric compound of any of embodiments 97-124, wherein the modified oligonucleotide comprises at least one modified nucleobase.
Embodiment 126. The oligomeric compound of embodiment 125, wherein the modified nucleobase is 5-methylcytosine.
Embodiment 127. The oligomeric compound of embodiment 126, wherein each cytosine is a 5-methylcytosine.
Embodiment 128. The oligomeric compound of any of embodiment 97-127, wherein the modified oligonucleotide comprises a deoxy region consisting of 5-12 contiguous 2′-deoxynucleosides.
Embodiment 129. The oligomeric compound of embodiment 128, wherein each nucleoside of the deoxy region is a 2′-β-D-deoxynucleoside.
Embodiment 130. The oligomeric compound of embodiment 128 or 129, wherein the deoxy region consists of 6, 7, 8, 9, 10, or 6-10 linked nucleosides.
Embodiment 131. The oligomeric compound of any of embodiments 128-130, wherein each nucleoside immediately adjacent to the deoxy region comprises a modified sugar moiety.
Embodiment 132. The oligomeric compound of any of embodiments 128-131, wherein the deoxy region is flanked on the 5′-side by a 5′-region consisting of 1-6 linked 5′-region nucleosides and on the 3′-side by a 3′-region consisting of 1-6 linked 3′-region nucleosides; wherein the 3′-most nucleoside of the 5′ external region comprises a modified sugar moiety; and the 5′-most nucleoside of the 3′ external region comprises a modified sugar moiety.
Embodiment 133. The oligomeric compound of embodiment 132, wherein each nucleoside of the 3′ external region comprises a modified sugar moiety.
Embodiment 134. The oligomeric compound of embodiment 132 or 133, wherein each nucleoside of the 5′ external region comprises a modified sugar moiety.
Embodiment 135. The oligomeric compound of any of embodiments 128-134, wherein the modified oligonucleotide has:
   a 5′ external region consisting of 1-6 linked nucleosides;
   a deoxy region consisting of 6-10 linked nucleosides; and
   a 3′ external region consisting of 1-6 linked nucleosides;
   wherein each of the 5′ external region nucleosides and each of the 3′ external region nucleosides is a cEt nucleoside or a 2′-MOE nucleoside; and each of the deoxy region nucleosides is a 2′-β-D-deoxynucleoside.
Embodiment 136. The oligomeric compound of any of embodiments 128-134, wherein the modified oligonucleotide has:
   a 5′ external region consisting of 3 linked nucleosides;
   a deoxy region consisting of 10 linked nucleosides; and
   a 3′ external region consisting of 3 linked nucleosides;
   wherein each of the 5′ external region nucleosides and each of the 3′ external region nucleosides is a cEt nucleoside and each of the deoxy region nucleosides is a 2′-β-D-deoxynucleoside.
Embodiment 137. The oligomeric compound of any of embodiments 128-134, wherein the modified oligonucleotide has:
   a 5′ external region consisting of 3-4 linked nucleosides;
   a deoxy region consisting of 8-10 linked nucleosides; and
   a 3′ external region consisting of 3-4 linked nucleosides;
   wherein each of the 5′ external region nucleosides and each of the 3′ external region nucleosides is a cEt nucleoside or a 2′-MOE nucleoside; and each of the deoxy region nucleosides is a 2′-β-D-deoxynucleoside.
Embodiment 138. The oligomeric compound of any of embodiments 128-134, wherein the modified oligonucleotide has a sugar motif comprising:
   a 5′ external region consisting of 3-6 linked nucleosides;
   a deoxy region consisting of 7-8 linked nucleosides; and
   a 3′ external region consisting of 3-6 linked nucleosides; wherein
   each of the 3′ external region nucleosides is selected from a 2′-MOE nucleoside and a cEt nucleoside, and the 5′ external region has the following formula:

(Nk)n(Nd)(Nx)
wherein each Nk is a bicyclic nucleoside, Nx 2′-OMe nucleoside and Nd is a 2′-β-D-deoxynucleoside; and n is from 1-4.
Embodiment 139. The oligomeric compound of any of embodiments 128-134, wherein the modified oligonucleotide has a sugar motif comprising:
   a 5′ external region consisting of 5 linked nucleosides;
   a deoxy region consisting of 8 linked nucleosides; and
   a 3′ external region consisting of 3 linked nucleosides; wherein
   each of the 3′ external region nucleosides is a cEt nucleoside, and the 5′ external region has the following formula:

(Nk)n(Nd)(Nx)
wherein each Nk is a cEt nucleoside, Nx 2′-OMe nucleoside and Nd is a 2′-β-D-deoxynucleoside;
and n is from 3;
and wherein each of the deoxy region nucleosides is a 2′-β-D-deoxynucleoside.
Embodiment 140. An oligomeric compound of any of embodiments 97-142, wherein the modified oligonucleotide has a sugar motif (5′ to 3′) selected from: kkkddddddddddkkk, ekkddddddddddkke, ekkddddddddddkkk, ekkddddddddddkkk, ekkkdddddddddkkk, kekddddddddddkkk, kkeddddddddddkkk, kkkddddddddddekk, kkkddddddddddkek, kkkdddddddddkkke, kkkddydddddddkkk, or kkkdddddddddekkk, wherein each “d” represents a 2′-β-D-deoxyribosyl sugar moiety, each “y” represents a 2′-OMe sugar moiety, each “e” represents a 2′-MOE sugar moiety, and each “k” represents a cEt sugar moiety.
Embodiment 141. The oligomeric compound of any one of embodiments 97-140, wherein the oligomeric compound comprises a conjugate group.
Embodiment 142. The oligomeric compound of embodiment 141, wherein the conjugate group comprises a conjugate moiety and a conjugate linker.
Embodiment 143. The oligomeric compound of embodiment 142, wherein the conjugate moiety is a lipophilic group.
Embodiment 144. The oligomeric compound of embodiment 143, wherein the conjugate moiety is selected from a C22 alkyl, C20 alkyl, C16 alkyl, C10 alkyl, C21 alkyl, C19 alkyl. C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.
Embodiment 145. The oligomeric compound of embodiment 144, wherein the conjugate moiety is a 6-palmitamidohexyl conjugate moiety.
Embodiment 146. The oligomeric compound of any of embodiments 142-145, wherein the conjugate linker consists of a single bond.
Embodiment 147. The oligomeric compound of any of embodiments 142-145, wherein the conjugate linker is cleavable.
Embodiment 148. The oligomeric compound of embodiment 147, wherein the conjugate linker comprises a phosphodiester linkage.
Embodiment 149. The oligomeric compound of any of embodiments 142-148, wherein the conjugate linker comprises 1-3 linker nucleosides.
Embodiment 150. The oligomeric compound of any of embodiments 142-148, wherein the conjugate linker does not comprise any linker nucleosides.
Embodiment 151. The oligomeric compound of any one of embodiments 142-148, wherein the conjugate group has the following structure:

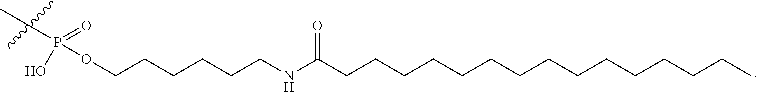 [SMILES: *P(=O)(O)OCCCCCCNC(=O)CCCCCCCCCCCCCCC]

Embodiment 152. The oligomeric compound of any of embodiments 142-151, wherein the conjugate group is attached to the modified oligonucleotide at the 5′-end of the modified oligonucleotide.
Embodiment 153. The oligomeric compound of any of embodiments 142-151, wherein the conjugate group is attached to the modified oligonucleotide at the 3′-end of the modified oligonucleotide.
Embodiment 154. The oligomeric compound of any of embodiments 142-153, wherein the conjugate group comprises a cell-targeting moiety.
Embodiment 155. The oligomeric compound of any of embodiments 142-154, comprising a terminal group.
Embodiment 156. An oligomeric compound comprising a modified oligonucleotide and a conjugate group according to the following chemical notation: (6-palmitamidohexyl) Aks Aks Aks Tds Ads ^{m}Cds Gds Ads Tds ^{m}Cds Tds Tds ^{m}Cds Tks Gks Gk (SEQ ID NO:239), wherein:
   A=an adenine nucleobase,
   ^{m}C=a 5-methyl cytosine nucleobase,
   G=a guanine nucleobase,
   T=a thymine nucleobase,
   k=a cEt sugar moiety,
   d=a 2′-β-D-deoxyribosyl sugar moiety, and
   s=a phosphorothioate internucleoside linkage.
Embodiment 157. An oligomeric compound according to the following chemical structure:

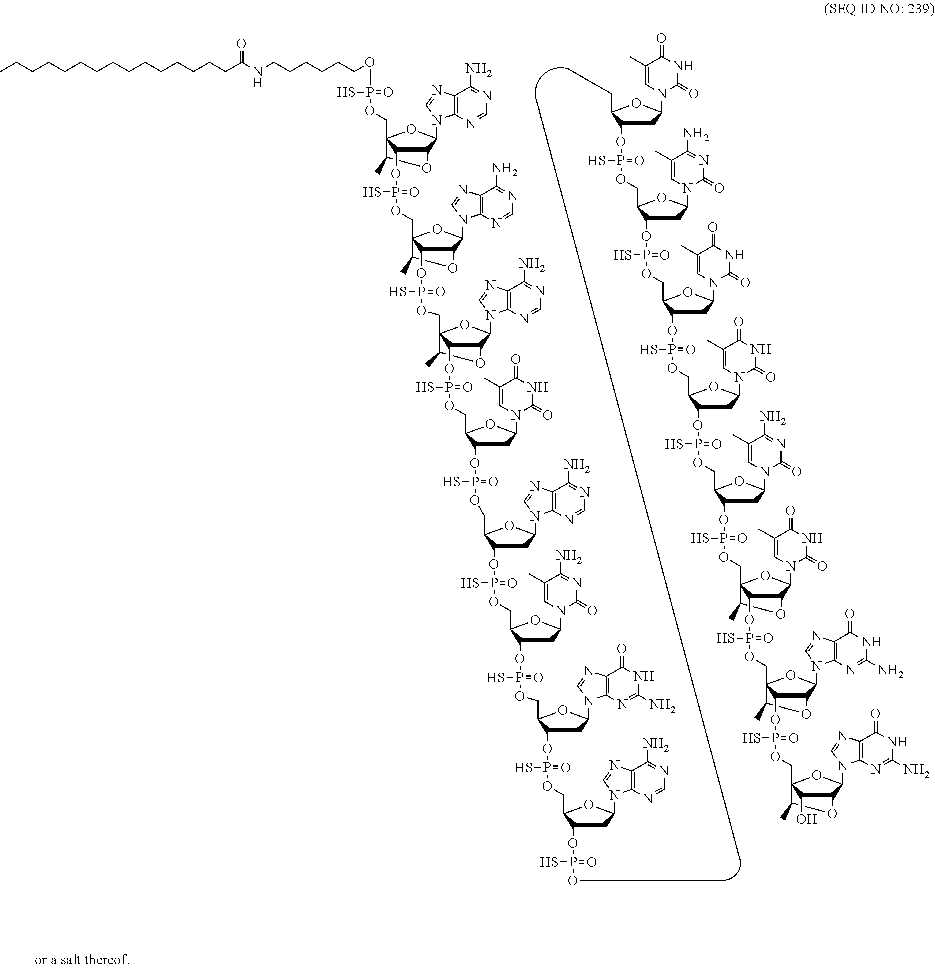

Embodiment 158. The oligomeric compound of embodiment 157, which is the sodium salt or the potassium salt.
Embodiment 159. An oligomeric compound according to the following chemical structure:

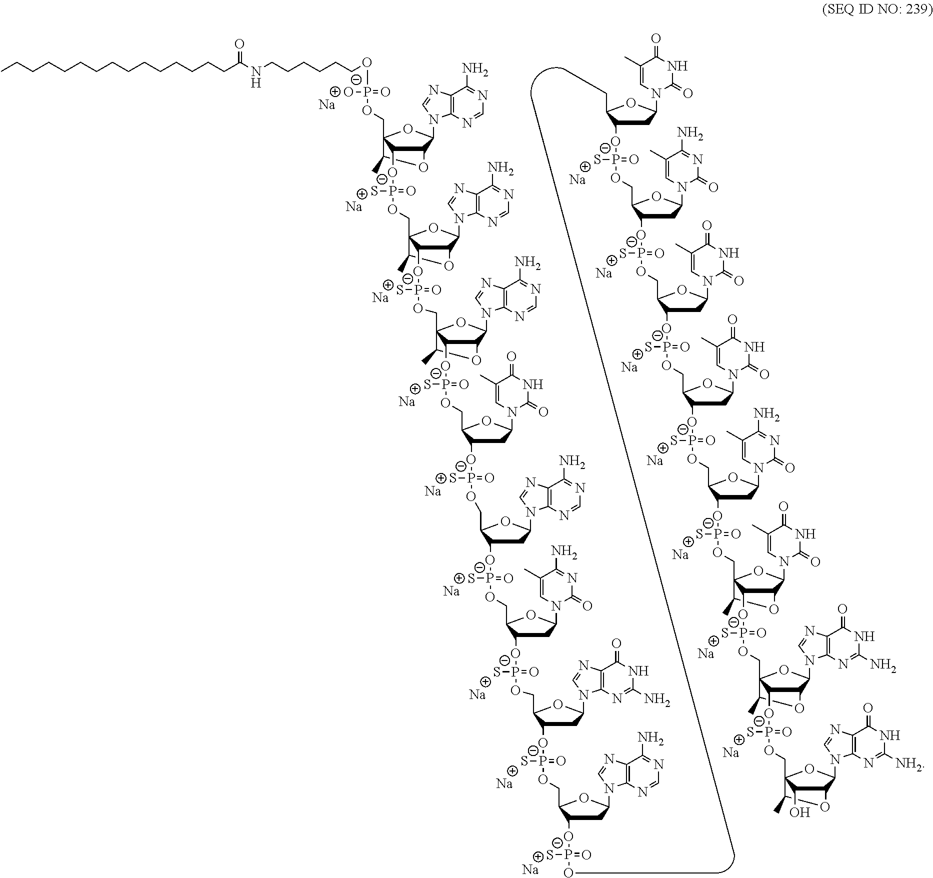

Embodiment 160. An oligomeric duplex, comprising a first oligomeric compound and a second oligomeric compound comprising a second modified oligonucleotide, wherein the first oligomeric compound is an oligomeric compound of any of embodiments 97-159.
Embodiment 161. The oligomeric duplex of embodiment 160, wherein the second oligomeric compound comprises a second modified oligonucleotide consisting of 8 to 80 linked nucleosides, and wherein the nucleobase sequence of the second modified oligonucleotide comprises a complementary region of at least 8 nucleobases that is at least 90% complementary to an equal length portion of the first modified oligonucleotide.
Embodiment 162. An oligomeric duplex comprising:
   a first oligomeric compound comprising a first modified oligonucleotide consisting of 19 to 29 linked nucleosides wherein the nucleobase sequence of the first modified oligonucleotide comprises at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 322-632; and
   a second oligomeric compound comprising a second modified oligonucleotide consisting of 15 to 29 linked nucleosides wherein the nucleobase sequence of the second modified oligonucleotide comprises a complementary region of at least 12 nucleobases that is at least 90% complementary to an equal length portion of the first modified oligonucleotide.
Embodiment 163. An oligomeric duplex comprising:
   a first oligomeric compound comprising a first modified oligonucleotide consisting of 19 to 29 linked nucleosides wherein the nucleobase sequence of the first modified oligonucleotide comprises at least 12, at least 13, at least 14, at least 15, at least 16, at least 17, at least 18, at least 19, at least 20, at least 21, at least 22, or at least 23 contiguous nucleobases of the nucleobase sequence of SEQ ID NO: 944; and
   a second oligomeric compound comprising a second modified oligonucleotide consisting of 15 to 29 linked nucleosides wherein the nucleobase sequence of the second modified oligonucleotide comprises a complementary region of at least 12 nucleobases that is at least 90% complementary to an equal length portion of the first modified oligonucleotide.
Embodiment 164. An oligomeric duplex comprising:
   a first oligomeric compound comprising a first modified oligonucleotide consisting of 23 linked nucleosides and has a nucleobase sequence of consisting of the nucleobase sequence of any of SEQ ID NOs: 322-632; and
   a second oligomeric compound comprising a second modified oligonucleotide consisting of 21 linked nucleosides, wherein the second modified oligonucleotide has a nucleobase sequence consisting of the nucleobase sequence of any of SEQ ID NOs: 633-943, and wherein the nucleobase sequence of the second modified oligonucleotide is at least 90% complementary to an equal length portion of the first modified oligonucleotide.
Embodiment 165. An oligomeric duplex comprising:
   a first oligomeric compound comprising a first modified oligonucleotide consisting of 22 linked nucleosides and has a nucleobase sequence of consisting of the nucleobase sequence of SEQ ID NO: 944; and
   a second oligomeric compound comprising a second modified oligonucleotide consisting of 20 linked nucleosides, wherein the second modified oligonucleotide has a nucleobase sequence consisting of the nucleobase sequence of SEQ ID NO: 945.
Embodiment 166. The oligomeric duplex of any of embodiments 160-165, wherein the modified oligonucleotide of the first oligomeric compound comprises a 5′-stabilized phosphate group.
Embodiment 167. The oligomeric duplex of embodiment 166, wherein the 5′-stabilized phosphate group comprises a cyclopropyl phosphonate or a vinyl phosphonate.
Embodiment 168. The oligomeric duplex of any of embodiments 160-167, wherein the modified oligonucleotide of the first oligomeric compound comprises a glycol nucleic acid (GNA) sugar surrogate.
Embodiment 169. The oligomeric duplex of any of embodiments 160-168, wherein the modified oligonucleotide of the first oligomeric compound comprises a 2′-NMA sugar moiety.
Embodiment 170. The oligomeric duplex of any of embodiments 160-169, wherein at least one nucleoside of the second modified oligonucleotide comprises a modified sugar moiety.
Embodiment 171. The oligomeric duplex of embodiment 170, wherein the modified sugar moiety of the second modified oligonucleotide comprises a bicyclic sugar moiety.
Embodiment 172. The oligomeric duplex of embodiment 171, wherein the bicyclic sugar moiety of the second modified oligonucleotide comprises a 2′-4′ bridge selected from —O—CH₂— and —O—CH(CH₃)—.
Embodiment 173. The oligomeric duplex of embodiment 170, wherein the modified sugar moiety of the second modified oligonucleotide comprises a non-bicyclic modified sugar moiety.
Embodiment 174. The oligomeric duplex of embodiment 173, wherein the non-bicyclic modified sugar moiety of the second modified oligonucleotide is a 2′-MOE sugar moiety, a 2′-F sugar moiety, or 2′-OMe sugar moiety.
Embodiment 175. The oligomeric duplex of any of embodiments 160-174, wherein at least one nucleoside of the second modified oligonucleotide comprises a sugar surrogate.
Embodiment 176. The oligomeric duplex of any of embodiments 160-175, wherein the second modified oligonucleotide comprises at least one modified internucleoside linkage.
Embodiment 177. The oligomeric duplex of embodiment 176, wherein at least one modified internucleoside linkage of the second modified oligonucleotide is a phosphorothioate internucleoside linkage.
Embodiment 178. The oligomeric duplex of any of embodiments 160-177, wherein the second modified oligonucleotide comprises at least one phosphodiester internucleoside linkage.
Embodiment 179. The oligomeric duplex of any of embodiments 160-178, wherein each internucleoside linkage of the second modified oligonucleotide is independently selected from a phosphodiester or a phosphorothioate internucleoside linkage.
Embodiment 180. The oligomeric duplex of any of embodiments 160-179, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.
Embodiment 181. The oligomeric duplex of any of embodiments 160-180, wherein the second modified oligonucleotide comprises at least one modified nucleobase.
Embodiment 182. The oligomeric duplex of embodiment 181, wherein the modified nucleobase of the second modified oligonucleotide is 5-methylcytosine.
Embodiment 183. The oligomeric duplex of any of embodiments 160-182, wherein the second modified oligonucleotide comprises a conjugate group.
Embodiment 184. The oligomeric duplex of embodiment 183, wherein the conjugate group comprises a conjugate linker and a conjugate moiety.
Embodiment 185. The oligomeric duplex of embodiment 183 or 184, wherein the conjugate group is attached to the second modified oligonucleotide at the 5′-end of the second modified oligonucleotide.
Embodiment 186. The oligomeric duplex of embodiment 183 or 184, wherein the conjugate group is attached to the second modified oligonucleotide at the 3′-end of the modified oligonucleotide.
Embodiment 187. The oligomeric duplex of any of embodiments 183-186, wherein the conjugate group comprises a C22 alkyl, C20 alkyl. C16 alkyl, C10 alkyl, C21 alkyl, C19 alkyl, C18 alkyl, C15 alkyl, C14 alkyl, C13 alkyl, C12 alkyl, C11 alkyl. C9 alkyl, C8 alkyl, C7 alkyl, C6 alkyl, C5 alkyl, C22 alkenyl, C20 alkenyl, C16 alkenyl, C10 alkenyl. C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.
Embodiment 188. The oligomeric duplex of any of embodiments 183-187, wherein the conjugate moiety is a 6-palmitamidohexyl conjugate moiety.
Embodiment 189. The oligomeric duplex of any of embodiments 183-190, wherein the conjugate group has the following structure:

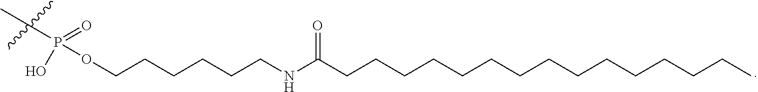 [SMILES: *P(=O)(O)OCCCCCCNC(=O)CCCCCCCCCCCCCCC]

Embodiment 190. The oligomeric duplex of any of embodiments 183-192, wherein the conjugate group comprises a cell-targeting moiety.
Embodiment 191. The oligomeric duplex of any of embodiments 160-190, wherein the second modified oligonucleotide comprises a terminal group.
Embodiment 192. The oligomeric duplex of embodiment 191, wherein the terminal group is an abasic sugar moiety.
Embodiment 193. The oligomeric duplex of any of embodiments 160-192, wherein the second modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.
Embodiment 194. The oligomeric duplex of any of embodiments 160-193, wherein the first modified oligonucleotide consists of 23 linked nucleosides and the second modified oligonucleotide consists of 21 linked nucleosides.
Embodiment 195. The oligomeric duplex of embodiments 194, wherein the modified oligonucleotide of the first oligomeric compound has a sugar motif (from 5′ to 3′) of: yfyfyfyfyfyfyfyfyyy and the second modified oligonucleotide has a sugar motif (from 5′ to 3′) of: fyfyfyfyfyfyfyfyfyfyf, wherein each “y” represents a 2′-OMe sugar moiety and each “f” represents a 2′-F sugar moiety.
Embodiment 196. The oligomeric duplex of any of embodiments 160-195, wherein the first modified oligonucleotide is at least 80% complementary to:
   an equal length portion of nucleobases 765-1043 of SEQ ID NO: 1 or
   an equal length portion of nucleobases 1753-1859 of SEQ ID NO: 1.
Embodiment 197. The oligomeric compound of any of embodiments 160-195, wherein the first modified oligonucleotide has a nucleobase sequence comprising at least 8, at least 9, at least 10, at least 11, at least 12, at least 13, at least 14, at least 15, at least 16, or at least 17 contiguous nucleobases of a sequence selected from:
   SEQ ID NOs: 555, 558-619, 623, 624; or
   SEQ ID NOs: 353-375.
Embodiment 198. An antisense agent comprising an antisense compound, wherein the antisense compound is the oligomeric compound of any of embodiments 94-159.
Embodiment 199. An antisense agent, wherein the antisense agent is the oligomeric duplex of any of embodiments 160-197.
Embodiment 200. The antisense agent of embodiment 198 or 199, wherein the antisense agent is: an RNase H agent capable of reducing the amount of PMP22 nucleic acid through activation of RNase H; or an RNAi agent capable of reducing the amount of PMP22 nucleic acid through activation of RISC/Ago2;
Embodiment 201. A chirally enriched population of oligomeric compounds of any of embodiments 97-159 or oligomeric duplexes of embodiments 160-197, wherein the population is enriched for modified oligonucleotides comprising at least one particular phosphorothioate intemucleoside linkage having a particular stereochemical configuration.
Embodiment 202. The chirally enriched population of embodiment 201 wherein the population is enriched for modified oligonucleotides having a particular, independently selected stereochemical configuration at each phosphorothioate internucleoside linkage.
Embodiment 203. The chirally enriched population of embodiment 201, wherein the population is enriched for modified oligonucleotides having the (Rp) configuration at one particular phosphorothioate internucleoside linkage and the (Sp) configuration at each of the remaining phosphorothioate internucleoside linkages.
Embodiment 204. The chirally enriched population of embodiment 201, wherein the population is enriched for modified oligonucleotides having at least 3 contiguous phosphorothioate internucleoside linkages in the Sp, Sp, and Rp configurations, in the 5′ to 3′ direction.
Embodiment 205. A population of oligomeric compounds comprising modified oligonucleotides of any of embodiments 94-159, or a population of oligomeric duplexes comprising modified oligonucleotides of any of embodiments 160-164, wherein all of the phosphorothioate internucleoside linkages of the modified oligonucleotides are stereorandom.
Embodiment 206. A pharmaceutical composition comprising the oligomeric compound of any of embodiments 94-159, the oligomeric duplex of any of embodiments 160-197, the population of any of embodiments 201-205, or the antisense agent of any of embodiments 198-200, and a pharmaceutically acceptable diluent or carrier.
Embodiment 207. The pharmaceutical composition of embodiment 206, wherein the pharmaceutically acceptable diluent is phosphate buffered saline.
Embodiment 208. The pharmaceutical composition of embodiment 207, wherein the pharmaceutical composition consists essentially of the modified oligonucleotide and phosphate buffered saline.
Embodiment 209. A method comprising administering to an animal a pharmaceutical composition of any of embodiments 206-208.
Embodiment 210. A method of treating a disease associated with PMP22 comprising administering to an individual having or at risk for developing a disease associated with PMP22 a therapeutically effective amount of a pharmaceutical composition according to any of embodiments 206-208; and thereby treating the disease associated with PMP22.
Embodiment 211. The method of embodiment 210, wherein the PMP22-associated disease is Dejerine-Sottas Syndrome.
Embodiment 212. The method of embodiment 210, wherein the PMP22-associated disease is Charcot-Marie-Tooth disease.
Embodiment 213. The method of embodiment 212, wherein the Charcot-Marie-Tooth disease is CMT1A.
Embodiment 214. The method of embodiment 212, wherein the Charcot-Marie-Tooth disease is CMT1E.
Embodiment 215. The method of any of embodiments 210-214, wherein at least one symptom or hallmark of the PMP22-associated disease is ameliorated.
Embodiment 216. The method of embodiment 215, wherein the symptom or hallmark is demyelination, progressive axonal damage and/or loss, weakness and wasting of foot and lower leg muscles, foot deformities, and weakness and atrophy in the hands.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modifed sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₁ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂₀N(Rₘ)(Rₙ) or OCH₂C(═O)—N(Rₘ)(Rₙ), where each Rₘ and R₁ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂₀N(Rₘ)(Rₙ), O(CH₂)₂O(CH₂)₂N(CH₃)₂, and N-substituted acetamide (OCH₂C(═O)—N(Rₘ)(R˜)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, and OCH₂C(═O)—N(H)CH₃ (“NMA”).

In certain embodiments, a 2′-substituted nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

Certain modifed sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′—(CH₂)₂-2′, 4′—(CH₂)₃-2′, 4′-CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2 ′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—S(═O)ₓ—, and —N(Rₐ)—;
wherein:
x is 0, 1, or 2;
n is 1,2,3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅—C, alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191: Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the R-D configuration.

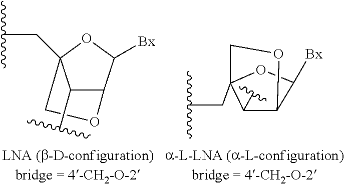

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

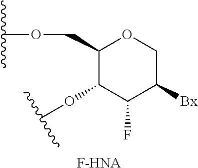

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

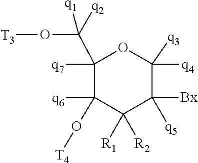

wherein, independently, for each of the modified THP nucleosides:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
   q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

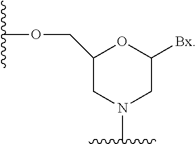

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil). 4-thioumcil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzovlcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any intemucleoside linkage. The two main classes of internucleoside linking groups are defined by the presence or absence of a phosphorus atom. Representative phosphorus-containing internucleoside linkages include but are not limited to phosphodiesters, which contain a phosphodiester bond (“P(O₂)═O”) (also referred to as unmodified or naturally occurring linkages), phosphotriesters, methylphosphonates, phosphoramidates, and phosphorothioates (“P(O₂)═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkage in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, apopulation of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

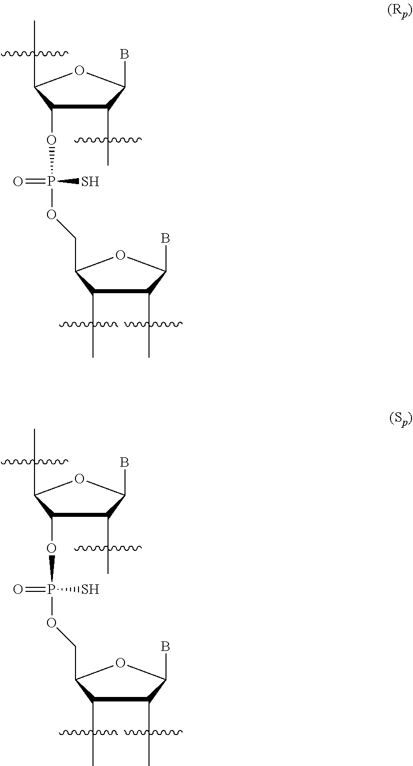

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral intemucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or portion thereof in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-deoxyribosyl sugar moiety. In certain embodiments, each nucleoside of each wing of a gapmer comprises a modified sugar moiety. In certain embodiments, one nucleoside of the gap comprises a modified sugar moiety and each remaining nucleoside of the gap comprises a 2′-deoxyribosyl sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprises a 2′-β-D-deoxyribosyl sugar moiety. Thus, a 5-10-5 MOE gapmer consists of 5 linked 2′-MOE nucleosides in the 5′-wing, 10 linked a 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked a 2′-β-D-deoxynucleosides in the gap, and 5 linked nucleosides comprising a modified sugar moiety in the 3′-wing. A 5-8-5 or 5-8-4 mixed wing gapmer has at least two different modified sugar moieties in the 5′- and/or the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides have a sugar motif selected from the following (5′ to 3′): ekkddddddddddkke, ekkddddddddddkkk, ekkddddddddddkkk, ekkkdddddddddkkk, kekddddddddddkkk, kkeddddddddddkkk, kkeddddddddddkkk, kkkddddddddddekk, kkkddddddddddkek, kkkdddddddddkkke, or kkkddydddddddkkk, wherein ‘d’ represents a 2′-deoxyribosyl sugar moiety, ‘e’ represents a 2′-MOE sugar moiety, ‘k’ represents a cEt sugar moiety, and ‘y’ represents a 2′-OMe sugar moiety.

2. Certain Nucleobase Motifs

In certain embodiments, oligonucleotides comprise modified and/or unmodified nucleobases arranged along the oligonucleotide or portion thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or portion thereof in a defined pattern or motif. In certain embodiments, each internucleoside linking group is a phosphodiester internucleoside linkage (P(O₂)═O). In certain embodiments, each internucleoside linking group of a modified oligonucleotide is a phosphorothioate internucleoside linkage (P(O₂)═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, all of the internucleoside linkages are either phosphodiester internucleoside linkages or phosphorothioate internucleoside linkages, and the chiral motif is (5′ to 3′): Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp, wherein each ‘Sp’ represents a (Sp) phosphorothioate internucleoside linkage, each ‘Rp’ is a Rp internucleoside linkage, and each ‘o’ represents a phosphodiester intemucleoside linkage. In certain embodiments, populations of modified oligonucleotides are enriched for modified oligonucleotides comprising such internucleoside linkage motifs.

In certain embodiments, modified oligonucleotides have an internucleoside linkage motif of sooosssssssssssooss, wherein each “s” represents a phosphorothioate internucleoside linkage and each “o” represents a phosphodiester intemucleoside linkage. In certain embodiments, modified oligonucleotides have an internucleoside linkage motif of (5′ to 3′): sooooossssssssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): sssosssssssssssosss, wherein each “s” represents a phosphorothioate internucleoside linkage and each “o” represents a phosphodiester internucleoside linkage. In certain embodiments, modified oligonucleotides have an intemucleoside linkage motif of (5′ to 3′): sssosssssssssoss, wherein each “s” represents a phosphorothioate internucleoside linkage and each “o” represents a phosphodiester internucleoside linkage.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Olionucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the present disclosure provides oligomeric compounds comprising a modified oligonucleotide and a conjugate group, wherein the conjugate group enhances delivery of the modified oligonucleotide.

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl. C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In some embodiments, the conjugate group comprises a 6-palmitamidohexyl conjugate moiety and a conjugate linker. In some embodiments, the conjugate group comprises a 6-palmitamidohexyl conjugate moiety having the following structure:

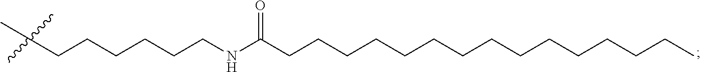 [SMILES: *CCCCCCNC(=O)CCCCCCCCCCCCCCC]

and a phosphodiester conjugate linker. In some embodiments, the conjugate comprises a 6-palmitamidohexyl conjugate moiety and a phosphodiester conjugate linker, wherein the 6-palmitamidohexyl conjugate moiety is attached to the 5′-OH of the modified oligonucleotide via the phosphodiester conjugate linker.

In some embodiments, the conjugate group comprises the following structure:

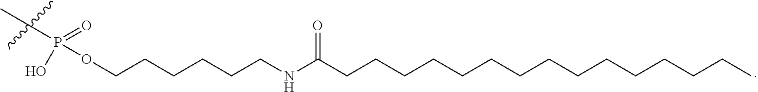 [SMILES: *P(=O)(O)OCCCCCCNC(=O)CCCCCCCCCCCCCCC]

In some embodiments, the conjugate group is attached to the modified oligonucleotide at the 5′-end of the modified oligonucleotide. In some embodiments, the conjugate group is attached to the modified oligonucleotide at the 3′-end of the modified oligonucleotide. In some embodiments, the conjugate group is a 6-palmitamidohexyl phosphate conjugate group attached to the 5′-OH of the modified oligonucleotide.

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. In certain embodiments, conjugate moieties impart a new property on the attached oligonucleotide, which may alter the oligonucleotides distribution or pharmacokinetic profile. For example, certain conjugate moieties selected from among lipids, vitamins, steroids, C₅-C₃₀ saturated alkyl groups, C₅-C₃₀ unsaturated alkyl groups, fatty acids, or lipophilic groups may increase the distribution of an oligonucleotide to various tissues or organs within a subject. In certain embodiments, certain conjugate moieties selected from among lipids, vitamins, steroids, C₅-C₃₀ saturated alkyl groups, C₅-C₃₀ unsaturated alkyl groups, fatty acids, or lipophilic groups increase affinity for an oligonucleotide with one or more serum proteins, such as albumin. In certain embodiments, certain conjugate moieties selected from among lipids, vitamins, steroids, C₅-C₃₀ saturated alkyl groups, C₅-C₃₀ unsaturated alkyl groups, fatty acids, or lipophilic groups increase affinity for an oligonucleotide to an extra-hepatic tissue. In some embodiments, the conjugate moiety is a 6-palmitamidohexyl conjugate moiety having the following structure:

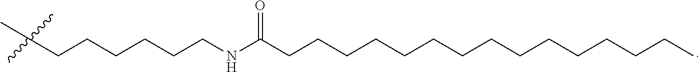 [SMILES: *CCCCCCNC(=O)CCCCCCCCCCCCCCC]

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphate or phosphorothioate linkage. In certain such embodiments, the cleavable moiety is 2′-deoxyadenosine.

3. Cell-Targeting Moieties

In certain embodiments, a conjugate group comprises a cell-targeting moiety. In certain embodiments, a conjugate group has the general formula:

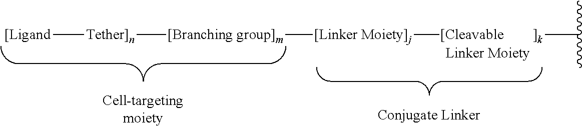

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

In certain embodiments, an oligomeric duplex comprises:

a first oligomeric compound comprising a first modified oligonucleotide consisting of 19 to 29 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 322-632; and a second oligomeric compound comprising a second modified oligonucleotide consisting of 15 to 29 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:

a first oligomeric compound comprising a first modified oligonucleotide consisting of 19 to 29 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 322-632; and a second oligomeric compound comprising a second modified oligonucleotide consisting of 15 to 29 linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any of SEQ ID NOs: 633-943, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:

a first oligomeric compound comprising a first modified oligonucleotide consisting of 23 linked nucleosides wherein the nucleobase sequence of the first modified oligonucleotide comprises the nucleobase sequence of any of SEQ ID NOs: 322-632; and
a second oligomeric compound comprising a second modified oligonucleotide consisting of 21 linked nucleosides wherein the nucleobase sequence of the second modified oligonucleotide comprises the nucleobase sequence of any of SEQ ID NOs: 633-943, wherein the nucleobase sequence of the second modified oligonucleotide is at least 90% complementary to an equal length portion of the first modified oligonucleotide.
In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage. In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, the internucleoside linkage motif of the second modified oligonucleotide can be ssooooooooooooooooss or, wherein each “o” represents a phosphodiester internucleoside linkage and each “s” represents a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of PMP22 nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### V. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, 8, 9, 10, or 11 from the 5′-end of the gap region. In certain embodiments, the mismatch is at position 1, 2, 3, 4, 5, or 6 from the 5′-end of the 5′ wing region or the 3′ wing region.

B. PMP22

In certain embodiments, oligomeric compounds comprise or consist of an oligonucleotide comprising a region that is complementary to a target nucleic acid, wherein the target nucleic acid is PMP22. In certain embodiments, PMP22 nucleic acid has the sequence set forth in SEQ ID NO: 1 (GENBANK Accession No. NM_000304.3), SEQ ID NO: 2 (GENBANK Accession No. NC_000017.11 truncated from nucleotides 15227001 to 15268000), SEQ ID NO: 3 (GENBANK Accession No. NM_153321.2), SEQ ID NO: 4 (GENBANK Accession No. NM_001281455.1), SEQ ID NO: 5 (GENBANK Accession No. NM_001281456.1), SEQ ID NO: 6 (GENBANK Accession No. NR_104017.1), SEQ ID NO:7 (GENBANK Accession No. NR_104018.1), or SEQ ID NO: 8 (GENBANK Accession No. AK300690.1).

In certain embodiments, contacting a cell with an oligomeric compound complementary to SEQ ID NO: 1. SEQ ID NO: 2, SEQ ID NO: 3, SEQ ID NO: 4, SEQ ID NO: 5, SEQ ID NO: 6. SEQ ID NO: 7, or SEQ ID NO: 8 reduces the amount of PMP22 RNA, and in certain embodiments reduces the amount of PMP22 protein. In certain embodiments, the oligomeric compound consists of a modified oligonucleotide. In certain embodiments, contacting a cell with an oligomeric compound complementary to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 results in reduced demyelination and/or reduced axonal damage and/or loss. In certain embodiments, the oligomeric compound consists of a modified oligonucleotide. In certain embodiments, the oligomeric compound consists of a modified oligonucleotide and a conjugate group.

C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the peripheral nervous system. Such tissues include the sciatic, tibial, peroneal, sural, radial, median, and ulnar nerves.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), intraneural, perineural, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1089870 equals the number of fully protonated molecules that weighs 10 mg. This would be equivalent to 10.57 mg of solvent-free, sodium-acetate free, anhydrous sodiated Compound No. 1089870. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### VII. Certain Compositions

1. Compound No. 1089870

In certain embodiments, Compound No. 1089870 is characterized as an oligomeric compound consisting of a conjugate group and a modified oligonucleotide, wherein the conjugate group is a 6-palmitamidohexyl phosphate conjugate group attached to the 5′-OH of the modified oligonucleotide, wherein the 6-palmitamidohexyl phosphate conjugate group is represented by the following structure:

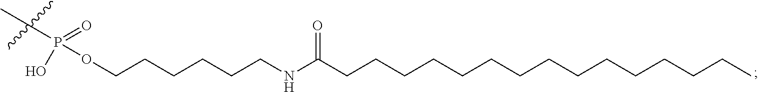 [SMILES: *P(=O)(O)OCCCCCCNC(=O)CCCCCCCCCCCCCCC]

and the modified oligonucleotide is a 3-10-3 cEt gapmer, having a sequence of (from 5′ to 3′) of AAATACGATCTTCTGG (SEQ ID NO: 239); wherein each of nucleosides 1-3 and 14-16 (from 5′ to 3′) comprise a cEt sugar moiety, and each of nucleosides 4-13 are 2′-β-D-deoxynucleosides; wherein each intemucleoside linkage is a phosphorothioate intemucleoside linkage; and wherein each cytosine is a 5-methyl cytosine.

In certain embodiments, Compound No. 1089870 is represented by the following chemical notation: (6-palmitamidohexyl)o Aks Aks Aks Tds Ads ^{m}Cds Gds Ads Tds ^{m}Cds Tds Tds ^{m}Cds Tks Gks Gk (SEQ ID NO: 239), wherein:
A=an adenine nucleobase,
^{m}C=a 5-methyl cytosine nucleobase,
G=a guanine nucleobase,
T=a thymine nucleobase,
k=a cEt sugar moiety,
d=a 2′-β-D-deoxyribosyl sugar moiety,
o=a phosphodiester linkage, and
s=a phosphorothioate intemucleoside linkage.

In certain embodiments, Compound No. 1089870 is represented by the following chemical structure:

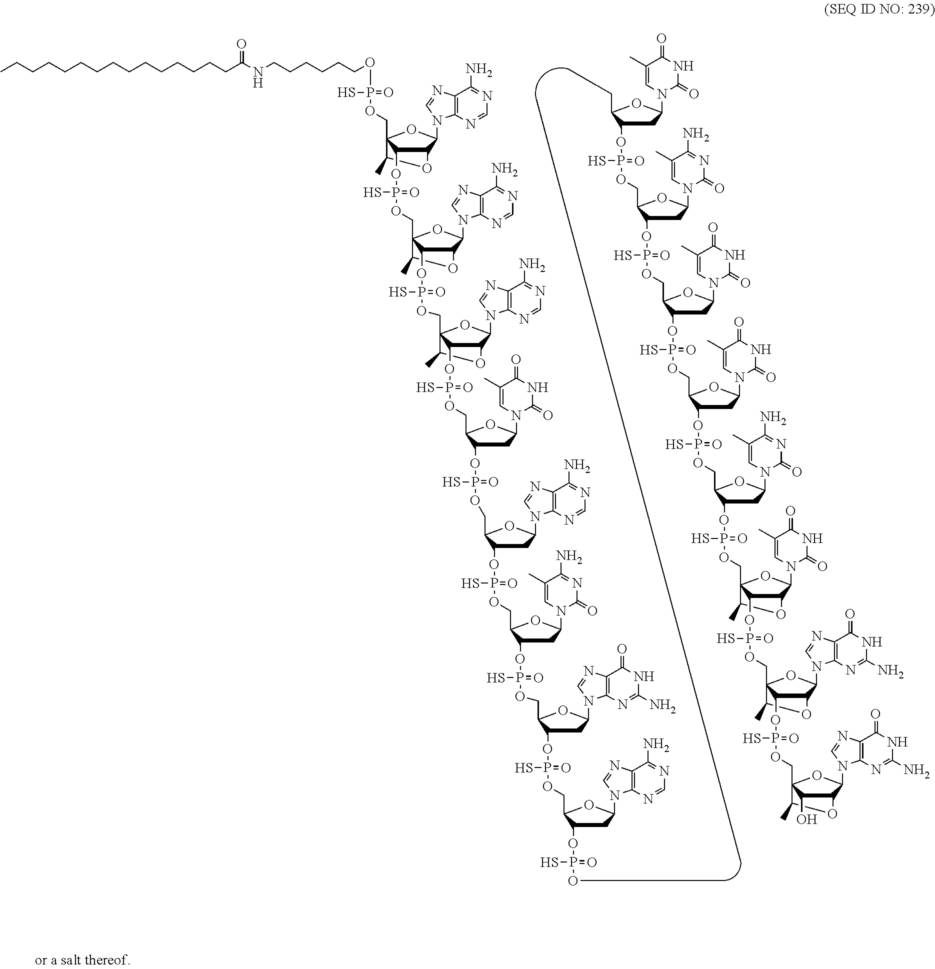

Structure 1. Compound No. 1089870

In certain embodiments, the sodium salt of Compound No. 1089870 is represented by the following chemical structure:

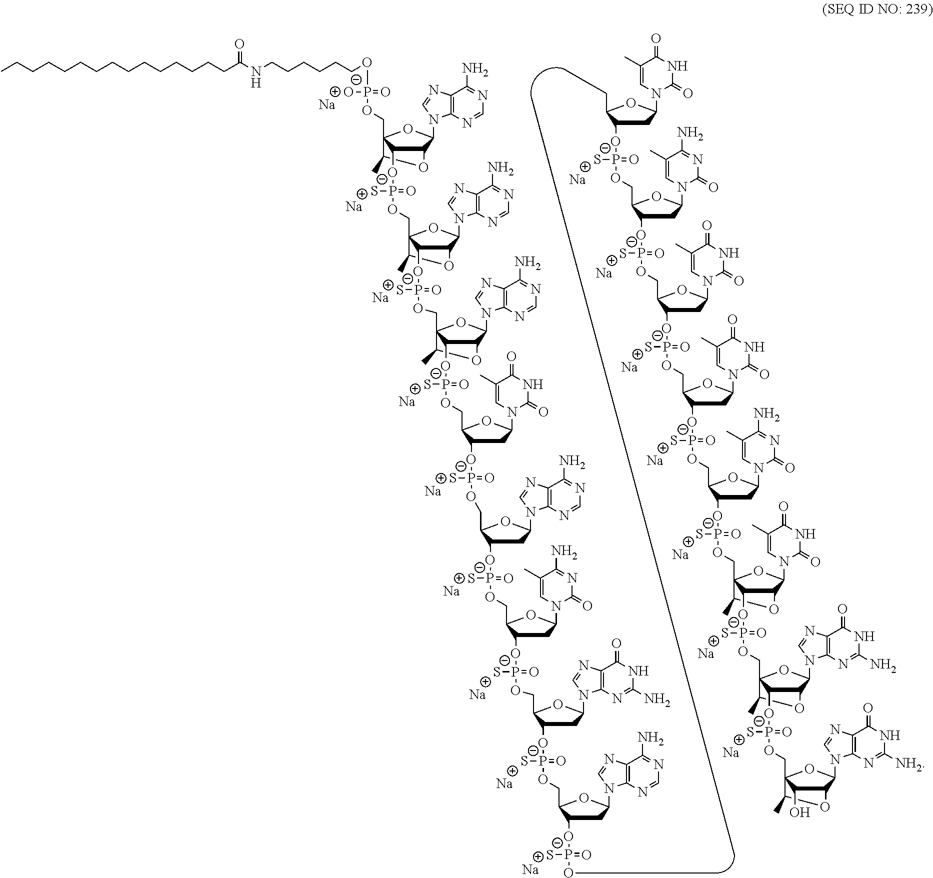

Structure 2. The Sodium Salt of Compounds No. 1089870

### VIII. Certain Hotspot Regions

In certain embodiments, nucleobases in the ranges specified below comprise a hotspot region of PMP22 nucleic acid. In certain embodiments, oligomeric duplexes comprising modified oligonucleotides that are complementary within a hotspot region of PMP22 nucleic acid achieve an average of more than 60% reduction of PMP22 RNA in vitro in the standard cell assay. In certain embodiments, such oligomeric duplexes are RNAi agents.

1. Nucleobases 765-1043 of SEO ID NO: 1

In certain embodiments, nucleobases 765-1043 of SEQ ID NO: 1 comprise a hotspot region. In certain embodiments, oligomeric duplexes comprise modified oligonucleotides that are complementary within nucleobases 765-1043 of SEQ ID NO: 1. In certain embodiments, oligomeric duplexes comprise a first oligomeric compound comprising a first modified oligonucleotide and a second oligomeric compound comprising a second modified oligonucleotide.

In certain embodiments, the first oligomeric compound comprises a first modified oligonucleotide that is 23 nucleobases in length. In certain embodiments, the second oligomeric compound comprises a second modified oligonucleotide that is 21 nucleobases in length. In certain embodiments, the second modified oligonucleotide is 100% complementary over its length to the first modified oligonucleotide.

The nucleobase sequences of SEQ ID Nos: 353-375 are complementary within nucleobases 765-1043 of SEQ ID NO: 1.

The nucleobase sequence of Compound Nos.: 1579683-1579687, 1579700-1579706, 1579720-1579723, 1579736-1579741 are complementary within nucleobases 765-1043 of SEQ ID NO: 1.

In certain embodiments, oligomeric duplexes comprising modified oligonucleotides complementary within nucleobases 765-1043 of SEQ ID NO: 1 achieve at least 37% reduction of PMP22 RNA in vitro in the standard cell assay. In certain embodiments, modified oligonucleotides complementary within nucleobases 765-1043 of SEQ ID NO: 1 achieve an average of 70% reduction of PMP22 RNA in vitro in the standard cell assay.

2. Nucleobases 1753-1859 of SEO ID NO: 1

In certain embodiments, nucleobases 1753-1859 of SEQ ID NO: 1 comprise a hotspot region. In certain embodiments, oligomeric duplexes comprise modified oligonucleotides that are complementary within nucleobases 1753-1859 of SEQ ID NO: 1. In certain embodiments, oligomeric duplexes comprise a first oligomeric compound comprising a first modified oligonucleotide and a second oligomeric compound comprising a second modified oligonucleotide.

In certain embodiments, the first oligomeric compound comprises a first modified oligonucleotide that is 23 nucleobases in length. In certain embodiments, the second oligomeric compound comprises a second modified oligonucleotide that is 21 nucleobases in length. In certain embodiments, the second modified oligonucleotide is 100% complementary over its length to the first modified oligonucleotide.

The nucleobase sequences of SEQ ID Nos: 1555, 558-619, 623, 624 are complementary within nucleobases 1753-1859 of SEQ ID NO: 1.

The nucleobase sequence of Compound Nos.: 1580287, 1580290, 1580303-1580308, 1580321-1580326, 1580339-1580344,1580357-1580362, 1580375-1580380,1580393-1580398, 1580411-1580416,1580429-1580434, 1580447-1580452, 1580465-1580470, 1580483, 1580487, 1580488 are complementary within nucleobases 1753-1859 of SEQ ID NO: 1.

In certain embodiments, oligomeric duplexes comprising modified oligonucleotides complementary within nucleobases 1753-1859 of SEQ ID NO: 1 achieve at least 37% reduction of PMP22 RNA in vitro in the standard cell assay. In certain embodiments, modified oligonucleotides complementary within nucleobases 1753-1859 of SEQ ID NO: 1 achieve an average of 64% reduction of PMP22 RNA in vitro in the standard cell assay.

### IX. Certain Comparator Compositions

In certain embodiments, Compound No. 684267, a 3-10-3 cEt gapmer having a sequence (from 5′ to 3′) of ATCTTCAATCAACAGC (SEQ ID NO: 18), wherein each internucleoside linkage is a phosphorothioate intemucleoside linkage, each cytosine is a 5-methyl cytosine, and wherein each of nucleosides 1-3 and 14-16 comprise a cEt modified sugar, which was previously described in WO2017156242, incorporated herein by reference, is a comparator compound.

In certain embodiments, Compound No. 684394, a 3-10-3 cEt gapmer having a sequence (from 5′ to 3′) of ATTATTCAGGTCTCCA (SEQ ID NO: 19), wherein each internucleoside linkage is a phosphorothioate internucleoside linkage, each cytosine is a 5-methyl cytosine, and wherein each of nucleosides 1-3 and 14-16 comprise a cEt modified sugar, which was previously described in WO2017156242, incorporated herein by reference, is a comparator compound.

As demonstrated in Example 2, Compound No. 684394 is more efficacious in vivo in transgenic mice than Compound No. 684267. For example, as provided in Table 4 Compound No. 684394 achieved an expression level of 29% control in a multi-dose study (three weekly doses of 50 mg/kg) in C₂₂ transgenic mice, while Compound No. 684267 achieved an expression level of 64% control in a multi-dose study in C₂₂ transgenic mice. Therefore, Compound No. 684394 is an appropriate comparator compound for in vivo efficacy in C₂₂ transgenic mice.

In certain embodiments, compounds described herein are superior relative to Compound No. 684394, because they demonstrate one or more improved properties, such as in vivo efficacy.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1089870, equals the number of fully protonated molecules that weighs 10 mg. This would be equivalent to 10.57 mg of solvent-free, sodium acetate-free, anhydrous sodiated Compound No. 1089870. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Modified Oligonucleotides Complementary to a Human PMP22 Nucleic Acid

Modified oligonucleotides complementary to a human PMP22 nucleic acid were designed, as described in the tables below.

The modified oligonucleotides in Table 1 are 3-10-3 cEt gapmers conjugated to a 6-palmitamidohexyl phosphate conjugate group attached to the 5′-OH of the oligonucleotide. The structure for the conjugate group is:

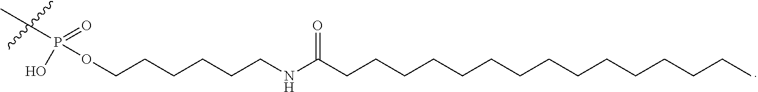 [SMILES: *P(=O)(O)OCCCCCCNC(=O)CCCCCCCCCCCCCCC]

The gapmers are 16 nucleosides in length, wherein the central gap segment consists of ten 2′-β-D-deoxynucleosides and the 5′ and 3′ wings each consists of three cEt nucleosides. The sugar motif for the gapmers is (from 5′ to 3′): kkkddddddddddkkk; wherein ‘d’ represents a 2′-β-D-deoxyribosyl sugar moiety; and ‘k’ represents a cEt sugar moiety. Each internucleoside linkages is a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the Tables below is 100% complementary to SEQ ID NO: 1 (GENBANK Accession No. NM_000304.3), or SEQ ID NO: 2 (GENBANK Accession No. NC_000017.11 truncated from nucleotides 15227001 to 15268000). ‘N/A’ indicates that the modified oligonucleotide is not 100% complementary to that particular target nucleic acid sequence.

TABLE 1
6-Palmitamidohexyl conjugated 3-10-3
cEt gapmers with PS internucleoside
linkages complementary to human PMP22

| | SEQ | SEQ | SEQ | SEQ | | |
|---|---|---|---|---|---|---|
| | ID | ID | ID | ID | | |
| | NO: | NO: | NO: | NO: | | |
| Com- | 1 | 1 | 2 | 2 | | SEQ |
| pound | Start | Stop | Start | Stop | Sequence | ID |
| ID | Site | Site | Site | Site | (5′ to 3′) | NO |
| 884288 | 864 | 879 | 37227 | 37242 | ATCTTCAATCAACAGC | 18 |
| 1054937 | 1489 | 1504 | 37852 | 37867 | ATTATTCAGGTCTCCA | 19 |
| 938327 | 230 | 245 | 7265 | 7280 | GGAGCATTCTGGCGGC | 20 |
| 938329 | 254 | 269 | 7289 | 7304 | GGACGATGATACTCAG | 21 |
| 938330 | 257 | 272 | 7292 | 7307 | GGAGGACGATGATACT | 22 |
| 938333 | 1012 | 1027 | 37375 | 37390 | GGATTATACTGTTAGG | 23 |
| 938336 | N/A | N/A | 4156 | 4171 | CCGTTATATGCCAAGC | 24 |
| 938337 | N/A | N/A | 4169 | 4184 | GCATTTACAGTGCCCG | 25 |
| 938340 | N/A | N/A | 11265 | 11280 | AGTTAAATGGTTCGCA | 26 |
| 938341 | N/A | N/A | 12664 | 12679 | TGTAAATAGGTGTAGG | 27 |
| 938348 | N/A | N/A | 31457 | 31472 | GTAGATTTCACATCCC | 28 |
| 955917 | N/A | N/A | 3978 | 3993 | AGGAAATAGTAATGCC | 29 |
| 955918 | N/A | N/A | 19600 | 19615 | GTCAAGGTATTCCAGC | 30 |
| 955919 | N/A | N/A | 28928 | 28943 | GCACATCAGGGCCATT | 31 |
| 955920 | N/A | N/A | 30905 | 30920 | CGCTTTTACATTCGGA | 32 |
| 955921 | N/A | N/A | 32372 | 32387 | GGATTAGGGACAGTTT | 33 |
| 955922 | N/A | N/A | 34301 | 34316 | CCAAGATAAGTGAGAC | 34 |
| 955924 | N/A | N/A | 35056 | 35071 | CCGTGATAAGCAGTAA | 35 |
| 955927 | N/A | N/A | 23258 | 23273 | CGTATAGACATCCACA | 36 |
| 955929 | N/A | N/A | 25154 | 25169 | GAAATGATGTAGGCTC | 37 |
| 955930 | N/A | N/A | 28834 | 28849 | CTGATTATGTGTCCAG | 38 |
| 955931 | N/A | N/A | 36829 | 36844 | GACAATTGCTGGGTAG | 39 |
| 955932 | N/A | N/A | 29157 | 29172 | TTAAGGAGACCTCTCA | 40 |
| 955934 | N/A | N/A | 14943 | 14958 | CATTCAATAGCAGGGC | 41 |
| 955935 | N/A | N/A | 21602 | 21617 | TGGATTCGACATGCAA | 42 |
| 955936 | N/A | N/A | 30679 | 30694 | GATGTAATGATGTTGC | 43 |
| 955938 | 1228 | 1243 | 37591 | 37606 | TCGGGCAGCGGCTGTT | 44 |
| 955943 | N/A | N/A | 19981 | 19996 | TCTCAGTATGAATGTC | 45 |
| 955944 | N/A | N/A | 21350 | 21365 | GTAGAACTTATGTTGA | 46 |
| 955945 | N/A | N/A | 25057 | 25072 | CCAGATAATTCCTCGG | 47 |
| 955946 | N/A | N/A | 28581 | 28596 | AAACTAATCATTCCGC | 48 |
| 955948 | 891 | 906 | 37254 | 37269 | GTTTTATAAACCGGAG | 49 |
| 955949 | N/A | N/A | 15955 | 15970 | TCTCATAAGAGCCTGT | 50 |
| 955950 | N/A | N/A | 19976 | 19991 | GTATGAATGTCATTCC | 51 |
| 955951 | N/A | N/A | 21349 | 21364 | TAGAACTTATGTTGAG | 52 |
| 955955 | N/A | N/A | 28801 | 28816 | GTTGATACGCCTGGCT | 53 |
| 955956 | N/A | N/A | 35191 | 35206 | GAGATACCAGATTCCA | 54 |
| 955960 | N/A | N/A | 30277 | 30292 | CATTTATCCTCTGGTG | 55 |
| 955961 | N/A | N/A | 15721 | 15736 | GCGATGATAGGAGACC | 56 |
| 955962 | N/A | N/A | 16719 | 16734 | TCCATTGGATCACCCT | 57 |
| 955963 | N/A | N/A | 19979 | 19994 | TCAGTATGAATGTCAT | 58 |
| 955964 | N/A | N/A | 3548 | 3563 | ACAACATACTCAGGAC | 59 |
| 955966 | N/A | N/A | 17751 | 17766 | GACAATATCTCCTGGC | 60 |
| 955967 | N/A | N/A | 23722 | 23737 | TGGATAATATCAGCAG | 61 |
| 955969 | N/A | N/A | 26833 | 26848 | GCACAACATATGCTTC | 62 |
| 955974 | N/A | N/A | 2967 | 2982 | GTCAATTCCAACACAA | 63 |
| 955976 | N/A | N/A | 30174 | 30189 | AACAAGGTTTGAGCGA | 64 |
| 955977 | N/A | N/A | 32576 | 32591 | TCCTTTTAGGTCTGTG | 65 |
| 955978 | N/A | N/A | 17355 | 17370 | CGTAGAGTCATCTAGA | 66 |
| 955980 | N/A | N/A | 12856 | 12871 | GGACTTATGGAACTGC | 67 |
| 955982 | N/A | N/A | 23200 | 23215 | ATTGATGTCAGTGGGT | 68 |
| 955983 | N/A | N/A | 17121 | 17136 | CATCATGTCCAGTCTG | 69 |
| 955986 | N/A | N/A | 4790 | 4805 | GGATAGCATGGTCTGG | 70 |
| 955988 | N/A | N/A | 2880 | 2895 | AGCATAGGCACACATC | 71 |
| 955990 | N/A | N/A | 3552 | 3567 | GATCACAACATACTCA | 72 |
| 955992 | 248 | 263 | 7283 | 7298 | TGATACTCAGCAACAG | 73 |
| 955994 | N/A | N/A | 7811 | 7826 | TGCCATAAAGCCGACA | 74 |
| 955995 | N/A | N/A | 10456 | 10471 | CAGTAGCGAGTACGGA | 75 |
| 955996 | N/A | N/A | 10740 | 10755 | TCCAACGGCAGAAGAC | 76 |
| 955998 | N/A | N/A | 11254 | 11269 | TCGCAATGAGCATAGT | 77 |
| 955999 | N/A | N/A | 16002 | 16017 | ACACATTCCGTCCTCT | 78 |
| 956000 | N/A | N/A | 16551 | 16566 | ACATAGGACAGGCTCA | 79 |
| 956001 | N/A | N/A | 18421 | 18436 | GCAAAGAGAGAGCGGC | 80 |
| 956002 | N/A | N/A | 23148 | 23163 | GGTCAAGAAGCCTTTC | 81 |
| 956003 | N/A | N/A | 28705 | 28720 | TCCCAAGTTCTAAGAC | 82 |
| 956004 | 628 | 643 | 36991 | 37006 | GGCGAAACCGTAGGAG | 83 |
| 956005 | N/A | N/A | 17261 | 17276 | CCCTAGCTAAGCCACC | 84 |
| 956007 | N/A | N/A | 14434 | 14449 | GGTCTAAGGAAATCAC | 85 |
| 956009 | N/A | N/A | 30823 | 30838 | GCAAGAGTGGATTAGT | 86 |
| 956010 | N/A | N/A | 3501 | 3516 | ATATCTAACTCAGGGT | 87 |
| 956011 | N/A | N/A | 19498 | 19513 | CTGCACTTTGACATCC | 88 |
| 956012 | 144 | 159 | 2787 | 2802 | GCCAAATGCAAGGGAT | 89 |
| 956013 | N/A | N/A | 3503 | 3518 | GCATATCTAACTCAGG | 90 |
| 956014 | N/A | N/A | 3550 | 3565 | TCACAACATACTCAGG | 91 |
| 956015 | N/A | N/A | 4360 | 4375 | TGTCATAGAAGCTCAT | 92 |
| 956017 | N/A | N/A | 7588 | 7603 | TCTGAAGTTACTTGGC | 93 |
| 956019 | N/A | N/A | 9487 | 9502 | CTGGATTAAGGACCCC | 94 |
| 956021 | N/A | N/A | 13849 | 13864 | GTAGATTCCGATGGGC | 95 |
| 956022 | N/A | N/A | 16117 | 16132 | GACTATAGATTCCAGG | 96 |
| 956023 | N/A | N/A | 16404 | 16419 | GGCTCGATGGGATAGG | 97 |
| 956024 | N/A | N/A | 16467 | 16482 | GGTAATAAGTTCCCCA | 98 |
| 956026 | N/A | N/A | 18995 | 19010 | GGTTTAGGAGGAATTC | 99 |
| 956027 | N/A | N/A | 19080 | 19095 | ACGGGAAAGGCAGTTG | 100 |
| 956028 | N/A | N/A | 19092 | 19107 | CCATAGAAAATGACGG | 101 |
| 956029 | N/A | N/A | 19267 | 19282 | GTGCATTGTACGATGA | 102 |
| 956030 | N/A | N/A | 27592 | 27607 | GGACAACCCGTATTTT | 103 |
| 956031 | N/A | N/A | 27704 | 27719 | GGTAAAGATCACGGGA | 104 |
| 956032 | N/A | N/A | 29566 | 29581 | TGACAACCAACTCAGA | 105 |
| 956034 | N/A | N/A | 32724 | 32739 | ACACATTTGACTTGAG | 106 |
| 956035 | N/A | N/A | 32847 | 32862 | TGCTCTATAGCTATAT | 107 |
| 956039 | N/A | N/A | 3019 | 3034 | GAGTATATATCCACCT | 108 |
| 956045 | N/A | N/A | 3564 | 3579 | CAACACTTATGTGATC | 109 |
| 956047 | N/A | N/A | 5018 | 5033 | CTAATAGAGGGCAGCG | 110 |
| 956048 | N/A | N/A | 9567 | 9582 | CCCTTAATTTGACCCT | 111 |
| 956050 | N/A | N/A | 10211 | 10226 | CCAATAGGACTGGGAC | 112 |
| 956051 | N/A | N/A | 16792 | 16807 | TCTAAATCTCAGACCA | 113 |
| 956053 | N/A | N/A | 26705 | 26720 | AGCATAGAGGTTCTTC | 114 |
| 956054 | N/A | N/A | 30611 | 30626 | GAAGATGAAGGTACTG | 115 |
| 956055 | N/A | N/A | 30636 | 30651 | GCCGAAACAGCTCAGC | 116 |
| 956058 | 1139 | 1154 | 37502 | 37517 | TTCTAAATGAGGTGGA | 117 |
| 956059 | 1709 | 1724 | 38072 | 38087 | CAATACAAGTCATTGC | 118 |
| 985154 | N/A | N/A | 7575 | 7590 | GGCTTAAATAGAGACC | 119 |
| 985156 | N/A | N/A | 9722 | 9737 | TCTTAACATCAATCGC | 120 |
| 985158 | N/A | N/A | 11251 | 11266 | CAATGAGCATAGTTCA | 121 |
| 985161 | N/A | N/A | 11268 | 11283 | ACAAGTTAAATGGTTC | 122 |
| 985165 | N/A | N/A | 15752 | 15767 | GATAAACAAGTCTGGG | 123 |
| 985174 | N/A | N/A | 21491 | 21506 | GTCAATTACAAACCTG | 124 |
| 985175 | N/A | N/A | 22444 | 22459 | GTCATAATGATCAAAC | 125 |
| 985176 | N/A | N/A | 30065 | 30080 | CATATTATCTTGAGGG | 126 |
| 985177 | N/A | N/A | 31231 | 31246 | CACGATAAGGGAACCA | 127 |
| 985178 | N/A | N/A | 8597 | 8612 | ACGATTATGTGCAGAG | 128 |
| 985180 | N/A | N/A | 9500 | 9515 | GAAATACGATCTTCTG | 129 |
| 985181 | N/A | N/A | 9502 | 9517 | GAGAAATACGATCTTC | 130 |
| 985182 | N/A | N/A | 9723 | 9738 | CTCTTAACATCAATCG | 131 |
| 985183 | N/A | N/A | 9845 | 9860 | AGCTGTATTTCCGTGG | 132 |
| 985186 | N/A | N/A | 11350 | 11365 | ACAAATCGATGTCAAT | 133 |
| 985188 | N/A | N/A | 12357 | 12372 | CTCTTATACACAATCA | 134 |
| 985190 | N/A | N/A | 19085 | 19100 | AAATGACGGGAAAGGC | 135 |
| 985191 | N/A | N/A | 19266 | 19281 | TGCATTGTACGATGAA | 136 |
| 985194 | N/A | N/A | 21533 | 21548 | AATGATTCGAGTTCAG | 137 |
| 985197 | N/A | N/A | 26072 | 26087 | GCATTAAGATAGTAAG | 138 |
| 985198 | N/A | N/A | 27616 | 27631 | ATAGGTATGGAAATCA | 139 |
| 985201 | N/A | N/A | 30525 | 30540 | TTATAGTACAGGCTTG | 140 |
| 985203 | N/A | N/A | 8998 | 9013 | AACGACATTCTGGCTT | 141 |
| 985206 | N/A | N/A | 14221 | 14236 | CCGAGATGTTCATATG | 142 |
| 985208 | N/A | N/A | 19261 | 19276 | TGTACGATGAAGGATA | 143 |
| 985209 | N/A | N/A | 19405 | 19420 | GATAATAGTAAGCTGT | 144 |
| 985211 | N/A | N/A | 19408 | 19423 | CTGGATAATAGTAAGC | 145 |
| 985213 | N/A | N/A | 19728 | 19743 | ACAAAGGCGATGAAGG | 146 |
| 985215 | N/A | N/A | 21244 | 21259 | CAATATCCAACCTTGG | 147 |
| 985216 | N/A | N/A | 21432 | 21447 | GCTAAACTATAGATTA | 148 |
| 985222 | N/A | N/A | 30163 | 30178 | AGCGAGGCACATCTCA | 149 |
| 985224 | N/A | N/A | 33291 | 33306 | ATCCATAATCATCCGT | 150 |
| 985228 | N/A | N/A | 10236 | 10251 | GACAAGATTAAGCACT | 151 |
| 985230 | N/A | N/A | 3018 | 3033 | AGTATATATCCACCTT | 152 |
| 985231 | N/A | N/A | 3416 | 3431 | CATATATTGGGTGCTA | 153 |
| 985232 | N/A | N/A | 4170 | 4185 | TGCATTTACAGTGCCC | 154 |
| 985233 | N/A | N/A | 5764 | 5779 | TCTTATAGACCATAAA | 155 |
| 985236 | N/A | N/A | 6970 | 6985 | AAACAAGCGGTTCGCA | 156 |
| 985238 | N/A | N/A | 9179 | 9194 | GTCGATATTTTTTTCA | 157 |
| 985239 | N/A | N/A | 9888 | 9903 | GAACATATATACTGGA | 158 |
| 985240 | N/A | N/A | 10027 | 10042 | GTATTACTGGGTACTG | 159 |
| 985241 | N/A | N/A | 12699 | 12714 | CCCAAGGCCGAATGAC | 160 |
| 985242 | N/A | N/A | 14557 | 14572 | GAAAATATACCGATGC | 161 |
| 985244 | N/A | N/A | 31814 | 31829 | ACCTAATGCAATCAAG | 162 |
| 985248 | N/A | N/A | 3799 | 3814 | CACTAAAGTAGCTTGT | 163 |
| 985249 | N/A | N/A | 3787 | 3802 | TTGTAACTCTGATAGG | 164 |
| 985250 | N/A | N/A | 9709 | 9724 | CGCTATGGCCTACCCA | 165 |
| 985251 | N/A | N/A | 4736 | 4751 | ACCGAATAAGCTCTAG | 166 |
| 985252 | N/A | N/A | 7741 | 7756 | TCCTAAAAGGGTGTTT | 167 |
| 985253 | N/A | N/A | 10237 | 10252 | AGACAAGATTAAGCAC | 168 |
| 985255 | N/A | N/A | 15753 | 15768 | TGATAAACAAGTCTGG | 169 |
| 985256 | N/A | N/A | 15892 | 15907 | AGCCACGAAGATTTTA | 170 |
| 985257 | N/A | N/A | 15917 | 15932 | GTAGAGATTATGGGTT | 171 |
| 985258 | N/A | N/A | 18921 | 18936 | ACGCATTATGGAAATG | 172 |
| 985260 | N/A | N/A | 20005 | 20020 | ACACAGATCGCCATTT | 173 |
| 985263 | N/A | N/A | 25197 | 25212 | GACTATAAGGGCAACA | 174 |
| 985264 | N/A | N/A | 27620 | 27635 | CTCTATAGGTATGGAA | 175 |
| 985265 | N/A | N/A | 34231 | 34246 | TCAATATAGTTCGATG | 176 |
| 1083583 | 1471 | 1486 | 37834 | 37849 | CTATCTTATGTTGTAA | 177 |
| 1083584 | 1476 | 1491 | 37839 | 37854 | CCATTCTATCTTATGT | 178 |
| 1083585 | 1478 | 1493 | 37841 | 37856 | CTCCATTCTATCTTAT | 179 |
| 1083586 | 1492 | 1507 | 37855 | 37870 | AGAATTATTCAGGTCT | 180 |
| 1083587 | 1488 | 1503 | 37851 | 37866 | TTATTCAGGTCTCCAT | 181 |
| 1083588 | 1490 | 1505 | 37853 | 37868 | AATTATTCAGGTCTCC | 182 |
| 1083589 | N/A | N/A | 23195 | 23210 | TGTCAGTGGGTTCCCC | 183 |
| 1083590 | N/A | N/A | 28712 | 28727 | TTCTAAGTCCCAAGTT | 184 |
| 1083591 | N/A | N/A | 31450 | 31465 | TCACATCCCATGAGTG | 185 |
| 1083592 | N/A | N/A | 19083 | 19098 | ATGACGGGAAAGGCAG | 186 |
| 1083593 | N/A | N/A | 19084 | 19099 | AATGACGGGAAAGGCA | 187 |
| 1083594 | N/A | N/A | 19090 | 19105 | ATAGAAAATGACGGGA | 188 |
| 1083595 | N/A | N/A | 23201 | 23216 | AATTGATGTCAGTGGG | 189 |
| 1083596 | N/A | N/A | 23206 | 23221 | TGTCAAATTGATGTCA | 190 |
| 1083597 | 1491 | 1506 | 37854 | 37869 | GAATTATTCAGGTCTC | 191 |
| 1083598 | 1493 | 1508 | 37856 | 37871 | CAGAATTATTCAGGTC | 192 |
| 1083599 | 1494 | 1509 | 37857 | 37872 | ACAGAATTATTCAGGT | 193 |
| 1083600 | 1495 | 1510 | 37858 | 37873 | CACAGAATTATTCAGG | 194 |
| 1083601 | 1496 | 1511 | 37859 | 37874 | ACACAGAATTATTCAG | 195 |
| 1083602 | 1498 | 1513 | 37861 | 37876 | TTACACAGAATTATTC | 196 |
| 1083603 | 1500 | 1515 | 37863 | 37878 | TATTACACAGAATTAT | 197 |
| 1083604 | 1501 | 1516 | 37864 | 37879 | ATATTACACAGAATTA | 198 |
| 1083605 | 1502 | 1517 | 37865 | 37880 | TATATTACACAGAATT | 199 |
| 1083606 | 1503 | 1518 | 37866 | 37881 | TTATATTACACAGAAT | 200 |
| 1083607 | 1504 | 1519 | 37867 | 37882 | TTTATATTACACAGAA | 201 |
| 1083608 | 1505 | 1520 | 37868 | 37883 | ATTTATATTACACAGA | 202 |
| 1083609 | 1506 | 1521 | 37869 | 37884 | CATTTATATTACACAG | 203 |
| 1083610 | 1507 | 1522 | 37870 | 37885 | CCATTTATATTACACA | 204 |
| 1083611 | 1508 | 1523 | 37871 | 37886 | ACCATTTATATTACAC | 205 |
| 1083612 | 1509 | 1524 | 37872 | 37887 | AACCATTTATATTACA | 206 |
| 1083613 | 1473 | 1488 | 37836 | 37851 | TTCTATCTTATGTTGT | 207 |
| 1083614 | 1470 | 1485 | 37833 | 37848 | TATCTTATGTTGTAAA | 208 |
| 1083615 | 1469 | 1484 | 37832 | 37847 | ATCTTATGTTGTAAAA | 209 |
| 1083616 | N/A | N/A | 31459 | 31474 | TTGTAGATTTCACATC | 210 |
| 1083617 | N/A | N/A | 31455 | 31470 | AGATTTCACATCCCAT | 211 |
| 1083618 | N/A | N/A | 31452 | 31467 | TTTCACATCCCATGAG | 212 |
| 1083619 | N/A | N/A | 23202 | 23217 | AAATTGATGTCAGTGG | 213 |
| 1083620 | N/A | N/A | 23204 | 23219 | TCAAATTGATGTCAGT | 214 |
| 1083621 | N/A | N/A | 23205 | 23220 | GTCAAATTGATGTCAG | 215 |
| 1083622 | N/A | N/A | 19086 | 19101 | AAAATGACGGGAAAGG | 216 |
| 1083623 | N/A | N/A | 19087 | 19102 | GAAAATGACGGGAAAG | 217 |
| 1083624 | N/A | N/A | 19088 | 19103 | AGAAAATGACGGGAAA | 218 |
| 1083625 | N/A | N/A | 19089 | 19104 | TAGAAAATGACGGGAA | 219 |
| 1083626 | N/A | N/A | 19079 | 19094 | CGGGAAAGGCAGTTGC | 220 |
| 1083627 | N/A | N/A | 19078 | 19093 | GGGAAAGGCAGTTGCA | 221 |
| 1083628 | N/A | N/A | 19077 | 19092 | GGAAAGGCAGTTGCAA | 222 |
| 1083629 | N/A | N/A | 28698 | 28713 | TTCTAAGACACATACA | 223 |
| 1083630 | N/A | N/A | 28697 | 28712 | TCTAAGACACATACAG | 224 |
| 1083631 | N/A | N/A | 28696 | 28711 | CTAAGACACATACAGG | 225 |
| 1083632 | N/A | N/A | 28695 | 28710 | TAAGACACATACAGGT | 226 |
| 1083641 | 1021 | 1036 | 37384 | 37399 | TACTGAGCTGGATTAT | 227 |
| 1083642 | 1180 | 1195 | 37543 | 37558 | AGGGCTTTTGGACATT | 228 |
| 1083643 | 1229 | 1244 | 37592 | 37607 | TTCGGGCAGCGGCTGT | 229 |
| 1083644 | 1232 | 1247 | 37595 | 37610 | AGGTTCGGGCAGCGGC | 230 |
| 1083645 | N/A | N/A | 12574 | 12589 | GAATCATGGATGAGAT | 231 |
| 1083646 | N/A | N/A | 12811 | 12826 | GTATGATTGGGTATGG | 232 |
| 1083647 | N/A | N/A | 14212 | 14227 | TCATATGGCTGGCTCC | 233 |
| 1083648 | N/A | N/A | 19678 | 19693 | GGGTATTTTAGCTAGA | 234 |
| 1083649 | N/A | N/A | 32384 | 32399 | AACCTTACAGTGGGAT | 235 |
| 1089867 | N/A | N/A | 9497 | 9512 | ATACGATCTTCTGGAT | 236 |
| 1089868 | N/A | N/A | 9538 | 9553 | TTCCATATCTCACAAG | 237 |
| 1089869 | N/A | N/A | 9498 | 9513 | AATACGATCTTCTGGA | 238 |
| 1089870 | N/A | N/A | 9499 | 9514 | AAATACGATCTTCTGG | 239 |
| 1089871 | N/A | N/A | 9501 | 9516 | AGAAATACGATCTTCT | 240 |
| 1089872 | N/A | N/A | 9505 | 9520 | GAGGAGAAATACGATC | 241 |
| 1089873 | N/A | N/A | 9566 | 9581 | CCTTAATTTGACCCTC | 242 |
| 1089874 | N/A | N/A | 9503 | 9518 | GGAGAAATACGATCTT | 243 |
| 1089876 | N/A | N/A | 9508 | 9523 | ACAGAGGAGAAATACG | 244 |
| 1089877 | N/A | N/A | 9509 | 9524 | GACAGAGGAGAAATAC | 245 |
| 1089878 | N/A | N/A | 9510 | 9525 | GGACAGAGGAGAAATA | 246 |
| 1089879 | N/A | N/A | 9512 | 9527 | GAGGACAGAGGAGAAA | 247 |
| 1089880 | N/A | N/A | 9515 | 9530 | GCTGAGGACAGAGGAG | 248 |
| 1089883 | N/A | N/A | 9496 | 9511 | TACGATCTTCTGGATT | 249 |
| 1089884 | N/A | N/A | 9492 | 9507 | ATCTTCTGGATTAAGG | 250 |
| 1089885 | N/A | N/A | 9483 | 9498 | ATTAAGGACCCCAAGG | 251 |
| 1089886 | N/A | N/A | 9482 | 9497 | TTAAGGACCCCAAGGC | 252 |
| 1089887 | N/A | N/A | 9480 | 9495 | AAGGACCCCAAGGCTT | 253 |
| 1120838 | N/A | N/A | 28790 | 28805 | TGGCTTGGGTGGGGAC | 254 |
| 1120839 | N/A | N/A | 9178 | 9193 | TCGATATTTTTTTCAA | 255 |
| 1120840 | N/A | N/A | 9177 | 9192 | CGATATTTTTTTCAAT | 256 |
| 1120841 | N/A | N/A | 9176 | 9191 | GATATTTTTTTCAATC | 257 |
| 1120842 | N/A | N/A | 9175 | 9190 | ATATTTTTTTCAATCA | 258 |
| 1120843 | N/A | N/A | 9174 | 9189 | TATTTTTTTCAATCAC | 259 |
| 1120844 | N/A | N/A | 9173 | 9188 | ATTTTTTTCAATCACA | 260 |
| 1120845 | N/A | N/A | 9172 | 9187 | TTTTTTTCAATCACAA | 261 |
| 1120846 | N/A | N/A | 9171 | 9186 | TTTTTTCAATCACAAA | 262 |
| 1120847 | N/A | N/A | 9170 | 9185 | TTTTTCAATCACAAAA | 263 |
| 1120848 | N/A | N/A | 9169 | 9184 | TTTTCAATCACAAAAA | 264 |
| 1120849 | N/A | N/A | 9180 | 9195 | GGTCGATATTTTTTTC | 265 |
| 1120853 | N/A | N/A | 9184 | 9199 | CCCAGGTCGATATTTT | 266 |
| 1120854 | N/A | N/A | 9185 | 9200 | TCCCAGGTCGATATTT | 267 |
| 1120863 | 1472 | 1487 | 37835 | 37850 | TCTATCTTATGTTGTA | 268 |
| 1120865 | N/A | N/A | 9506 | 9521 | AGAGGAGAAATACGAT | 269 |
| 1120873 | N/A | N/A | 9495 | 9510 | ACGATCTTCTGGATTA | 270 |
| 1120875 | N/A | N/A | 9493 | 9508 | GATCTTCTGGATTAAG | 271 |
| 1120877 | N/A | N/A | 9490 | 9505 | CTTCTGGATTAAGGAC | 272 |
| 1120881 | 1497 | 1512 | 37860 | 37875 | TACACAGAATTATTCA | 273 |
| 1120882 | 1499 | 1514 | 37862 | 37877 | ATTACACAGAATTATT | 274 |
| 1120890 | 1475 | 1490 | 37838 | 37853 | CATTCTATCTTATGTT | 275 |
| 1120892 | N/A | N/A | 31460 | 31475 | CTTGTAGATTTCACAT | 276 |
| 1120894 | N/A | N/A | 31462 | 31477 | CGCTTGTAGATTTCAC | 277 |
| 1120895 | N/A | N/A | 31463 | 31478 | ACGCTTGTAGATTTCA | 278 |
| 1120896 | N/A | N/A | 31464 | 31479 | CACGCTTGTAGATTTC | 279 |
| 1120897 | N/A | N/A | 31465 | 31480 | GCACGCTTGTAGATTT | 280 |
| 1120898 | N/A | N/A | 31466 | 31481 | TGCACGCTTGTAGATT | 281 |
| 1120899 | N/A | N/A | 31467 | 31482 | ATGCACGCTTGTAGAT | 282 |
| 1263229 | 873 | 888 | 37236 | 37251 | ATTATATACATCTTCA | 283 |
| 1263254 | N/A | N/A | 29769 | 29784 | GTATTGAAATGTTGTA | 284 |
| 1263256 | N/A | N/A | 10283 | 10298 | CGTAATTTCTGGGATG | 285 |
| 1263258 | N/A | N/A | 14985 | 15000 | GATATTTGGATCTTGG | 286 |
| 1263259 | N/A | N/A | 10058 | 10073 | TTTATAATGCTTCAGC | 287 |
| 1263261 | N/A | N/A | 22457 | 22472 | ATTTTATTGGGTTGTC | 288 |
| 1263271 | N/A | N/A | 29880 | 29895 | GCATAATTAGTCATTT | 289 |
| 1263272 | N/A | N/A | 8628 | 8643 | GTAAATGGAGAAGTTC | 290 |
| 1263275 | N/A | N/A | 21538 | 21553 | AAAATAATGATTCGAG | 291 |
| 1263277 | N/A | N/A | 23272 | 23287 | AAACTATGTAATTGCG | 292 |
| 1263280 | N/A | N/A | 30803 | 30818 | ATACTTAAATCGCAAT | 293 |
| 1263281 | N/A | N/A | 25304 | 25319 | ATAGATTACATAAGAG | 294 |
| 1263282 | N/A | N/A | 24037 | 24052 | GATAATACACAACATC | 295 |
| 1263304 | N/A | N/A | 29898 | 29913 | TAGAAATGATATCTCC | 296 |
| 1263305 | N/A | N/A | 26239 | 26254 | CATAAGTTATTTCTCG | 297 |
| 1263308 | N/A | N/A | 13009 | 13024 | TTACTTATACCTGGAG | 298 |
| 1263312 | N/A | N/A | 30068 | 30083 | ATCCATATTATCTTGA | 299 |
| 1263317 | N/A | N/A | 27055 | 27070 | AGAATTTTAAGGGTGC | 300 |
| 1263332 | N/A | N/A | 23950 | 23965 | TAAATCTCTAGTTCGC | 301 |
| 1263334 | N/A | N/A | 23419 | 23434 | ATTATGGTATATCCTC | 302 |
| 1263362 | N/A | N/A | 11267 | 11282 | CAAGTTAAATGGTTCG | 303 |
| 1263363 | N/A | N/A | 27054 | 27069 | GAATTTTAAGGGTGCA | 304 |
| 1263365 | N/A | N/A | 14986 | 15001 | TGATATTTGGATCTTG | 305 |
| 1263368 | N/A | N/A | 29744 | 29759 | TTAATGAGAAGTGGTT | 306 |
| 1263369 | N/A | N/A | 19186 | 19201 | TCAAAACTTAGTGTCT | 307 |
| 1263370 | N/A | N/A | 22456 | 22471 | TTTTATTGGGTTGTCA | 308 |
| 1263378 | N/A | N/A | 19570 | 19585 | TTATTCAATGAACTGC | 309 |
| 1263380 | N/A | N/A | 29745 | 29760 | TTTAATGAGAAGTGGT | 310 |
| 1263381 | N/A | N/A | 15612 | 15627 | ATTGATAGTCATGAGT | 311 |
| 1263382 | N/A | N/A | 12372 | 12387 | TCATTTGAAGATGTTC | 312 |
| 1263384 | N/A | N/A | 32285 | 32300 | CTATTAAATGAGAGTC | 313 |
| 1263385 | N/A | N/A | 27604 | 27619 | ATCAATTTGCTTGGAC | 314 |
| 1263386 | N/A | N/A | 29454 | 29469 | ATGAATTTGGACCACA | 315 |
| 1263390 | N/A | N/A | 23162 | 23177 | ACGAAGAAGAGTTTGG | 316 |
| 1263400 | N/A | N/A | 19168 | 19183 | CAAACGGATTTATCAG | 317 |
| 1263768 | N/A | N/A | 32561 | 32576 | GTCTAGATTAATTTCT | 318 |
| 1263770 | N/A | N/A | 12239 | 12254 | GTATTGACTTAAATCC | 319 |
| 1263771 | N/A | N/A | 29770 | 29785 | AGTATTGAAATGTTGT | 320 |

Modified oligonucleotides complementary to a human PMP22 nucleic acid were designed as described in Table 2. The modified oligonucleotides in Table 2 are 16-mer gapmers with mixed sugar motifs as indicated, wherein ‘d’ represents a 2′-β-D-deoxyribosyl sugar moiety; ‘e’ represents a 2′-MOE sugar moiety; ‘k’ represents to a cEt sugar moiety; and ‘y’ represents a 2′-OMe sugar moiety. All internucleoside linkages are phosphorothioate internucleoside linkages. Each cytosine residues is a 5-methylcytosine. Modified oligonucleotides in Table 2 are conjugated to a 6-palmitamidohexyl phosphate conjugate group attached to the 5′-OH of the modified oligonucleotide. The structure for the conjugate group is:

TABLE 2
6-Palmitamidohexyl conjugated 3-10-3 mixed sugar gapmers with uniform PS internucleoside
linkages complementary to human PMP22

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | Sugar Motif | SEQ ID |
| ID | Site | Site | Site | Site | Sequence (5′ to 3′) | (5′ to 3′) | NO |
| 1182273 | 1489 | 1504 | 37852 | 37867 | ATTATUCAGGTCTCCA | kkkddydddddddkkk | 321 |
| 1421291 | N/A | N/A | 9498 | 9513 | AATACGATCTTCTGGA | ekkddddddddddkkk | 238 |
| 1421292 | N/A | N/A | 9499 | 9514 | AAATACGATCTTCTGG | ekkddddddddddkkk | 239 |
| 1421296 | N/A | N/A | 9499 | 9514 | AAATACGATCTTCTGG | kekddddddddddkkk | 239 |
| 1421314 | N/A | N/A | 9498 | 9513 | AATACGATCTTCTGGA | kkeddddddddddkkk | 238 |
| 1421315 | N/A | N/A | 9499 | 9514 | AAATACGATCTTCTGG | kkeddddddddddkkk | 239 |
| 1421318 | N/A | N/A | 9498 | 9513 | AATACGATCTTCTGGA | kkkddddddddddekk | 238 |
| 1421329 | N/A | N/A | 9499 | 9514 | AAATACGATCTTCTGG | kkkddddddddddkek | 239 |
| 1421336 | N/A | N/A | 9498 | 9513 | AATACGATCTTCTGGA | ekkddddddddddkke | 238 |
| 1421341 | N/A | N/A | 9499 | 9514 | AAATACGATCTTCTGG | ekkkdddddddddkkk | 239 |
| 1421345 | N/A | N/A | 9499 | 9514 | AAATACGATCTTCTGG | kkkdddddddddkkke | 239 |

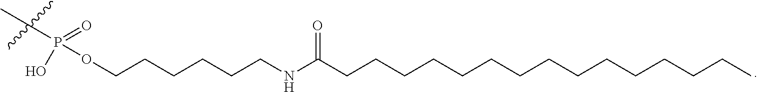 [SMILES: *P(=O)(O)OCCCCCCNC(=O)CCCCCCCCCCCCCCC]

Modified oligonucleotides complementary to a human PMP22 nucleic acid were designed as described in Table 3. The modified oligonucleotides in Table 3 are 3-10-3 cEt gapmers conjugated to a 6-palmitamidohexyl phosphate conjugate group attached to the 5′-OH of the oligonucleotide. The structure for the conjugate group is:

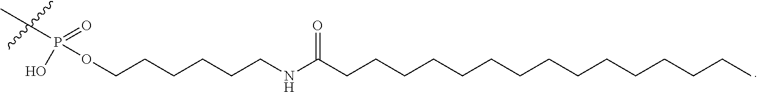 [SMILES: *P(=O)(O)OCCCCCCNC(=O)CCCCCCCCCCCCCCC]

The gapmers are 16 nucleosides in length, wherein the central gap segment consists of ten 2′-β-D-deoxynucleosides and the 5′ and 3′ wings each consists of three cEt nucleosides. The sugar motif for the gapmers is (from 5′ to 3′): kkkddddddddddkkk; wherein ‘d’ represents a 2′-β-D-deoxyribosyl sugar moiety; and ‘k’ represents a cEt sugar moiety. The intemucleoside linkage motifs for the gapmers are described in the table below, wherein ‘s’ refers to a phosphorothioate internucleoside linkage; and ‘q’ refers to a methoxypropyl phosphonate (MOP) internucleoside linkage. Each cytosine residues is a 5-methylcytosine.

TABLE 3
6-Palmitamidohexyl conjugated 3-10-3 cEt gapmers with mixed PS/MOP internucleoside linkages
complementary to human PMP22

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | Sugar Motif | SEQ ID |
| ID | Site | Site | Site | Site | Sequence (5′ to 3′) | (5′ to 3′) | NO |
| 1182269 | 1489 | 1504 | 37852 | 37867 | ATTATTCAGGTCTCCA | ssssqssssssssss | 19 |
| 1182270 | 1489 | 1504 | 37852 | 37867 | ATTATTCAGGTCTCCA | sssssqsssssssss | 19 |
| 1182271 | 1489 | 1504 | 37852 | 37867 | ATTATTCAGGTCTCCA | ssssssqssssssss | 19 |
| 1182285 | N/A | N/A | 9499 | 9514 | AAATACGATCTTCTGG | ssssqssssssssss | 239 |
| 1182286 | N/A | N/A | 9499 | 9514 | AAATACGATCTTCTGG | sssssqsssssssss | 239 |
| 1182287 | N/A | N/A | 9499 | 9514 | AAATACGATCTTCTGG | ssssssqssssssss | 239 |

### Example 2: Effect of Modified Oligonucleotides on Human PMP22 in Transgenic Mice

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

C22 mice were divided into groups of 1-4 mice each and administered 50 mg/kg of modified oligonucleotide by subcutaneous injection once a week for a total of three injections. A group of 1-4 mice was administered subcutaneous injections of PBS once a week for a total of three injections. This group serves as the control group to which other groups were compared. The number of mice in a treatment group in each experiment is noted in each experimental table below as the “n” number. Mice were sacrificed 72 hours after the final injection and total RNA was isolated from the sciatic nerve for analysis. Levels of human PMP22 RNA were measured by quantitative real-time RTPCR using human primer probe set RTS4579 (forward sequence CTTGCTGGTCTGTGCGTGAT, designated herein as SEQ ID NO: 9; reverse sequence ACCGTAGGAGTAATCCGAGTTGAG, designated herein as SEQ ID NO: 10; probe sequence CATCTACACGGTGAGGCACCCGG, designated herein as SEQ ID NO: 11). Results are presented as percent human PMP22 RNA relative to PBS control, normalized to mouse cyclophilin A. Cyclophilin A was amplified using mouse primer probe set mcyclo24 (forward sequence TCGCCGCTTGCTGCA, designated herein as SEQ ID NO: 12; reverse sequence ATCGGCCGTGATGTCGA, designated herein as SEQ ID NO: 13; probe sequence CCATGGTCAACCCCACCGTGTTC, designated herein as SEQ ID NO: 14). The values marked with the symbol “I” indicate that the modified oligonucleotide is complementary to the amplicon region of the primer probe set. Additional assays may be used to measure the potency and efficacy of the modified oligonucleotides complementary to the amplicon region.

TABLE 4
Reduction of human PMP22 in C22 transgenic mice, n = 4

| | PMP22 |
|---|---|
| | RNA (% |
| Compound | Control) |
| ID | RTS4579 |
| PBS | 100 |
| 684267 | 64 |
| 684394 | 29 |
| 938327 | 71 |
| 938329 | 78 |
| 938330 | 84 |
| 938333 | 78 |
| 938336 | 89 |
| 938340 | 96 |
| 938341 | 82 |
| 955917 | 92 |
| 955918 | 97 |
| 955919 | 86 |
| 955920 | 84 |
| 955921 | 91 |
| 955922 | 95 |
| 955924 | 104 |
| 955927 | 101 |
| 955929 | 113 |
| 955930 | 99 |
| 955931 | 102 |
| 955932 | 111 |
| 955934 | 80 |
| 955935 | 86 |

TABLE 5
Reduction of human PMP22 in C22 transgenic mice, n = 4

| | PMP22 |
|---|---|
| | RNA (% |
| Compound | Control) |
| ID | RTS4579 |
| PBS | 100 |
| 684267 | 76 |
| 684394 | 38 |
| 955943 | 94 |
| 955944 | 77 |
| 955946 | 89 |
| 955948 | 79 |
| 955949 | 86 |
| 955950 | 75 |
| 955951 | 83 |
| 955990 | 91 |
| 955996 | 92 |
| 955998 | 83 |
| 955999 | 74 |
| 956000 | 85 |
| 956004 | 77^{≠} |
| 956007 | 79 |

TABLE 6
Reduction of human PMP22 in C22 transgenic mice, n = 4

| | PMP22 |
|---|---|
| | RNA (% |
| Compound | Control) |
| ID | RTS4579 |
| PBS | 100 |
| 684267 | 82 |
| 684394 | 57 |
| 955936 | 84 |
| 955945 | 90 |
| 955955 | 61 |
| 955960 | 84 |
| 955964 | 68 |
| 955966 | 80 |
| 955967† | 61 |
| 955974 | 72 |
| 955976 | 79 |
| 955986 | 76 |
| 955988 | 68 |
| 955994 | 81 |
| 955995 | 89 |
| 956002 | 81 |
| 956003 | 55 |
| 956010 | 79 |
| 956012 | 76 |
| 956013 | 74 |
| 956014† | 97 |
| 956015 | 89 |
| 956017† | 70 |
| 956021 | 109 |
| 956022 | 83 |
| 956023 | 88 |
| 956024 | 80 |
| 956028 | 79 |
| 956029 | 80 |
| 956030 | 74 |
| 956031 | 78 |
| 956034 | 61^{≠} |
| 956035 | 99 |

†Group had fewer than 3 animals at end of study

TABLE 7
Reduction of human PMP22 in C22 transgenic mice, n = 4

| | PMP22 |
|---|---|
| | RNA (% |
| Compound | Control) |
| ID | RTS4579 |
| PBS | 100 |
| 684267 | 70 |
| 684394 | 37 |
| 938337 | 77 |
| 938348 | 48 |
| 955961 | 79 |
| 955962 | 81 |
| 955963 | 72 |
| 955969† | 61 |
| 955977 | 62 |
| 955978 | 71 |
| 955980 | 70 |
| 955982 | 63 |
| 955983 | 62 |
| 956005 | 54 |
| 956009 | 77 |
| 956011 | 77 |
| 956019 | 73 |
| 956026† | 77 |
| 956027 | 60 |
| 956032 | 75 |
| 956045 | 86 |
| 956047 | 94 |
| 956048 | 87 |
| 956050 | 80 |
| 956051 | 91 |
| 956053 | 70 |
| 956054 | 83 |
| 956055 | 97 |
| 956058 | 70 |
| 956059† | 92 |

†Group had fewer than 3 animals at end of study

TABLE 8
Reduction of human PMP22 in C22 transgenic mice, n = 1

| Compound | PMP22 |
|---|---|
| ID | RNA (% |
| | Control) |
| | RTS4579 |
| PBS | 100 |
| 684394 | 52 |
| 955956 | 49 |
| 955992 | 74 |
| 956001 | 89 |
| 956039 | 79 |
| 985154 | 78 |
| 985156 | 64 |
| 985158 | 76 |
| 985161 | 74 |
| 985165 | 79 |
| 985174 | 97 |
| 985175 | 77 |
| 985176 | 87 |
| 985177 | 64 |
| 985178 | 80 |
| 985180 | 38 |
| 985181 | 83 |
| 985182 | 84 |
| 985183 | 80 |
| 985186 | 92 |
| 985188 | 72 |
| 985191 | 75 |
| 985194 | 87 |
| 985197 | 75 |
| 985198 | 86 |
| 985201 | 68 |
| 985203 | 73 |
| 985206 | 87 |
| 985208 | 77 |
| 985209 | 93 |
| 985211 | 80 |
| 985213 | 81 |
| 985215 | 86 |
| 985216 | 87 |
| 985222 | 66 |
| 985224 | 88 |
| 985228 | 66 |
| 985230 | 77 |
| 985231 | 75 |
| 985232 | 76 |
| 985233 | 94 |
| 985236 | 101 |
| 985238 | 59 |
| 985239 | 78 |
| 985240 | 73 |
| 985241 | 92 |
| 985242 | 69 |
| 985244 | 77 |
| 985248 | 84 |
| 985249 | 84 |
| 985250 | 77 |
| 985251 | 83 |
| 985252 | 82 |
| 985253 | 82 |
| 985255 | 79 |
| 985256 | 90 |
| 985257 | 72 |
| 985258 | 93 |
| 985260 | 85 |
| 985263 | 70 |
| 985264 | 81 |
| 985265 | 71 |

TABLE 9
Reduction of human PMP22 in C22 transgenic mice, n = 3

| | PMP22 |
|---|---|
| | RNA (% |
| Compound | Control) |
| ID | RTS4579 |
| PBS | 100 |
| 684394 | 52 |
| 985156 | 87 |
| 985180 | 44 |
| 985201 | 86 |
| 985238 | 90 |
| 985263 | 102 |

TABLE 10
Reduction of human PMP22 in C22 transgenic mice, n = 1

| | PMP22 |
|---|---|
| | RNA (% |
| Compound | Control) |
| ID | RTS4579 |
| PBS | 100 |
| 955938 | 64 |
| 1083641 | 83 |
| 1083642 | 82 |
| 1083643 | 75 |
| 1083644 | 84 |
| 1083645 | 89 |
| 1083646 | 76 |
| 1083647 | 67 |
| 1083648 | 83 |
| 1083649 | 84 |

### Example 3: Effect of Modified Oligonucleotides on Human PMP22 in Transgenic Mice

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

C22 mice were divided into groups of 1-3 mice each and administered a single dose of 50 mg/kg of modified oligonucleotide by intravenous injection. A group of 1-3 mice was administered a single dose of PBS by intravenous injection. This group serves as the control group to which other groups were compared. Mice were sacrificed 17 days post treatment. The number of mice in a treatment group in each experiment is noted in each experimental table below as the “n” number. Total RNA was isolated from the sciatic nerve for analysis. Levels of human PMP22 RNA were measured by quantitative real-time RTPCR using human primer probe set RTS4579 (described herein above). Results are presented as percent human PMP22 RNA relative to PBS control, normalized to mouse cyclophilin A. Cyclophilin A was amplified using mouse primer probe set mcyclo24 (described herein above).

The values marked with the symbol “‡” indicate that the modified oligonucleotide is complementary to the amplicon region of the primer probe set. Additional assays may be used to measure the potency and efficacy of the modified oligonucleotides complementary to the amplicon region. In such instances, an additional qRTPCR assay using human primer probe set RTS35670 (forward sequence AGAAATCTGCTTGGAAGAAGGG, designated herein as SEQ ID NO: 15; reverse sequence ACGTGGAGGACGATGATACT, designated herein as SEQ ID NO: 16; probe sequence AGCAACAGGAGGAGCATTCTGGC, designated herein as SEQ ID NO: 17) was used to measure the potency and efficacy of such modified oligonucleotides.

TABLE 11
Reduction of human PMP22 in C22 transgenic mice,
n = 3, 50 mpk for 17 days

| Compound ID | PMP22 | PMP22 |
|---|---|---|
| | RNA (% | RNA (% |
| | Control) | Control) |
| | RTS4579 | RTS35670 |
| PBS | 100 | 100 |
| 1054937 | 15 | 22 |
| 938348 | 75 | 83 |
| 955955 | 50 | 62 |
| 955956 | 83 | 91 |
| 955967 | 77 | 85 |
| 955982 | 66 | 73 |
| 955983† | 85 | 88 |
| 956003 | 38 | 35 |
| 956005 | 80 | 87 |
| 956027 | 80 | 79 |
| 956034 | 82^{≠} | 86 |
| 985180 | 61 | 69 |
| 985201 | 84 | 89 |
| 985238 | 56 | 61 |

†Group had less than 3 animals at end of study

TABLE 12
Reduction of human PMP22 in C22 transgenic mice, n = 1, 50 mpk, 17 days

| Compound | PMP22 |
|---|---|
| ID | RNA (% |
| | Control) |
| | RTS4579 |
| PBS | 100 |
| 985190 | 84 |
| 1083583 | 65 |
| 1083584 | 65 |
| 1083585 | 78 |
| 1083586 | 150 |
| 1083587 | 99 |
| 1083588 | 106 |
| 1083589 | 111 |
| 1083590 | 89 |
| 1083591 | 63 |
| 1083592 | 34 |
| 1083593 | 65 |
| 1083594 | 71 |
| 1083595 | 94 |
| 1083596 | 72 |
| 1083597 | 108 |
| 1083598 | 63 |
| 1083599 | 107 |
| 1083600 | 80 |
| 1083601 | 80 |
| 1083602 | 65 |
| 1083603 | 61 |
| 1083604 | 48 |
| 1083605 | 65 |
| 1083606 | 59 |
| 1083607 | 72 |
| 1083608 | 70 |
| 1083609 | 64 |
| 1083610 | 53 |
| 1083611 | 56 |
| 1083612 | 69 |
| 1083613 | 52 |
| 1083614 | 62 |
| 1083615 | 76 |
| 1083616 | 63 |
| 1083617 | 59 |
| 1083618 | 87 |
| 1083619 | 66 |
| 1083620 | 115 |
| 1083621 | 68 |
| 1083622 | 80 |
| 1083623 | 84 |
| 1083624 | 81 |
| 1083625 | 74 |
| 1083626 | 46 |
| 1083627 | 41 |
| 1083628 | 79 |
| 1083629 | 75 |

TABLE 13
Reduction of human PMP22 in C22 transgenic mice,
n = 1, 50 mpk, 17 days

| | PMP22 |
|---|---|
| | RNA (% |
| Compound | Control) |
| ID | RTS4579 |
| PBS | 100 |
| 955938 | 80 |
| 1083641 | 108 |
| 1083643 | 84 |
| 1083644 | 69 |
| 1083645 | 86 |
| 1083646 | 102 |
| 1083647 | 89 |
| 1083648 | 76 |
| 1083649 | 91 |
| 1083630 | 120 |
| 1083631 | 106 |
| 1083632 | 122 |

TABLE 14
Reduction of human PMP22 in C22 transgenic mice,
n = 1, 50 mpk, 17 days

| | PMP22 |
|---|---|
| | RNA (% |
| Compound | Control) |
| ID | RTS4579 |
| PBS | 100 |
| 956048 | 93 |
| 985181 | 95 |
| 1089867 | 90 |
| 1089868 | 89 |
| 1089869 | 33 |
| 1089870 | 22 |
| 1089871 | 94 |
| 1089872 | 78 |
| 1089873 | 87 |
| 1089874 | 98 |
| 1089876 | 97 |
| 1089877 | 78 |
| 1089878 | 114 |
| 1089879 | 86 |
| 1089880 | 96 |
| 1089883 | 95 |
| 1089884 | 82 |
| 1089885 | 114 |
| 1089886 | 87 |
| 1089887 | 77 |

TABLE 15
Reduction of human PMP22 in C22 transgenic mice,
n = 3, 50 mpk, 17 days

| | PMP22 |
|---|---|
| | RNA (% |
| Compound | Control) |
| ID | RTS4579 |
| PBS | 100 |
| 1083584 | 76 |
| 1083592 | 66 |
| 1083593 | 94 |
| 1083594† | 75 |
| 1083595 | 82 |
| 1083596 | 94 |
| 1083597 | 67 |
| 1083598 | 81 |
| 1083599 | 81 |
| 1083600 | 94 |
| 1083601 | 93 |
| 1083602 | 93 |
| 1083603 | 85 |
| 1083604 | 48 |
| 1083605 | 45 |
| 1083606 | 84 |
| 1083607 | 92 |
| 1083608 | 100 |
| 1083609 | 84 |
| 1083610 | 92 |
| 1083611† | 91 |
| 1083612 | 38 |
| 1083613 | 86 |
| 1083615 | 97 |
| 1083616 | 106 |
| 1083621 | 90 |
| 1083624 | 85 |
| 1083627 | 78 |
| 1083629 | 99 |
| 1089869 | 31 |
| 1089870 | 17 |

†Group had fewer than 3 animals at end of study

TABLE 16
Reduction of human PMP22 in C22 transgenic mice,
n = 3, 50 mpk, 17 days

| | PMP22 |
|---|---|
| | RNA (% |
| Compound | Control) |
| ID | RTS4579 |
| PBS | 100 |
| 1083644 | 71 |
| 1083645 | 83 |
| 1083646 | 73 |
| 1083647 | 65 |
| 1083648 | 72 |
| 1089884 | 81 |
| 1089886 | 68 |
| 1089887 | 93 |

TABLE 17
Reduction of human PMP22 in C22 transgenic mice,
n = 3, 50 mpk, 17 days

| | PMP22 |
|---|---|
| | RNA (% |
| Compound | Control) |
| ID | RTS4579 |
| PBS | 100 |
| 1120849 | 73 |
| 1120853 | 91 |
| 1120854 | 98 |
| 1120863† | 84 |
| 1120894 | 80 |
| 1120895 | 90 |
| 1120896 | 87 |
| 1120897 | 89 |
| 1120898 | 90 |
| 1120899 | 93 |

†Group had fewer than 3 animals at end of study

TABLE 18
Reduction of human PMP22 in C22
transgenic mice, n = 3, 50 mpk, 17 days

| | PMP22 | PMP22 |
|---|---|---|
| | RNA (% | RNA (% |
| Compound | Control) | Control) |
| ID | RTS4579 | RTS35670 |
| PBS | 100 | 100 |
| 884288 | 85 | 99 |
| 955922† | 84 | 83 |
| 955948† | 78 | 97 |
| 955955 | 48 | 58 |
| 956003 | 86 | 89 |
| 956004† | 85‡ | 112 |
| 956055† | 91 | 110 |
| 956058† | 86 | 99 |
| 985176† | 64 | 80 |
| 985177† | 84 | 105 |
| 985180 | 45 | 62 |
| 985181 | 70 | 89 |
| 985222† | 82 | 103 |
| 1083592 | 75 | 98 |
| 1083604 | 83 | 110 |
| 1083605 | 65 | 93 |
| 1083612 | 59 | 88 |
| 1083626 | 38 | 54 |
| 1089869 | 17 | 24 |
| 1089870 | 16 | 21 |
| 1089871 | 88 | 100 |

†treatment groups of n = 1

### Example 4: Effect of cEt Modified Oligonucleotides on Human PMP22 in Transgenic Mice

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

Groups containing 1-2 C22 mice each were administered a single dose of 50 mg/kg of modified oligonucleotide by intravenous injection. A group of 1-2 C22 mice was administered a single dose of PBS by intravenous injection. This mouse serves as the control group to which other groups were compared. Mice were sacrificed 14 days post treatment. The number of mice in a treatment group in each experiment is noted in each experimental table below as the “n” number. Total RNA was isolated from the sciatic nerve for analysis. Levels of human PMP22 RNA were measured by quantitative real-time RTPCR using human primer probe set RTS4579 (described herein above). Results are presented as percent human PMP22 RNA relative to PBS control, normalized to mouse cyclophilin A. Cyclophilin A was amplified using mouse primer probe set mcyclo24 (described herein above).

TABLE 19
Reduction of human PMP22 in C22 transgenic mice,
n = 1, 50 mpk, 14 days

| | PMP22 |
|---|---|
| | RNA (% |
| Compound | Control) |
| ID | RTS4579 |
| PBS | 100 |
| 1054937 | 17 |
| 1120838 | 90 |
| 1120839 | 84 |
| 1120840 | 95 |
| 1120841 | 100 |
| 1120842 | 88 |
| 1120843 | 117 |
| 1120844 | 88 |
| 1120845 | 90 |
| 1120846 | 74 |
| 1120847 | 79 |
| 1120848 | 95 |
| 1120849 | 65 |
| 1120853 | 75 |
| 1120854 | 89 |
| 1120863 | 101 |
| 1120865 | 80 |
| 1120873 | 75 |
| 1120875 | 73 |
| 1120877 | 91 |
| 1120881 | 81 |
| 1120882 | 95 |
| 1120890 | 78 |
| 1120892 | 91 |
| 1120894 | 54 |
| 1120895 | 81 |
| 1120896 | 82 |
| 1120897 | 68 |
| 1120898 | 74 |
| 1120899 | 87 |
| 938348 | 49 |
| 955955 | 96 |
| 956003 | 77 |
| 985180 | 98 |
| 985238 | 81 |

TABLE 20
Reduction of human PMP22 in C22 transgenic mice,
n = 2, 50 mpk, 14 days

| | PMP22 |
|---|---|
| | RNA (% |
| Compound | Control) |
| ID | RTS4579 |
| PBS | 100 |
| 923867 | 42 |
| 1421291 | 33 |
| 1421292 | 46 |
| 1421296 | 16 |
| 1421314 | 25 |
| 1421315 | 21 |
| 1421318 | 61 |
| 1421329 | 58 |
| 1421336 | 77 |
| 1421341 | 66 |
| 1421345 | 57 |

### Example 5: Effect of Modified Oligonucleotides on Human PMP22 in Transgenic Mice

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

C22 mice were divided into groups of 1-3 mice each and administered a single dose of 30 mg/kg of modified oligonucleotide by intravenous injection. A group of 1-3 mice was administered a single dose of PBS by intravenous injection. This group serves as the control group to which other groups were compared. Mice were sacrificed 14 days post treatment. The number of mice in a treatment group in each experiment is noted in each experimental table below as the “n” number. Total RNA was isolated from the sciatic nerve for analysis. Levels of human PMP22 RNA were measured by quantitative real-time RTPCR using human primer probe set RTS4579 (described herein above). Results are presented as percent human PMP22 RNA relative to PBS control, normalized to mouse cyclophilin A. Cyclophilin A was amplified using mouse primer probe set mcyclo24 (described herein above).

TABLE 21
Reduction of human PMP22 in C22 transgenic mice,
n = 3, 30 mpk, 14 days

| | PMP22 |
|---|---|
| | RNA (% |
| Compound | Control) |
| ID | RTS4579 |
| PBS | 100 |
| 684394 | 73 |
| 923867 | 34 |
| 1054937 | 63 |
| 1089870 | 32 |
| 1182269 | 84 |
| 1182270 | 95 |
| 1182271† | 76 |
| 1182273 | 94 |
| 1182285 | 112 |
| 1182286† | 118 |
| 1182287 | 106 |
| 1263229 | 96 |
| 1263254 | 97 |
| 1263256 | 94 |
| 1263258 | 74 |
| 1263259 | 87 |
| 1263261 | 90 |
| 1263271 | 96 |
| 1263272 | 78 |

†Group had fewer than 3 animals at end of study

TABLE 22
Reduction of human PMP22 in C22 transgenic mice,
n = 3, 30 mpk, 14 days

| | PMP22 |
|---|---|
| | RNA (% |
| Compound | Control) |
| ID | RTS4579 |
| PBS | 100 |
| 1054937 | 62 |
| 1263275 | 104 |
| 1263277 | 101 |
| 1263280 | 106 |
| 1263281 | 108 |
| 1263282 | 104 |
| 1263304 | 96 |
| 1263305 | 117 |
| 1263308 | 121 |
| 1263312 | 135 |
| 1263332 | 129 |
| 1263334 | 132 |

TABLE 23
Reduction of human PMP22 in C22 transgenic mice,
n = 3, 30 mpk, 14 days

| | PMP22 |
|---|---|
| | RNA (% |
| Compound | Control) |
| ID | RTS4579 |
| PBS | 100 |
| 1054937 | 56 |
| 1263317 | 91 |
| 1263362 | 84 |
| 1263363 | 67 |
| 1263365 | 85 |
| 1263368 | 79 |
| 1263369 | 60 |
| 1263370 | 80 |
| 1263378 | 76 |
| 1263380 | 91 |
| 1263381 | 86 |
| 1263382 | 102 |
| 1263384 | 101 |
| 1263385 | 105 |
| 1263386 | 81 |
| 1263390 | 95 |
| 1263400 | 99 |
| 1263768 | 96 |
| 1263770 | 98 |
| 1263771 | 93 |

### Example 6: Effect of Modified Oligonucleotides on Human PMP22 in Transgenic Mice, Multiple Doses

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

C22 mice were divided into groups of 3 mice each and administered a single dose of modified oligonucleotide by intravenous injection at the doses indicated in the table below. A group of 3 mice was administered a single dose of PBS by intravenous injection. This group serves as the control group to which other groups were compared. Mice were sacrificed 14-18 days post treatment. Total RNA was isolated from the sciatic nerve for analysis. Levels of human PMP22 RNA were measured by quantitative real-time RTPCR using human primer probe set RTS4579 (described herein above). Results are presented as percent human PMP22 RNA relative to PBS control, normalized to mouse cyclophilin A. Cyclophilin A was amplified using mouse primer probe set mcyclo24 (described herein above).

TABLE 24
Reduction of human PMP22 in C22 transgenic mice,
multi-dose study, 14 days

| | | PMP22 |
|---|---|---|
| | | RNA (% |
| | Dose | Control) |
| Compound ID | (mg/kg) | RTS4579 |
| PBS | 0 | 100 |
| 955936† | 50 | 94 |
| 884288 | 50 | 90 |
| | 150 | 64 |
| 1083612 | 50 | 102 |
| | 150 | 74 |

†Group had fewer than 3 animals at end of study

TABLE 25
Reduction of human PMP22 in C22 transgenic mice,
multi-dose study, 18 days

| | | PMP22 | |
|---|---|---|---|
| Compound | Dose | % control | |
| ID | (mg/kg) | RTS4579 | ED50 |
| PBS | 0 | 100 | |
| 684394 | 1.9 | 104 | 127 |
| | 5.6 | 91 | |
| | 16.7 | 92 | |
| | 50 | 74 | |
| | 150 | 45 | |
| 1054937 | 1.9 | 94 | 28 |
| | 5.6 | 95 | |
| | 16.7 | 76 | |
| | 50 | 29 | |
| | 150 | 10 | |

### Example 7: Tolerability of Modified Oligonucleotides Targeting Human PMP22 in Balb/c Mice

Balb/c mice are a multipurpose mouse model frequently utilized for safety and efficacy testing. The mice were treated with modified oligonucleotides selected from studies described above and evaluated for changes in the levels of various plasma chemistry markers.

Groups of 2-3 female Balb/c mice were injected subcutaneously with a single dose of 150 mg/kg of modified oligonucleotides. One group of 2-4 female CD-1 mice was injected with PBS. Mice were euthanized 72-96 hours following treatment. The number of mice in a treatment group in each experiment is noted in each experimental table below as the “n” number.

To evaluate the effect of modified oligonucleotides on liver and kidney function, plasma levels of aspartate aminotransferase (AST), alanine aminotmnsferase (ALT) and blood urea nitrogen (BUN) were measured using an automated clinical chemistry analyzer (Hitachi Olympus AU400c, Melville, NY). The results are presented in the table below. Each table represents results from an individual experiment.

TABLE 26
Plasma chemistry markers in female Balb/c mice, n = 2, 72 hrs

| | | Plasma clinical chemistry | | |
|---|---|---|---|---|
| Compound | Dose | ALT | AST | BUN |
| No. | (mpk) | (U/L) | (U/L) | (mg/dL) |
| PBS | | 39 | 136 | 22 |
| 1054937† | 150 | 779 | 1096 | 15 |
| 1182269 | 150 | 42 | 138 | 22 |
| 1182270 | 150 | 47 | 102 | 18 |
| 1182271 | 150 | 23 | 112 | 21 |
| 1182273 | 150 | 31 | 109 | 23 |
| 884288† | 150 | 1476 | 1178 | 20 |
| 1089870† | 150 | 313 | 536 | 15 |
| 1182285 | 150 | 32 | 73 | 23 |
| 1182287 | 150 | 57 | 121 | 21 |
| 923867^{\|} | 150 | 18 | 80 | 16 |

†groups with n = 3

TABLE 27
Plasma chemistry markers in female Balb/c mice, n = 2, 96 hrs

| | | Plasma clinical chemistry | | |
|---|---|---|---|---|
| Compound | Dose | ALT | AST | BUN |
| No. | (mpk) | (U/L) | (U/L) | (mg/dL) |
| PBS | 0 | 38 | 60 | 18 |
| 1263229 | 150 | 54 | 104 | 20 |
| 1263254 | 150 | 31 | 46 | 23 |
| 1263256 | 150 | 44 | 62 | 23 |
| 1263258 | 150 | 82 | 80 | 18 |
| 1263259 | 150 | 25 | 38 | 23 |
| 1263261 | 150 | 56 | 92 | 21 |
| 1263271 | 150 | 37 | 58 | 17 |
| 1263272 | 150 | 38 | 74 | 20 |

TABLE 28
Plasma chemistry markers in female Balb/c mice, n = 2, 96 hrs

| | | Plasma clinical chemistry | | |
|---|---|---|---|---|
| Compound | Dose | ALT | AST | BUN |
| No. | (mpk) | (U/L) | (U/L) | (mg/dL) |
| PBS | 0 | 37 | 80 | 24 |
| 1263275 | 150 | 42 | 109 | 22 |
| 1263277 | 150 | 42 | 79 | 28 |
| 1263280 | 150 | 36 | 93 | 21 |
| 1263281 | 150 | 24 | 76 | 22 |
| 1263282 | 150 | 89 | 115 | 28 |
| 1263304 | 150 | 50 | 100 | 25 |
| 1263305 | 150 | 92 | 189 | 30 |
| 1263308 | 150 | 33 | 93 | 20 |
| 1263312 | 150 | 50 | 105 | 24 |

TABLE 29
Plasma chemistry markers in female Balb/c mice, n = 2, 96 hrs

| | | Plasma clinical chemistry | | |
|---|---|---|---|---|
| Compound | Dose | ALT | AST | BUN |
| No. | (mpk) | (U/L) | (U/L) | (mg/dL) |
| PBS | 0 | 25 | 39 | 20 |
| 1263332 | 150 | 84 | 81 | 22 |
| 1263334 | 150 | 28 | 38 | 22 |
| 1263362 | 150 | 139 | 85 | 27 |
| 1263363 | 150 | 74 | 109 | 19 |
| 1263365 | 150 | 26 | 43 | 18 |
| 1263368 | 150 | 27 | 28 | 17 |
| 1263369 | 150 | 33 | 43 | 18 |
| 1263370 | 150 | 52 | 56 | 20 |

TABLE 30
Plasma chemistry markers in female Balb/c mice, n = 2, 96 hrs

Plasma clinical chemistry

| Compound | Dose | ALT | AST |
|---|---|---|---|
| No. | (mpk) | (U/L) | (U/L) |
| PBS | 0 | 31 | 59 |
| 1263317 | 150 | 39 | 74 |
| 1263378 | 150 | 35 | 71 |
| 1263380 | 150 | 40 | 103 |
| 1263381 | 150 | 70 | 81 |
| 1263382 | 150 | 45 | 68 |
| 1263384 | 150 | 23 | 44 |
| 1263385 | 150 | 33 | 55 |
| 1263386 | 150 | 106 | 133 |
| 1263390 | 150 | 25 | 63 |
| 1263400 | 150 | 21 | 52 |
| 1263768 | 150 | 13 | 49 |
| 1263770 | 150 | 35 | 74 |
| 1263771 | 150 | 55 | 110 |

TABLE 31
Plasma chemistry markers in female Balb/c mice, n = 2, 96 hrs

Plasma clinical chemistry

| Compound | Dose | ALT | AST |
|---|---|---|---|
| No. | (mpk) | (U/L) | (U/L) |
| PBS | 0 | 29 | 67 |
| 1421291 | 150 | 266 | 352 |
| 1421292 | 150 | 283 | 460 |
| 1421296 | 150 | 168 | 226 |
| 1421314 | 150 | 102 | 361 |
| 1421315 | 150 | 153 | 237 |
| 1421318 | 150 | 60 | 164 |
| 1421329 | 150 | 169 | 326 |
| 1421336 | 150 | 195 | 320 |
| 1421341 | 150 | 99 | 135 |
| 1421345 | 150 | 144 | 244 |

### Example 8: Design of Modified Oligonucleotides Complementary to a Human PMP22 Nucleic Acid

Modified oligonucleotides complementary to a human PMP22 nucleic acid were designed, as described in the tables below.

The modified oligonucleotides in Table 32 are 3-10-3 cEt gapmers with phosphorothioate internucleoside linkages. The gapmers are 16 nucleosides in length, wherein the central gap segment consists of ten 2′-β-D-deoxynucleosides and the 5′ and 3′ wings each consists of three cEt nucleosides. The motif for the gapmers is (from 5′ to 3′): kkkddddddddddkkk; wherein ‘d’ represents a 2′-β-D-deoxyribosyl sugar moiety, and ‘k’ represents a cEt sugar moiety. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the Tables below is 100% complementary to SEQ ID NO: 1 (GENBANK Accession No. NM_000304.3), or SEQ ID NO: 2 (GENBANK Accession No. NC_000017.11 truncated from nucleotides 15227001 to 15268000). ‘N/A’ indicates that the modified oligonucleotide is not 100% complementary to that particular target nucleic acid sequence.

TABLE 32
3-10-3 cEt gapmers with PS internucleoside linkages complementary to human PMP22

| | SEQ ID | SEQ ID | SEQ ID | SEQ ID | | |
|---|---|---|---|---|---|---|
| | NO: 1 | NO: 1 | NO: 2 | NO: 2 | | |
| Compound | Start | Stop | Start | Stop | | SEQ ID |
| ID | Site | Site | Site | Site | Sequence (5′ to 3′) | NO |
| 1078137 | 1494 | 1509 | 37857 | 37872 | ACAGAATTATTCAGGT | 193 |
| 1078138 | 1495 | 1510 | 37858 | 37873 | CACAGAATTATTCAGG | 194 |
| 1078139 | 1496 | 1511 | 37859 | 37874 | ACACAGAATTATTCAG | 195 |
| 1078141 | 1498 | 1513 | 37861 | 37876 | TTACACAGAATTATTC | 196 |
| 1078143 | 1500 | 1515 | 37863 | 37878 | TATTACACAGAATTAT | 197 |
| 1078145 | 1502 | 1517 | 37865 | 37880 | TATATTACACAGAATT | 199 |
| 1078146 | 1503 | 1518 | 37866 | 37881 | TTATATTACACAGAAT | 200 |
| 1078147 | 1504 | 1519 | 37867 | 37882 | TTTATATTACACAGAA | 201 |
| 1078148 | 1505 | 1520 | 37868 | 37883 | ATTTATATTACACAGA | 202 |
| 1078149 | 1506 | 1521 | 37869 | 37884 | CATTTATATTACACAG | 203 |
| 1078150 | 1507 | 1522 | 37870 | 37885 | CCATTTATATTACACA | 204 |
| 1078151 | 1508 | 1523 | 37871 | 37886 | ACCATTTATATTACAC | 205 |
| 1078161 | 1473 | 1488 | 37836 | 37851 | TTCTATCTTATGTTGT | 207 |
| 1078162 | 1470 | 1485 | 37833 | 37848 | TATCTTATGTTGTAAA | 208 |
| 1078163 | 1469 | 1484 | 37832 | 37847 | ATCTTATGTTGTAAAA | 209 |
| 1078911 | N/A | N/A | 31459 | 31474 | TTGTAGATTTCACATC | 210 |
| 1078921 | N/A | N/A | 31455 | 31470 | AGATTTCACATCCCAT | 211 |
| 1078924 | N/A | N/A | 31452 | 31467 | TTTCACATCCCATGAG | 212 |
| 1078977 | N/A | N/A | 23202 | 23217 | AAATTGATGTCAGTGG | 213 |
| 1078979 | N/A | N/A | 23204 | 23219 | TCAAATTGATGTCAGT | 214 |
| 1078980 | N/A | N/A | 23205 | 23220 | GTCAAATTGATGTCAG | 215 |
| 1079047 | N/A | N/A | 19086 | 19101 | AAAATGACGGGAAAGG | 216 |
| 1079048 | N/A | N/A | 19087 | 19102 | GAAAATGACGGGAAAG | 217 |
| 1079049 | N/A | N/A | 19088 | 19103 | AGAAAATGACGGGAAA | 218 |
| 1079052 | N/A | N/A | 19078 | 19093 | GGGAAAGGCAGTTGCA | 221 |
| 1079053 | N/A | N/A | 19077 | 19092 | GGAAAGGCAGTTGCAA | 222 |
| 1079093 | N/A | N/A | 28698 | 28713 | TTCTAAGACACATACA | 223 |
| 1079094 | N/A | N/A | 28697 | 28712 | TCTAAGACACATACAG | 224 |
| 1079095 | N/A | N/A | 28696 | 28711 | CTAAGACACATACAGG | 225 |
| 1079096 | N/A | N/A | 28695 | 28710 | TAAGACACATACAGGT | 226 |

Modified oligonucleotides complementary to a human PMP22 nucleic acid were designed, as described in Table 33 below. The modified oligonucleotides in Table 33 are 16-mer gapmers with mixed sugar motifs as indicated in the table below, wherein ‘d’ represents a 2′-β-D-deoxyribosyl sugar moiety; ‘e’ represents a 2′-MOE sugar moiety, ‘k’ represents a cEt sugar moiety; and ‘y’ represents a 2′-OMe sugar moiety. All internucleoside linkages are phosphorothioate internucleoside linkages. Each cytosine residue is a 5-methylcytosine, unless indicated by a bold underlined ‘C’, in which case, the cytosine is not methylated.

TABLE 33
Modified oligonucleotide gapmers with mixed sugar moieties and uniform PS internucleoside
linkages complementary to human PMP22

| | SEQ ID | SEQ ID | SEQ ID | SEQ ID | | | |
|---|---|---|---|---|---|---|---|
| | NO: 1 | NO: 1 | NO: 2 | NO: 2 | | | |
| Compound | Start | Stop | Start | Stop | | Sugar Motif | SEQ ID |
| ID | Site | Site | Site | Site | Sequence (5′ to 3′) | (5′ to 3′) | NO |
| 1421217 | N/A | N/A | 9499 | 9514 | AAATACGATCTTCTGG | eekkddddddddkkee | 239 |
| 1421216 | N/A | N/A | 9498 | 9513 | AATACGATCTTCTGGA | eekkdddddddkdkee | 238 |
| 1421268 | 1489 | 1504 | 37852 | 37867 | ATTATTCAGGTCTCCA | ekkddddddddddkke | 19 |
| 1421270 | N/A | N/A | 9498 | 9513 | AATACGATCTTCTGGA | ekkddddddddddkke | 238 |
| 1421271 | N/A | N/A | 9499 | 9514 | AAATACGATCTTCTGG | ekkddddddddddkke | 239 |
| 1421219 | 1489 | 1504 | 37852 | 37867 | ATTATTCAGGTCTCCA | ekkddddddddddkkk | 19 |
| 1421221 | N/A | N/A | 9498 | 9513 | AATACGATCTTCTGGA | ekkddddddddddkkk | 238 |
| 1421222 | N/A | N/A | 9499 | 9514 | AAATACGATCTTCTGG | ekkddddddddddkkk | 239 |
| 1421274 | N/A | N/A | 9498 | 9513 | AATACGATCTTCTGGA | ekkkdddddddddkkk | 238 |
| 1421275 | N/A | N/A | 9499 | 9514 | AAATACGATCTTCTGG | ekkkdddddddddkkk | 239 |
| 1421223 | 1489 | 1504 | 37852 | 37867 | ATTATTCAGGTCTCCA | kekddddddddddkkk | 19 |
| 1421225 | N/A | N/A | 9498 | 9513 | AATACGATCTTCTGGA | kekddddddddddkkk | 238 |
| 1421226 | N/A | N/A | 9499 | 9514 | AAATACGATCTTCTGG | kekddddddddddkkk | 239 |
| 1421235 | N/A | N/A | 19079 | 19094 | CGGGAAAGGCAGTTGC | kkeddddddddddkkk | 220 |
| 1421236 | N/A | N/A | 9498 | 9513 | AATACGATCTTCTGGA | kkeddddddddddkkk | 238 |
| 1421237 | N/A | N/A | 9499 | 9514 | AAATACGATCTTCTGG | kkeddddddddddkkk | 239 |
| 1421240 | N/A | N/A | 9498 | 9513 | AATACGATCTTCTGGA | kkkddddddddddekk | 238 |
| 1421241 | N/A | N/A | 9499 | 9514 | AAATACGATCTTCTGG | kkkddddddddddekk | 239 |
| 1421244 | N/A | N/A | 9498 | 9513 | AATACGATCTTCTGGA | kkkddddddddddkek | 238 |
| 1421245 | N/A | N/A | 9499 | 9514 | AAATACGATCTTCTGG | kkkddddddddddkek | 239 |
| 1421266 | N/A | N/A | 9498 | 9513 | AATACGATCTTCTGGA | kkkddddddddddkke | 238 |
| 1421267 | N/A | N/A | 9499 | 9514 | AAATACGATCTTCTGG | kkkddddddddddkke | 239 |
| 1421284 | 1489 | 1504 | 37852 | 37867 | ATTATTCAGGTCTCCA | kkkdddddddddekkk | 19 |
| 1421285 | N/A | N/A | 19079 | 19094 | CGGGAAAGGCAGTTGC | kkkdddddddddekkk | 220 |
| 1421286 | N/A | N/A | 9498 | 9513 | AATACGATCTTCTGGA | kkkdddddddddekkk | 238 |
| 1421287 | N/A | N/A | 9499 | 9514 | AAATACGATCTTCTGG | kkkdddddddddekkk | 239 |
| 1421276 | 1489 | 1504 | 37852 | 37867 | ATTATTCAGGTCTCCA | kkkdddddddddkkke | 19 |
| 1421278 | N/A | N/A | 9498 | 9513 | AATACGATCTTCTGGA | kkkdddddddddkkke | 238 |
| 1421279 | N/A | N/A | 9499 | 9514 | AAATACGATCTTCTGG | kkkdddddddddkkke | 239 |
| 1421133 | N/A | N/A | 9498 | 9513 | AATACGATCTTCTGGA | kkkdyddddddddkkk | 238 |
| 1421134 | N/A | N/A | 9499 | 9514 | AAATACGATCTTCTGG | kkkdyddddddddkkk | 239 |
| 1421280 | 1489 | 1504 | 37852 | 37867 | ATTATTCAGGTCTCCA | kkkedddddddddkkk | 19 |
| 1421281 | N/A | N/A | 19079 | 19094 | CGGGAAAGGCAGTTGC | kkkedddddddddkkk | 220 |
| 1421282 | N/A | N/A | 9498 | 9513 | AATACGATCTTCTGGA | kkkedddddddddkkk | 238 |
| 1421283 | N/A | N/A | 9499 | 9514 | AAATACGATCTTCTGG | kkkedddddddddkkk | 239 |

### Example 9: Effect of Modified Oligonucleotides on Human PMP22 in Transgenic Mice

C22 mice, described in Huxley et al., Human Molecular Genetics, 5, 563-569 (1996) and Verhamme et al., Journal of Neuropathology and Experimental Neurology, 70, 386-398 (2011), express endogenous mouse PMP22 and overexpress a human PMP22 transgene. The effect of modified oligonucleotides on human PMP22 RNA was tested in symptomatic C22 mice.

C22 mice were divided into groups of 1-2 mice each and administered a single dose of 50 mg/kg of modified oligonucleotide by intravenous injection as indicated in the tables below. A group of 1-3 mice was administered a single dose of PBS by intravenous injection. This group serves as the control group to which other groups were compared. Mice were sacrificed 14-17 days post treatment. The number of mice in a treatment group in each experiment is noted in each experimental table below as the “n” number. Total RNA was isolated from the sciatic nerve for analysis. Levels of human PMP22 RNA were measured by quantitative real-time RTPCR using human primer probe set RTS4579 (forward sequence CTTGCTGGTCTGTGCGTGAT, designated herein as SEQ ID NO: 9; reverse sequence ACCGTAGGAGTAATCCGAGTTGAG, designated herein as SEQ ID NO: 10; probe sequence CATCTACACGGTGAGGCACCCGG, designated herein as SEQ ID NO: 11). Data were normalized to the control group and are presented in the table below.

TABLE 34
Reduction of human PMP22 in C22
transgenic mice, n = 1, 17 days

| | PMP22 RNA |
|---|---|
| | (% Control) |
| Compound ID | RTS4579 |
| PBS | 100 |
| 1078137 | 123 |
| 1078138 | 117 |
| 1078139 | 116 |
| 1078141 | 90 |
| 1078143 | 100 |
| 1078145 | 123 |
| 1078146 | 173 |
| 1078147 | 112 |
| 1078148 | 131 |
| 1078149 | 103 |
| 1078150 | 107 |
| 1078151 | 112 |
| 1078161 | 105 |
| 1078162 | 116 |
| 1078163 | 95 |
| 1078911 | 101 |
| 1078921 | 86 |
| 1078924 | 94 |
| 1078977 | 99 |
| 1078979 | 113 |
| 1078980 | 108 |
| 1079047 | 105 |
| 1079048 | 197 |
| 1079049 | 120 |
| 1079053 | 171 |
| 1079093 | 87 |
| 1079094 | 103 |
| 1079095 | 82 |
| 1079096 | 106 |
| 1079052 | 103 |

TABLE 35
Reduction of human PMP22 in C22
transgenic mice, n = 2, 14 days

| | PMP22 RNA |
|---|---|
| | (% Control) |
| Compound ID | RTS4579 |
| PBS | 100 |
| 1421133 | 106 |
| 1421134 | 106 |
| 1421216 | 92 |
| 1421217 | 95 |
| 1421219 | 103 |
| 1421221 | 80 |
| 1421222 | 71 |
| 1421223 | 86 |
| 1421225 | 77 |
| 1421226 | 68 |
| 1421235 | 115 |
| 1421236 | 76 |
| 1421237 | 45 |
| 1421240 | 71 |
| 1421241 | 62 |
| 1421244 | 71 |
| 1421245 | 63 |
| 1421266 | 70 |
| 1421267 | 49 |
| 1421268 | 86 |
| 1421270 | 86 |
| 1421271 | 83 |
| 1421274 | 100 |
| 1421275 | 50 |
| 1421276 | 91 |
| 1421278 | 71 |
| 1421279 | 66 |
| 1421280 | 90 |
| 1421281 | 85 |
| 1421282 | 93 |
| 1421283 | 36 |
| 1421284 | 63 |
| 1421285 | 91 |
| 1421286 | 41 |
| 1421287 | 39 |

### Example 10: Design of Modified Oligonucleotides Complementary to a Human PMP22 Nucleic Acid

Modified oligonucleotides complementary to a human PMP22 nucleic acid were designed and synthesized. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO:2 (described herein above).

The modified oligonucleotides in the table below are 16 nucleosides in length. The sugar motif for the modified oligonucleotides are described in the column labeled “Sugar Motif (5′ to 3′)” in the table below, wherein each “d” represents a 2′-β-D-deoxyribosyl sugar moiety, each “k” represents a cEt sugar moiety, and each “e” represents a 2′-MOE sugar moiety. The internucleoside linkage motifs for the modified oligonucleotides are described in the column labeled “Internucleoside Linkage Motif (5′ to 3′)” in the table below, wherein each “s” represents a phosphorothioate internucleoside linkage and each “z” represents a mesyl phosphoramidate internucleoside linkage. Each cytosine residue is a 5-methylcytosine.

The modified oligonucleotides in the table below are conjugated to a 6-palmitamidohexyl phosphate conjugate group attached to the 5′-OH of the oligonucleotide. The structure for the conjugate group is:

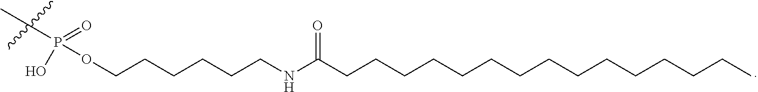 [SMILES: *P(=O)(O)OCCCCCCNC(=O)CCCCCCCCCCCCCCC]

The modified oligonucleotides in the table below are 16 nucleosides in length. The sugar motifs for the modified oligonucleotides are described in the column labeled “Sugar Motif (5′ to 3′)” in the table below, wherein each “d” represents a 2′-β-D-deoxyribosyl sugar moiety, each “k” represents a cEt sugar moiety, and each “e” represents a 2′-MOE sugar moiety. The intemucleoside linkage motifs for the modified oligonucleotides are described in the column labeled “Intemucleoside Linkage Motif (5′ to 3′)” in the table below, wherein each “s” represents a phosphorothioate intemucleoside linkage, and each “z” represents a mesyl phosphoramidate internucleoside linkage. Each cytosine residue is a 5-methylcytosine.

TABLE 36
6-palmitamidohexyl phosphate conjugated 3-10-3 cEt or mixed cEt/MOE modified oligonucleotides
with either uniform PS or mixed backbone internucleoside linkages complementary to human PMP22

| | | SEQ ID | SEQ ID | | | |
|---|---|---|---|---|---|---|
| | | NO: 2 | NO: 2 | | Internucleoside | |
| Compound | Antisense Sequence | Antisense | Antisense | Sugar Motif | Linkage Motif | SEQ ID |
| No. | (5′ to 3′) | Start Site | Stop Site | (5′ to 3′) | (5′ to 3′) | NO. |
| 1089870 | AAATACGATCTTCT | 9499 | 9514 | kkkdddddddddd | sssssssssssssss | 239 |
| | GG | | | kkk | | |
| 1421315 | AAATACGATCTTCT | 9499 | 9514 | kkedddddddddd | sssssssssssssss | 239 |
| | GG | | | kkk | | |
| 1421353 | AAATACGATCTTCT | 9499 | 9514 | kkkddddddddde | sssssssssssssss | 239 |
| | GG | | | kkk | | |

### Example 11: Effect of Modified Oligonucleotides on Human PMP22 RNA In Vitro, Multiple Doses

Modified oligonucleotides selected from the example above were tested at various doses in A431 cells. Cultured A431 cells at a density of 10,000 cells per well were treated by free uptake with various concentrations of modified oligonucleotides specified in the table below. After a treatment period of approximately 72 hours, RNA was isolated from the cells and PMP22 RNA levels were measured by quantitative real-time RTPCR. Human primer probe set RTS4579 (described herein above) was used to measure RNA levels. PMP22 RNA levels were normalized to total RNA content, as measured by GAPDH (forward sequence GAAGGTGAAGGTCGGAGTC, designated herein as SEQ ID NO: 946; reverse sequence GAAGATGGTGATGGGATTTC, designated herein as SEQ ID NO: 947; probe sequence CAAGCTTCCCGTTCTCAGCCX, designated herein as SEQ ID NO: 948). Results are presented as percent PMP22 RNA relative to the amount in untreated control cells (% UTC).

TABLE 37
Dose-dependent reduction of human PMP22 RNA
in A431 cells by modified oligonucleotides

| Compound | Concentration | PMP22 | IC50 |
|---|---|---|---|
| No. | (nM) | (% UTC) | (μM) |
| 923867 | 20000 | 5 | 0.11 |
| | 8000 | 5 | |
| | 3200 | 6 | |
| | 1280 | 9 | |
| | 512 | 17 | |
| | 205 | 33 | |
| | 82 | 56 | |
| | 33 | 81 | |
| | 13 | 89 | |
| | 5 | 96 | |
| 1089870 | 20000 | 5 | 0.20 |
| | 8000 | 7 | |
| | 3200 | 11 | |
| | 1280 | 18 | |
| | 512 | 23 | |
| | 205 | 45 | |
| | 82 | 70 | |
| | 33 | 94 | |
| | 13 | 101 | |
| | 5 | 99 | |
| 1421315 | 20000 | 7 | 0.15 |
| | 8000 | 10 | |
| | 3200 | 13 | |
| | 1280 | 17 | |
| | 512 | 25 | |
| | 205 | 42 | |
| | 82 | 62 | |
| | 33 | 78 | |
| | 13 | 82 | |
| | 5 | 91 | |
| 1421353 | 20000 | 3 | 0.14 |
| | 8000 | 7 | |
| | 3200 | 9 | |
| | 1280 | 15 | |
| | 512 | 20 | |
| | 205 | 38 | |
| | 82 | 73 | |
| | 33 | 80 | |
| | 13 | 84 | |
| | 5 | 98 | |

### Example 12: Design of RNAi Compounds with Antisense RNAi Oligonucleotides Complementary to a Human PMP22 Nucleic Acid

RNAi compounds comprising antisense RNAi oligonucleotides complementary to a human PMP22 nucleic acid and sense RNAi oligonucleotides complementary to the antisense RNAi oligonucleotides were designed as follows.

“Start site” indicates the 5′-most nucleoside to which the antisense RNAi oligonucleotides is complementary in the human gene sequence. “Stop site” indicates the 3′-most nucleoside to which the antisense RNAi oligonucleotide is complementary in the human gene sequence. Each modified antisense RNAi oligonucleoside listed in the tables below is 100% complementary to SEQ ID NO: 1 (described herein above).

The RNAi compounds in the tables below consist of an antisense RNAi oligonucleotide and a sense RNAi oligonucleotide. In the table below, each antisense RNAi oligonucleotide is 23 nucleosides in length; has a sugar motif (from 5′ to 3′) of: yfyfyfyfyfyfyfyfyfyfyyy, wherein each “y” represents a 2′-O-Me sugar moiety, and each “f” represents a 2′-F sugar moiety; and has an intemucleoside linkage motif (from 5′ to 3′) of: ssooooooooooooooooooss, wherein each “o” represents a phosphodiester intemucleoside linkage, and each “s” represents a phosphorothioate intemucleoside linkage. Each sense RNAi oligonucleotide in the table below is 21 nucleosides in length; has a sugar motif (from 5′ to 3′) of: fyfyfyfyfyfyfyfyfyfyf, wherein each “y” represents a 2′-O—OMe sugar moiety, and each “f” represents a 2′-F sugar moiety; and has an intemucleoside linkage motif (from 5′ to 3′) of: ssooooooooooooooooss, wherein each “o” represents a phosphodiester intemucleoside linkage, and each “s” represents a phosphorothioate intemucleoside linkage. Each antisense RNAi oligonucleotide is complementary to the target nucleic acid (PMP22), and each sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 38
RNAi compounds targeting human PMP22 SEQ ID NO: 1

| | | | | SEQ ID | SEQ ID | | | |
|---|---|---|---|---|---|---|---|---|
| | | | | NO: 1 | NO: 1 | | | |
| | | Antisense | SEQ | Antisense | Antisense | | Sense | SEQ |
| Compound | Antisense | Sequence | ID | Start | Stop | Sense | Sequence | ID |
| Number | ID | (5′ to 3′) | NO. | Site | Site | ID | (5′ to 3′) | NO. |
| 1579592 | 1579604 | CAAGAUCACAUA | 322 | 684 | 706 | 1579603 | GGUGUCAUCUA | 633 |
| | | GAUGACACCGC | | | | | UGUGAUCUUG | |
| 1579593 | 1579609 | GACACCGCUGAG | 323 | 669 | 691 | 1579602 | CUGGCCCUUCU | 634 |
| | | AAGGGCCAGGG | | | | | CAGCGGUGUC | |
| 1579594 | 1579606 | GGCCAGGGGGAA | 324 | 654 | 676 | 1579598 | UGGGUGGCCUU | 635 |
| | | GGCCACCCAGG | | | | | CCCCCUGGCC | |
| 1579595 | 1579608 | CACCCAGGCCAG | 325 | 639 | 661 | 1579600 | GCCUACAUCCU | 636 |
| | | GAUGUAGGCGA | | | | | GGCCUGGGUG | |
| 1579596 | 1579605 | GAGAUGCCACUC | 326 | 594 | 616 | 1579599 | AGGCACCCGGA | 637 |
| | | CGGGUGCCUCA | | | | | GUGGCAUCUC | |
| 1579597 | 1579607 | UCAUUCGCGUUU | 327 | 699 | 721 | 1579601 | AUCUUGCGGAA | 638 |
| | | CCGCAAGAUCA | | | | | ACGCGAAUGA | |
| 1579610 | 1579627 | GUAGGCGAAACC | 328 | 624 | 646 | 1579619 | UACUCCUACGG | 639 |
| | | GUAGGAGUAAU | | | | | UUUCGCCUAC | |
| 1579611 | 1579622 | AGCAAGAAUUUG | 329 | 534 | 556 | 1579616 | GGAAUCUUCCA | 640 |
| | | GAAGAUUCCAG | | | | | AAUUCUUGCU | |
| 1579612 | 1579626 | CAUCACGCACAG | 330 | 549 | 571 | 1579617 | CUUGCUGGUCU | 641 |
| | | ACCAGCAAGAA | | | | | GUGCGUGAUG | |
| 1579613 | 1579623 | GAUGGCCGCAGC | 331 | 564 | 586 | 1579620 | GUGAUGAGUGC | 642 |
| | | ACUCAUCACGC | | | | | UGCGGCCAUC | |
| 1579614 | 1579625 | GUGCCUCACCGU | 332 | 579 | 601 | 1579618 | GCCAUCUACAC | 643 |
| | | GUAGAUGGCCG | | | | | GGUGAGGCAC | |
| 1579615 | 1579624 | GGAGUAAUCCGA | 333 | 609 | 631 | 1579621 | CAUCUCAACUC | 644 |
| | | GUUGAGAUGCC | | | | | GGAUUACUCC | |
| 1579628 | 1579643 | GAUUCCAGUGAU | 334 | 519 | 541 | 1579636 | AGGUUUUACAU | 645 |
| | | GUAAAACCUGC | | | | | CACUGGAAUC | |
| 1579629 | 1579640 | GCUGAAGAUGAU | 335 | 444 | 466 | 1579635 | AUCCUGUCGAU | 646 |
| | | CGACAGGAUCA | | | | | CAUCUUCAGC | |
| 1579630 | 1579641 | GAACAGAGACAG | 336 | 459 | 481 | 1579634 | UUCAGCAUUCU | 647 |
| | | AAUGCUGAAGA | | | | | GUCUCUGUUC | |
| 1579631 | 1579645 | UUGGCAGAAGAA | 337 | 474 | 496 | 1579638 | CUGUUCCUGUU | 648 |
| | | CAGGAACAGAG | | | | | CUUCUGCCAA | |
| 1579632 | 1579644 | GGUGAGGGUGAA | 338 | 489 | 511 | 1579639 | UGCCAACUCUU | 649 |
| | | GAGUUGGCAGA | | | | | CACCCUCACC | |
| 1579633 | 1579642 | AAACCUGCCCCC | 339 | 504 | 526 | 1579637 | CUCACCAAGGG | 650 |
| | | CUUGGUGAGGG | | | | | GGGCAGGUUU | |
| 1579646 | 1579659 | CAGGAUCAUGGU | 340 | 429 | 451 | 1579656 | GUCCAGGCCAC | 651 |
| | | GGCCUGGACAG | | | | | CAUGAUCCUG | |
| 1579647 | 1579663 | GGAAGAGGUGCU | 341 | 354 | 376 | 1579653 | CAGAACUGUAG | 652 |
| | | ACAGUUCUGCC | | | | | CACCUCUUCC | |
| 1579648 | 1579661 | GUGGACAUUUCC | 342 | 369 | 391 | 1579654 | UCUUCCUCAGG | 653 |
| | | UGAGGAAGAGG | | | | | AAAUGUCCAC | |
| 1579649 | 1579660 | CUGGACAGACUG | 343 | 414 | 436 | 1579657 | GAAUGGCUGCA | 654 |
| | | CAGCCAUUCGU | | | | | GUCUGUCCAG | |
| 1579650 | 1579658 | UGAUGAGAAACA | 344 | 384 | 406 | 1579652 | GUCCACCACUG | 655 |
| | | GUGGUGGACAU | | | | | UUUCUCAUCA | |
| 1579651 | 1579662 | CCAUUCGUUUGG | 345 | 399 | 421 | 1579655 | UCAUCAUCACC | 656 |
| | | UGAUGAUGAGA | | | | | AAACGAAUGG | |
| 1579664 | 1579677 | GUUCUGCCAGAG | 346 | 339 | 361 | 1579670 | GCAACUGAUCU | 657 |
| | | AUCAGUUGCGU | | | | | CUGGCAGAAC | |
| 1579665 | 1579676 | GCUGACGAUCGU | 347 | 294 | 316 | 1579671 | UUCGUCUCCAC | 658 |
| | | GGAGACGAACA | | | | | GAUCGUCAGC | |
| 1579666 | 1579680 | GCCCACGAUCCA | 348 | 309 | 331 | 1579673 | GUCAGCCAAUG | 659 |
| | | UUGGCUGACGA | | | | | GAUCGUGGGC | |
| 1579667 | 1579679 | AGUUGCGUGUCC | 349 | 324 | 346 | 1579672 | GUGGGCAAUGG | 660 |
| | | AUUGCCCACGA | | | | | ACACGCAACU | |
| 1579668 | 1579678 | CAGCACCGCGAC | 350 | 264 | 286 | 1579674 | GUCCUCCACGU | 661 |
| | | GUGGAGGACGA | | | | | CGCGGUGCUG | |
| 1579669 | 1579681 | GACGAACAGCAG | 351 | 279 | 301 | 1579675 | GUGCUGGUGCU | 662 |
| | | CACCAGCACCG | | | | | GCUGUUCGUC | |
| 1579682 | 1579694 | GAGGACGAUGAU | 352 | 249 | 271 | 1579690 | UUGCUGAGUAU | 663 |
| | | ACUCAGCAACA | | | | | CAUCGUCCUC | |
| 1579683 | 1579695 | AUGUUUAUUUUA | 353 | 1825 | 1847 | 1579688 | AAGAUGCAUUA | 664 |
| | | AUGCAUCUUAG | | | | | AAAUAAACAU | |
| 1579684 | 1579696 | UCUGAUGUUUAU | 354 | 1829 | 1851 | 1579689 | UGCAUUAAAAU | 665 |
| | | UUUAAUGCAUC | | | | | AAACAUCAGA | |
| 1579685 | 1579698 | UGAGUUACUCUG | 355 | 1837 | 1859 | 1579693 | AAUAAACAUCA | 666 |
| | | AUGUUUAUUUU | | | | | GAGUAACUCA | |
| 1579686 | 1579697 | UUACUCUGAUGU | 356 | 1833 | 1855 | 1579691 | UUAAAAUAAAC | 667 |
| | | UUAUUUUAAUG | | | | | AUCAGAGUAA | |
| 1579687 | 1579699 | UUAUUUUAAUGC | 357 | 1821 | 1843 | 1579692 | GACUAAGAUGC | 668 |
| | | AUCUUAGUCCA | | | | | AUUAAAAUAA | |
| 1579700 | 1579711 | UUUAAUGCAUCU | 358 | 1817 | 1839 | 1579704 | UGUGGACUAAG | 669 |
| | | UAGUCCACACA | | | | | AUGCAUUAAA | |
| 1579701 | 1579717 | UAGUCCACACAG | 359 | 1805 | 1827 | 1579708 | UUAUACCAACU | 670 |
| | | UUGGUAUAAAA | | | | | GUGUGGACUA | |
| 1579702 | 1579714 | CCACACAGUUGG | 360 | 1801 | 1823 | 1579712 | GAUUUUAUACC | 671 |
| | | UAUAAAAUCAG | | | | | AACUGUGUGG | |
| 1579703 | 1579716 | AUCUUAGUCCAC | 361 | 1809 | 1831 | 1579710 | ACCAACUGUGU | 672 |
| | | ACAGUUGGUAU | | | | | GGACUAAGAU | |
| 1579705 | 1579713 | AUGCAUCUUAGU | 362 | 1813 | 1835 | 1579709 | ACUGUGUGGAC | 673 |
| | | CCACACAGUUG | | | | | UAAGAUGCAU | |
| 1579706 | 1579715 | ACAGUUGGUAUA | 363 | 1797 | 1819 | 1579707 | UUCUGAUUUUA | 674 |
| | | AAAUCAGAAAA | | | | | UACCAACUGU | |
| 1579718 | 1579730 | UUGGUAUAAAAU | 364 | 1793 | 1815 | 1579724 | CAUUUUCUGAU | 675 |
| | | CAGAAAAUGCA | | | | | UUUAUACCAA | |
| 1579719 | 1579732 | AAAUGCAAAGCA | 365 | 1777 | 1799 | 1579725 | UUUUGUUUUUG | 676 |
| | | AAAACAAAAGG | | | | | CUUUGCAUUU | |
| 1579720 | 1579735 | GCAAAGCAAAAA | 366 | 1773 | 1795 | 1579729 | GACCUUUUGUU | 677 |
| | | CAAAAGGUCUG | | | | | UUUGCUUUGC | |
| 1579721 | 1579733 | CAGAAAAUGCAA | 367 | 1781 | 1803 | 1579728 | GUUUUUGCUUU | 678 |
| | | AGCAAAAACAA | | | | | GCAUUUUCUG | |
| 1579722 | 1579731 | AAAUCAGAAAAU | 368 | 1785 | 1807 | 1579726 | UUGCUUUGCAU | 679 |
| | | GCAAAGCAAAA | | | | | UUUCUGAUUU | |
| 1579723 | 1579734 | UAUAAAAUCAGA | 369 | 1789 | 1811 | 1579727 | UUUGCAUUUUC | 680 |
| | | AAAUGCAAAGC | | | | | UGAUUUUAUA | |
| 1579736 | 1579748 | AGCAAAAACAAA | 370 | 1769 | 1791 | 1579742 | UCCAGACCUUU | 681 |
| | | AGGUCUGGAGU | | | | | UGUUUUUGCU | |
| 1579737 | 1579749 | AAAACAAAAGGU | 371 | 1765 | 1787 | 1579743 | AGACUCCAGAC | 682 |
| | | CUGGAGUCUUA | | | | | CUUUUGUUUU | |
| 1579738 | 1579751 | AGGUCUGGAGUC | 372 | 1757 | 1779 | 1579746 | UGAUGCUAAGA | 683 |
| | | UUAGCAUCAGA | | | | | CUCCAGACCU | |
| 1579739 | 1579753 | CAAAAGGUCUGG | 373 | 1761 | 1783 | 1579744 | GCUAAGACUCC | 684 |
| | | AGUCUUAGCAU | | | | | AGACCUUUUG | |
| 1579740 | 1579752 | AGUCUUAGCAUC | 374 | 1749 | 1771 | 1579747 | UGCCCUUCUGA | 685 |
| | | AGAAGGGCACC | | | | | UGCUAAGACU | |
| 1579741 | 1579750 | CUGGAGUCUUAG | 375 | 1753 | 1775 | 1579745 | CUUCUGAUGCU | 686 |
| | | CAUCAGAAGGG | | | | | AAGACUCCAG | |
| 1579754 | 1579763 | UUAGCAUCAGAA | 376 | 1745 | 1767 | 1579760 | AUGGUGCCCUU | 687 |
| | | GGGCACCAUAU | | | | | CUGAUGCUAA | |
| 1579755 | 1579769 | AGAAGGGCACCA | 377 | 1737 | 1759 | 1579764 | AUGUAUAUAUG | 688 |
| | | UAUAUACAUCU | | | | | GUGCCCUUCU | |
| 1579756 | 1579768 | CAUCAGAAGGGC | 378 | 1741 | 1763 | 1579762 | AUAUAUGGUGC | 689 |
| | | ACCAUAUAUAC | | | | | CCUUCUGAUG | |
| 1579757 | 1579770 | UAUAUACAUCUA | 379 | 1725 | 1747 | 1579761 | CACCAACUGUA | 690 |
| | | CAGUUGGUGGC | | | | | GAUGUAUAUA | |
| 1579758 | 1579767 | ACCAUAUAUACA | 380 | 1729 | 1751 | 1579766 | AACUGUAGAUG | 691 |
| | | UCUACAGUUGG | | | | | UAUAUAUGGU | |
| 1579759 | 1579771 | GGGCACCAUAUA | 381 | 1733 | 1755 | 1579765 | GUAGAUGUAUA | 692 |
| | | UACAUCUACAG | | | | | UAUGGUGCCC | |
| 1579772 | 1579780 | UACAUCUACAGU | 382 | 1721 | 1743 | 1579776 | UGGCCACCAAC | 693 |
| | | UGGUGGCCAAU | | | | | UGUAGAUGUA | |
| 1579773 | 1579778 | CAGUUGGUGGCC | 383 | 1713 | 1735 | 1579775 | ACUUGUAUUGG | 694 |
| | | AAUACAAGUCA | | | | | CCACCAACUG | |
| 1579774 | 1579779 | UCUACAGUUGGU | 384 | 1717 | 1739 | 1579777 | GUAUUGGCCAC | 695 |
| | | GGCCAAUACAA | | | | | CAACUGUAGA | |
| 1579781 | 1579787 | UGGUGGCCAAUA | 385 | 1709 | 1731 | 1579784 | AAUGACUUGUA | 696 |
| | | CAAGUCAUUGC | | | | | UUGGCCACCA | |
| 1579782 | 1579791 | AAUACAAGUCAU | 386 | 1701 | 1723 | 1579785 | UGUCUGGCAAU | 697 |
| | | UGCCAGACAGU | | | | | GACUUGUAUU | |
| 1579783 | 1579795 | UGCCAGACAGUC | 387 | 1689 | 1711 | 1579786 | GCCUCCAAGGA | 698 |
| | | CUUGGAGGCAC | | | | | CUGUCUGGCA | |
| 1579788 | 1579798 | GGCCAAUACAAG | 388 | 1705 | 1727 | 1579792 | UGGCAAUGACU | 699 |
| | | UCAUUGCCAGA | | | | | UGUAUUGGCC | |
| 1579789 | 1579797 | CAAGUCAUUGCC | 389 | 1697 | 1719 | 1579793 | GGACUGUCUGG | 700 |
| | | AGACAGUCCUU | | | | | CAAUGACUUG | |
| 1579790 | 1579796 | UCAUUGCCAGAC | 390 | 1693 | 1715 | 1579794 | CCAAGGACUGU | 701 |
| | | AGUCCUUGGAG | | | | | CUGGCAAUGA | |
| 1579799 | 1579814 | AGACAGUCCUUG | 391 | 1685 | 1707 | 1579808 | CUGUGCCUCCA | 702 |
| | | GAGGCACAGAA | | | | | AGGACUGUCU | |
| 1579800 | 1579813 | GAACAGCCUAGA | 392 | 1665 | 1687 | 1579806 | UUGGCUGGGUC | 703 |
| | | CCCAGCCAAGC | | | | | UAGGCUGUUC | |
| 1579801 | 1579812 | CACAGAACAGCC | 393 | 1669 | 1691 | 1579807 | CUGGGUCUAGG | 704 |
| | | UAGACCCAGCC | | | | | CUGUUCUGUG | |
| 1579802 | 1579811 | CUUGGAGGCACA | 394 | 1677 | 1699 | 1579805 | AGGCUGUUCUG | 705 |
| | | GAACAGCCUAG | | | | | UGCCUCCAAG | |
| 1579803 | 1579816 | AGUCCUUGGAGG | 395 | 1681 | 1703 | 1579809 | UGUUCUGUGCC | 706 |
| | | CACAGAACAGC | | | | | UCCAAGGACU | |
| 1579804 | 1579815 | GAGGCACAGAAC | 396 | 1673 | 1695 | 1579810 | GUCUAGGCUGU | 707 |
| | | AGCCUAGACCC | | | | | UCUGUGCCUC | |
| 1579817 | 1579830 | AGCCUAGACCCA | 397 | 1661 | 1683 | 1579825 | GAGCUUGGCUG | 708 |
| | | GCCAAGCUCUA | | | | | GGUCUAGGCU | |
| 1579818 | 1579829 | AGCUCUAGGAAC | 398 | 1645 | 1667 | 1579824 | GGACCGUGAGU | 709 |
| | | UCACGGUCCCA | | | | | UCCUAGAGCU | |
| 1579819 | 1579831 | GCCAAGCUCUAG | 399 | 1649 | 1671 | 1579823 | CGUGAGUUCCU | 710 |
| | | GAACUCACGGU | | | | | AGAGCUUGGC | |
| 1579820 | 1579833 | CCCAGCCAAGCU | 400 | 1653 | 1675 | 1579826 | AGUUCCUAGAG | 711 |
| | | CUAGGAACUCA | | | | | CUUGGCUGGG | |
| 1579821 | 1579832 | UAGACCCAGCCA | 401 | 1657 | 1679 | 1579827 | CCUAGAGCUUG | 712 |
| | | AGCUCUAGGAA | | | | | GCUGGGUCUA | |
| 1579822 | 1579834 | CUAGGAACUCAC | 402 | 1641 | 1663 | 1579828 | CUUGGGACCGU | 713 |
| | | GGUCCCAAGGA | | | | | GAGUUCCUAG | |
| 1579835 | 1579848 | GGUCCCAAGGAG | 403 | 1629 | 1651 | 1579841 | GCGUCUAGACU | 714 |
| | | UCUAGACGCUU | | | | | CCUUGGGACC | |
| 1579836 | 1579847 | UCACGGUCCCAA | 404 | 1633 | 1655 | 1579842 | CUAGACUCCUU | 715 |
| | | GGAGUCUAGAC | | | | | GGGACCGUGA | |
| 1579837 | 1579851 | GAACUCACGGUC | 405 | 1637 | 1659 | 1579846 | ACUCCUUGGGA | 716 |
| | | CCAAGGAGUCU | | | | | CCGUGAGUUC | |
| 1579838 | 1579850 | UCUAGACGCUUG | 406 | 1617 | 1639 | 1579843 | GCAUCAGAACA | 717 |
| | | UUCUGAUGCUC | | | | | AGCGUCUAGA | |
| 1579839 | 1579852 | GGAGUCUAGACG | 407 | 1621 | 1643 | 1579845 | CAGAACAAGCG | 718 |
| | | CUUGUUCUGAU | | | | | UCUAGACUCC | |
| 1579840 | 1579849 | CCAAGGAGUCUA | 408 | 1625 | 1647 | 1579844 | ACAAGCGUCUA | 719 |
| | | GACGCUUGUUC | | | | | GACUCCUUGG | |
| 1579853 | 1579867 | GACGCUUGUUCU | 409 | 1613 | 1635 | 1579862 | CGGAGCAUCAG | 720 |
| | | GAUGCUCCGAC | | | | | AACAAGCGUC | |
| 1579854 | 1579866 | GACCGUAAGAAA | 410 | 1593 | 1615 | 1579860 | UCCCACAUUUU | 721 |
| | | AAUGUGGGAGU | | | | | UCUUACGGUC | |
| 1579855 | 1579865 | CUCCGACCGUAA | 411 | 1597 | 1619 | 1579859 | ACAUUUUUCUU | 722 |
| | | GAAAAAUGUGG | | | | | ACGGUCGGAG | |
| 1579856 | 1579870 | UUCUGAUGCUCC | 412 | 1605 | 1627 | 1579861 | CUUACGGUCGG | 723 |
| | | GACCGUAAGAA | | | | | AGCAUCAGAA | |
| 1579857 | 1579869 | GAUGCUCCGACC | 413 | 1601 | 1623 | 1579864 | UUUUCUUACGG | 724 |
| | | GUAAGAAAAAU | | | | | UCGGAGCAUC | |
| 1579858 | 1579868 | CUUGUUCUGAUG | 414 | 1609 | 1631 | 1579863 | CGGUCGGAGCA | 725 |
| | | CUCCGACCGUA | | | | | UCAGAACAAG | |
| 1579871 | 1579884 | GUAAGAAAAAUG | 415 | 1589 | 1611 | 1579880 | UCACUCCCACA | 726 |
| | | UGGGAGUGAUG | | | | | UUUUUCUUAC | |
| 1579872 | 1579886 | AGUGAUGAAGGC | 416 | 1573 | 1595 | 1579878 | AAUCAUAAAGC | 727 |
| | | UUUAUGAUUUA | | | | | CUUCAUCACU | |
| 1579873 | 1579882 | UGGGAGUGAUGA | 417 | 1577 | 1599 | 1579877 | AUAAAGCCUUC | 728 |
| | | AGGCUUUAUGA | | | | | AUCACUCCCA | |
| 1579874 | 1579887 | AAUGUGGGAGUG | 418 | 1581 | 1603 | 1579881 | AGCCUUCAUCA | 729 |
| | | AUGAAGGCUUU | | | | | CUCCCACAUU | |
| 1579875 | 1579885 | GAAAAAUGUGGG | 419 | 1585 | 1607 | 1579879 | UUCAUCACUCC | 730 |
| | | AGUGAUGAAGG | | | | | CACAUUUUUC | |
| 1579876 | 1579888 | AUGAAGGCUUUA | 420 | 1569 | 1591 | 1579883 | AGUAAAUCAUA | 731 |
| | | UGAUUUACUCA | | | | | AAGCCUUCAU | |
| 1579889 | 1579904 | UUUAUGAUUUAC | 421 | 1561 | 1583 | 1579897 | CUAUAAUGAGU | 732 |
| | | UCAUUAUAGUA | | | | | AAAUCAUAAA | |
| 1579890 | 1579905 | UCAUUAUAGUAA | 422 | 1549 | 1571 | 1579899 | CUGCUAUUAUU | 733 |
| | | UAAUAGCAGCC | | | | | ACUAUAAUGA | |
| 1579891 | 1579902 | UAUAGUAAUAAU | 423 | 1545 | 1567 | 1579896 | UAGGCUGCUAU | 734 |
| | | AGCAGCCUAGC | | | | | UAUUACUAUA | |
| 1579892 | 1579901 | UGAUUUACUCAU | 424 | 1557 | 1579 | 1579895 | AUUACUAUAAU | 735 |
| | | UAUAGUAAUAA | | | | | GAGUAAAUCA | |
| 1579893 | 1579903 | AGGCUUUAUGAU | 425 | 1565 | 1587 | 1579898 | AAUGAGUAAAU | 736 |
| | | UUACUCAUUAU | | | | | CAUAAAGCCU | |
| 1579894 | 1579906 | UUACUCAUUAUA | 426 | 1553 | 1575 | 1579900 | UAUUAUUACUA | 737 |
| | | GUAAUAAUAGC | | | | | UAAUGAGUAA | |
| 1579907 | 1579920 | GUAAUAAUAGCA | 427 | 1541 | 1563 | 1579914 | UAGCUAGGCUG | 738 |
| | | GCCUAGCUAGG | | | | | CUAUUAUUAC | |
| 1579908 | 1579919 | AGCUAGGUACAA | 428 | 1525 | 1547 | 1579913 | UAACUGCUUUU | 739 |
| | | AAGCAGUUAUA | | | | | GUACCUAGCU | |
| 1579909 | 1579921 | GCCUAGCUAGGU | 429 | 1529 | 1551 | 1579915 | UGCUUUUGUAC | 740 |
| | | ACAAAAGCAGU | | | | | CUAGCUAGGC | |
| 1579910 | 1579923 | UAAUAGCAGCCU | 430 | 1537 | 1559 | 1579916 | UACCUAGCUAG | 741 |
| | | AGCUAGGUACA | | | | | GCUGCUAUUA | |
| 1579911 | 1579924 | AGCAGCCUAGCU | 431 | 1533 | 1555 | 1579917 | UUUGUACCUAG | 742 |
| | | AGGUACAAAAG | | | | | CUAGGCUGCU | |
| 1579912 | 1579922 | AGGUACAAAAGC | 432 | 1521 | 1543 | 1579918 | UUUAUAACUGC | 743 |
| | | AGUUAUAAACC | | | | | UUUUGUACCU | |
| 1579925 | 1579939 | ACAAAAGCAGUU | 433 | 1517 | 1539 | 1579933 | AUGGUUUAUAA | 744 |
| | | AUAAACCAUUU | | | | | CUGCUUUUGU | |
| 1579926 | 1579938 | UUUAUAUUACAC | 434 | 1497 | 1519 | 1579936 | AAUAAUUCUGU | 745 |
| | | AGAAUUAUUCA | | | | | GUAAUAUAAA | |
| 1579927 | 1579937 | ACCAUUUAUAUU | 435 | 1501 | 1523 | 1579931 | AUUCUGUGUAA | 746 |
| | | ACACAGAAUUA | | | | | UAUAAAUGGU | |
| 1579928 | 1579942 | AUAAACCAUUUA | 436 | 1505 | 1527 | 1579932 | UGUGUAAUAUA | 747 |
| | | UAUUACACAGA | | | | | AAUGGUUUAU | |
| 1579929 | 1579941 | AGUUAUAAACCA | 437 | 1509 | 1531 | 1579934 | UAAUAUAAAUG | 748 |
| | | UUUAUAUUACA | | | | | GUUUAUAACU | |
| 1579930 | 1579940 | AAGCAGUUAUAA | 438 | 1513 | 1535 | 1579935 | AUAAAUGGUUU | 749 |
| | | ACCAUUUAUAU | | | | | AUAACUGCUU | |
| 1579943 | 1579957 | UAUUACACAGAA | 439 | 1493 | 1515 | 1579951 | CCUGAAUAAUU | 750 |
| | | UUAUUCAGGUC | | | | | CUGUGUAAUA | |
| 1579944 | 1579955 | GUCUCCAUUCUA | 440 | 1473 | 1495 | 1579952 | AACAUAAGAUA | 751 |
| | | UCUUAUGUUGU | | | | | GAAUGGAGAC | |
| 1579945 | 1579960 | UCAGGUCUCCAU | 441 | 1477 | 1499 | 1579954 | UAAGAUAGAAU | 752 |
| | | UCUAUCUUAUG | | | | | GGAGACCUGA | |
| 1579946 | 1579958 | UUAUUCAGGUCU | 442 | 1481 | 1503 | 1579953 | AUAGAAUGGAG | 753 |
| | | CCAUUCUAUCU | | | | | ACCUGAAUAA | |
| 1579947 | 1579956 | AGAAUUAUUCAG | 443 | 1485 | 1507 | 1579950 | AAUGGAGACCU | 754 |
| | | GUCUCCAUUCU | | | | | GAAUAAUUCU | |
| 1579948 | 1579959 | ACACAGAAUUAU | 444 | 1489 | 1511 | 1579949 | GAGACCUGAAU | 755 |
| | | UCAGGUCUCCA | | | | | AAUUCUGUGU | |
| 1579961 | 1579974 | UGUAAAAUUGUU | 445 | 1453 | 1475 | 1579967 | AAUCCAAUUAA | 756 |
| | | AAUUGGAUUUC | | | | | CAAUUUUACA | |
| 1579962 | 1579973 | CCAUUCUAUCUU | 446 | 1469 | 1491 | 1579968 | UUACAACAUAA | 757 |
| | | AUGUUGUAAAA | | | | | GAUAGAAUGG | |
| 1579963 | 1579975 | AUGUUGUAAAAU | 447 | 1457 | 1479 | 1579969 | CAAUUAACAAU | 758 |
| | | UGUUAAUUGGA | | | | | UUUACAACAU | |
| 1579964 | 1579978 | UCUUAUGUUGUA | 448 | 1461 | 1483 | 1579970 | UAACAAUUUUA | 759 |
| | | AAAUUGUUAAU | | | | | CAACAUAAGA | |
| 1579965 | 1579976 | UCUAUCUUAUGU | 449 | 1465 | 1487 | 1579971 | AAUUUUACAAC | 760 |
| | | UGUAAAAUUGU | | | | | AUAAGAUAGA | |
| 1579966 | 1579977 | AAAUUGUUAAUU | 450 | 1449 | 1471 | 1579972 | UGGAAAUCCAA | 761 |
| | | GGAUUUCCAGU | | | | | UUAACAAUUU | |
| 1579979 | 1579989 | UUCCAGUGAGUU | 451 | 1433 | 1455 | 1579983 | CAUCUAAACAA | 762 |
| | | GUUUAGAUGAU | | | | | CUCACUGGAA | |
| 1579980 | 1579991 | AGUUGUUUAGAU | 452 | 1425 | 1447 | 1579984 | UCACUAAUCAU | 763 |
| | | GAUUAGUGAUA | | | | | CUAAACAACU | |
| 1579981 | 1579990 | AGUGAGUUGUUU | 453 | 1429 | 1451 | 1579985 | UAAUCAUCUAA | 764 |
| | | AGAUGAUUAGU | | | | | ACAACUCACU | |
| 1579982 | 1579992 | GGAUUUCCAGUG | 454 | 1437 | 1459 | 1579986 | UAAACAACUCA | 765 |
| | | AGUUGUUUAGA | | | | | CUGGAAAUCC | |
| 1579987 | 1579996 | UGUUAAUUGGAU | 455 | 1445 | 1467 | 1579994 | UCACUGGAAAU | 766 |
| | | UUCCAGUGAGU | | | | | CCAAUUAACA | |
| 1579988 | 1579995 | AAUUGGAUUUCC | 456 | 1441 | 1463 | 1579993 | CAACUCACUGG | 767 |
| | | AGUGAGUUGUU | | | | | AAAUCCAAUU | |
| 1579997 | 1580009 | GUUUAGAUGAUU | 457 | 1421 | 1443 | 1580003 | AUUAUCACUAA | 768 |
| | | AGUGAUAAUAA | | | | | UCAUCUAAAC | |
| 1579998 | 1580014 | AUAAUAAGGAAU | 458 | 1405 | 1427 | 1580007 | GGAUUUACCAU | 769 |
| | | GGUAAAUCCAU | | | | | UCCUUAUUAU | |
| 1579999 | 1580012 | AGUGAUAAUAAG | 459 | 1409 | 1431 | 1580006 | UUACCAUUCCU | 770 |
| | | GAAUGGUAAAU | | | | | UAUUAUCACU | |
| 1580000 | 1580010 | GAUUAGUGAUAA | 460 | 1413 | 1435 | 1580004 | CAUUCCUUAUU | 771 |
| | | UAAGGAAUGGU | | | | | AUCACUAAUC | |
| 1580001 | 1580011 | AGAUGAUUAGUG | 461 | 1417 | 1439 | 1580005 | CCUUAUUAUCA | 772 |
| | | AUAAUAAGGAA | | | | | CUAAUCAUCU | |
| 1580002 | 1580013 | UAAGGAAUGGUA | 462 | 1401 | 1423 | 1580008 | CUAUGGAUUUA | 773 |
| | | AAUCCAUAGCA | | | | | CCAUUCCUUA | |
| 1580015 | 1580027 | GAAUGGUAAAUC | 463 | 1397 | 1419 | 1580024 | GGUGCUAUGGA | 774 |
| | | CAUAGCACCAU | | | | | UUUACCAUUC | |
| 1580016 | 1580032 | CAUUUCAAAGAC | 464 | 1377 | 1399 | 1580022 | UGACAACAAGU | 775 |
| | | UUGUUGUCACU | | | | | CUUUGAAAUG | |
| 1580017 | 1580029 | GCACCAUUUCAA | 465 | 1381 | 1403 | 1580021 | AACAAGUCUUU | 776 |
| | | AGACUUGUUGU | | | | | GAAAUGGUGC | |
| 1580018 | 1580030 | CAUAGCACCAUU | 466 | 1385 | 1407 | 1580026 | AGUCUUUGAAA | 777 |
| | | UCAAAGACUUG | | | | | UGGUGCUAUG | |
| 1580019 | 1580031 | GGUAAAUCCAUA | 467 | 1393 | 1415 | 1580023 | AAAUGGUGCUA | 778 |
| | | GCACCAUUUCA | | | | | UGGAUUUACC | |
| 1580020 | 1580028 | AAUCCAUAGCAC | 468 | 1389 | 1411 | 1580025 | UUUGAAAUGGU | 779 |
| | | CAUUUCAAAGA | | | | | GCUAUGGAUU | |
| 1580033 | 1580045 | UCAAAGACUUGU | 469 | 1373 | 1395 | 1580039 | UCAGUGACAAC | 780 |
| | | UGUCACUGAUU | | | | | AAGUCUUUGA | |
| 1580034 | 1580050 | AUUUCUCAUUUA | 470 | 1353 | 1375 | 1580041 | UCACACAUCUA | 781 |
| | | GAUGUGUGACG | | | | | AAUGAGAAAU | |
| 1580035 | 1580048 | ACUGAUUUCUCA | 471 | 1357 | 1379 | 1580042 | ACAUCUAAAUG | 782 |
| | | UUUAGAUGUGU | | | | | AGAAAUCAGU | |
| 1580036 | 1580046 | UGUCACUGAUUU | 472 | 1361 | 1383 | 1580040 | CUAAAUGAGAA | 783 |
| | | CUCAUUUAGAU | | | | | AUCAGUGACA | |
| 1580037 | 1580047 | UUGUUGUCACUG | 473 | 1365 | 1387 | 1580044 | AUGAGAAAUCA | 784 |
| | | AUUUCUCAUUU | | | | | GUGACAACAA | |
| 1580038 | 1580049 | AGACUUGUUGUC | 474 | 1369 | 1391 | 1580043 | GAAAUCAGUGA | 785 |
| | | ACUGAUUUCUC | | | | | CAACAAGUCU | |
| 1580051 | 1580067 | CUCAUUUAGAUG | 475 | 1349 | 1371 | 1580061 | UUCGUCACACA | 786 |
| | | UGUGACGAAGA | | | | | UCUAAAUGAG | |
| 1580052 | 1580063 | AGAUACUCCACC | 476 | 1329 | 1351 | 1580058 | GCCCUUACAGG | 787 |
| | | UGUAAGGGCAA | | | | | UGGAGUAUCU | |
| 1580053 | 1580068 | ACGAAGAUACUC | 477 | 1333 | 1355 | 1580062 | UUACAGGUGGA | 788 |
| | | CACCUGUAAGG | | | | | GUAUCUUCGU | |
| 1580054 | 1580064 | UGUGACGAAGAU | 478 | 1337 | 1359 | 1580057 | AGGUGGAGUAU | 789 |
| | | ACUCCACCUGU | | | | | CUUCGUCACA | |
| 1580055 | 1580065 | GAUGUGUGACGA | 479 | 1341 | 1363 | 1580060 | GGAGUAUCUUC | 790 |
| | | AGAUACUCCAC | | | | | GUCACACAUC | |
| 1580056 | 1580066 | UUUAGAUGUGUG | 480 | 1345 | 1367 | 1580059 | UAUCUUCGUCA | 791 |
| | | ACGAAGAUACU | | | | | CACAUCUAAA | |
| 1580069 | 1580085 | ACUCCACCUGUA | 481 | 1325 | 1347 | 1580080 | ACUUGCCCUUA | 792 |
| | | AGGGCAAGUAU | | | | | CAGGUGGAGU | |
| 1580070 | 1580084 | CAAGUAUGCCAA | 482 | 1309 | 1331 | 1580077 | CUUGUGGCAUU | 793 |
| | | UGCCACAAGCC | | | | | GGCAUACUUG | |
| 1580071 | 1580081 | UAUGCCAAUGCC | 483 | 1305 | 1327 | 1580079 | ACGGCUUGUGG | 794 |
| | | ACAAGCCGUGU | | | | | CAUUGGCAUA | |
| 1580072 | 1580086 | AGGGCAAGUAUG | 484 | 1313 | 1335 | 1580078 | UGGCAUUGGCA | 795 |
| | | CCAAUGCCACA | | | | | UACUUGCCCU | |
| 1580073 | 1580083 | UGUAAGGGCAAG | 485 | 1317 | 1339 | 1580076 | AUUGGCAUACU | 796 |
| | | UAUGCCAAUGC | | | | | UGCCCUUACA | |
| 1580074 | 1580082 | CACCUGUAAGGG | 486 | 1321 | 1343 | 1580075 | GCAUACUUGCC | 797 |
| | | CAAGUAUGCCA | | | | | CUUACAGGUG | |
| 1580087 | 1580100 | UGCCACAAGCCG | 487 | 1297 | 1319 | 1580094 | GCAAAAACACG | 798 |
| | | UGUUUUUGCAA | | | | | GCUUGUGGCA | |
| 1580088 | 1580101 | UGUUUUUGCAAG | 488 | 1285 | 1307 | 1580093 | ACUGGAGCCCU | 799 |
| | | GGCUCCAGUUU | | | | | UGCAAAAACA | |
| 1580089 | 1580099 | GCCGUGUUUUUG | 489 | 1289 | 1311 | 1580095 | GAGCCCUUGCA | 800 |
| | | CAAGGGCUCCA | | | | | AAAACACGGC | |
| 1580090 | 1580103 | ACAAGCCGUGUU | 490 | 1293 | 1315 | 1580098 | CCUUGCAAAAA | 801 |
| | | UUUGCAAGGGC | | | | | CACGGCUUGU | |
| 1580091 | 1580102 | CCAAUGCCACAA | 491 | 1301 | 1323 | 1580096 | AAACACGGCUU | 802 |
| | | GCCGUGUUUUU | | | | | GUGGCAUUGG | |
| 1580092 | 1580104 | UUUGCAAGGGCU | 492 | 1281 | 1303 | 1580097 | CCAAACUGGAG | 803 |
| | | CCAGUUUGGGC | | | | | CCCUUGCAAA | |
| 1580105 | 1580120 | CAAGGGCUCCAG | 493 | 1277 | 1299 | 1580112 | AUGCCCAAACU | 804 |
| | | UUUGGGCAUUU | | | | | GGAGCCCUUG | |
| 1580106 | 1580119 | UUUUGUCCGUGU | 494 | 1257 | 1279 | 1580111 | UUUACGCGCAC | 805 |
| | | GCGCGUAAAGC | | | | | ACGGACAAAA | |
| 1580107 | 1580117 | UUUGGGCAUUUU | 495 | 1265 | 1287 | 1580114 | CACACGGACAA | 806 |
| | | GUCCGUGUGCG | | | | | AAUGCCCAAA | |
| 1580108 | 1580121 | GGCAUUUUGUCC | 496 | 1261 | 1283 | 1580116 | CGCGCACACGG | 807 |
| | | GUGUGCGCGUA | | | | | ACAAAAUGCC | |
| 1580109 | 1580122 | CCAGUUUGGGCA | 497 | 1269 | 1291 | 1580113 | CGGACAAAAUG | 808 |
| | | UUUUGUCCGUG | | | | | CCCAAACUGG | |
| 1580110 | 1580118 | GGCUCCAGUUUG | 498 | 1273 | 1295 | 1580115 | CAAAAUGCCCA | 809 |
| | | GGCAUUUUGUC | | | | | AACUGGAGCC | |
| 1580123 | 1580135 | GUCCGUGUGCGC | 499 | 1253 | 1275 | 1580129 | AAGCUUUACGC | 810 |
| | | GUAAAGCUUCA | | | | | GCACACGGAC | |
| 1580124 | 1580134 | UCACACAGAGGU | 500 | 1233 | 1255 | 1580128 | GCUGCCCGAAC | 811 |
| | | UCGGGCAGCGG | | | | | CUCUGUGUGA | |
| 1580125 | 1580136 | AGCUUCACACAG | 501 | 1237 | 1259 | 1580130 | CCCGAACCUCU | 812 |
| | | AGGUUCGGGCA | | | | | GUGUGAAGCU | |
| 1580126 | 1580137 | GUAAAGCUUCAC | 502 | 1241 | 1263 | 1580131 | AACCUCUGUGU | 813 |
| | | ACAGAGGUUCG | | | | | GAAGCUUUAC | |
| 1580127 | 1580138 | GCGCGUAAAGCU | 503 | 1245 | 1267 | 1580132 | UCUGUGUGAAG | 814 |
| | | UCACACAGAGG | | | | | CUUUACGCGC | |
| 1580133 | 1580140 | GUGUGCGCGUAA | 504 | 1249 | 1271 | 1580139 | UGUGAAGCUUU | 815 |
| | | AGCUUCACACA | | | | | ACGCGCACAC | |
| 1580141 | 1580153 | AGGUUCGGGCAG | 505 | 1225 | 1247 | 1580147 | AAACAGCCGCU | 816 |
| | | CGGCUGUUUCU | | | | | GCCCGAACCU | |
| 1580142 | 1580154 | UCGGGCAGCGGC | 506 | 1221 | 1243 | 1580148 | ACAGAAACAGC | 817 |
| | | UGUUUCUGUUG | | | | | CGCUGCCCGA | |
| 1580143 | 1580157 | ACAGAGGUUCGG | 507 | 1229 | 1251 | 1580149 | AGCCGCUGCCC | 818 |
| | | GCAGCGGCUGU | | | | | GAACCUCUGU | |
| 1580144 | 1580156 | UGUUUCUGUUGG | 508 | 1209 | 1231 | 1580151 | CCAGUGCAUCC | 819 |
| | | AUGCACUGGGU | | | | | AACAGAAACA | |
| 1580145 | 1580158 | CGGCUGUUUCUG | 509 | 1213 | 1235 | 1580150 | UGCAUCCAACA | 820 |
| | | UUGGAUGCACU | | | | | GAAACAGCCG | |
| 1580146 | 1580155 | GCAGCGGCUGUU | 510 | 1217 | 1239 | 1580152 | UCCAACAGAAA | 821 |
| | | UCUGUUGGAUG | | | | | CAGCCGCUGC | |
| 1580159 | 1580176 | UCUGUUGGAUGC | 511 | 1205 | 1227 | 1580168 | UGACCCAGUGC | 822 |
| | | ACUGGGUCACC | | | | | AUCCAACAGA | |
| 1580160 | 1580175 | ACCCACCAGAAA | 512 | 1185 | 1207 | 1580165 | AAAAGCCCUUU | 823 |
| | | AGGGCUUUUGG | | | | | UCUGGUGGGU | |
| 1580161 | 1580173 | GGUCACCCACCA | 513 | 1189 | 1211 | 1580166 | GCCCUUUUCUG | 824 |
| | | GAAAAGGGCUU | | | | | GUGGGUGACC | |
| 1580162 | 1580171 | AUGCACUGGGUC | 514 | 1197 | 1219 | 1580167 | CUGGUGGGUGA | 825 |
| | | ACCCACCAGAA | | | | | CCCAGUGCAU | |
| 1580163 | 1580174 | UUGGAUGCACUG | 515 | 1201 | 1223 | 1580170 | UGGGUGACCCA | 826 |
| | | GGUCACCCACC | | | | | GUGCAUCCAA | |
| 1580164 | 1580172 | ACUGGGUCACCC | 516 | 1193 | 1215 | 1580169 | UUUUCUGGUGG | 827 |
| | | ACCAGAAAAGG | | | | | GUGACCCAGU | |
| 1580177 | 1580191 | ACCAGAAAAGGG | 517 | 1181 | 1203 | 1580188 | GUCCAAAAGCC | 828 |
| | | CUUUUGGACAU | | | | | CUUUUCUGGU | |
| 1580178 | 1580193 | CAUUUGGGGUUU | 518 | 1161 | 1183 | 1580186 | UGUGGGUAGAA | 829 |
| | | CUACCCACACU | | | | | ACCCCAAAUG | |
| 1580179 | 1580190 | UGGACAUUUGGG | 519 | 1165 | 1187 | 1580187 | GGUAGAAACCC | 830 |
| | | GUUUCUACCCA | | | | | CAAAUGUCCA | |
| 1580180 | 1580194 | AGGGCUUUUGGA | 520 | 1173 | 1195 | 1580185 | CCCCAAAUGUC | 831 |
| | | CAUUUGGGGUU | | | | | CAAAAGCCCU | |
| 1580181 | 1580192 | CUUUUGGACAUU | 521 | 1169 | 1191 | 1580184 | GAAACCCCAAA | 832 |
| | | UGGGGUUUCUA | | | | | UGUCCAAAAG | |
| 1580182 | 1580189 | GAAAAGGGCUUU | 522 | 1177 | 1199 | 1580183 | AAAUGUCCAAA | 833 |
| | | UGGACAUUUGG | | | | | AGCCCUUUUC | |
| 1580195 | 1580209 | GUUUCUACCCAC | 523 | 1153 | 1175 | 1580202 | AACCAAAGUGU | 834 |
| | | ACUUUGGUUUU | | | | | GGGUAGAAAC | |
| 1580196 | 1580207 | ACUUUGGUUUUC | 524 | 1141 | 1163 | 1580201 | CCUCAUUUAGA | 835 |
| | | UAAAUGAGGUG | | | | | AAACCAAAGU | |
| 1580197 | 1580212 | CCACACUUUGGU | 525 | 1145 | 1167 | 1580205 | AUUUAGAAAAC | 836 |
| | | UUUCUAAAUGA | | | | | CAAAGUGUGG | |
| 1580198 | 1580210 | CUACCCACACUU | 526 | 1149 | 1171 | 1580204 | AGAAAACCAAA | 837 |
| | | UGGUUUUCUAA | | | | | GUGUGGGUAG | |
| 1580199 | 1580211 | UGGGGUUUCUAC | 527 | 1157 | 1179 | 1580206 | AAAGUGUGGGU | 838 |
| | | CCACACUUUGG | | | | | AGAAACCCCA | |
| 1580200 | 1580208 | UGGUUUUCUAAA | 528 | 1137 | 1159 | 1580203 | UCCACCUCAUU | 839 |
| | | UGAGGUGGACU | | | | | UAGAAAACCA | |
| 1580213 | 1580225 | UUUCUAAAUGAG | 529 | 1133 | 1155 | 1580221 | CCAGUCCACCU | 840 |
| | | GUGGACUGGGA | | | | | CAUUUAGAAA | |
| 1580214 | 1580227 | GGAGGGAGGUAU | 530 | 1113 | 1135 | 1580220 | UGAAAGAAGAU | 841 |
| | | CUUCUUUCAGA | | | | | ACCUCCCUCC | |
| 1580215 | 1580226 | ACUGGGAGGGAG | 531 | 1117 | 1139 | 1580219 | AGAAGAUACCU | 842 |
| | | GUAUCUUCUUU | | | | | CCCUCCCAGU | |
| 1580216 | 1580228 | UGAGGUGGACUG | 532 | 1125 | 1147 | 1580223 | CCUCCCUCCCAG | 843 |
| | | GGAGGGAGGUA | | | | | UCCACCUCA | |
| 1580217 | 1580230 | GUGGACUGGGAG | 533 | 1121 | 1143 | 1580222 | GAUACCUCCCU | 844 |
| | | GGAGGUAUCUU | | | | | CCCAGUCCAC | |
| 1580218 | 1580229 | UAAAUGAGGUGG | 534 | 1129 | 1151 | 1580224 | CCUCCCAGUCC | 845 |
| | | ACUGGGAGGGA | | | | | ACCUCAUUUA | |
| 1580231 | 1580243 | GGAGGUAUCUUC | 535 | 1109 | 1131 | 1580238 | CAUCUGAAAGA | 846 |
| | | UUUCAGAUGAA | | | | | AGAUACCUCC | |
| 1580232 | 1580246 | GAAAGGGAAGGG | 536 | 1089 | 1111 | 1580239 | CCAUCUCGCCCC | 847 |
| | | GCGAGAUGGAG | | | | | UUCCCUUUC | |
| 1580233 | 1580248 | AGAUGAAAGGGA | 537 | 1093 | 1115 | 1580237 | CUCGCCCCUUCC | 848 |
| | | AGGGGCGAGAU | | | | | CUUUCAUCU | |
| 1580234 | 1580244 | CUUCUUUCAGAU | 538 | 1101 | 1123 | 1580241 | UUCCCUUUCAU | 849 |
| | | GAAAGGGAAGG | | | | | CUGAAAGAAG | |
| 1580235 | 1580247 | UUUCAGAUGAAA | 539 | 1097 | 1119 | 1580240 | CCCCUUCCCUU | 850 |
| | | GGGAAGGGGCG | | | | | UCAUCUGAAA | |
| 1580236 | 1580245 | GUAUCUUCUUUC | 540 | 1105 | 1127 | 1580242 | CUUUCAUCUGA | 851 |
| | | AGAUGAAAGGG | | | | | AAGAAGAUAC | |
| 1580249 | 1580266 | AGGGGCGAGAUG | 541 | 1081 | 1103 | 1580256 | AGAUAACUCCA | 852 |
| | | GAGUUAUCUUA | | | | | UCUCGCCCCU | |
| 1580250 | 1580263 | UAUCUUAUUUCU | 542 | 1065 | 1087 | 1580259 | GUUUUACCCAG | 853 |
| | | GGGUAAAACAA | | | | | AAAUAAGAUA | |
| 1580251 | 1580265 | GAUGGAGUUAUC | 543 | 1073 | 1095 | 1580257 | CAGAAAUAAGA | 854 |
| | | UUAUUUCUGGG | | | | | UAACUCCAUC | |
| 1580252 | 1580261 | GAGUUAUCUUAU | 544 | 1069 | 1091 | 1580260 | UACCCAGAAAU | 855 |
| | | UUCUGGGUAAA | | | | | AAGAUAACUC | |
| 1580253 | 1580264 | GCGAGAUGGAGU | 545 | 1077 | 1099 | 1580258 | AAUAAGAUAAC | 856 |
| | | UAUCUUAUUUC | | | | | UCCAUCUCGC | |
| 1580254 | 1580262 | GGGAAGGGGCGA | 546 | 1085 | 1107 | 1580255 | AACUCCAUCUC | 857 |
| | | GAUGGAGUUAU | | | | | GCCCCUUCCC | |
| 1580267 | 1580282 | UUCUGGGUAAAA | 547 | 1057 | 1079 | 1580277 | UUUGUUUUGUU | 858 |
| | | CAAAACAAACA | | | | | UUACCCAGAA | |
| 1580268 | 1580284 | ACAAACAAAAAA | 548 | 1037 | 1059 | 1580273 | UUUGUUUUGUU | 859 |
| | | CAAAACAAAAA | | | | | UUUUGUUUGU | |
| 1580269 | 1580279 | CAAAACAAACAA | 549 | 1045 | 1067 | 1580274 | GUUUUUUGUUU | 860 |
| | | ACAAAAAACAA | | | | | GUUUGUUUUG | |
| 1580270 | 1580283 | AAAACAAAACAA | 550 | 1049 | 1071 | 1580275 | UUUGUUUGUUU | 861 |
| | | ACAAACAAAAA | | | | | GUUUUGUUUU | |
| 1580271 | 1580281 | GGGUAAAACAAA | 551 | 1053 | 1075 | 1580276 | UUUGUUUGUUU | 862 |
| | | ACAAACAAACA | | | | | UGUUUUACCC | |
| 1580272 | 1580280 | UUAUUUCUGGGU | 552 | 1061 | 1083 | 1580278 | UUUUGUUUUAC | 863 |
| | | AAAACAAAACA | | | | | CCAGAAAUAA | |
| 1580285 | 1580302 | AAAACAAAACAA | 553 | 1029 | 1051 | 1580294 | UCAGUAUUUUU | 864 |
| | | AAAUACUGAGC | | | | | GUUUUGUUUU | |
| 1580286 | 1580300 | CAAAACAAAAAU | 554 | 1025 | 1047 | 1580291 | CAGCUCAGUAU | 865 |
| | | ACUGAGCUGGA | | | | | UUUUGUUUUG | |
| 1580287 | 1580297 | AAAUACUGAGCU | 555 | 1017 | 1039 | 1580293 | GUAUAAUCCAG | 866 |
| | | GGAUUAUACUG | | | | | CUCAGUAUUU | |
| 1580288 | 1580298 | ACAAACAAACAA | 556 | 1041 | 1063 | 1580292 | UUUUGUUUUUU | 867 |
| | | AAAACAAAACA | | | | | GUUUGUUUGU | |
| 1580289 | 1580299 | ACAAAAAACAAA | 557 | 1033 | 1055 | 1580296 | UAUUUUUGUUU | 868 |
| | | ACAAAAAUAçU | | | | | UGUUUUUUGU | |
| 1580290 | 1580301 | ACAAAAAUACUG | 558 | 1021 | 1043 | 1580295 | AAUCCAGCUCA | 869 |
| | | AGCUGGAUUAU | | | | | GUAUUUUUGU | |
| 1580303 | 1580319 | ACUGAGCUGGAU | 559 | 1013 | 1035 | 1580314 | AACAGUAUAAU | 870 |
| | | UAUACUGUUAG | | | | | CCAGCUCAGU | |
| 1580304 | 1580315 | UAGGAUGUAAAG | 560 | 993 | 1015 | 1580312 | GCUAAGGAACU | 871 |
| | | UUCCUUAGCUA | | | | | UUACAUCCUA | |
| 1580305 | 1580317 | CUGUUAGGAUGU | 561 | 997 | 1019 | 1580313 | AGGAACUUUAC | 872 |
| | | AAAGUUCCUUA | | | | | AUCCUAACAG | |
| 1580306 | 1580316 | UAUACUGUUAGG | 562 | 1001 | 1023 | 1580309 | ACUUUACAUCC | 873 |
| | | AUGUAAAGUUC | | | | | UAACAGUAUA | |
| 1580307 | 1580320 | AGCUGGAUUAUA | 563 | 1009 | 1031 | 1580310 | UCCUAACAGUA | 874 |
| | | CUGUUAGGAUG | | | | | UAAUCCAGCU | |
| 1580308 | 1580318 | GGAUUAUACUGU | 564 | 1005 | 1027 | 1580311 | UACAUCCUAAC | 875 |
| | | UAGGAUGUAAA | | | | | AGUAUAAUCC | |
| 1580321 | 1580337 | AUGUAAAGUUCC | 565 | 989 | 1011 | 1580331 | AGUAGCUAAGG | 876 |
| | | UUAGCUACUUC | | | | | AACUUUACAU | |
| 1580322 | 1580334 | UUCUUUAAGGCU | 566 | 969 | 991 | 1580329 | CUCGUGUUGAG | 877 |
| | | CAACACGAGGC | | | | | CCUUAAAGAA | |
| 1580323 | 1580333 | CUACUUCUUUAA | 567 | 973 | 995 | 1580327 | UGUUGAGCCUU | 878 |
| | | GGCUCAACACG | | | | | AAAGAAGUAG | |
| 1580324 | 1580336 | UUAGCUACUUCU | 568 | 977 | 999 | 1580328 | GAGCCUUAAAG | 879 |
| | | UUAAGGCUCAA | | | | | AAGUAGCUAA | |
| 1580325 | 1580335 | UUCCUUAGCUAC | 569 | 981 | 1003 | 1580330 | CUUAAAGAAGU | 880 |
| | | UUCUUUAAGGC | | | | | AGCUAAGGAA | |
| 1580326 | 1580338 | AAAGUUCCUUAG | 570 | 985 | 1007 | 1580332 | AAGAAGUAGCU | 881 |
| | | CUACUUCUUUA | | | | | AAGGAACUUU | |
| 1580339 | 1580351 | UUAAGGCUCAAC | 571 | 965 | 987 | 1580345 | CAGCCUCGUGU | 882 |
| | | ACGAGGCUGAU | | | | | UGAGCCUUAA | |
| 1580340 | 1580352 | ACGAGGCUGAUG | 572 | 953 | 975 | 1580346 | UAUGUUGACCA | 883 |
| | | GUCAACAUAAA | | | | | UCAGCCUCGU | |
| 1580341 | 1580353 | GAUGGUCAACAU | 573 | 945 | 967 | 1580347 | UUGCUUUUUAU | 884 |
| | | AAAAAGCAAAC | | | | | GUUGACCAUC | |
| 1580342 | 1580356 | CAACACGAGGCU | 574 | 957 | 979 | 1580350 | UUGACCAUCAG | 885 |
| | | GAUGGUCAACA | | | | | CCUCGUGUUG | |
| 1580343 | 1580354 | GGCUCAACACGA | 575 | 961 | 983 | 1580349 | CCAUCAGCCUC | 886 |
| | | GGCUGAUGGUC | | | | | GUGUUGAGCC | |
| 1580344 | 1580355 | GGCUGAUGGUCA | 576 | 949 | 971 | 1580348 | UUUUUAUGUUG | 887 |
| | | ACAUAAAAAGC | | | | | ACCAUCAGCC | |
| 1580357 | 1580371 | GUCAACAUAAAA | 577 | 941 | 963 | 1580366 | UUGUUUGCUUU | 888 |
| | | AGCAAACAAUA | | | | | UUAUGUUGAC | |
| 1580358 | 1580369 | AACAAUACUAUG | 578 | 925 | 947 | 1580363 | AUAUAUGUACA | 889 |
| | | UACAUAUAUGU | | | | | UAGUAUUGUU | |
| 1580359 | 1580372 | UACAUAUAUGUA | 579 | 913 | 935 | 1580367 | AACACUUUUUA | 890 |
| | | AAAAGUGUUAU | | | | | CAUAUAUGUA | |
| 1580360 | 1580374 | AGCAAACAAUAC | 580 | 929 | 951 | 1580364 | AUGUACAUAGU | 891 |
| | | UAUGUACAUAU | | | | | AUUGUUUGCU | |
| 1580361 | 1580373 | AAAAAGCAAACA | 581 | 933 | 955 | 1580368 | ACAUAGUAUUG | 892 |
| | | AUACUAUGUAC | | | | | UUUGCUUUUU | |
| 1580362 | 1580370 | ACAUAAAAAGCA | 582 | 937 | 959 | 1580365 | AGUAUUGUUUG | 893 |
| | | AACAAUACUAU | | | | | CUUUUUAUGU | |
| 1580375 | 1580389 | AUACUAUGUACA | 583 | 921 | 943 | 1580383 | UUACAUAUAUG | 894 |
| | | UAUAUGUAAAA | | | | | UACAUAGUAU | |
| 1580376 | 1580387 | AAAAGUGUUAUA | 584 | 901 | 923 | 1580382 | AAACCUAUUUA | 895 |
| | | AAUAGGUUUUA | | | | | UAACACUUUU | |
| 1580377 | 1580388 | UGUAAAAAGUGU | 585 | 905 | 927 | 1580381 | CUAUUUAUAAC | 896 |
| | | UAUAAAUAGGU | | | | | ACUUUUUACA | |
| 1580378 | 1580390 | UAUAUGUAAAAA | 586 | 909 | 931 | 1580384 | UUAUAACACUU | 897 |
| | | GUGUUAUAAAU | | | | | UUUACAUAUA | |
| 1580379 | 1580392 | UAUGUACAUAUA | 587 | 917 | 939 | 1580385 | CUUUUUACAUA | 898 |
| | | UGUAAAAAGUG | | | | | UAUGUACAUA | |
| 1580380 | 1580391 | UAUAAAUAGGUU | 588 | 893 | 915 | 1580386 | GGUUUAUAAAA | 899 |
| | | UUAUAAACCGG | | | | | CCUAUUUAUA | |
| 1580393 | 1580408 | AUACAUCUUCAA | 589 | 861 | 883 | 1580401 | UGCUGUUGAUU | 900 |
| | | UCAACAGCAAC | | | | | GAAGAUGUAU | |
| 1580394 | 1580406 | GUGUUAUAAAUA | 590 | 897 | 919 | 1580403 | UAUAAAACCUA | 901 |
| | | GGUUUUAUAAA | | | | | UUUAUAACAC | |
| 1580395 | 1580410 | AAACCGGAGAUA | 591 | 877 | 899 | 1580400 | UGUAUAUAAUA | 902 |
| | | UUAUAUACAUC | | | | | UCUCCGGUUU | |
| 1580396 | 1580409 | AAUAGGUUUUAU | 592 | 889 | 911 | 1580399 | CUCCGGUUUAU | 903 |
| | | AAACCGGAGAU | | | | | AAAACCUAUU | |
| 1580397 | 1580405 | UUAUAAACCGGA | 593 | 881 | 903 | 1580402 | UAUAAUAUCUC | 904 |
| | | GAUAUUAUAUA | | | | | CGGUUUAUAA | |
| 1580398 | 1580407 | GGUUUUAUAAAC | 594 | 885 | 907 | 1580404 | AUAUCUCCGGU | 905 |
| | | CGGAGAUAUUA | | | | | UUAUAAAACC | |
| 1580411 | 1580426 | GAUAUUAUAUAC | 595 | 869 | 891 | 1580422 | AUUGAAGAUGU | 906 |
| | | AUCUUCAAUCA | | | | | AUAUAAUAUC | |
| 1580412 | 1580424 | UCAACAGCAACC | 596 | 849 | 871 | 1580420 | GGAGGUGGGGG | 907 |
| | | CCCACCUCCAC | | | | | UUGCUGUUGA | |
| 1580413 | 1580425 | AUCUUCAAUCAA | 597 | 857 | 879 | 1580417 | GGGUUGCUGUU | 908 |
| | | CAGCAACCCCC | | | | | GAUUGAAGAU | |
| 1580414 | 1580427 | UCAAUCAACAGC | 598 | 853 | 875 | 1580418 | GUGGGGGUUGC | 909 |
| | | AACCCCCACCU | | | | | UGUUGAUUGA | |
| 1580415 | 1580428 | CGGAGAUAUUAU | 599 | 873 | 895 | 1580419 | AAGAUGUAUAU | 910 |
| | | AUACAUCUUCA | | | | | AAUAUCUCCG | |
| 1580416 | 1580423 | UUAUAUACAUCU | 600 | 865 | 887 | 1580421 | GUUGAUUGAAG | 911 |
| | | UCAAUCAACAG | | | | | AUGUAUAUAA | |
| 1580429 | 1580441 | CAGCAACCCCCA | 601 | 845 | 867 | 1580435 | CAGUGGAGGUG | 912 |
| | | CCUCCACUGCU | | | | | GGGGUUGCUG | |
| 1580430 | 1580445 | AACCCCCACCUC | 602 | 841 | 863 | 1580436 | AAAGCAGUGGA | 913 |
| | | CACUGCUUUCU | | | | | GGUGGGGGUU | |
| 1580431 | 1580444 | CACUGCUUUCUG | 603 | 829 | 851 | 1580437 | CAAACCAAACA | 914 |
| | | UUUGGUUUGGU | | | | | GAAAGCAGUG | |
| 1580432 | 1580443 | CCCACCUCCACU | 604 | 837 | 859 | 1580439 | ACAGAAAGCAG | 915 |
| | | GCUUUCUGUUU | | | | | UGGAGGUGGG | |
| 1580433 | 1580442 | CCUCCACUGCUU | 605 | 833 | 855 | 1580438 | CCAAACAGAAA | 916 |
| | | UCUGUUUGGUU | | | | | GCAGUGGAGG | |
| 1580434 | 1580446 | GCUUUCUGUUUG | 606 | 825 | 847 | 1580440 | AAACCAAACCA | 917 |
| | | GUUUGGUUUGA | | | | | AACAGAAAGC | |
| 1580447 | 1580463 | UCUGUUUGGUUU | 607 | 821 | 843 | 1580453 | ACUCAAACCAA | 918 |
| | | GGUUUGAGUUU | | | | | ACCAAACAGA | |
| 1580448 | 1580461 | GGCUAGCUCUUU | 608 | 789 | 811 | 1580457 | ACAAAGAAAAA | 919 |
| | | UUUCUUUGUCU | | | | | AGAGCUAGCC | |
| 1580449 | 1580460 | UGAGUUUGGGAU | 609 | 805 | 827 | 1580458 | UAGCCCAAAAU | 920 |
| | | UUUGGGCUAGC | | | | | CCCAAACUCA | |
| 1580450 | 1580464 | GGUUUGAGUUUG | 610 | 809 | 831 | 1580456 | CCAAAAUCCCA | 921 |
| | | GGAUUUUGGGC | | | | | AACUCAAACC | |
| 1580451 | 1580459 | UUUGGUUUGGUU | 611 | 817 | 839 | 1580455 | CCAAACUCAAA | 922 |
| | | UGAGUUUGGGA | | | | | CCAAACCAAA | |
| 1580452 | 1580462 | GUUUGGUUUGAG | 612 | 813 | 835 | 1580454 | AAUCCCAAACU | 923 |
| | | UUUGGGAUUUU | | | | | CAAACCAAAC | |
| 1580465 | 1580480 | UUUGGGAUUUUG | 613 | 801 | 823 | 1580473 | GAGCUAGCCCA | 924 |
| | | GGCUAGCUCUU | | | | | AAAUCCCAAA | |
| 1580466 | 1580478 | UUUCUUUGUCUG | 614 | 777 | 799 | 1580476 | AACAGAAAGCA | 925 |
| | | CUUUCUGUUUU | | | | | GACAAAGAAA | |
| 1580467 | 1580477 | CUUUUUUCUUUG | 615 | 781 | 803 | 1580475 | GAAAGCAGACA | 926 |
| | | UCUGCUUUCUG | | | | | AAGAAAAAAG | |
| 1580468 | 1580481 | AGCUCUUUUUUC | 616 | 785 | 807 | 1580474 | GCAGACAAAGA | 927 |
| | | UUUGUCUGCUU | | | | | AAAAAGAGCU | |
| 1580469 | 1580482 | UUUGGGCUAGCU | 617 | 793 | 815 | 1580471 | AGAAAAAAGAG | 928 |
| | | CUUUUUUCUUU | | | | | CUAGCCCAAA | |
| 1580470 | 1580479 | GGAUUUUGGGCU | 618 | 797 | 819 | 1580472 | AAAAGAGCUAG | 929 |
| | | AGCUCUUUUUU | | | | | CCCAAAAUCC | |
| 1580483 | 1580500 | UUUGUCUGCUUU | 619 | 773 | 795 | 1580490 | GGAAAACAGAA | 930 |
| | | CUGUUUUCCCU | | | | | AGCAGACAAA | |
| 1580484 | 1580498 | CCUCCCUUCCCU | 620 | 749 | 771 | 1580491 | AGCGUACAUAG | 931 |
| | | AUGUACGCUCA | | | | | GGAAGGGAGG | |
| 1580485 | 1580495 | CCUUCCUCCCUU | 621 | 753 | 775 | 1580489 | UACAUAGGGAA | 932 |
| | | CCCUAUGUACG | | | | | GGGAGGAAGG | |
| 1580486 | 1580497 | UUUCCCUUCCUC | 622 | 757 | 779 | 1580492 | UAGGGAAGGGA | 933 |
| | | CCUUCCCUAUG | | | | | GGAAGGGAAA | |
| 1580487 | 1580499 | CUUUCUGUUUUC | 623 | 765 | 787 | 1580494 | GGAGGAAGGGA | 934 |
| | | CCUUCCUCCCU | | | | | AAACAGAAAG | |
| 1580488 | 1580496 | UCUGCUUUCUGU | 624 | 769 | 791 | 1580493 | GAAGGGAAAAC | 935 |
| | | UUUCCCUUCCU | | | | | AGAAAGCAGA | |
| 1580501 | 1580517 | CCUUCCCUAUGU | 625 | 745 | 767 | 1580512 | UCUGAGCGUAC | 936 |
| | | ACGCUCAGAGC | | | | | AUAGGGAAGG | |
| 1580502 | 1580514 | AGCCUCAGACAG | 626 | 725 | 747 | 1580507 | CCAGACGGUCU | 937 |
| | | ACCGUCUGGGC | | | | | GUCUGAGGCU | |
| 1580503 | 1580515 | UCAGAGCCUCAG | 627 | 729 | 751 | 1580511 | ACGGUCUGUCU | 938 |
| | | ACAGACCGUCU | | | | | GAGGCUCUGA | |
| 1580504 | 1580518 | AUGUACGCUCAG | 628 | 737 | 759 | 1580509 | UCUGAGGCUCU | 939 |
| | | AGCCUCAGACA | | | | | GAGCGUACAU | |
| 1580505 | 1580516 | ACGCUCAGAGCC | 629 | 733 | 755 | 1580510 | UCUGUCUGAGG | 940 |
| | | UCAGACAGACC | | | | | CUCUGAGCGU | |
| 1580506 | 1580513 | CCCUAUGUACGC | 630 | 741 | 763 | 1580508 | AGGCUCUGAGC | 941 |
| | | UCAGAGCCUCA | | | | | GUACAUAGGG | |
| 1580520 | 1580526 | CUGUUUUCCCUU | 631 | 761 | 783 | 1580523 | GAAGGGAGGAA | 942 |
| | | CCUCCCUUCCC | | | | | GGGAAAACAG | |
| 1580521 | 1580527 | CUCAGACAGACC | 632 | 722 | 744 | 1580524 | CGCCCAGACGG | 943 |
| | | GUCUGGGCGCC | | | | | UCUGUCUGAG | |

represents a phosphodiester intemucleoside linkage, and each “s” represents a phosphorothioate intemucleoside linkage. The antisense RNAi oligonucleotide is complementary to the target nucleic acid (PMP22), and the sense RNAi oligonucleotide is complementary to the first of the 20 nucleosides of the antisense RNAi oligonucleotide (from 5′ to 3′), wherein the last two 3′-nucleosides of the antisense RNAi oligonucleotides are not paired with the sense RNAi oligonucleotide (are overhanging nucleosides).

TABLE 39
RNAi compounds targeting human PMP22 SEQ ID NO: 1

| | | | | SEQ ID | SEQ ID | | | |
|---|---|---|---|---|---|---|---|---|
| | | | | NO: 1 | NO: 1 | | | |
| | | Antisense | SEQ | Antisense | Antisense | | Sense | SEQ |
| Compound | Antisense | Sequence | ID | Start | Stop | Sense | Sequence | ID |
| Number | ID | (5′ to 3′) | NO. | Site | Site | ID | (5′ to 3′) | NO. |
| 1580519 | 1580525 | UCAGACAGACCG | 944 | 722 | 743 | 1580522 | CGCCCAGACGG | 945 |
| | | UCUGGGCGCC | | | | | UCUGUCUGA | |

### Example 13: Effect of RNAi Compounds on Human PMP22 RNA In Vitro, Single Dose

Double-stranded RNAi compounds described above were tested in a series of experiments under the same culture conditions. The results for each experiment are presented in separate tables below.

Cultured A431 cells at a density of 20,000 cells per well were transfected using Lipofectamine 2000 with 20 nM of RNAi compound. After a treatment period of approximately 24 hours, RNA was isolated from the cells and PMP22 RNA levels were measured by quantitative real-time RTPCR. Human primer probe set RTS4579 (described herein above) was used to measure RNA levels. PMP22 RNA levels were normalized to total RNA content, as measured by RIBOGREEN®. Results are presented as percent PMP22 RNA relative to the amount in untreated control cells (% UTC). The values marked with a “†” indicate that the antisense RNAi oligonucleotide is complementary to the amplicon region of the primer probe set. Additional assays may be used to measure the potency and efficacy of RNAi compounds for which the antisense RNAi oligonucleotide is complementary to the amplicon region.

TABLE 40
Reduction of PMP22 RNA by RNAi compounds

| Compound | PMP22 |
|---|---|
| Number | (% UTC) |
| 1580285 | 64 |
| 1580286 | 54 |
| 1580290 | 46 |
| 1580303 | 34 |
| 1580304 | 17 |
| 1580305 | 40 |
| 1580306 | 19 |
| 1580307 | 24 |
| 1580308 | 34 |
| 1580321 | 19 |
| 1580322 | 35 |
| 1580323 | 18 |
| 1580324 | 21 |
| 1580325 | 24 |
| 1580326 | 19 |
| 1580339 | 24 |
| 1580340 | 26 |
| 1580341 | 22 |
| 1580342 | 63 |
| 1580343 | 34 |
| 1580344 | 33 |
| 1580357 | 24 |
| 1580358 | 30 |
| 1580359 | 25 |
| 1580360 | 24 |
| 1580361 | 21 |
| 1580362 | 37 |
| 1580375 | 47 |
| 1580376 | 28 |
| 1580377 | 28 |
| 1580378 | 24 |
| 1580379 | 34 |
| 1580380 | 30 |
| 1580393 | 19 |
| 1580394 | 42 |
| 1580395 | 18 |
| 1580396 | 18 |
| 1580397 | 40 |
| 1580398 | 35 |
| 1580411 | 19 |
| 1580412 | 45 |
| 1580413 | 20 |
| 1580414 | 38 |
| 1580415 | 20 |
| 1580416 | 37 |
| 1580429 | 59 |
| 1580430 | 54 |
| 1580431 | 20 |
| 1580432 | 39 |
| 1580433 | 28 |
| 1580434 | 47 |
| 1580447 | 30 |
| 1580448 | 21 |
| 1580449 | 29 |
| 1580450 | 25 |
| 1580451 | 33 |
| 1580452 | 32 |
| 1580465 | 48 |
| 1580466 | 14 |
| 1580467 | 19 |
| 1580468 | 16 |
| 1580469 | 27 |
| 1580470 | 17 |
| 1580483 | 14 |
| 1580484 | 69 |
| 1580485 | 69 |
| 1580486 | 94 |
| 1580487 | 31 |
| 1580488 | 28 |
| 1580501 | 34 |
| 1580502 | 115 |
| 1580503 | 58 |
| 1580504 | 82 |
| 1580505 | 76 |
| 1580506 | 61 |
| 1580519 | 119 |
| 1580520 | 73 |
| 1580521 | 116 |

TABLE 41
Reduction of PMP22 RNA by RNAi compounds

| Compound | PMP22 |
|---|---|
| Number | (% UTC) |
| 1580051 | 51 |
| 1580053 | 54 |
| 1580056 | 36 |
| 1580069 | 93 |
| 1580070 | 54 |
| 1580071 | 71 |
| 1580072 | 81 |
| 1580073 | 74 |
| 1580074 | 55 |
| 1580087 | 92 |
| 1580088 | 50 |
| 1580089 | 53 |
| 1580090 | 68 |
| 1580091 | 100 |
| 1580092 | 95 |
| 1580105 | 72 |
| 1580106 | 108 |
| 1580107 | 51 |
| 1580108 | 101 |
| 1580109 | 36 |
| 1580110 | 54 |
| 1580123 | 111 |
| 1580124 | 105 |
| 1580125 | 82 |
| 1580126 | 104 |
| 1580127 | 98 |
| 1580133 | 103 |
| 1580141 | 97 |
| 1580142 | 98 |
| 1580143 | 91 |
| 1580144 | 79 |
| 1580145 | 57 |
| 1580146 | 64 |
| 1580159 | 103 |
| 1580160 | 98 |
| 1580161 | 98 |
| 1580162 | 92 |
| 1580163 | 102 |
| 1580164 | 106 |
| 1580177 | 40 |
| 1580178 | 83 |
| 1580179 | 58 |
| 1580180 | 42 |
| 1580181 | 74 |
| 1580182 | 77 |
| 1580195 | 44 |
| 1580196 | 28 |
| 1580197 | 50 |
| 1580198 | 59 |
| 1580199 | 44 |
| 1580200 | 27 |
| 1580213 | 65 |
| 1580214 | 40 |
| 1580215 | 89 |
| 1580216 | 98 |
| 1580217 | 86 |
| 1580218 | 97 |
| 1580231 | 26 |
| 1580232 | 108 |
| 1580233 | 89 |
| 1580234 | 40 |
| 1580235 | 96 |
| 1580236 | 40 |
| 1580249 | 79 |
| 1580250 | 46 |
| 1580251 | 25 |
| 1580252 | 27 |
| 1580253 | 39 |
| 1580254 | 100 |
| 1580267 | 32 |
| 1580268 | 110 |
| 1580269 | 62 |
| 1580270 | 75 |
| 1580271 | 48 |
| 1580272 | 58 |
| 1580287 | 45 |
| 1580288 | 89 |
| 1580289 | 101 |

TABLE 42
Reduction of PMP22 RNA by RNAi compounds

| Compound | PMP22 |
|---|---|
| Number | (% UTC) |
| 1579820 | 131 |
| 1579821 | 104 |
| 1579822 | 123 |
| 1579835 | 136 |
| 1579836 | 113 |
| 1579837 | 124 |
| 1579838 | 70 |
| 1579839 | 72 |
| 1579840 | 127 |
| 1579853 | 96 |
| 1579854 | 39 |
| 1579855 | 78 |
| 1579856 | 65 |
| 1579857 | 119 |
| 1579858 | 117 |
| 1579871 | 57 |
| 1579872 | 40 |
| 1579873 | 63 |
| 1579874 | 36 |
| 1579875 | 96 |
| 1579876 | 47 |
| 1579889 | 43 |
| 1579890 | 31 |
| 1579891 | 43 |
| 1579892 | 43 |
| 1579893 | 31 |
| 1579894 | 34 |
| 1579907 | 100 |
| 1579908 | 44 |
| 1579909 | 86 |
| 1579910 | 78 |
| 1579911 | 130 |
| 1579912 | 57 |
| 1579925 | 36 |
| 1579926 | 34 |
| 1579927 | 71 |
| 1579928 | 37 |
| 1579929 | 42 |
| 1579930 | 37 |
| 1579943 | 42 |
| 1579944 | 39 |
| 1579945 | 43 |
| 1579946 | 71 |
| 1579947 | 35 |
| 1579948 | 44 |
| 1579961 | 34 |
| 1579962 | 47 |
| 1579963 | 40 |
| 1579964 | 42 |
| 1579965 | 38 |
| 1579966 | 33 |
| 1579979 | 50 |
| 1579980 | 34 |
| 1579981 | 45 |
| 1579982 | 83 |
| 1579987 | 33 |
| 1579988 | 54 |
| 1579997 | 58 |
| 1579998 | 58 |
| 1579999 | 30 |
| 1580000 | 43 |
| 1580001 | 26 |
| 1580002 | 40 |
| 1580015 | 36 |
| 1580016 | 28 |
| 1580017 | 45 |
| 1580018 | 53 |
| 1580019 | 60 |
| 1580020 | 29 |
| 1580033 | 70 |
| 1580034 | 60 |
| 1580035 | 47 |
| 1580036 | 36 |
| 1580037 | 40 |
| 1580038 | 40 |
| 1580052 | 89 |
| 1580054 | 94 |
| 1580055 | 93 |

TABLE 43
Reduction of PMP22 RNA by RNAi compounds

| Compound | PMP22 |
|---|---|
| Number | (% UTC) |
| 1579592 | 42 |
| 1579593 | 102 |
| 1579594 | 118 |
| 1579595 | 107 |
| 1579596 | 111† |
| 1579597 | 100 |
| 1579610 | 90† |
| 1579611 | 49† |
| 1579612 | 51† |
| 1579613 | 96† |
| 1579614 | 78† |
| 1579615 | 70† |
| 1579628 | 97 |
| 1579629 | 160 |
| 1579630 | 55 |
| 1579631 | 42 |
| 1579632 | 113 |
| 1579633 | 107 |
| 1579646 | 113 |
| 1579647 | 73 |
| 1579648 | 75 |
| 1579649 | 94 |
| 1579650 | 18 |
| 1579651 | 44 |
| 1579664 | 38 |
| 1579665 | 90 |
| 1579666 | 95 |
| 1579667 | 106 |
| 1579668 | 119 |
| 1579669 | 50 |
| 1579682 | 67 |
| 1579683 | 25 |
| 1579684 | 30 |
| 1579685 | 34 |
| 1579686 | 36 |
| 1579687 | 27 |
| 1579700 | 63 |
| 1579701 | 31 |
| 1579702 | 56 |
| 1579703 | 37 |
| 1579705 | 38 |
| 1579706 | 36 |
| 1579718 | 25 |
| 1579719 | 47 |
| 1579720 | 26 |
| 1579721 | 39 |
| 1579722 | 24 |
| 1579723 | 35 |
| 1579736 | 29 |
| 1579737 | 33 |
| 1579738 | 33 |
| 1579739 | 44 |
| 1579740 | 51 |
| 1579741 | 49 |
| 1579754 | 72 |
| 1579755 | 85 |
| 1579756 | 72 |
| 1579757 | 42 |
| 1579758 | 55 |
| 1579759 | 72 |
| 1579772 | 66 |
| 1579773 | 86 |
| 1579774 | 64 |
| 1579781 | 58 |
| 1579782 | 34 |
| 1579783 | 39 |
| 1579788 | 45 |
| 1579789 | 47 |
| 1579790 | 46 |
| 1579799 | 78 |
| 1579800 | 105 |
| 1579801 | 93 |
| 1579802 | 105 |
| 1579803 | 74 |
| 1579804 | 89 |
| 1579817 | 115 |
| 1579818 | 69 |
| 1579819 | 77 |

### Example 14: Dose-Dependent Inhibition of Human PMP22 in A431 Cells by RNAi Compounds

RNAi compounds selected from the examples above were tested at various doses in A431 cells. Cultured A431 cells at a density of 20,000 cells per well were treated using Lipofectamine 2000 with various concentrations of RNAi compounds as specified in the tables below. After a treatment period of approximately 24 hours, total RNA was isolated from the cells, and PMP22 RNA levels were measured by quantitative real-time RTPCR. Human PMP22 primer-probe set RTS4579 (described herein above) was used to measure RNA levels as described above. PMP22 RNA levels were normalized to total RNA content, as measured by RIBOGREEN®. Reduction of PMP22 RNA is presented in the tables below as percent PMP22 RNA, relative to untreated control cells (% UTC).

The half maximal inhibitory concentration (IC₅₀) of each RNAi compound was calculated using GraphPad Prism 6 software (GraphPad Software, San Diego, CA) and is also presented in the table below. IC₅₀ values were calculated from dose and PMP22 RNA levels by least squares fit to equation: log(inhibitor) vs. normalized response—Variable slope, Y=100/(1+10\hat{ }((LogIC50−X)*HillSlope)).

TABLE 44
Dose-dependent reduction of human PMP22
RNA in A431 cells by RNAi compounds

| Compound | PMP22 RNA (% UTC) | | | | IC₅₀ |
|---|---|---|---|---|---|
| No. | 0.025 nM | 0.25 nM | 2.5 nM | 25 nM | (nM) |
| 1580304 | 40 | 23 | 8 | 12 | <0.025 |
| 1580306 | 38 | 14 | 11 | 9 | <0.025 |
| 1580321 | 35 | 17 | 11 | 11 | <0.025 |
| 1580323 | 73 | 41 | 13 | 12 | 0.13 |
| 1580324 | 78 | 19 | 19 | 13 | 0.08 |
| 1580326 | 38 | 17 | 13 | 11 | <0.025 |
| 1580393 | 130 | 60 | 25 | 14 | 0.63 |
| 1580395 | 52 | 19 | 10 | 14 | <0.025 |
| 1580396 | 40 | 15 | 10 | 11 | <0.025 |
| 1580411 | 59 | 16 | 12 | 9 | 0.04 |
| 1580413 | 33 | 16 | 9 | 10 | <0.025 |
| 1580415 | 56 | 22 | 12 | 16 | 0.03 |
| 1580431 | 90 | 58 | 24 | 15 | 0.48 |
| 1580448 | 75 | 34 | 13 | 9 | 0.11 |
| 1580466 | 64 | 24 | 13 | 15 | 0.05 |
| 1580467 | 71 | 55 | 24 | 13 | 0.26 |
| 1580468 | 53 | 24 | 10 | 8 | 0.03 |
| 1580470 | 69 | 24 | 17 | 8 | 0.07 |
| 1580483 | 52 | 20 | 14 | 14 | <0.025 |

TABLE 45
Dose-dependent reduction of human PMP22
RNA in A431 cells by RNAi compounds

| Compound | PMP22 RNA (% UTC) | | | | IC₅₀ |
|---|---|---|---|---|---|
| No. | 0.025 nM | 0.25 nM | 2.5 nM | 25 nM | (nM) |
| 1580307 | 85 | 26 | 16 | 16 | 0.11 |
| 1580325 | 40 | 28 | 14 | 14 | <0.025 |
| 1580339 | 51 | 20 | 15 | 20 | <0.025 |
| 1580340 | 62 | 46 | 21 | 14 | 0.11 |
| 1580341 | 74 | 38 | 22 | 15 | 0.15 |
| 1580357 | 58 | 41 | 19 | 20 | 0.06 |
| 1580359 | 78 | 48 | 26 | 18 | 0.29 |
| 1580360 | 51 | 27 | 14 | 18 | <0.025 |
| 1580361 | 47 | 24 | 22 | 19 | <0.025 |
| 1580376 | 30 | 20 | 15 | 16 | <0.025 |
| 1580377 | 37 | 23 | 17 | 15 | <0.025 |
| 1580378 | 29 | 15 | 13 | 13 | <0.025 |
| 1580380 | 56 | 24 | 18 | 13 | 0.03 |
| 1580433 | 99 | 114 | 46 | 22 | 2.88 |
| 1580449 | 108 | 50 | 22 | 16 | 0.40 |
| 1580450 | 80 | 43 | 16 | 15 | 0.19 |
| 1580466 | 53 | 18 | 13 | 8 | 0.03 |
| 1580469 | 62 | 25 | 13 | 13 | 0.05 |
| 1580488 | 74 | 55 | 28 | 17 | 0.35 |

TABLE 46
Dose-dependent reduction of human PMP22
RNA in A431 cells by RNAi compounds

| Compound | PMP22 RNA (% UTC) | | | | IC₅₀ |
|---|---|---|---|---|---|
| No. | 0.025 nM | 0.25 nM | 2.5 nM | 25 nM | (nM) |
| 1579650 | 54 | 37 | 13 | 15 | 0.04 |
| 1579683 | 32 | 23 | 18 | 24 | <0.025 |
| 1579687 | 51 | 28 | 24 | 30 | <0.025 |
| 1579718 | 68 | 26 | 15 | 18 | 0.07 |
| 1579720 | 75 | 62 | 22 | 17 | 0.39 |
| 1579722 | 47 | 36 | 16 | 27 | <0.025 |
| 1579736 | 83 | 40 | 21 | 33 | 0.28 |
| 1579999 | 41 | 13 | 10 | 13 | <0.025 |
| 1580001 | 54 | 24 | 15 | 17 | <0.025 |
| 1580016 | 109 | 76 | 42 | 36 | 3.15 |
| 1580020 | 51 | 24 | 16 | 20 | <0.025 |
| 1580196 | 84 | 62 | 35 | 31 | 1.06 |
| 1580200 | 110 | 88 | 40 | 42 | 4.63 |
| 1580231 | 67 | 29 | 22 | 30 | 0.07 |
| 1580251 | 54 | 37 | 22 | 30 | 0.03 |
| 1580252 | 63 | 28 | 25 | 28 | 0.05 |
| 1580358 | 134 | 46 | 32 | 36 | 1.08 |
| 1580452 | 71 | 49 | 30 | 26 | 0.29 |
| 1580466 | 49 | 22 | 11 | 12 | <0.025 |

1.-120. (canceled)

121. An oligomeric duplex comprising:
a first oligomeric compound comprising a first modified oligonucleotide consisting of 19 to 29 linked nucleosides wherein the nucleobase sequence of the first modified oligonucleotide comprises at least 14, at least 15, at least 16, at least 17, at least 18, at least 19, at least 20, at least 21, at least 22, or 23 contiguous nucleobases of the nucleobase sequence of SEQ ID NO: 352; and
a second oligomeric compound comprising a second modified oligonucleotide consisting of 15 to 29 linked nucleosides wherein the nucleobase sequence of the second modified oligonucleotide comprises a complementary region of at least 12 nucleobases that is at least 90% complementary to an equal length portion of the first modified oligonucleotide.

122. The oligomeric duplex of claim 121, wherein at least one nucleoside of the first modified oligonucleotide comprises a modified sugar moiety.

123. The oligomeric duplex of claim 122, wherein the modified sugar moiety comprises a non-bicyclic modified sugar moiety.

124. The oligomeric duplex of claim 123, wherein the non-bicyclic modified sugar moiety is a 2′-MOE sugar moiety, a 2′-OMe sugar moiety, or a 2′-F sugar moiety.

125. The oligomeric duplex of claim 121, wherein at least one modified internucleoside linkage of the first modified oligonucleotide is a phosphorothioate internucleoside linkage.

126. The oligomeric duplex of claim 121, wherein each internucleoside linkage of the first modified oligonucleotide is independently selected from a phosphodiester internucleoside linkage and a phosphorothioate internucleoside linkage.

127. The oligomeric duplex of claim 121, wherein the first oligomeric compound comprises a terminal group.

128. The oligomeric duplex of claim 127, wherein the terminal group comprises a 5′-vinylphosphonate.

129. The oligomeric duplex of claim 121, wherein the nucleobase sequence of the second modified oligonucleotide comprises at least 12, at least 13, at least 14, at least 15, at least 16, at least 17, at least 18, at least 19, at least 20, or 21 contiguous nucleobases of the nucleobase sequence of SEQ ID NO: 663.

130. The oligomeric duplex of claim 121, wherein at least one nucleoside of the second modified oligonucleotide comprises a modified sugar moiety.

131. The oligomeric duplex of claim 130, wherein the modified sugar moiety comprises a non-bicyclic modified sugar moiety.

132. The oligomeric duplex of claim 131, wherein the non-bicyclic modified sugar moiety is a 2′-MOE sugar moiety, a 2′-OMe sugar moiety, or a 2′-F sugar moiety.

133. The oligomeric duplex of claim 121, wherein at least one modified internucleoside linkage of the second modified oligonucleotide is a phosphorothioate internucleoside linkage.

134. The oligomeric duplex of claim 121, wherein each internucleoside linkage of the second modified oligonucleotide is independently selected from a phosphodiester internucleoside linkage and a phosphorothioate internucleoside linkage.

135. The oligomeric duplex of claim 121, wherein the second oligomeric compound comprises a conjugate group.

136. The oligomeric duplex of claim 135, wherein the conjugate group comprises a conjugate moiety and a conjugate linker.

137. The oligomeric duplex of claim 136, wherein the conjugate moiety is a lipophilic group.

138. The oligomeric duplex of claim 136, wherein the conjugate moiety comprises a C16 alkyl group.

139. The oligomeric duplex of claim 135, wherein the conjugate group is attached to the second modified oligonucleotide at the 3′-end.

140. The oligomeric duplex of claim 121, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.

141. The oligomeric duplex of claim 121, wherein the first modified oligonucleotide consists of 23 linked nucleosides.

142. The oligomeric duplex of claim 121, wherein the second modified oligonucleotide consists of 21 linked nucleosides.

143. An oligomeric duplex comprising:
a first oligomeric compound comprising a first modified oligonucleotide consisting of 23 linked nucleosides wherein the nucleobase sequence of the first modified oligonucleotide comprises at least 14 contiguous nucleobases of the nucleobase sequence of SEQ ID NO: 352; and
a second oligomeric compound comprising a second modified oligonucleotide consisting of 21 linked nucleosides wherein the nucleobase sequence of the second modified oligonucleotide comprises at least 12 contiguous nucleobases of the nucleobase sequence of SEQ ID NO: 663.

144. The oligomeric duplex of claim 143, wherein at least one nucleoside of the first modified oligonucleotide and at least one nucleoside of the second modified oligonucleotide each independently comprises a modified sugar moiety selected from a 2′-OMe sugar moiety and a 2′-F sugar moiety.

145. The oligomeric duplex of claim 143, wherein each internucleoside linkage of the first modified oligonucleotide and each internucleoside linkage of the second modified oligonucleotide is independently selected from a phosphodiester internucleoside linkage and a phosphorothioate internucleoside linkage.

146. The oligomeric duplex of claim 143, wherein the second oligomeric compound comprises a conjugate group comprising a conjugate moiety.

147. The oligomeric duplex of claim 146, wherein the conjugate moiety comprises a C16 alkyl group.

148. The oligomeric duplex of claim 143, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.

149. The oligomeric duplex of claim 143, wherein the first oligomeric compound comprises a terminal group.

150. The oligomeric duplex of claim 149, wherein the terminal group comprises a 5′-vinylphosphonate.


SEQUENCE LISTING (948 sequences)

SEQ ID NO: 1 - DNA, 1861 bp, Homo sapiens
aataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 2 - DNA, 41000 bp, Homo sapiens
acaaataggacattaaatgatagtgtctatggaagcatgatgagaagatatgctaggaacgtgctgggcacaattaggggcacggtagaatgtgagatagtccaggctgtgagtacgggatctgtagccagaatgcctggttggaatccagcctctgtcatttctttgcagtgtgtcttgagaaagtgactgaacatctcgctgtgcctcagtttctccctcttctaaaagaggatgatggggtgcctatttcatacggttgtgaggattaagtgatttgatgcatataaaatattcaagatggtgcctggcatatgctaaacacgaagtaaatataacctattatcatcattatcaaggacttgcggataactgatgctactgagccgatggagcgtggtgtagcagaacagctcaggtgctagaaatagccagtctcattgactcaactgtgtttcctcagagaatcccgattctcatttttcctatctgtaaatggaagggaggatagcaaataataataaatgttatttggtgactattctatgtgtttattttgcaagaggctcagtgggatatagcctacagctttgcagagacattgctcaacacagatgcatgggataggggtcccatttggctgagaaaaggttattggaaatgggagacgaggaaggagttttcctcacctgactctgggaagggaagagagtctggcccagatgggtaaagacaagtgtgttggcatctggtagacagggccagtggcacacaacagtgaacagctctactgctgacagccaacagggacttcggggtttggctgctggagatggaggacctgggcaaaagagacgtgaggccaagagagcaggtctcagaatgtggagctcatggaaggagagagggagggctaatcagactgctcctatctggccagaagccatgccccacactcaccaggccctaccccttttcctctgaatctttagctcttagatcagaaataatcttacgtgtcttcaggcttgatcctggcctgacggacagcagcctctgtcgtgtagcctttggagctgatcatggcagtacccaggaaaagactgtctggagaggctgcaaaaaccatgctcactgttcccagtccccgtacagaccatgtcagaggaggactgagggaggctcctctctctctctctctgtcacacacacgcacacgcgcacacacacacacacagatacacgcgcacacacacacaccgttttgcctggcagaacagacttataacttgcataatactgcagcagcccacactgtacctcatctccatttgggttccacaacagaagggagctggaaggaaatcagcgacaatctaactccacatgacaacctggctggacaacgaaacatccagcggggagaaagacactggaaattacccatacctgagccccatcccaaaacacctgagtcagcttctctgaccatggcccaggctaggtcatttgcaaagctcttgagtgattctaaagaaccactgctgtgaaaaataaaaatgatttcagcaaagagagaaaggtgaaactattttattggcactggctgtgatgctggctgtgagcagatgctggctgtgggcagaacaggggagagggccattgagaggcccttgtgactggagagtccaatcaaacgtaaagtgtgtgacagggagggactcctccaaattctgcaggtctctccagactggggatccctaacacaagaggaagtcttatgccccaagtgccagaattccaggaaactcatgcaatgaaaccagtcctcttgtgtcagagtttgcagctagaagcggttcaagggatggaggactctctgggagctggagtggtacgctgcaaagaacctaaatatgccataacttcctttacttccctctccaagaatttccaattgctcttccagcctattctgcctgacaggttagaaagttttcaatagttttcaatagaggttcgtttacttgcattgaattatctcatgaaggtcagcttgattcgggcacattaaagacagagtcggaagaacacatttccctaagttttattctaatgtacaacacatgtgatcatttgcgatggggtctaggcagacaaggcaggtaacagggagtccttccaaccaggggttggaacaaaggaggcttgatttggtgactcttgagacatttggctcactgctgtgatgctgtgagagattagctgtgcaatgtttgggctccttaaaaggaagtttatttaaaataaaacttacctgcacgtatgtaacactgtagacacagatccttcccacagtatatttaatctctgcagaattcactgggaggggaggggagccagtgggacctcttggctattacacaggttggcacttccagagagaacagtcttggcatcacaggcttcaggcatactcaaagctcttctcccttctgattccagtttctccatgccctgcagggcctcttgggattattgtattctggaaagcaaacaaagttggacactgtctctttaaataatagaggctgagaacctctcaggccaccatgacatatcccagcattggaccagcccctgaataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggtgagttttattggcaaactgtgcctctgggtgatgtgtgcctatgctttacaagaattgcctaatttcccaccccctgcaagccgcaaatgaaaaggattgcaggagagatggtgcatttgtgttggaattgactggagattcagagggctttttatatccttggttaaaaggtggatatatactctggctggggaggtggggggccatctgagagcatctagaaaccctaatgcatccagattgaaaggaagaaaggaatctagatgctttttcccccatggaaaatagctgtgcacacacagctggcaggtggccttggtaagcaggttagggagaagctgccacctgtggcagagccttggccagccgggctctgggtgtggcaagcctggtcagcaggcactgagtagcacttcctctgccaccattgagaaccaggcaggaggccagaggcagtgagggacagatgggttgggtttacctgtctggcagtatatggtgtggctgtgacctgggtggttattgattaaactattgggttcaaaagaaaggaagaagcgagctgtagcacccaatatatgcacttttctgtatgtgtattagactttattaaaaagtttatttgaaaatcacaaaggaagggaaagaaaaccctgagttagatatgctcatatttctaaagtgcttacttagaacagtcctgagtatgttgtgatcacataagtgttggttaaataaataaatgtccaaatggcataaaacaaagtaataatttctagagcaaatctaacttataaaatgacctgtggaggaaagaaaacactgccatgtctttagacttttttttctacttgcatatgcactttcatttataaatctttatctatctatctatatctatctatctatctatctatctacctacctacctacctacctatcagagttacaagctactttagtgcagaggtggtcaggtcttccctgaaaggttgatgtgagagttaaatgtgatataataatagtggtggcaatttgggcactgggcccttattatgttgcagacacgatgttaactgctttgtacatattgtctccttcaatcaccaccataaacctttaaagcaggcattactatttccttttgcacctgagaaaactgaagctcagtgaacttgagaaattgcccaaattcacaacaaaagtaagcagggtgattaggtttgttggagcccagagccagagtgcctaaccactgcaccatactgcctctcacagaccatgcatataaagtccaggcaaagtgcttggcatataacgggcactgtaaatgcagtgtttacaagtacacttcattttaatctgataacacgatagttttaaaaagatcattgtttgaagatctttttcaaatttatttttacttaccgtaagaatatacaattagctaaaatagcagctcctttcaaatcgtaaaataatatagaattatttgaatcattgagagtgatgagcttctatgacacagtacactagaggtagggaagtgtgtgtgtgtgtgtgtgtgcgcgcgcgtgcgtgaactgctgcattttcaggcagaaacctttaacatccacattcctgctccctgtcccgtgcctcaaggctggcctgcgcacgggagtctcagttgggcgcgcctctgccaagccgagactgaagggggctagcctctctccctgtaacgctgggcgggccacgttaggaggctatgaatcagctgatttccttggctgctccaaccccacctcaaatggccacctcgcacccgcccgccaaaccccatggccaggactccagccaaggctgacagccaagcccgactgctgcaggaacactttgcctagagcttattcggtggtagtctggttttgcctaggctaggaggagcccaatcccagaccatgctatccagtagtcggccggacttttcttcccctaattcgcacccaagaggagcccgctagatcaatccccgccaatcctaggaagctggcatgcttcgtaggtgcagacagtaatagcggggaccggcgcggggcaggtgtgtccggctaagacgccaggacagggcaggggctccaggacccgagaggagagggacttcttccagcgctcaagcgcccgctgccctctattagtgggaaaggaatccagcctcagccccgcgcgggcgcggtcggcgtcggcgggcccagaagcccagccctgggcatccgctgagctacatttggctgggtcttcccagagtgggctgaggagccagtttctcggtcaacactaggtctccacggggccaggggagaagggaggtgggaggtgagaaagctcagccgcctctggtttcgagtaaaagtcgccgcggttttgcagggaccgactttttcttgaggcgcatttaaggccaagtgactgtctcctgccctccctctctcctgccccctctcctccctgagtcccgccctcccgcacacgctgacccagggacacaccctactgcagcgacgcaaacagggcgttgttcccgttaaaggggaacgccaggagcctcccactgcccccttgcttcgcgcgcgcgcagccccgcagcgcagctttggcggcgccagcagcggagccaacgcacccgagtttgtgtttgaggccaccctgaggatcgggacagctgttcctttgggctgtaagtgatttggtggggagagtgaaggagatggaggagaaatgtgggcatcttccagtgagggtgccagaaagcgcagcgcaggcgcggggctttggccagctccctggggcttctgtttaggggcacagggtcccctgtgtgtcctgtttccctccaatgggtcttggagtaatacaacgaggagagcctttatggtctataagaccttacagggcaagggtgatggtgctggtgtctggatagcggataagggcggcccagttctcgccttgctgaagaggcgttgactctgggacacactggcaaaacagtccctgtggcggacatcccgagagtgttggactcctgtggttccccagttcccagcctcctgccatcggcttaattcaaaccctctgggagtcatcagaaatccttgtttatttcttccagtgcattccagagtttctttgaatgtgatgcttgtggagaggaacagaggggcctgggattgggtactttccaaactgggttattgaaacgttgtctccctggcccaagactctgcctaaaatggtccccagagtctcagccagatctttcacagccatgcatgtcctctaacaggtcatcccagccttcagttcccccaaagtttaaaatgaggggcgaggaccgcattacttgggataaatgtcctgccggctctgaccggctgaaattctcggactcagccttttaccctctgtctctctctcccgtccgctggacatctactcccttcggtccaggctcctgggctcctgtcccaccgcacccagaccggagctcaggcttgttggggttcgtgtcctggctttgagtgcctggggtgcagactggacccctaggcgagcacgtgggtctagcgcaagagcagaatgccccctgaccccaggggctgcctggggaagcgcgcggtggacgggaagcgcagggtccgggcaaccctctcgagccattcttttctttccacactactctggctctgcaccacctctccggagccgccagagtctgcgcgaggctgagctggggccaggaccgttcctctacgctggcagagttgggtggaaacttggagacaaacggaatgcggagagcactggggtctgggaaaccagcctcctcgccgctgtctcccccaccgcatgccacggctgtctccaggtctcagacggaaatctgggcacctactcccctcacccctacctccaccccataggggaattcaccatctgaaccggggtctcggagaacgtgacctctcaccttccagggaggtcgccgggaggtgcttggggtgggtggaagcgtgcagtggcctctgctcatattttctggaaacccctccgttccctgggtggttttagacgtgcgaaccgcttgttttgtttccaaaagcaaaagatgttccgttgcaggcgggcccggctgggcgctgggcactgggcgctggtcctgcaggcggctgctgccccctctcggcggcaggcggcgcgaaggctcctgacccgcgcgggcggtcgggctgcgggcgctgggccaggccgggccttccgctagtgcgcgggaccctccctctgcgcgcgcctccgtcgctcggcccagtgcgttcggcctcacgcccagcgctctcctcgcaggcagaaactccgctgagcagaacttgccgccagaatgctcctcctgttgctgagtatcatcgtcctccacgtcgcggtgctggtgctgctgttcgtctccacgatcgtcagcgtgagtgcctggcggggaggctccctgcgcggcccgcccttccccatctgggttcccagcccgtgctcctgctggttcaggactgtgttatttgcagacagttggaagtctcagacgtcccagggaagtttctggcaatctgcccccttccagttgctttgagaaaacgagagcagattcagtgataggaaccaagcgagcgctgggctgggttagctgcgcaggtctctatttaagccaagtaacttcagagccgatctagggtccccgaccttcatttgacaagattgtttaacttttttttttcttgatgcagcctcgttgaagaagaggagtatttatgatttttttttctccaaaccatagtcagatgggtgaattaatttataaaacacccttttaggacttgaaattggaaagttaaaatgggcttttctggaaaagagttcacaggtcccatgtcggctttatggcatgaacacaacacattgttagtaaagcttccactggtaggaacaggccatcagagtaacttctgttacaaaactgtccagcccattagatcttaaagttattttcttggcgatgaagatgagcagacattcaggcagttttccagtggtggctttcacttcttaatttaaggcacattgtaagcatgattttctttttcgttttcttttcttttcttttctttttttttttttttttgagatggagtctccagcctgtcacccgggctggagtgcagtgccatgatcttggcttactgcaacctctgccttccgggttcaagccattctcctgcctcagcctgggattacaggtgcccgccaccacgcctggctaattttttttttttttgtatttttagtagagacggggtttcactatgttggccaggcttgtctcaaactcctgacctcatgatccgcccacctctgcctcccaaagtgctgggattacaggtgtgagccaccgtgcccggcctgtaagcatgattttctagtctctgtgagaaataattatgttggggatttgtgacttagtttaacatttacaagcatcttcacagtatctccattgtgcctggtgatgatgatatcagactgataggagctataaacaaggaaatagactgagagaggcgggcttggggaatgatcccgagatgctgggaggagagaaggcttgaatgcaggtgtctaaggttgagttcatggctccttctactatagtaaacatctctgcacataatcgtttctgtgtgcatgtggaacttctccatttacaaggtgcttttaagtcataaaacgttggctcttaccatgcaggggtgggcggtgtggctaggtggatgcgggtgcttttcgccatccctgggcctttctccttccccttttccttcactcctccctccctccctgactcaggatatctatctgattctctctagcaatggatcgtgggcaatggacacgcaactgatctctggcagaactgtagcacctcttcctcaggaaatgtccaccactgtttctcatcatcaccaaacggtgaggctggttttgtgctccatgagcttgtcctcagacgcctgtaacacgtttctcagcccagagctggaaacgcttattggaagacacaagccagaatgtcgttgctggggtggggtgggatgtgacagggagccatgagtccagggaaaatgggaggggtggtgatctcgctggggaagctgagatcctgggcatcattgtgattcactctacctagagacatgcccttagtgccctcctgatcacttaggataatgctttttgtgattgaaaaaaatatcgacctgggagctgtgaaggtggtatgatagctaagcctttggcagacccccttgggacattggatcaaacgtgtatctttggtttggtggctgtctctattcagaaatgtcctgcccctttctgctgcagtgacagtggggctagctcactgcaccgtcagttacatggacacaagactggctgcctttccaggtcttgggcctcagtgatgccacctagcatcaaaacctttgaatgtttccttgttgcaagatggggacttgaagctatttgcaaaaccctaagctaagccttggggtccttaatccagaagatcgtatttctcctctgtcctcagcatctttgcttgtgagatatggaagccaggcttcatgagggtcaaattaagggataattggcagcagaagcaccaactaggtacccaaggcacccagagggaaaggaacacccagccaggagccctgtgtgtttggatgcagacaggtggaggtaaacaggatgagctgttttggtggctgggtaggccatagcgattgatgttaagagcccgtggacctggatccacatgatcttttgttccagaagatgtgtcctcaggctggaaggacctagagatacagcagagctttcctaacatcctgggtttaaaaggccacggaaatacagctttaaatgttgctggagctactcatagatccagtatatatgttcaagatgctacaccagacgagagaaaccagtttagatccagttcagtaaaacaggcagtgaagtggcctttggtcctggctttgcataatccagttgctttgctggactttccctcttacagctcagtacccagtaatacccaagcatgtccccagctgaagcattataaaccaaattccttgaaccacagtacaaatagaaactgctaaaataataatttggtaatttacatggagaagatacgcaaggagtagaaaaccctgctttatgtttttaccttctgggttggggagggagcagagaaaaggtcccagtcctattggagtgagaacagtgcttaatcttgtctctgagtgtgtgaaaggcattgaattaaagccatcccagaaattacgtggtgggtttgcatggtgagttggcaactgagtaaatgaaatactcatgagtgcctgagaatgtgaccgtagcactgcttgtccccaaagccacagcatccggtttctccccctttagggtctggctgaagttcccggagcactccggatgggagtctccgtactcgctactgacacctggtggttacaagtagtgactcagcccgaaaagggcaggctttggtgctcacaggcgccatccccaagtggcatctggccacgcggcttggcacagaatcctggactcttgggcccggaagggggcagttttggcaggcgtgttaaaacccggcgaagtttcagcagaaacgaggcggcagaggagttgctaagttgtgcttaaaccatctgcaggaaagaaagcaatattgacctgtagcatgttacagattgacatttggtgtcttctgccgttggaaatagctgtcagttggtgctagaagcagatctcaaagaggcactagggttattttaatcaggaaacttcttatctctcaacttgttccttctgtgccaagctcatgttcttgagttcatttacaatgcctccttagtgtgggtttcaaatctgcctcgttgctttccgtgaggaccccagagtgtctgttttccacatctcccttctcagctctcacaatcagggcattttgaaaacatctggacactgcacctgaacatgctgggtttgttttcacgccactcaggcttaatctaaatttgaaatttccatttacacttccccagtagtggttatctctgccgctatcttccccaacgtggcagcacttgctacgacttcagccttaagtggcgaatcctccagggcctctttgattgagtttaacaattgtggctgcagacaataagctgaaaaaaatgtgtactttttaaaacattatactgtctttacaaatgaactatgctcattgcgaaccatttaacttgtctgtatttgctcttagaaagtatatttgaaaaaaaatacttgttaagtaatcaaaagtaatttgttattgacatcgatttgtgaagaatatgttgcaagtgtgaaaaaataaatagtggtgcaatctcggctgactacaacctccacctcctgggttcaagcgattttcctgcctcagcctcccgagtagctgggattataggcgcccaacaccacgcctggctaatttttgtatttttagtagagaggggtttcatcatgttgggcaggctggtctcgaactcctgacctcaggagatccacccacctcacctcccaaagtgctgggattagaggtgtgagccactgcgccatgtgacttggtatttttttctgaaaggactcatttttctggtgcatgaccatgacacttcacctctctctgggggttgccactcatgttgttttctacatgaatggggcctcctggcatcgtgccatgaggcagtctaacatctaggttgtgtagtttgaaagagacacaccagatcttgcccccaggctacatcttaatattagaaatagttttagatttttaactaactgactttgactccgcctgccttctcgtttttctaaatgtgtttgtatactttctggcaccccctccttatctgcctcgtgtgggcagaggtaagagaggcttagtgtggggatattcgttatgatattgtatccagtgcctctccggcccagggaatacaaggatctggaatgcatgggccttgccttgatggtcccgagcctggtcgaagagtcaggagatagatatggaagggtgaatcactcccttggaaggcagagcaggccaggtgcccaaagggaggcaggcagacagcgtctgagttcagggaggaagcggatgggaaatctctggtgggaggtagaatctgatccatcctccaaaggatgggcaggatttaagtcaatacaggcaacggggaggagaatattccctgtgacctaaggtaagggtagaacaaggcaggggatggggtagactggttggctagtgttctggttaggattaggattgattgtgtataagagaacatcttcaaatgataggggcttaagtaagatcatttatttttctctcttttcaaagaagtctggaagtaggtagcctgagagaggtatggaggttccatgaaattgtcagagatgcagacactttctagctcatttctttgccatctcttgggtttggctgtcatcttcaggaaacaaaaggctgctagagctccagccatctcatccatgattcaagcagtagggtggagaaaggaggtggggaagaatgaatctccttttcttttaatgaaatgtgacatggtcacacctacacctatttacaagagaggctgagaaatatagtcattcggccttgggggaagcatggcttttcaaatcatagaaacctggcagcaaatacctctctgtaagcctggtgtggaatactttccttgagggtttgaccatcccctcccatacccaatcatactcttttgcaatagctcagttgggccctatgcagttccataagtcccagttatttcagcattcttcaaagagtccatctctgctgcatcctgggctctgtcagccaccccaccattctcacatgggctcttctcagaaactttaggacctgttgaggcttctctctctctatttctaccacctctccaggtataagtaagtcttattattattattgagacagagtttcactcttgttgcccaggctggagttcagtggcgccatcttggctcactgcaacctccgcttcccgggttcaagcaattctcctgcctcagcctcccgggtagctgggattacaggcatgcgccaccacgcttggctaattttttgtatttttagtagagacgggctttctccatgttggtcaggctggtctcgaactcccgacctcaggtgatccacctgcctcagcctcccaaagtgctgagattacaagcgtgagccaccatgcctggccatcttattattatttaatgagtccagacctctcttctgaatcccagacctgtttatccagctgtctcctggccacttcccttgggatatctaagagaggcccccaagtcactaggccagagacagtccttatcttcctctgccaattcaacccctctgtcactttctagtattttagtgaatgctgcttcaccagccagcaagccaaacatctagagtctgccttgacttgacgcgtttccttctttctcttctcagcattgtcacgggtccctcaggccaccccattgttccaatgcctgttgcccacctgtggtcacgctgcttcctcgtttgtcagaggcgccagggcagcctcctaactagtttgcctgaaccctttcttgctcctctgtgactcatttttcatatggaatccaggctgatatttaaaaaagcacaaacctaattatatcactcctcattttgaaacctttcagaggccgttcattggtcttaatcttcagatgaaaatctttaacaaggcccatcggaatctacctgaatgatccactcctgcttctgttaagtctcacctcccatctctcttcccctcatcttggaatttcagcgttttctcacgtcctgaaatgtgaacttcccacttcgggagtctctccatttctcttctgatctttcatgcaggactatttcttgaacaactctcgtgtaccagagactattgcaggtgctaaggttacaagagtgaacaagagcttatgttttactggacaaaccagtcagtaaacatggagaatttgggggcaatggtaagaactatataagacagggtaatgggataaagagggattgcagtggtgaaggaaggtctccttgaggagtggccaggagccagccatatgaacatctcggggaggttccatacagaaggaccgtcaagtgcaagatcgctggggagggaaggagcttggcgtagttgagggacagagggcagggcagactagtgggaacagagagtgaggggtaagtagaatcacagaccatctcaaaggtaccccccacccccactaggaaggagttgggtgcacatggatgcatttctaatctttgtgatttccttagacctgctgtcttccacgggtttctcagtggtccatttgattccaagttggagtgacagtccagaagcctctataaggcagaatatatcagaaattgtacaaggtagaaatgcatcggtatattttcagtgattattttgggtgacgggattcttatcactttggctgttttctttcttttgctgccttacctgttttcctcattgagatcttttattttcacaatcagaaaataactgtgaacattttatgttagaaaattggttttgagtagtaagtaggttccctctgccttgattaagagagtggagccctggggcatggcacgcagggtccatggcgtcgctttgaatgaggccagttcctggagctttagtttgcaaactcatacaatctggaaggctctggtgagggggaggggtgtgccaagcctcctccccaagaccctatgggtgagtttaaaatctactgacccaaaggattcaggcaaaacatttgccctgctattgaatgaaccagtgctttttgttttgaagtctccaagatccaaatatcaagtccaagttctcacaagcattcccctgctttttgtttaatttgtgtggacctgacgtgtagcagaatgggtcagaatactggaattaagccacaggcggcttgggagcccaagagctagaagggcacagtcctggcttgccctggcccctggctggtcttcatcagcttcagtgatgaggcaggacccaggcagtttgtcttcagtaaaatctgtctctcagggcctgtccctgctcacaaagtcctctcttgtctcagaagaaaaggtagttgataaactttttcatgctgaggtgttaggcacaaaaaatgtgcccctttttgaatcacttttgtttttaaaaaggaggccagtgctcaacaccaaagcatgaagggaattgttgagctttcctgtaatctgtccccaaacgtccttggccacctccattgtctaagggcagtacctgcagcccctcatttcccagtgacaacaggtgggtctgctctgccacagtgtgtgggcgggaccagcaccagacgctgagaagatgaccacttactactcttcttttccatttaaacagcagcagcagcagcagcagcagcagcagcagcagcaacagcaaactcatgactatcaataggttgtgtgaggaattagatcaatgctttgggttgaaaattagagaaactaaatcttgccagtctcagcagcccccaacctgagcccttggtggtctcctatcatcgctgtcttcaaaacgatcccagacttgtttatcaatttggtgacactgtcattttggcaaggacaccagacaatttccttgtctgtggctcagcttttggccagattttagaaaacacgggagaccttctgcatttatgggttcatgttttacattttaaaatcttcgtggctccctcagccaacccataatctctacccaaggaatggctctggattttacaggctcttatgagatgctgagaccctggtggtacaagggaggtacagaggacggaatgtgttgtcaacatgccaggatttcaacccttagggtttctctgatgccaatacccaggaattagtatgaaatgtttgtttgaacctgtgtctgaactgtatgacctggaatctatagtctttgctttaaaaaaacttcaattgtatccccaagatttcaccagcaccaagaaaacatcccttgcatttcaggagcagggactggccatggtgcagcctgaggttgtggatgttgatctttggtgcccgagagaaggctgtctgggatgaagccttctgactgtggtcaggtccctgccagtgctgggggccacatgagtgtgcagtcatccacacacaagtggcccccaacactggcagggacccggagtcatctggcccatccctccttcctatcccatcgagccacagacccctctgcaatatgcctaccacatgttcatcctaccactgatggggaacttattaccttcaagagagcctcttttgtttttgaagagctaatagtgagatccattcatgtgttcttcaactctattgagcctgtcctatgtactgggcactgttctagaaatggggtgcacagagatgaacaagaaagagcaggtccctgccattgtgcagctcacaaggagttggaggaaacaggcaatacacaatacatgcatcactatgggtagtggaacatgctctgcaggaaacaaatagggtgatccaatggaaagtcatcactggaggaatgagctgctcagggaaggcctcttccttccatagtgatgtggtctgagatttagaagacagggaaggaccagttataggaagaaccatgggaagaaaattctaggaggtaaagggaagaaccaaggccctgagttagctgaagaaaggagggtggggaatgcgggagagagggatgggagcaggatcgctggtgcctcacaggctgtggtactgagaaggacagttgggacatcattgaagggttttaagcagaagagtgtcatgatttgattcatgttttgtaagctcattctggctgttgagaatgaattctggggagaggcaagagtggaaactggggaccagtgaggagtttgtggtcatagtccagactggacatgatgatgacactgagatgacaatgatgacaatgacaatgatgttaagaatacaaacagctcacgtaagtgccaggcattctttgaaaagcaatgcatttaatcctcacaagaactctaggaggtagctggtggcttagctagggaggggaagtagagaagaaaacaggtctcatgacagggtttgctgatgggttagatgtagggagttagggaagaatgattctagatgactctacgttttgggcttgagcaactggtgtgtaactgaatcttggactgagatggggagactggaggaggaacagaagatttgcgggaaggtgtagaaatcaggatttatattttgaccatcagagaggtcagcaaatcacttcaatttagaagtctttggcttaggggagaggtctgggctggaaattcaaagtcaagagtcattcctctataccgaagggtattcaatgttgtggtgctgaatacagttacttggggaggagttgagagcagaaggcagaaaaggggaacagggttcagagctaagtcttgggcatgccaggggttaacgagggtacaggaaggagaggagactggggatgaacagccagtgatgagggaggaatgccaggagatattgtcacttaagaaattacctcggctgggcgcggtggctcacgcctataatcccagcactttgggaggctgaggcgggcggattacaaggtcaggagattgagaccatcctggctaacacggtgaaaccctgtctctactaaaaatacaaaacaaaattagccgggcgtggtggccggtgcctgtagtcccagctactcgggaggctgaggcaggagaatggcgtgaacccgggaggcggagcttgcagtgagccgagatcgcaccactgcacgccagcctgagcgacagagtgagactccgtctcaaaaaaagaaattaccttgttaaacccaacactgcccctcttttgtccattcacttctagttgccttcattcatgcctgatgtgctggacttgggttttatcaagttgcttggcacgtccttaaagaatttaaatctccacagtgcctcctggacaatgactgcagggtgacccccgtctctcctctacacgatgactcttcaaacagttgacgattgcagtttttctttccttccttctttcagtggcttctctggtgctggtcccagctgcataagcagggcctcttcttgcccttctgaagtttggtcagtggtttggcttggcaggctgatgagcagagaataagggaagccgctctctctttgccacaccattctccttccaccctctctagtttttgtggattctaagtctgaagaatggtggtagaataatttaattcattagattgcaagcatatcattgacttctctgcacccccagtgtgttgtagtggaaaaactagtagtctgggcatcagggaatccttgtttttctgcaccgggtctgcctgtaacaaattaggtaacctggggcaggtaccctggtttcctaaaagcagagcaggcattgaggtaggacctgaagttgaggtaggacctgcacattgcatgggtcccggagcctctgttcatgttcaggatgtaattgtggcatcgagggatcctaaaaggaggtggcctctgagctgggccttggcaatggggtagaattgcagtaggcagagggagcagagatgggttttgtgcttgtaaacgggcattggtttactgtcactggtgggggtagcagctcttttgttctagctcttcatttccataatgcgtgttcttttttcatactttaggggaaagaaggaggagaactctattatttctatggggagaattcctcctaaacctgaagatcttaaaactaagcgaattattccctgttcattctccactgatgcagttcatgactgcaattgcaactgcctttcccgtcattttctatggccaaactcagtgtttttaagtgagctcttcttttaaaaaacaaaaacaaaaacaagtctcctgataaatccgtttgaaagacactaagttttgaaagttaagcaggctttgatcattcatggtacattgtgaattaccaaggagggagattgatatccttcatcgtacaatgcactcccctcctttttcttttgtatctggtggagtaagtcttccaagagcaattcttagagaaacaaagcctactctgtttccctgtttctagagttttccaacaagggtataaaaaagcctcagacagcttactattatccagtaacctcaggtacctcagtggctgcgtgttattccctttagaagtccaaaaactcactagcagagcaagaatatggatgtcaaagtgcagaactagaccctgacaaactagctctgtgcctgccacaaagcctcttaattaggaatgcagttcattgaataactgtatcaaagttagctggaataccttgacatgaagattccttccacttactgagcagtttgtgcccactagtggccagacacaggctttgctctagctaaaatacccccaggattacctgttgagagagccgcccacgtcaccttcatcgcctttgtgagctccatgctggcacatagttgtctccatctgttttgctttccgcatgaatcataggcaaagttagcctgaccagcaagcccatttcaaagccaccagctgggggagaagttgaagcccagggggcaagacccacgctgggctttgggcatgtttgagctggtgggcgaagcatatgggcaaaggccacattgtttaggatggaggcctttcagagactcagctatttctggaatgacattcatactgagaaataaggaaaatggcgatctgtgtgatggttgtgggttgggaggtttgggcgtgggagtcctggtcttggggtcatgtgttttgaaaacagtctagcactatgcaaatgggaggtgttaataactctttgcctctgtgatttaccctctcatggctttttctctcttggcctctccaaggtctcttctgactcttgcaactgtccttcctttcactttcaacaaaacctgttctcttgagtcagaacagttttatgaattgcccagagtgggacttgatatggggcagggttggttccatgctggtaatgaaagatgcacataaactgttacattgaaaagtgcttcatactttctgagcccttaactcactaggttgtaggccctgggctaagaatatagctgagggggctgatgtatttccttcattgctcagctgctcctacagcaggaatctgaccttgaaatgagctttgatctttgtgcaactcgagcttcccactatctgctgggggagctaggtctgggtgctgccacggcccatgtcagaaagctgtcagcagctcttataccggacactctcagaacaggaggcctgcaaagagctttagctttagttttgttgttgttgttgtttttgagacagtctcgctctgtcgcccaggctggagtgcagtggcgccatctcggctcactgcaagctccacctcccgggttcacaccattctcctgcctcagcctcccaagtagttgggactacaggtgcccgccaccacgcccagctaattttttgtatttttagtagagacgaggtttcaccgtgttagccaggatggtctcaatctcctgacctcgtgatccacccgccttggcctcccaaagtgctgggattacaggtgtgagccacggcgcccggccttttttttttttttttttttgagacgagtcttgctctgtttccaggctggagtgcagtggtgtgatctcggctcactgcagtctccgccttccgggttcaagtgattctcctgctccagcctcccaaatagctgggattacaggcacatgccaccacacccagctaatttttgtatttttagtagagacggggtttcaccatgttggccagtatgatctcgatctcctgaccttgtgatccacccgctttggcctcccaaagtgctgggattacaggcatgagccaccacgcccggccagagctttagtttttaattccagtgctcagcaccaaggttggatattggcttaaaacttccactggaaactccacaaatgtttagcaggcctccactttgcaccaggaactgagtgtggcctggaggacagcaggtactcaacataagttctactatttattgcatacttgcaaattgggtggggcgtggggaagagtatcgccacactggggcttgtgttaatctatagtttagccacttgggatgttggtgtcacactaggtttgatctaaatagaacaggtttgtaattgacagcattttcacttgcattatatttctctgaactcgaatcattattttctgtacagctgacttgttttgctgtagcagagtttggcagataatagcttgcatgtcgaatccaacctgcttttgtaaataagattttattggataaatggctattgaaggaatgggtcacacccactcatttacatcttgtccatggctatggctgattttgtgatacaaaggcagaattgagtagttgttaagaagaccgtatgacctgcaaagcctaaagtatttgctctctgattctttatagatagtttgctatctgtgcagatatttgcactctgactctgtagcacccttatgctggagaggagatatgaattgataccaaagttgtatcaaaacacttttggagttctggccaatgtgctaataacctggcttgtgtgaacattgtggctctcattttcttttttcttttttttttttttgagacgagtcttgctctgtcgcccaggctggagtgcagtggcgcagtggcgcgatctcggctcactgcaagctccgcctccagggttcacgccattctcctgcctcagcctccagagtagctgggactacaggcgcccgccaccacgcccggctaatttttttttttttgtatttttagtagagacgggttttcactgtgttagccagcatggtctcgatctcctgacctcgtgatctgccctcctgggcctcccaagtgctgggattacaggcgtgacccactgcgcccggcctgtggctcccattttcatactgcttggtgtgacccagatggaaatgctccagaacctggcccctcaataacagcctgtaaatgcttgtttaccctgttttctcacatcagccttttaggtggggttatttgcactatcctttaacagcgcttcaataacaactcttttgtttgatcattatgacaacccaataaaatatattagtgcctcaagtacgtaaagcagaattcaaggccaaaaaaatgtatgtggcaagttaggtgggcggtcagaagaacttggcaacagagcccagccctgcctgggctgtcctaaccagagcacataaatgctggagagagtcaggctttgcacgtgctcacacgggacccttaccttggacaggatcaaagccctgtcatttctggagtgtgtgatatctgctgtctcttactgcgctggttgttacctgtctattccagttctgatgaatggctctgattggggagcagcatcatctgagcttagaaatgtcctcagggccctgtggggttccctttgagcaacaacgagagctcttgaatcttgcaccagccttttggagctggaatggagtttgctttctcctgggctgggctctgttttttttcctctcgctgtccttgtgtagcttttgatcaatggcactggaggaaaggaaaatcccttgttgttttcctgggcatgtggaagcccagtcctgcccagaattccactagccctttgagttttggaatcctgactcttagtaagtcaatagagtgctgcatctgtttttacctttgtaatcttttaaattggttttaattatttaagtaatacaagtattataatacatgaaaatatcctcaactgaaaggcttcttgaccaaactcttcttcgttgcaacagtagttcctgggggaacccactgacatcaatttgacacaactttggctctttctctacgaatatttgcatgcctgtggatgtctatacgcaattacatagttttttggtgtttgttttcataatgatgacatgttgtgtgtgcaccttactcttttcattgtcttggagacctttcaaagactcttcattttctaactcatgatttttttaactgctgcaaaatatttcattgtgaggatataccataatttattttccctcatagaaatgctacaatcaatgtccttatttattttcctctgagtatatgttcactgtttttccaggatatagtagataagcacttgaacacatatagtgtcattacgctagaggtgttacaatttttaaactacttttaatggcaaaaattacaattacttttgtatcaacctaatatattctgccacaaaatggctggtcacaaaaggattataataataatattattattattcctgtgaatggtgtctgcaggtagctatcacttatttgttctgctgatattatccattttttttgaattttgcctattggctggatgaaaattggtgtcaccctcctcatgggtttctcttgcatttccttggttgctagtgaggttgggcaccttttacgatgtttattggctgttcatatttctcctttgctaatcacatggcttttcaattttatacttagctgttttctctttgttctgtctgtaatttaattttgttaatgatgcgaactagagatttaattttatgttttaccaaatgaatagccaacactatgtattgtctgttgtatttttccccactggtttatatgatgttgtgtattatctaccaaatttacatgtatgcttccaaagtcttattccatcccattcttttatttttctttcatgattaacaccatatttttgaattgctaaaactttataatataaatataagctttgtatgtctataaataaaatacattttattaattataattatatgttatataattataatataagcacttgaacatatatacttctataattatatattatataattatatatcatacttctatgatatgttctataattatatattgtatatatcatagttctatgatatgttctataattatatattataaagttacaattaattgtatatttaatttataccatatactgtaataagcaaaatatagaaatatgttttaaatctggtaggtggtatcttgactcattggttttatatgtttttctgattttctttttgttcatttgttcgttttggtttttagttgctgttgtgtttacttcaaattttttcttatctgtcattgctcattgttttttagatgaatttttaattgatttttttccagttccagttgcaatatggattaggacagcattgaatttgtagattattttggatgaaattggcatctttataactttgagtgtttctatccaggaacttagtatgtttctttagtacttcttttatgtacttgaataaaatttgtctaacttgttgatgtatccccaaagaaccagacatttttattaaatgtattttaatatttttttctgtctcattagtctgtgcttttatccatattaagtcaattaatttttggacttatttcattgttttttttctggcttcttgagttgaaagtgtagttcactctttttaaggatttttctagcaaatgcttagaagtgtatacattgtcctctgagaactgctttggttacaacccacaggtgtgccatgtggctctctatataattccttgctgaagaagtttttacttttcatcttgttttttttaatccgaggaattatctggaacactgtttttaaatttccaaatttattttggctagtttgtttttaatgtctaatttctgtttgcctttaggtatatgttgagcctacatcatttctgcttctatatttattgagatattctttgttgcccttatagtcagtttgaaaagctgtcccatgtgtgtttgaaaataactttgctatatagacacaagttttctatacatctattaggacagatttgttaatcctcttatgtaatctatattctttcttctttttggtctacctgacatctattttgaggaaggaagctaaagtcttctactgttattgtgattttaacaatttcttctcatattttgattactttttgattttaactttttcaggccatgttattatatgcataatgatttattttcttcagtcatagatcatgtaaaccagaagtaaagatcaaataaaccaaatctttcctctatgaaacaaccaaacaactctctggccacttttatcctttcaacataagttctattttttattattaatttatcttctttcttttttgttaacatttgcctgaagcttcttttcagaatcttttttgtttttgactgttgcttgttttaggtgtatctattatattagaatatagctacaatgtctctcttttttttttttttttttttttttgagacagagtctctctctgtgcccaggctggagtgcagtggtgtgatcttggctcactacaacctccacctcctgagttcaagcgattctctgcctcaacctcccaagtagctgggactataggtgtccaccagcacacctggccaatttttttgtatttttcgtagagatggggttttggcatgttggccaggctggtcttgaactcctggcctcaagtgatctacctgcctcagcctcccaaaatgtcttaatcaaagataaaattccatatcttatgagtgccttgaacccattcacatataatgtggttgatgatagacttactatcttaatgcatgtgttttatttattgtgcttatggttgaaaaccatcttcttcatctctggatcatcagagtcaaccagagggcttggctcaaagcagtaggtattcaacaaatgctcattgaataaaaatgtccttgatcctctaattctgatttgattcgagaaataacttatggcaaagagctcaattgtaatggggttgggactaaaattcaagttaatgttgaaagatgtttcaagaagtatttaggttatattattaaatattagacactttctgtttatatagatttaacatataaagattttaagttttctgtgtatgtgaggtctcgtagtagtttctgctgaaaatgacaggggcgcggggttaagggtgaagacagtatctcaggattggagaaggaaatctaagatattgtctccaccagccatactcctcatgaacatgcacatggaaggacatttcccagccaaagtgaatccctttatttgttttagtttcagatttgagtctcagatttctttgtaattccagctgcatctaccaaagtgctagataatttttttattttaagcaaacaatattatgagaacattatctttattgcaaagtgctcctccagaagaacctctatgctggataaagacaaacatttgattaaggttatgtaagagtgaaatcaggatggctcacaacccattagccgtcattttcttactttaattgacaaagaaattgaaaaaatgaaagaagcatatgttgtgcttgaatgtgaagagtgctagactagaatcaaaggcctcttagatttggctgtgtcatttgggctcatgaaaacacctctggaaatctctttcagtgcaaaggagctggactagttgatctctcaggtttcttccaactctaaaatatctccagtctgtgccttggaaacatcttaggtgaaaatctaggaacagttaacctaatttgcacccttaaaattctgccatgagctgcttacaactcaaaacaagtttatcttactcagttactaattataaaccatccagatttcagagctgtgagtactgggtgaagcattgaaggtatgcttttgaagccattacatatggcagttactgagctgaaaggattaaatgctgcagcttccccagttgcccttcctccatgagagcagtgcctgcccccagcattctgtggcacttggaagacaagacagaggccaaatgcagatttttaccctgggcttccctctacagtgtggaactcaggttgtttcttctttcctccctgaaatgacatgagtttgcagcggatggtgaactgaagaaaccataggaggctctgtcttcttgcctgaatttcagttggaagcttggagatttggggttcaacagagataaggaagtgtaagccttcatcccgtctggtggttggcgatcacacaccgctctgtgctgaggctaatggccatgatcagagttgaccaaaaaaaaaaaaaaaaaaaaatacgggttgtccaagcaaattgatttccatacctatagagagcatacctttcttcatcagtattttcttccattcttccaaaaaattactttgggctctaacagccattcccgtgatctttacctctccttggggaatgcagataatttgaatagtggttttaagctatttttcttggaatacagaagttctgataagcccttcaaagacccctgagggcaagaagagggaaggtagtaggcagggctcaggccctctttaacacagacacatgtacataagtaacacatttgcaccaacactggaaggaaattcaatatattttaaaatgactttaactcctagtgatggaactatagatatttttttacagcccttccctgaatgttcacatgtgttttttatatatatgtgtgtgtcacgtatttgtatatgtgtatatacacatatatatagaaacacgtacatatttgtatgcatatttgtacatataggtatatatgtatgtaatgtgtacatattggcacatgtgtgtgtttgtatatgtatatatcagtacatatagatgtatgtatatatgtatatattttttcctccaaaataaacaataaagaatgtcttcttagattgcaaaagtgaattgtctgagttctgctaaggaaaaaggacttctgcttctgctgcctgtgaggactgtgctgtcagcagtcatggcccttcaggccctgcacctcgaggcagagccctcccccggccatggccagctctcctaaccaagtgtctctttcccccagaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtaagttgtggatggtaaagtccatgtggaagcggggtgcatccaagtctgcggaatgattagtttagtagaaggatgtggcctcagaatgactgatgttcatgagtctccccactggatgctttccataaagtgagggtgggtgcttgtatgtgtgggtgtgtacctgtatgtgtcttagaacttgggacttagaactctccccttctccctggaatgagatgcatatgaaagagaacttagaggatctggaaggaaggtccccacccaagccaggcgtatcaacaggaatgaaactgcaatctggacacataatcagaggtgaatactgaggctatctgtagagcaaaggtcaggcttgagagctgtttctgtagattacattatgcctccagaaaatggccctgatgtgctaagaactagcaaagtagttatcaggtatgtgtcttccaccaataggtagtgatgaagccacactgacaaattctcaccttccttgcttccagttcctagattctactgggctttgattgactgttgtcatcctctggtgtcttcattttgacactcttgtgtcacatattgtcatttccaaacatggggctatgacaacacatgaaaacacatgagaggtctccttaatctcctgcctaaactgtcttcaagttccctctttaaatacgttattaatatgcatagtgtgcagagtcctagaaacttctgttacacagggtgacatcttccaactttgtctctggattctgcctagcatcttacatgcttacatcttacatcttacatcttacatcttgcattctgcctagcatcttacattagctcttacatgtctgtctgttgacttactgttgactgaaccagcagggcattggagagaagtaagagctagatgtagtggtggattctgtggtccaaattcatagatcacaaacttcatatgtaccagagtatgtctaggtactgggagatgttctcaattctgaccctctgagagggcaaaggatgtagcatctcttctctgagttggttgtcagaatgcccatggtaccatttcaccactctgtccccaggagcagtcattggaaggttgacgtaaatagggttgtatgggaagacacagcccaaggttagatgttggtgaccttgtctagaagacagagagttcccctttcctgaaaaaaggaagtaaatgattaaccacttctcattaaacactcaaatacaacatttcaatactcatggttttgagatttcaaaaccagacagtgctttgctacttacacatgtcttatgacaccaagccaagctcctggatggttgctggctctgttaaatgactaattatgcaaggagatatcatttctaggtacgttaaagtgaagagttacccttactcaattttcagttggaataaaaacaactgtaacatattctggggtttctttttttttttctcactcgttttagtttgatatcaaatcaaataatgatcatatccattgcatcagtggatatgccctcaagataatatggatttagaaccagaactttcataatgtatttctattgaaatgttagtttcataagcgatgattgggttttcatgcccatgtgtgagatgtgcctcgctcaaaccttgttatgatttggcacgttacccatctgatgtgaaaaaaattacattttatttgtacaggctcgttattttactgatgaataatttgagcccaccagaggataaatgaatgaccaaggtcacccagctcatgacagggacggttgagtgttacactgaatatagtgaggtacttcttatattttaaagacagaatgcaccaaaaaatttaaagaacacaaaatccaaggcagaagctctgccttttatattatcttttattggaactgatttacaatggaaggtaaatgcaaatttgcaccatgtattattctgaagttccaaacatctgtgatgaatacaagcctgtactataagacccagtcacattgaaaatatggagctgagaagaggtaagctgctgttgaatgggctccttgggatagccagtaccttcatcttcattcatcctgctgagctgtttcggctttaagttctttaacaatgtctttttagcaacatcattacatcattttaggccaaaactcaaagtccagagataagaacccttaagtcactcatgtaactgcactgtgtgttaaaagtatttcagttcagccaaacactcttctcctaggtattgcgatttaagtatatttactaatccactcttgcttcactattttcattctcctccaaagtcaatacaagatgtttagaactgtgctggaagtgcagaattccgaatgtaaaagcgcatgactttgtcctctttatcccctttacatctagctgcttacgtctcatgaaactgaattttcagttatctgttggtccacatttgaataagaaattatcttgaatttgaaatgctgagctgtaatagcagttgtaatttgtaagtcctgagagtgtcctgtcctccgttgttaatcccagtcaagatcatctgagagttggtctccagggaacctcagatctctaggatgttgcactggaaatggctgcaggatctttccactaattctgagaactgaaagagttaggaactctatttggagagttcctggttcccttatcgtgtgacagttctaagtcaattttgtcatgtggttttctgactcacagactagtaagaagttagtaattagagagctaagtagattagggttgttggagatgagaaacccacgttttgggaaaacctggcaagtgacaacttaacatcaaggaagtagtcagaaaagctaaaactgacaaacagaaggtagaagaaagtagctccacactcatgggatgtgaaatctacaagcgtgcatgcccggcaaatgcctctccaatgcactggagcgttttaagtggaaaccactagaatctctgtgtagtctccggaagtggctgtgagggctgcattatctctgcacagcttcctcttggtgggcccagctgtgatctttatggatggcacacatcagctttcaggaaaagcacatgaaaggtgctagggctcttggagctgactgtaggtttgggagttgtctgtctccttgctctagattacagctctgtgtgttgtgtggggtctccatggtttgccaaattatctcttcctcacttagccacaaggctgacagttaggaactatctcttcttgattgcattaggttggctgcttcctgaatgcatatcaaaaggctccttcctttagttcagtgctttcaacctgagctgtgcatcagaatcacctgaggtcctttaaaaaaaaaaaaaagacagtgggcggggcgcagtggctcacgcctgtaatcccagcactttgggaggctgaggtgggcagatcacttgaggtcaggagttcaagaccagcccggccaacatggtgaaaccctgtctctactaaaaataaaaataaaaaaatagccaggcgtggtggtgcgtgcctgtagtcccagctacttgggaaactgaggcagagaagaatcacttgaacctaggaggtggaggttgcagtgagccaagatcatgccactgcactccagcctgggggtgacagagtaagactgtctcaaaaaaaaagaaaaaagaaaagaaaaaagacagtattcaggtctcatccctggagactctcatttaatagttgtgggatggatccactgcctaggtgactctgatgtgcatttagggttgggaaccactgacatagccattaaactgtccctaatcccactgtaaggttttctaggatattttcccagaaataactaaaccaccttcttagagaaggaacatcctgatcctgcgtctggactttggagtcattcttattttcagaacctatagccatctattccttgacaagatctgttgggttgggttgcactagactggaaaacatcaagaaattaatctagacacagacctaaaaggaagatttgcacgttttgatttattttactgcttacacccagcatcaagctccatgtggggcacatagaggagcctgaataaaactattattggttggatgattagataattgtgtttcctcggcagaatcaaactcaagtcaaatgtgtggttaccgggattaagaacagaaagaatagaggggactcctgagtgagtttcactttctctctctttttgttattgttgttcatttgttttgttaatagaaagatcaatatagctatagagcatttaaaataaaaggatttgggccaggtgtggtggcgaatgcctgtaatcccaacactttgaaagactgaggtgagaggattgcttaaagccaggagttcaagaccagcctgggcaacaaagcgagatgccatctctacaaaaaatgatttaaaaattagtggggtgccttggagcacgcctgtggtcccagctattcaggaggctgaggcaggaggattgcttgaggctgagaggtcaagactacagtgaactatgatcaggcccctgcacttgctccagcctggtgacagagtgagaccctgcctctctaggaaaaaaaaaaaaagaatttggtggggggggattaataaaagttcagtgccatcctgttcagagtgattcaaaggtggcttaagtcaaataccacatttagaaatttttgttagggacggatgattatggatgtctcactggccatgcccaaatcagagtcaatgctatgggtggctttctaggcagcaatggtcatttggatactgaaaatgtaaaaggagtgggcttcaatcacagaaacacagagaagtctcttgctttcagaggaacaggcctcacagccccttcctgccctcccttgttgccctgaggatgaagtgggagggagaaaaaggcacccactcatcaacccaacatcatagctgcaccttccagtgaccaccctggtggtctcactgacatccctttccagccaacttctctctgacttctgccccaggctctcaaatgaaagtgcttttgcgaggttaccaaagtacactttgaagtctttaatttgttggatcactctgctcctttcaatactgttagctacttactctttgaagctctattcccctggcttctgagacatcgctgtgttcccacttgcctgagtgaacacaccaggcggtcatgctggcctctcctctctccctcaccccacagccagccttctactgaatcctgcatttgggtagcatcctccgtaggcattacgcccatttctttgtcttgactcactaaccccagtgcagctatgcaaaggcctcctcatgcaccttccagctacctgccttgtccctgcccctgaatccattttctgcaccatggccagagtgatcattctaaaacgtgcgtctgatcatgtttctcctctgctgaagtttcctcagaggcttctcatcacagcttccttaacatgtcatgtaaggccctctctttcctgacccttagtgagctaatcagcctattatatgagagtccgtgtggcttctccagttctccagtttggactcagcgcccgtctatgctcttggggcatctggtgcatttccttccctctctcgaaattccatcgaactatattgaaattatatgcaaatgtctgtcttctagacacacagtccttaaggtaggagccatgtctcacttatcttggtacatctagagcagcagctagaacagggcccgcatgcaataggcactcattaaatgttcactgaaccaaagggaatggagatgataaggatgtactgggaattccctcagctacttcctttgaccttggccccttgggtgcttccttccagaggctctgggctctaatcactctcatggggttgcagtcactgttaaaggagcttaagactttctcttcaaaagggctttaggcagaacttgcaggaagttgttgcagacctacccatcctgagacagggagaaactcttgtaagttgaatgctcagcacatttgtattgtctggacagggtcagtgtccttctgcttaaagatgacctatgctcccagactcaggcccctaccagggagtcccggtcattgccaaagagcagaacatctgcctgtgcctcagggcctcacttacccttgccacagggacctggtgatacatctccacctactggttgctgagaaagtgagtcacagtccacttactaggggtatttggcttttggagatgactactggatgacattaactgtcttgggttgcagacagagggaacccagccaaagaatcatccttttctctatttcagggtacatctattgctttacatgcagacaatcttgtaataatatattccctaaaacatcagtctcatgacaacaatcatataaagtgtgtatcctctctttggctttgtagatatctaagtttactgcttatcacggttaagcttagagtcattcacttctgagattctactaagatgaaagtcatacacattaagctgtgtagttagtcgctgcacttctctatactgcctctttctatccctcttgttcaaggatggaatctggtatctcctcttagtcagtgaaatcggggagtagctggtccactcagctcctgaggcatttctcatgccagatctggttagacagcccatttgggagccctgcctgcatgatgataagtgttgtcctgccctgctagcactctgttaacacctttctctctgccaaggtcatgtttccttttttatagccctgccttcattagataagtcagacagagcccaagagacaacttcagtatttcttaaagacaagagttagcactttggagatgcaagccgtggaagagtggggcaagataattaaggatcctggacctcaaggcaaataaagtgtgtccccagcagatcaaaactgctgttttctgcaatcaacaattaacccgcaatgatttcgctcctgacatggccatctttgctgtcttctgaacttcctgtaggtggcaggtcctggatgctgtcccattttggttccaagactattgtgtggctctcttgacaggggccagtcaagtggcagttttctcctctgagttgcaggaggacaggcttaacagggtggttcatgtggcgtattcccacttctgcactgtcttcaaacaaagttgcagtgtccagtaaaacagcagcccaaatagcagtgaggttagatgtaggtggccgggcggtgggggggcgcgggtgcattgggaactctggaactgcatgaatcccacacggcatactggatttataatagagtcactcagagcttctcaaagaccctgttttcagggaggaaacagaccaggctttgtgcagctccaggctggggtgctgacatcactggacccctccttgtggggggacgctgtctgtcacgtgagctggggccatcagtttgagcgtggctgctccacagggcccaagatggaggcttttcttctctttccaacaccgaagggctgcattggtgggccagggagggtggcctgacccacctgcatatccagtgccctttagccttctttgggcttcaaatgcaaccttggggcacccagggaaatggagcttgcctttgatcatcttccccctctgcagcgtggcttcccagtcatactgtcggctgcatccttattcctcactttctttttccctctccacacatttctttgtctgtttggcagtgcccataggcattgcttgcttcacctggtatgtggcaactctgttagggggaccataggcccctgtggtctgcatggagcttaatccttttaccatggcgtgactccaataatggtaaaatgtcacttcacttccattggctttaggtaatgccaccactcacttgtagagtggcatatagatttccaggggtttccatacccaaccacccttggaaagagacaggtaggtttttcttgcagcctgaatgcctgctggccatgtagtgtggcctgggaattggtgacagtgaaagaaagagaaatagggcagaaaatgtaaagaaaaccctcaggtattaaggcctaggtggccaagattggaaatagagagcaagttgttttccttgttcccatatttttgtgctacctcctttcaattctggacctggaagcagatttccttccagaggtagaaatgttcttcctacccagcaattgtcagcatccggggtggcggagagggggccgctctgccatggactctccgtcacccagcttgtccctctccttccccaggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcactggtctctcttgagtttttgtgggaacttgggtgtgggtacaaggtgcctgttggagcaggatgaagagcttcagtggaaagcaaagttgagctggagatttgggttacaagatggtgctaaggcaccaaaatcatagctgttttacaagctacagggagcacatgatgccttcatgatgtctgtgcaaaggtttgctaattggagacagttgggaattgcagctgaaatggaggctgtaaatggactggaaggcacttgagtattcatttagttcaaccctctctggtgctctgttgtttgatgacccacatggaaaacatggaggcatgtcatgagctgccctgatatactgggaatgttggcagaagcatctcagatccctcaccctgaggtatgatatggtgaaaggtctgaatgatggtgacttgaggccacacttgcagataaaacattggttggcaccaactaagctcctaatgccatcctgaaggagagaaaaggccaccattgtcacccagctcagggtttctgagttgtgtctcatgcgtccatatttttacccgtcgaccttttgttgtgtgtctgcaaaataacaagcaaatgatgtggttgatagtccttaatatttattgtgcaactattttgaattgggtaccaaagataaagaagtaatgacatatatcttaacatcgaaaggtcacattttagtgagaggtacaagtaaacaagtgactatattataataaggttaagagcatagaaatgagagcctgggtttgagcctgtcctttgctgcttaccagccataggggcctgaaaaggttacgtaaacactaagattcagttttctcatctataaaatggggatgccaataacaagggtatagggctgaggggatttcaaggaagttatatggataaagtgttcagcatagtgactagtacctagaaagactctatagattagagtggttatcacatatgttacaacagaggtatatttgaaagagcatgtaggagagacattgactgttatgcaggattctcagtatcagtctgcacactggaatctcccatgcaggaattcccattagagatttggattccctgtttccatgatgggacccacccatcatggtttaaagaacacttctggtgattacagcatgcaactggggatgacacctctgagctagtgcaggaggtctcatacttcaacatgctagtgaatcacctgggaatctgatagcatgcagattctgaaactttaggcccaggtggggattgagattctgcatttccaattagtgcctgggtgaggtggatgctgcttgtccaccgaccacactttgagtagcaagggcctagcataaagccaccttcaggtaccttgtgctcttctgtccttgggtgtctgtgacggaaggacttctcttttctgaggttttcagtagtgactgctactctgcttttgactcagatagttttaaggtggcacaggtggccatgagatgcatgtgagattaagaaaactaggaattttatttcacgagctggtactttgaaaacatacacttttatttcataaacttttctaacatttcattaaaaattaatagggataagaataaagaagaaaagcaaaaacggcccctaaatctccctacccaggtaacactcttggcatttaaaaaaaaaccatatatctaggattataacatcctatcttgtatctagagggaaaaatacaatctgaaagcctctttcccacctacccatagtcttttgccaaaggtaaccgtaagacatagtttcttatgacttcttccaggaaagaaaattgcccacatatgtacatccatctatgtattttttaaacgtacacaaaagtgatcatactactcacttctgcaccttttcatttaatgataaaaatttgagatcattctataccattctataccagcatatacctcatgctttttagtgaacactattttattctttcaaaattctgcatagtattttattgtattgttgtacaaacctttatttaccagccctccattgatagatattttggttgtgtctactttttattattaatcaaaatatttctgtggacatccttgtacatgaatattggtgacttttaaaaagtatatccaaatcttaaatttctggtaaggatgttgccaaaacagagggtgtgtatattttgaagaatctaataggtattttcagcttagtctccaggaaaattacaccaactcagttgacagtggagccttacattctacatggaactcatggagtccccccaaacagagaagggttagaataaaacacttgagtaaaaacaacttatctgctatcagttccttgggtcatgattgttattgatctctcatctatggggacagcttcgcttccttgggaacttgtctgccttgaggatctgtaatgatagaagggacattatactgaccctacacaaacaacaacaaaaaattacaaagaaatgctatgaacaattgtatacaacaaattaggcaccttagatgaaatggacaaattccccagacacaattactgaaactgactcaatatgaaatagataataagaataggcctataatgagtgaagagattgatctattaatactactactaataaattattcacaaacaaaaaagcccaggcccaaatggcttcactactgagttctaccaaatatttaaagaagaattaataccaattcttaacaaactcct

SEQ ID NO: 3 - DNA, 1818 bp, Homo sapiens
tgttcccgttaaaggggaacgccaggagcctcccactgcccccttgcttcgcgcgcgcgcagccccgcagcgcagctttggcggcgccagcagcggagccaacgcacccgagtttgtgtttgaggccaccctgaggatcgggacagctgttcctttgggctgcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 4 - DNA, 1822 bp, Homo sapiens
tgttcccgttaaaggggaacgccaggagcctcccactgcccccttgcttcgcgcgcgcgcagccccgcagcgcagctttggcggcgccagcagcggagccaacgcacccgagtttgtgtttgaggccaccctgaggatcgggacagctgttcctttgggctgtaagcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 5 - DNA, 1857 bp, Homo sapiens
aataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccggaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 6 - DNA, 1749 bp, Homo sapiens
aataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccggcaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 7 - DNA, 1649 bp, Homo sapiens
aataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccggaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 8 - DNA, 1236 bp, Homo sapiens
acccgagtttgtgtttgaggccaccctgaggatcgggacagctgttcctttgggctgcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatccccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccat

SEQ ID NO: 9 - DNA, 20 bp, Artificial sequence
  Primer
cttgctggtctgtgcgtgat

SEQ ID NO: 10 - DNA, 24 bp, Artificial sequence
  Primer
accgtaggagtaatccgagttgag

SEQ ID NO: 11 - DNA, 23 bp, Artificial sequence
  Probe
catctacacggtgaggcacccgg

SEQ ID NO: 12 - DNA, 15 bp, Artificial sequence
  Primer
tcgccgcttgctgca

SEQ ID NO: 13 - DNA, 17 bp, Artificial sequence
  Primer
atcggccgtgatgtcga

SEQ ID NO: 14 - DNA, 23 bp, Artificial sequence
  Probe
ccatggtcaaccccaccgtgttc

SEQ ID NO: 15 - DNA, 22 bp, Artificial sequence
  Primer
agaaatctgcttggaagaaggg

SEQ ID NO: 16 - DNA, 20 bp, Artificial sequence
  Primer
acgtggaggacgatgatact

SEQ ID NO: 17 - DNA, 23 bp, Artificial sequence
  Probe
agcaacaggaggagcattctggc

SEQ ID NO: 18 - DNA, 16 bp, Artificial sequence
  Synthetic oligonucleotide
atcttcaatcaacagc

SEQ ID NO: 19 - DNA, 16 bp, Artificial sequence
  Synthetic oligonucleotide
attattcaggtctcca

SEQ ID NO: 20 - DNA, 16 bp, Artificial sequence
  Synthetic oligonucleotide
ggagcattctggcggc

SEQ ID NO: 21 - DNA, 16 bp, Artificial sequence
  Synthetic oligonucleotide
ggacgatgatactcag

SEQ ID NO: 22 - DNA, 16 bp, Artificial sequence
  Synthetic oligonucleotide
ggaggacgatgatact

SEQ ID NO: 23 - DNA, 16 bp, Artificial sequence
  Synthetic oligonucleotide
ggattatactgttagg

SEQ ID NO: 24 - DNA, 16 bp, Artificial sequence
  Synthetic oligonucleotide
ccgttatatgccaagc

SEQ ID NO: 25 - DNA, 16 bp, Artificial sequence
  Synthetic oligonucleotide
gcatttacagtgcccg

SEQ ID NO: 26 - DNA, 16 bp, Artificial sequence
  Synthetic oligonucleotide
agttaaatggttcgca

SEQ ID NO: 27 - DNA, 16 bp, Artificial sequence
  Synthetic oligonucleotide
tgtaaataggtgtagg

SEQ ID NO: 28 - DNA, 16 bp, Artificial sequence
  Synthetic oligonucleotide
gtagatttcacatccc

SEQ ID NO: 29 - DNA, 16 bp, Artificial sequence
  Synthetic oligonucleotide
aggaaatagtaatgcc

SEQ ID NO: 30 - DNA, 16 bp, Artificial sequence
  Synthetic oligonucleotide
gtcaaggtattccagc

SEQ ID NO: 31 - DNA, 16 bp, Artificial sequence
  Synthetic oligonucleotide
gcacatcagggccatt

SEQ ID NO: 32 - DNA, 16 bp, Artificial sequence
  Synthetic oligonucleotide
cgcttttacattcgga

SEQ ID NO: 33 - DNA, 16 bp, Artificial sequence
  Synthetic oligonucleotide
ggattagggacagttt

SEQ ID NO: 34 - DNA, 16 bp, Artificial sequence
  Synthetic oligonucleotide
ccaagataagtgagac

SEQ ID NO: 35 - DNA, 16 bp, Artificial sequence
  Synthetic oligonucleotide
ccgtgataagcagtaa

SEQ ID NO: 36 - DNA, 16 bp, Artificial sequence
  Synthetic oligonucleotide
cgtatagacatccaca

SEQ ID NO: 37 - DNA, 16 bp, Artificial sequence
  Synthetic oligonucleotide
gaaatgatgtaggctc

SEQ ID NO: 38 - DNA, 16 bp, Artificial sequence
  Synthetic oligonucleotide
ctgattatgtgtccag

SEQ ID NO: 39 - DNA, 16 bp, Artificial sequence
  Synthetic oligonucleotide
gacaattgctgggtag

SEQ ID NO: 40 - DNA, 16 bp, Artificial sequence
  Synthetic oligonucleotide
ttaaggagacctctca

SEQ ID NO: 41 - DNA, 16 bp, Artificial sequence
  Synthetic oligonucleotide
cattcaatagcagggc

SEQ ID NO: 42 - DNA, 16 bp, Artificial sequence
  Synthetic oligonucleotide
tggattcgacatgcaa

SEQ ID NO: 43 - DNA, 16 bp, Artificial sequence
  Synthetic oligonucleotide
gatgtaatgatgttgc

SEQ ID NO: 44 - DNA, 16 bp, Artificial sequence
  Synthetic oligonucleotide
tcgggcagcggctgtt

SEQ ID NO: 45 - DNA, 16 bp, Artificial sequence
  Synthetic oligonucleotide
tctcagtatgaatgtc

SEQ ID NO: 46 - DNA, 16 bp, Artificial sequence
  Synthetic oligonucleotide
gtagaacttatgttga

SEQ ID NO: 47 - DNA, 16 bp, Artificial sequence
  Synthetic oligonucleotide
ccagataattcctcgg

SEQ ID NO: 48 - DNA, 16 bp, Artificial sequence
  Synthetic oligonucleotide
aaactaatcattccgc

SEQ ID NO: 49 - DNA, 16 bp, Artificial sequence
  Synthetic oligonucleotide
gttttataaaccggag

SEQ ID NO: 50 - DNA, 16 bp, Artificial sequence
  Synthetic oligonucleotide
tctcataagagcctgt

SEQ ID NO: 51 - DNA, 16 bp, Artificial sequence
  Synthetic oligonucleotide
gtatgaatgtcattcc

SEQ ID NO: 52 - DNA, 16 bp, Artificial sequence
  Synthetic oligonucleotide
tagaacttatgttgag

SEQ ID NO: 53 - DNA, 16 bp, Artificial sequence
  Synthetic oligonucleotide
gttgatacgcctggct

SEQ ID NO: 54 - DNA, 16 bp, Artificial sequence
  Synthetic oligonucleotide
gagataccagattcca

SEQ ID NO: 55 - DNA, 16 bp, Artificial sequence
  Synthetic oligonucleotide
catttatcctctggtg

SEQ ID NO: 56 - DNA, 16 bp, Artificial sequence
  Synthetic oligonucleotide
gcgatgataggagacc

SEQ ID NO: 57 - DNA, 16 bp, Artificial sequence
  Synthetic oligonucleotide
tccattggatcaccct

SEQ ID NO: 58 - DNA, 16 bp, Artificial sequence
  Synthetic oligonucleotide
tcagtatgaatgtcat

SEQ ID NO: 59 - DNA, 16 bp, Artificial sequence
  Synthetic oligonucleotide
acaacatactcaggac

SEQ ID NO: 60 - DNA, 16 bp, Artificial sequence
  Synthetic oligonucleotide
gacaatatctcctggc

SEQ ID NO: 61 - DNA, 16 bp, Artificial sequence
  Synthetic oligonucleotide
tggataatatcagcag

SEQ ID NO: 62 - DNA, 16 bp, Artificial sequence
  Synthetic oligonucleotide
gcacaacatatgcttc

SEQ ID NO: 63 - DNA, 16 bp, Artificial sequence
  Synthetic oligonucleotide
gtcaattccaacacaa

SEQ ID NO: 64 - DNA, 16 bp, Artificial sequence
  Synthetic oligonucleotide
aacaaggtttgagcga

SEQ ID NO: 65 - DNA, 16 bp, Artificial sequence
  Synthetic oligonucleotide
tccttttaggtctgtg

SEQ ID NO: 66 - DNA, 16 bp, Artificial sequence
  Synthetic oligonucleotide
cgtagagtcatctaga

SEQ ID NO: 67 - DNA, 16 bp, Artificial sequence
  Synthetic oligonucleotide
ggacttatggaactgc

SEQ ID NO: 68 - DNA, 16 bp, Artificial sequence
  Synthetic oligonucleotide
attgatgtcagtgggt

SEQ ID NO: 69 - DNA, 16 bp, Artificial sequence
  Synthetic oligonucleotide
catcatgtccagtctg

SEQ ID NO: 70 - DNA, 16 bp, Artificial sequence
  Synthetic oligonucleotide
ggatagcatggtctgg

SEQ ID NO: 71 - DNA, 16 bp, Artificial sequence
  Synthetic oligonucleotide
agcataggcacacatc

SEQ ID NO: 72 - DNA, 16 bp, Artificial sequence
  Synthetic oligonucleotide
gatcacaacatactca

SEQ ID NO: 73 - DNA, 16 bp, Artificial sequence
  Synthetic oligonucleotide
tgatactcagcaacag

SEQ ID NO: 74 - DNA, 16 bp, Artificial sequence
  Synthetic oligonucleotide
tgccataaagccgaca

SEQ ID NO: 75 - DNA, 16 bp, Artificial sequence
  Synthetic oligonucleotide
cagtagcgagtacgga

SEQ ID NO: 76 - DNA, 16 bp, Artificial sequence
  Synthetic oligonucleotide
tccaacggcagaagac

SEQ ID NO: 77 - DNA, 16 bp, Artificial sequence
  Synthetic oligonucleotide
tcgcaatgagcatagt

SEQ ID NO: 78 - DNA, 16 bp, Artificial sequence
  Synthetic oligonucleotide
acacattccgtcctct

SEQ ID NO: 79 - DNA, 16 bp, Artificial sequence
  Synthetic oligonucleotide
acataggacaggctca

SEQ ID NO: 80 - DNA, 16 bp, Artificial sequence
  Synthetic oligonucleotide
gcaaagagagagcggc

SEQ ID NO: 81 - DNA, 16 bp, Artificial sequence
  Synthetic oligonucleotide
ggtcaagaagcctttc

SEQ ID NO: 82 - DNA, 16 bp, Artificial sequence
  Synthetic oligonucleotide
tcccaagttctaagac

SEQ ID NO: 83 - DNA, 16 bp, Artificial sequence
  Synthetic oligonucleotide
ggcgaaaccgtaggag

SEQ ID NO: 84 - DNA, 16 bp, Artificial sequence
  Synthetic oligonucleotide
ccctagctaagccacc

SEQ ID NO: 85 - DNA, 16 bp, Artificial sequence
  Synthetic oligonucleotide
ggtctaaggaaatcac

SEQ ID NO: 86 - DNA, 16 bp, Artificial sequence
  Synthetic oligonucleotide
gcaagagtggattagt

SEQ ID NO: 87 - DNA, 16 bp, Artificial sequence
  Synthetic oligonucleotide
atatctaactcagggt

SEQ ID NO: 88 - DNA, 16 bp, Artificial sequence
  Synthetic oligonucleotide
ctgcactttgacatcc

SEQ ID NO: 89 - DNA, 16 bp, Artificial sequence
  Synthetic oligonucleotide
gccaaatgcaagggat

SEQ ID NO: 90 - DNA, 16 bp, Artificial sequence
  Synthetic oligonucleotide
gcatatctaactcagg

SEQ ID NO: 91 - DNA, 16 bp, Artificial sequence
  Synthetic oligonucleotide
tcacaacatactcagg

SEQ ID NO: 92 - DNA, 16 bp, Artificial sequence
  Synthetic oligonucleotide
tgtcatagaagctcat

SEQ ID NO: 93 - DNA, 16 bp, Artificial sequence
  Synthetic oligonucleotide
tctgaagttacttggc

SEQ ID NO: 94 - DNA, 16 bp, Artificial sequence
  Synthetic oligonucleotide
ctggattaaggacccc

SEQ ID NO: 95 - DNA, 16 bp, Artificial sequence
  Synthetic oligonucleotide
gtagattccgatgggc

SEQ ID NO: 96 - DNA, 16 bp, Artificial sequence
  Synthetic oligonucleotide
gactatagattccagg

SEQ ID NO: 97 - DNA, 16 bp, Artificial sequence
  Synthetic oligonucleotide
ggctcgatgggatagg

SEQ ID NO: 98 - DNA, 16 bp, Artificial sequence
  Synthetic oligonucleotide
ggtaataagttcccca

SEQ ID NO: 99 - DNA, 16 bp, Artificial sequence
  Synthetic oligonucleotide
ggtttaggaggaattc

SEQ ID NO: 100 - DNA, 16 bp, Artificial sequence
  Synthetic oligonucleotide
acgggaaaggcagttg

SEQ ID NO: 101 - DNA, 16 bp, Artificial sequence
  Synthetic oligonucleotide
ccatagaaaatgacgg

SEQ ID NO: 102 - DNA, 16 bp, Artificial sequence
  Synthetic oligonucleotide
gtgcattgtacgatga

SEQ ID NO: 103 - DNA, 16 bp, Artificial sequence
  Synthetic oligonucleotide
ggacaacccgtatttt

SEQ ID NO: 104 - DNA, 16 bp, Artificial sequence
  Synthetic oligonucleotide
ggtaaagatcacggga

SEQ ID NO: 105 - DNA, 16 bp, Artificial sequence
  Synthetic oligonucleotide
tgacaaccaactcaga

SEQ ID NO: 106 - DNA, 16 bp, Artificial sequence
  Synthetic oligonucleotide
acacatttgacttgag

SEQ ID NO: 107 - DNA, 16 bp, Artificial sequence
  Synthetic oligonucleotide
tgctctatagctatat

SEQ ID NO: 108 - DNA, 16 bp, Artificial sequence
  Synthetic oligonucleotide
gagtatatatccacct

SEQ ID NO: 109 - DNA, 16 bp, Artificial sequence
  Synthetic oligonucleotide
caacacttatgtgatc

SEQ ID NO: 110 - DNA, 16 bp, Artificial sequence
  Synthetic oligonucleotide
ctaatagagggcagcg

SEQ ID NO: 111 - DNA, 16 bp, Artificial sequence
  Synthetic oligonucleotide
cccttaatttgaccct

SEQ ID NO: 112 - DNA, 16 bp, Artificial sequence
  Synthetic oligonucleotide
ccaataggactgggac

SEQ ID NO: 113 - DNA, 16 bp, Artificial sequence
  Synthetic oligonucleotide
tctaaatctcagacca

SEQ ID NO: 114 - DNA, 16 bp, Artificial sequence
  Synthetic oligonucleotide
agcatagaggttcttc

SEQ ID NO: 115 - DNA, 16 bp, Artificial sequence
  Synthetic oligonucleotide
gaagatgaaggtactg

SEQ ID NO: 116 - DNA, 16 bp, Artificial sequence
  Synthetic oligonucleotide
gccgaaacagctcagc

SEQ ID NO: 117 - DNA, 16 bp, Artificial sequence
  Synthetic oligonucleotide
ttctaaatgaggtgga

SEQ ID NO: 118 - DNA, 16 bp, Artificial sequence
  Synthetic oligonucleotide
caatacaagtcattgc

SEQ ID NO: 119 - DNA, 16 bp, Artificial sequence
  Synthetic oligonucleotide
ggcttaaatagagacc

SEQ ID NO: 120 - DNA, 16 bp, Artificial sequence
  Synthetic oligonucleotide
tcttaacatcaatcgc

SEQ ID NO: 121 - DNA, 16 bp, Artificial sequence
  Synthetic oligonucleotide
caatgagcatagttca

SEQ ID NO: 122 - DNA, 16 bp, Artificial sequence
  Synthetic oligonucleotide
acaagttaaatggttc

SEQ ID NO: 123 - DNA, 16 bp, Artificial sequence
  Synthetic oligonucleotide
gataaacaagtctggg

SEQ ID NO: 124 - DNA, 16 bp, Artificial sequence
  Synthetic oligonucleotide
gtcaattacaaacctg

SEQ ID NO: 125 - DNA, 16 bp, Artificial sequence
  Synthetic oligonucleotide
gtcataatgatcaaac

SEQ ID NO: 126 - DNA, 16 bp, Artificial sequence
  Synthetic oligonucleotide
catattatcttgaggg

SEQ ID NO: 127 - DNA, 16 bp, Artificial sequence
  Synthetic oligonucleotide
cacgataagggaacca

SEQ ID NO: 128 - DNA, 16 bp, Artificial sequence
  Synthetic oligonucleotide
acgattatgtgcagag

SEQ ID NO: 129 - DNA, 16 bp, Artificial sequence
  Synthetic oligonucleotide
gaaatacgatcttctg

SEQ ID NO: 130 - DNA, 16 bp, Artificial sequence
  Synthetic oligonucleotide
gagaaatacgatcttc

SEQ ID NO: 131 - DNA, 16 bp, Artificial sequence
  Synthetic oligonucleotide
ctcttaacatcaatcg

SEQ ID NO: 132 - DNA, 16 bp, Artificial sequence
  Synthetic oligonucleotide
agctgtatttccgtgg

SEQ ID NO: 133 - DNA, 16 bp, Artificial sequence
  Synthetic oligonucleotide
acaaatcgatgtcaat

SEQ ID NO: 134 - DNA, 16 bp, Artificial sequence
  Synthetic oligonucleotide
ctcttatacacaatca

SEQ ID NO: 135 - DNA, 16 bp, Artificial sequence
  Synthetic oligonucleotide
aaatgacgggaaaggc

SEQ ID NO: 136 - DNA, 16 bp, Artificial sequence
  Synthetic oligonucleotide
tgcattgtacgatgaa

SEQ ID NO: 137 - DNA, 16 bp, Artificial sequence
  Synthetic oligonucleotide
aatgattcgagttcag

SEQ ID NO: 138 - DNA, 16 bp, Artificial sequence
  Synthetic oligonucleotide
gcattaagatagtaag

SEQ ID NO: 139 - DNA, 16 bp, Artificial sequence
  Synthetic oligonucleotide
ataggtatggaaatca

SEQ ID NO: 140 - DNA, 16 bp, Artificial sequence
  Synthetic oligonucleotide
ttatagtacaggcttg

SEQ ID NO: 141 - DNA, 16 bp, Artificial sequence
  Synthetic oligonucleotide
aacgacattctggctt

SEQ ID NO: 142 - DNA, 16 bp, Artificial sequence
  Synthetic oligonucleotide
ccgagatgttcatatg

SEQ ID NO: 143 - DNA, 16 bp, Artificial sequence
  Synthetic oligonucleotide
tgtacgatgaaggata

SEQ ID NO: 144 - DNA, 16 bp, Artificial sequence
  Synthetic oligonucleotide
gataatagtaagctgt

SEQ ID NO: 145 - DNA, 16 bp, Artificial sequence
  Synthetic oligonucleotide
ctggataatagtaagc

SEQ ID NO: 146 - DNA, 16 bp, Artificial sequence
  Synthetic oligonucleotide
acaaaggcgatgaagg

SEQ ID NO: 147 - DNA, 16 bp, Artificial sequence
  Synthetic oligonucleotide
caatatccaaccttgg

SEQ ID NO: 148 - DNA, 16 bp, Artificial sequence
  Synthetic oligonucleotide
gctaaactatagatta

SEQ ID NO: 149 - DNA, 16 bp, Artificial sequence
  Synthetic oligonucleotide
agcgaggcacatctca

SEQ ID NO: 150 - DNA, 16 bp, Artificial sequence
  Synthetic oligonucleotide
atccataatcatccgt

SEQ ID NO: 151 - DNA, 16 bp, Artificial sequence
  Synthetic oligonucleotide
gacaagattaagcact

SEQ ID NO: 152 - DNA, 16 bp, Artificial sequence
  Synthetic oligonucleotide
agtatatatccacctt

SEQ ID NO: 153 - DNA, 16 bp, Artificial sequence
  Synthetic oligonucleotide
catatattgggtgcta

SEQ ID NO: 154 - DNA, 16 bp, Artificial sequence
  Synthetic oligonucleotide
tgcatttacagtgccc

SEQ ID NO: 155 - DNA, 16 bp, Artificial sequence
  Synthetic oligonucleotide
tcttatagaccataaa

SEQ ID NO: 156 - DNA, 16 bp, Artificial sequence
  Synthetic oligonucleotide
aaacaagcggttcgca

SEQ ID NO: 157 - DNA, 16 bp, Artificial sequence
  Synthetic oligonucleotide
gtcgatatttttttca

SEQ ID NO: 158 - DNA, 16 bp, Artificial sequence
  Synthetic oligonucleotide
gaacatatatactgga

SEQ ID NO: 159 - DNA, 16 bp, Artificial sequence
  Synthetic oligonucleotide
gtattactgggtactg

SEQ ID NO: 160 - DNA, 16 bp, Artificial sequence
  Synthetic oligonucleotide
cccaaggccgaatgac

SEQ ID NO: 161 - DNA, 16 bp, Artificial sequence
  Synthetic oligonucleotide
gaaaatataccgatgc

SEQ ID NO: 162 - DNA, 16 bp, Artificial sequence
  Synthetic oligonucleotide
acctaatgcaatcaag

SEQ ID NO: 163 - DNA, 16 bp, Artificial sequence
  Synthetic oligonucleotide
cactaaagtagcttgt

SEQ ID NO: 164 - DNA, 16 bp, Artificial sequence
  Synthetic oligonucleotide
ttgtaactctgatagg

SEQ ID NO: 165 - DNA, 16 bp, Artificial sequence
  Synthetic oligonucleotide
cgctatggcctaccca

SEQ ID NO: 166 - DNA, 16 bp, Artificial sequence
  Synthetic oligonucleotide
accgaataagctctag

SEQ ID NO: 167 - DNA, 16 bp, Artificial sequence
  Synthetic oligonucleotide
tcctaaaagggtgttt

SEQ ID NO: 168 - DNA, 16 bp, Artificial sequence
  Synthetic oligonucleotide
agacaagattaagcac

SEQ ID NO: 169 - DNA, 16 bp, Artificial sequence
  Synthetic oligonucleotide
tgataaacaagtctgg

SEQ ID NO: 170 - DNA, 16 bp, Artificial sequence
  Synthetic oligonucleotide
agccacgaagatttta

SEQ ID NO: 171 - DNA, 16 bp, Artificial sequence
  Synthetic oligonucleotide
gtagagattatgggtt

SEQ ID NO: 172 - DNA, 16 bp, Artificial sequence
  Synthetic oligonucleotide
acgcattatggaaatg

SEQ ID NO: 173 - DNA, 16 bp, Artificial sequence
  Synthetic oligonucleotide
acacagatcgccattt

SEQ ID NO: 174 - DNA, 16 bp, Artificial sequence
  Synthetic oligonucleotide
gactataagggcaaca

SEQ ID NO: 175 - DNA, 16 bp, Artificial sequence
  Synthetic oligonucleotide
ctctataggtatggaa

SEQ ID NO: 176 - DNA, 16 bp, Artificial sequence
  Synthetic oligonucleotide
tcaatatagttcgatg

SEQ ID NO: 177 - DNA, 16 bp, Artificial sequence
  Synthetic oligonucleotide
ctatcttatgttgtaa

SEQ ID NO: 178 - DNA, 16 bp, Artificial sequence
  Synthetic oligonucleotide
ccattctatcttatgt

SEQ ID NO: 179 - DNA, 16 bp, Artificial sequence
  Synthetic oligonucleotide
ctccattctatcttat

SEQ ID NO: 180 - DNA, 16 bp, Artificial sequence
  Synthetic oligonucleotide
agaattattcaggtct

SEQ ID NO: 181 - DNA, 16 bp, Artificial sequence
  Synthetic oligonucleotide
ttattcaggtctccat

SEQ ID NO: 182 - DNA, 16 bp, Artificial sequence
  Synthetic oligonucleotide
aattattcaggtctcc

SEQ ID NO: 183 - DNA, 16 bp, Artificial sequence
  Synthetic oligonucleotide
tgtcagtgggttcccc

SEQ ID NO: 184 - DNA, 16 bp, Artificial sequence
  Synthetic oligonucleotide
ttctaagtcccaagtt

SEQ ID NO: 185 - DNA, 16 bp, Artificial sequence
  Synthetic oligonucleotide
tcacatcccatgagtg

SEQ ID NO: 186 - DNA, 16 bp, Artificial sequence
  Synthetic oligonucleotide
atgacgggaaaggcag

SEQ ID NO: 187 - DNA, 16 bp, Artificial sequence
  Synthetic oligonucleotide
aatgacgggaaaggca

SEQ ID NO: 188 - DNA, 16 bp, Artificial sequence
  Synthetic oligonucleotide
atagaaaatgacggga

SEQ ID NO: 189 - DNA, 16 bp, Artificial sequence
  Synthetic oligonucleotide
aattgatgtcagtggg

SEQ ID NO: 190 - DNA, 16 bp, Artificial sequence
  Synthetic oligonucleotide
tgtcaaattgatgtca

SEQ ID NO: 191 - DNA, 16 bp, Artificial sequence
  Synthetic oligonucleotide
gaattattcaggtctc

SEQ ID NO: 192 - DNA, 16 bp, Artificial sequence
  Synthetic oligonucleotide
cagaattattcaggtc

SEQ ID NO: 193 - DNA, 16 bp, Artificial sequence
  Synthetic oligonucleotide
acagaattattcaggt

SEQ ID NO: 194 - DNA, 16 bp, Artificial sequence
  Synthetic oligonucleotide
cacagaattattcagg

SEQ ID NO: 195 - DNA, 16 bp, Artificial sequence
  Synthetic oligonucleotide
acacagaattattcag

SEQ ID NO: 196 - DNA, 16 bp, Artificial sequence
  Synthetic oligonucleotide
ttacacagaattattc

SEQ ID NO: 197 - DNA, 16 bp, Artificial sequence
  Synthetic oligonucleotide
tattacacagaattat

SEQ ID NO: 198 - DNA, 16 bp, Artificial sequence
  Synthetic oligonucleotide
atattacacagaatta

SEQ ID NO: 199 - DNA, 16 bp, Artificial sequence
  Synthetic oligonucleotide
tatattacacagaatt

SEQ ID NO: 200 - DNA, 16 bp, Artificial sequence
  Synthetic oligonucleotide
ttatattacacagaat

SEQ ID NO: 201 - DNA, 16 bp, Artificial sequence
  Synthetic oligonucleotide
tttatattacacagaa

SEQ ID NO: 202 - DNA, 16 bp, Artificial sequence
  Synthetic oligonucleotide
atttatattacacaga

SEQ ID NO: 203 - DNA, 16 bp, Artificial sequence
  Synthetic oligonucleotide
catttatattacacag

SEQ ID NO: 204 - DNA, 16 bp, Artificial sequence
  Synthetic oligonucleotide
ccatttatattacaca

SEQ ID NO: 205 - DNA, 16 bp, Artificial sequence
  Synthetic oligonucleotide
accatttatattacac

SEQ ID NO: 206 - DNA, 16 bp, Artificial sequence
  Synthetic oligonucleotide
aaccatttatattaca

SEQ ID NO: 207 - DNA, 16 bp, Artificial sequence
  Synthetic oligonucleotide
ttctatcttatgttgt

SEQ ID NO: 208 - DNA, 16 bp, Artificial sequence
  Synthetic oligonucleotide
tatcttatgttgtaaa

SEQ ID NO: 209 - DNA, 16 bp, Artificial sequence
  Synthetic oligonucleotide
atcttatgttgtaaaa

SEQ ID NO: 210 - DNA, 16 bp, Artificial sequence
  Synthetic oligonucleotide
ttgtagatttcacatc

SEQ ID NO: 211 - DNA, 16 bp, Artificial sequence
  Synthetic oligonucleotide
agatttcacatcccat

SEQ ID NO: 212 - DNA, 16 bp, Artificial sequence
  Synthetic oligonucleotide
tttcacatcccatgag

SEQ ID NO: 213 - DNA, 16 bp, Artificial sequence
  Synthetic oligonucleotide
aaattgatgtcagtgg

SEQ ID NO: 214 - DNA, 16 bp, Artificial sequence
  Synthetic oligonucleotide
tcaaattgatgtcagt

SEQ ID NO: 215 - DNA, 16 bp, Artificial sequence
  Synthetic oligonucleotide
gtcaaattgatgtcag

SEQ ID NO: 216 - DNA, 16 bp, Artificial sequence
  Synthetic oligonucleotide
aaaatgacgggaaagg

SEQ ID NO: 217 - DNA, 16 bp, Artificial sequence
  Synthetic oligonucleotide
gaaaatgacgggaaag

SEQ ID NO: 218 - DNA, 16 bp, Artificial sequence
  Synthetic oligonucleotide
agaaaatgacgggaaa

SEQ ID NO: 219 - DNA, 16 bp, Artificial sequence
  Synthetic oligonucleotide
tagaaaatgacgggaa

SEQ ID NO: 220 - DNA, 16 bp, Artificial sequence
  Synthetic oligonucleotide
cgggaaaggcagttgc

SEQ ID NO: 221 - DNA, 16 bp, Artificial sequence
  Synthetic oligonucleotide
gggaaaggcagttgca

SEQ ID NO: 222 - DNA, 16 bp, Artificial sequence
  Synthetic oligonucleotide
ggaaaggcagttgcaa

SEQ ID NO: 223 - DNA, 16 bp, Artificial sequence
  Synthetic oligonucleotide
ttctaagacacataca

SEQ ID NO: 224 - DNA, 16 bp, Artificial sequence
  Synthetic oligonucleotide
tctaagacacatacag

SEQ ID NO: 225 - DNA, 16 bp, Artificial sequence
  Synthetic oligonucleotide
ctaagacacatacagg

SEQ ID NO: 226 - DNA, 16 bp, Artificial sequence
  Synthetic oligonucleotide
taagacacatacaggt

SEQ ID NO: 227 - DNA, 16 bp, Artificial sequence
  Synthetic oligonucleotide
tactgagctggattat

SEQ ID NO: 228 - DNA, 16 bp, Artificial sequence
  Synthetic oligonucleotide
agggcttttggacatt

SEQ ID NO: 229 - DNA, 16 bp, Artificial sequence
  Synthetic oligonucleotide
ttcgggcagcggctgt

SEQ ID NO: 230 - DNA, 16 bp, Artificial sequence
  Synthetic oligonucleotide
aggttcgggcagcggc

SEQ ID NO: 231 - DNA, 16 bp, Artificial sequence
  Synthetic oligonucleotide
gaatcatggatgagat

SEQ ID NO: 232 - DNA, 16 bp, Artificial sequence
  Synthetic oligonucleotide
gtatgattgggtatgg

SEQ ID NO: 233 - DNA, 16 bp, Artificial sequence
  Synthetic oligonucleotide
tcatatggctggctcc

SEQ ID NO: 234 - DNA, 16 bp, Artificial sequence
  Synthetic oligonucleotide
gggtattttagctaga

SEQ ID NO: 235 - DNA, 16 bp, Artificial sequence
  Synthetic oligonucleotide
aaccttacagtgggat

SEQ ID NO: 236 - DNA, 16 bp, Artificial sequence
  Synthetic oligonucleotide
atacgatcttctggat

SEQ ID NO: 237 - DNA, 16 bp, Artificial sequence
  Synthetic oligonucleotide
ttccatatctcacaag

SEQ ID NO: 238 - DNA, 16 bp, Artificial sequence
  Synthetic oligonucleotide
aatacgatcttctgga

SEQ ID NO: 239 - DNA, 16 bp, Artificial sequence
  Synthetic oligonucleotide
aaatacgatcttctgg

SEQ ID NO: 240 - DNA, 16 bp, Artificial sequence
  Synthetic oligonucleotide
agaaatacgatcttct

SEQ ID NO: 241 - DNA, 16 bp, Artificial sequence
  Synthetic oligonucleotide
gaggagaaatacgatc

SEQ ID NO: 242 - DNA, 16 bp, Artificial sequence
  Synthetic oligonucleotide
ccttaatttgaccctc

SEQ ID NO: 243 - DNA, 16 bp, Artificial sequence
  Synthetic oligonucleotide
ggagaaatacgatctt

SEQ ID NO: 244 - DNA, 16 bp, Artificial sequence
  Synthetic oligonucleotide
acagaggagaaatacg

SEQ ID NO: 245 - DNA, 16 bp, Artificial sequence
  Synthetic oligonucleotide
gacagaggagaaatac

SEQ ID NO: 246 - DNA, 16 bp, Artificial sequence
  Synthetic oligonucleotide
ggacagaggagaaata

SEQ ID NO: 247 - DNA, 16 bp, Artificial sequence
  Synthetic oligonucleotide
gaggacagaggagaaa

SEQ ID NO: 248 - DNA, 16 bp, Artificial sequence
  Synthetic oligonucleotide
gctgaggacagaggag

SEQ ID NO: 249 - DNA, 16 bp, Artificial sequence
  Synthetic oligonucleotide
tacgatcttctggatt

SEQ ID NO: 250 - DNA, 16 bp, Artificial sequence
  Synthetic oligonucleotide
atcttctggattaagg

SEQ ID NO: 251 - DNA, 16 bp, Artificial sequence
  Synthetic oligonucleotide
attaaggaccccaagg

SEQ ID NO: 252 - DNA, 16 bp, Artificial sequence
  Synthetic oligonucleotide
ttaaggaccccaaggc

SEQ ID NO: 253 - DNA, 16 bp, Artificial sequence
  Synthetic oligonucleotide
aaggaccccaaggctt

SEQ ID NO: 254 - DNA, 16 bp, Artificial sequence
  Synthetic oligonucleotide
tggcttgggtggggac

SEQ ID NO: 255 - DNA, 16 bp, Artificial sequence
  Synthetic oligonucleotide
tcgatatttttttcaa

SEQ ID NO: 256 - DNA, 16 bp, Artificial sequence
  Synthetic oligonucleotide
cgatatttttttcaat

SEQ ID NO: 257 - DNA, 16 bp, Artificial sequence
  Synthetic oligonucleotide
gatatttttttcaatc

SEQ ID NO: 258 - DNA, 16 bp, Artificial sequence
  Synthetic oligonucleotide
atatttttttcaatca

SEQ ID NO: 259 - DNA, 16 bp, Artificial sequence
  Synthetic oligonucleotide
tatttttttcaatcac

SEQ ID NO: 260 - DNA, 16 bp, Artificial sequence
  Synthetic oligonucleotide
atttttttcaatcaca

SEQ ID NO: 261 - DNA, 16 bp, Artificial sequence
  Synthetic oligonucleotide
tttttttcaatcacaa

SEQ ID NO: 262 - DNA, 16 bp, Artificial sequence
  Synthetic oligonucleotide
ttttttcaatcacaaa

SEQ ID NO: 263 - DNA, 16 bp, Artificial sequence
  Synthetic oligonucleotide
tttttcaatcacaaaa

SEQ ID NO: 264 - DNA, 16 bp, Artificial sequence
  Synthetic oligonucleotide
ttttcaatcacaaaaa

SEQ ID NO: 265 - DNA, 16 bp, Artificial sequence
  Synthetic oligonucleotide
ggtcgatatttttttc

SEQ ID NO: 266 - DNA, 16 bp, Artificial sequence
  Synthetic oligonucleotide
cccaggtcgatatttt

SEQ ID NO: 267 - DNA, 16 bp, Artificial sequence
  Synthetic oligonucleotide
tcccaggtcgatattt

SEQ ID NO: 268 - DNA, 16 bp, Artificial sequence
  Synthetic oligonucleotide
tctatcttatgttgta

SEQ ID NO: 269 - DNA, 16 bp, Artificial sequence
  Synthetic oligonucleotide
agaggagaaatacgat

SEQ ID NO: 270 - DNA, 16 bp, Artificial sequence
  Synthetic oligonucleotide
acgatcttctggatta

SEQ ID NO: 271 - DNA, 16 bp, Artificial sequence
  Synthetic oligonucleotide
gatcttctggattaag

SEQ ID NO: 272 - DNA, 16 bp, Artificial sequence
  Synthetic oligonucleotide
cttctggattaaggac

SEQ ID NO: 273 - DNA, 16 bp, Artificial sequence
  Synthetic oligonucleotide
tacacagaattattca

SEQ ID NO: 274 - DNA, 16 bp, Artificial sequence
  Synthetic oligonucleotide
attacacagaattatt

SEQ ID NO: 275 - DNA, 16 bp, Artificial sequence
  Synthetic oligonucleotide
cattctatcttatgtt

SEQ ID NO: 276 - DNA, 16 bp, Artificial sequence
  Synthetic oligonucleotide
cttgtagatttcacat

SEQ ID NO: 277 - DNA, 16 bp, Artificial sequence
  Synthetic oligonucleotide
cgcttgtagatttcac

SEQ ID NO: 278 - DNA, 16 bp, Artificial sequence
  Synthetic oligonucleotide
acgcttgtagatttca

SEQ ID NO: 279 - DNA, 16 bp, Artificial sequence
  Synthetic oligonucleotide
cacgcttgtagatttc

SEQ ID NO: 280 - DNA, 16 bp, Artificial sequence
  Synthetic oligonucleotide
gcacgcttgtagattt

SEQ ID NO: 281 - DNA, 16 bp, Artificial sequence
  Synthetic oligonucleotide
tgcacgcttgtagatt

SEQ ID NO: 282 - DNA, 16 bp, Artificial sequence
  Synthetic oligonucleotide
atgcacgcttgtagat

SEQ ID NO: 283 - DNA, 16 bp, Artificial sequence
  Synthetic oligonucleotide
attatatacatcttca

SEQ ID NO: 284 - DNA, 16 bp, Artificial sequence
  Synthetic oligonucleotide
gtattgaaatgttgta

SEQ ID NO: 285 - DNA, 16 bp, Artificial sequence
  Synthetic oligonucleotide
cgtaatttctgggatg

SEQ ID NO: 286 - DNA, 16 bp, Artificial sequence
  Synthetic oligonucleotide
gatatttggatcttgg

SEQ ID NO: 287 - DNA, 16 bp, Artificial sequence
  Synthetic oligonucleotide
tttataatgcttcagc

SEQ ID NO: 288 - DNA, 16 bp, Artificial sequence
  Synthetic oligonucleotide
attttattgggttgtc

SEQ ID NO: 289 - DNA, 16 bp, Artificial sequence
  Synthetic oligonucleotide
gcataattagtcattt

SEQ ID NO: 290 - DNA, 16 bp, Artificial sequence
  Synthetic oligonucleotide
gtaaatggagaagttc

SEQ ID NO: 291 - DNA, 16 bp, Artificial sequence
  Synthetic oligonucleotide
aaaataatgattcgag

SEQ ID NO: 292 - DNA, 16 bp, Artificial sequence
  Synthetic oligonucleotide
aaactatgtaattgcg

SEQ ID NO: 293 - DNA, 16 bp, Artificial sequence
  Synthetic oligonucleotide
atacttaaatcgcaat

SEQ ID NO: 294 - DNA, 16 bp, Artificial sequence
  Synthetic oligonucleotide
atagattacataagag

SEQ ID NO: 295 - DNA, 16 bp, Artificial sequence
  Synthetic oligonucleotide
gataatacacaacatc

SEQ ID NO: 296 - DNA, 16 bp, Artificial sequence
  Synthetic oligonucleotide
tagaaatgatatctcc

SEQ ID NO: 297 - DNA, 16 bp, Artificial sequence
  Synthetic oligonucleotide
cataagttatttctcg

SEQ ID NO: 298 - DNA, 16 bp, Artificial sequence
  Synthetic oligonucleotide
ttacttatacctggag

SEQ ID NO: 299 - DNA, 16 bp, Artificial sequence
  Synthetic oligonucleotide
atccatattatcttga

SEQ ID NO: 300 - DNA, 16 bp, Artificial sequence
  Synthetic oligonucleotide
agaattttaagggtgc

SEQ ID NO: 301 - DNA, 16 bp, Artificial sequence
  Synthetic oligonucleotide
taaatctctagttcgc

SEQ ID NO: 302 - DNA, 16 bp, Artificial sequence
  Synthetic oligonucleotide
attatggtatatcctc

SEQ ID NO: 303 - DNA, 16 bp, Artificial sequence
  Synthetic oligonucleotide
caagttaaatggttcg

SEQ ID NO: 304 - DNA, 16 bp, Artificial sequence
  Synthetic oligonucleotide
gaattttaagggtgca

SEQ ID NO: 305 - DNA, 16 bp, Artificial sequence
  Synthetic oligonucleotide
tgatatttggatcttg

SEQ ID NO: 306 - DNA, 16 bp, Artificial sequence
  Synthetic oligonucleotide
ttaatgagaagtggtt

SEQ ID NO: 307 - DNA, 16 bp, Artificial sequence
  Synthetic oligonucleotide
tcaaaacttagtgtct

SEQ ID NO: 308 - DNA, 16 bp, Artificial sequence
  Synthetic oligonucleotide
ttttattgggttgtca

SEQ ID NO: 309 - DNA, 16 bp, Artificial sequence
  Synthetic oligonucleotide
ttattcaatgaactgc

SEQ ID NO: 310 - DNA, 16 bp, Artificial sequence
  Synthetic oligonucleotide
tttaatgagaagtggt

SEQ ID NO: 311 - DNA, 16 bp, Artificial sequence
  Synthetic oligonucleotide
attgatagtcatgagt

SEQ ID NO: 312 - DNA, 16 bp, Artificial sequence
  Synthetic oligonucleotide
tcatttgaagatgttc

SEQ ID NO: 313 - DNA, 16 bp, Artificial sequence
  Synthetic oligonucleotide
ctattaaatgagagtc

SEQ ID NO: 314 - DNA, 16 bp, Artificial sequence
  Synthetic oligonucleotide
atcaatttgcttggac

SEQ ID NO: 315 - DNA, 16 bp, Artificial sequence
  Synthetic oligonucleotide
atgaatttggaccaca

SEQ ID NO: 316 - DNA, 16 bp, Artificial sequence
  Synthetic oligonucleotide
acgaagaagagtttgg

SEQ ID NO: 317 - DNA, 16 bp, Artificial sequence
  Synthetic oligonucleotide
caaacggatttatcag

SEQ ID NO: 318 - DNA, 16 bp, Artificial sequence
  Synthetic oligonucleotide
gtctagattaatttct

SEQ ID NO: 319 - DNA, 16 bp, Artificial sequence
  Synthetic oligonucleotide
gtattgacttaaatcc

SEQ ID NO: 320 - DNA, 16 bp, Artificial sequence
  Synthetic oligonucleotide
agtattgaaatgttgt

SEQ ID NO: 321 - DNA, 16 bp, Artificial sequence
  Synthetic oligonucleotide
  misc_feature (6)..(6): bases at this position are RNA
attatucaggtctcca

SEQ ID NO: 322 - RNA, 23 nt, Artificial sequence
  Synthetic oligonucleotide
caagaucacauagaugacaccgc

SEQ ID NO: 323 - RNA, 23 nt, Artificial sequence
  Synthetic oligonucleotide
gacaccgcugagaagggccaggg

SEQ ID NO: 324 - RNA, 23 nt, Artificial sequence
  Synthetic oligonucleotide
ggccagggggaaggccacccagg

SEQ ID NO: 325 - RNA, 23 nt, Artificial sequence
  Synthetic oligonucleotide
cacccaggccaggauguaggcga

SEQ ID NO: 326 - RNA, 23 nt, Artificial sequence
  Synthetic oligonucleotide
gagaugccacuccgggugccuca

SEQ ID NO: 327 - RNA, 23 nt, Artificial sequence
  Synthetic oligonucleotide
ucauucgcguuuccgcaagauca

SEQ ID NO: 328 - RNA, 23 nt, Artificial sequence
  Synthetic oligonucleotide
guaggcgaaaccguaggaguaau

SEQ ID NO: 329 - RNA, 23 nt, Artificial sequence
  Synthetic oligonucleotide
agcaagaauuuggaagauuccag

SEQ ID NO: 330 - RNA, 23 nt, Artificial sequence
  Synthetic oligonucleotide
caucacgcacagaccagcaagaa

SEQ ID NO: 331 - RNA, 23 nt, Artificial sequence
  Synthetic oligonucleotide
gauggccgcagcacucaucacgc

SEQ ID NO: 332 - RNA, 23 nt, Artificial sequence
  Synthetic oligonucleotide
gugccucaccguguagauggccg

SEQ ID NO: 333 - RNA, 23 nt, Artificial sequence
  Synthetic oligonucleotide
ggaguaauccgaguugagaugcc

SEQ ID NO: 334 - RNA, 23 nt, Artificial sequence
  Synthetic oligonucleotide
gauuccagugauguaaaaccugc

SEQ ID NO: 335 - RNA, 23 nt, Artificial sequence
  Synthetic oligonucleotide
gcugaagaugaucgacaggauca

SEQ ID NO: 336 - RNA, 23 nt, Artificial sequence
  Synthetic oligonucleotide
gaacagagacagaaugcugaaga

SEQ ID NO: 337 - RNA, 23 nt, Artificial sequence
  Synthetic oligonucleotide
uuggcagaagaacaggaacagag

SEQ ID NO: 338 - RNA, 23 nt, Artificial sequence
  Synthetic oligonucleotide
ggugagggugaagaguuggcaga

SEQ ID NO: 339 - RNA, 23 nt, Artificial sequence
  Synthetic oligonucleotide
aaaccugccccccuuggugaggg

SEQ ID NO: 340 - RNA, 23 nt, Artificial sequence
  Synthetic oligonucleotide
caggaucaugguggccuggacag

SEQ ID NO: 341 - RNA, 23 nt, Artificial sequence
  Synthetic oligonucleotide
ggaagaggugcuacaguucugcc

SEQ ID NO: 342 - RNA, 23 nt, Artificial sequence
  Synthetic oligonucleotide
guggacauuuccugaggaagagg

SEQ ID NO: 343 - RNA, 23 nt, Artificial sequence
  Synthetic oligonucleotide
cuggacagacugcagccauucgu

SEQ ID NO: 344 - RNA, 23 nt, Artificial sequence
  Synthetic oligonucleotide
ugaugagaaacagugguggacau

SEQ ID NO: 345 - RNA, 23 nt, Artificial sequence
  Synthetic oligonucleotide
ccauucguuuggugaugaugaga

SEQ ID NO: 346 - RNA, 23 nt, Artificial sequence
  Synthetic oligonucleotide
guucugccagagaucaguugcgu

SEQ ID NO: 347 - RNA, 23 nt, Artificial sequence
  Synthetic oligonucleotide
gcugacgaucguggagacgaaca

SEQ ID NO: 348 - RNA, 23 nt, Artificial sequence
  Synthetic oligonucleotide
gcccacgauccauuggcugacga

SEQ ID NO: 349 - RNA, 23 nt, Artificial sequence
  Synthetic oligonucleotide
aguugcguguccauugcccacga

SEQ ID NO: 350 - RNA, 23 nt, Artificial sequence
  Synthetic oligonucleotide
cagcaccgcgacguggaggacga

SEQ ID NO: 351 - RNA, 23 nt, Artificial sequence
  Synthetic oligonucleotide
gacgaacagcagcaccagcaccg

SEQ ID NO: 352 - RNA, 23 nt, Artificial sequence
  Synthetic oligonucleotide
gaggacgaugauacucagcaaca

SEQ ID NO: 353 - RNA, 23 nt, Artificial sequence
  Synthetic oligonucleotide
auguuuauuuuaaugcaucuuag

SEQ ID NO: 354 - RNA, 23 nt, Artificial sequence
  Synthetic oligonucleotide
ucugauguuuauuuuaaugcauc

SEQ ID NO: 355 - RNA, 23 nt, Artificial sequence
  Synthetic oligonucleotide
ugaguuacucugauguuuauuuu

SEQ ID NO: 356 - RNA, 23 nt, Artificial sequence
  Synthetic oligonucleotide
uuacucugauguuuauuuuaaug

SEQ ID NO: 357 - RNA, 23 nt, Artificial sequence
  Synthetic oligonucleotide
uuauuuuaaugcaucuuagucca

SEQ ID NO: 358 - RNA, 23 nt, Artificial sequence
  Synthetic oligonucleotide
uuuaaugcaucuuaguccacaca

SEQ ID NO: 359 - RNA, 23 nt, Artificial sequence
  Synthetic oligonucleotide
uaguccacacaguugguauaaaa

SEQ ID NO: 360 - RNA, 23 nt, Artificial sequence
  Synthetic oligonucleotide
ccacacaguugguauaaaaucag

SEQ ID NO: 361 - RNA, 23 nt, Artificial sequence
  Synthetic oligonucleotide
aucuuaguccacacaguugguau

SEQ ID NO: 362 - RNA, 23 nt, Artificial sequence
  Synthetic oligonucleotide
augcaucuuaguccacacaguug

SEQ ID NO: 363 - RNA, 23 nt, Artificial sequence
  Synthetic oligonucleotide
acaguugguauaaaaucagaaaa

SEQ ID NO: 364 - RNA, 23 nt, Artificial sequence
  Synthetic oligonucleotide
uugguauaaaaucagaaaaugca

SEQ ID NO: 365 - RNA, 23 nt, Artificial sequence
  Synthetic oligonucleotide
aaaugcaaagcaaaaacaaaagg

SEQ ID NO: 366 - RNA, 23 nt, Artificial sequence
  Synthetic oligonucleotide
gcaaagcaaaaacaaaaggucug

SEQ ID NO: 367 - RNA, 23 nt, Artificial sequence
  Synthetic oligonucleotide
cagaaaaugcaaagcaaaaacaa

SEQ ID NO: 368 - RNA, 23 nt, Artificial sequence
  Synthetic oligonucleotide
aaaucagaaaaugcaaagcaaaa

SEQ ID NO: 369 - RNA, 23 nt, Artificial sequence
  Synthetic oligonucleotide
uauaaaaucagaaaaugcaaagc

SEQ ID NO: 370 - RNA, 23 nt, Artificial sequence
  Synthetic oligonucleotide
agcaaaaacaaaaggucuggagu

SEQ ID NO: 371 - RNA, 23 nt, Artificial sequence
  Synthetic oligonucleotide
aaaacaaaaggucuggagucuua

SEQ ID NO: 372 - RNA, 23 nt, Artificial sequence
  Synthetic oligonucleotide
aggucuggagucuuagcaucaga

SEQ ID NO: 373 - RNA, 23 nt, Artificial sequence
  Synthetic oligonucleotide
caaaaggucuggagucuuagcau

SEQ ID NO: 374 - RNA, 23 nt, Artificial sequence
  Synthetic oligonucleotide
agucuuagcaucagaagggcacc

SEQ ID NO: 375 - RNA, 23 nt, Artificial sequence
  Synthetic oligonucleotide
cuggagucuuagcaucagaaggg

SEQ ID NO: 376 - RNA, 23 nt, Artificial sequence
  Synthetic oligonucleotide
uuagcaucagaagggcaccauau

SEQ ID NO: 377 - RNA, 23 nt, Artificial sequence
  Synthetic oligonucleotide
agaagggcaccauauauacaucu

SEQ ID NO: 378 - RNA, 23 nt, Artificial sequence
  Synthetic oligonucleotide
caucagaagggcaccauauauac

SEQ ID NO: 379 - RNA, 23 nt, Artificial sequence
  Synthetic oligonucleotide
uauauacaucuacaguugguggc

SEQ ID NO: 380 - RNA, 23 nt, Artificial sequence
  Synthetic oligonucleotide
accauauauacaucuacaguugg

SEQ ID NO: 381 - RNA, 23 nt, Artificial sequence
  Synthetic oligonucleotide
gggcaccauauauacaucuacag

SEQ ID NO: 382 - RNA, 23 nt, Artificial sequence
  Synthetic oligonucleotide
uacaucuacaguugguggccaau

SEQ ID NO: 383 - RNA, 23 nt, Artificial sequence
  Synthetic oligonucleotide
caguugguggccaauacaaguca

SEQ ID NO: 384 - RNA, 23 nt, Artificial sequence
  Synthetic oligonucleotide
ucuacaguugguggccaauacaa

SEQ ID NO: 385 - RNA, 23 nt, Artificial sequence
  Synthetic oligonucleotide
ugguggccaauacaagucauugc

SEQ ID NO: 386 - RNA, 23 nt, Artificial sequence
  Synthetic oligonucleotide
aauacaagucauugccagacagu

SEQ ID NO: 387 - RNA, 23 nt, Artificial sequence
  Synthetic oligonucleotide
ugccagacaguccuuggaggcac

SEQ ID NO: 388 - RNA, 23 nt, Artificial sequence
  Synthetic oligonucleotide
ggccaauacaagucauugccaga

SEQ ID NO: 389 - RNA, 23 nt, Artificial sequence
  Synthetic oligonucleotide
caagucauugccagacaguccuu

SEQ ID NO: 390 - RNA, 23 nt, Artificial sequence
  Synthetic oligonucleotide
ucauugccagacaguccuuggag

SEQ ID NO: 391 - RNA, 23 nt, Artificial sequence
  Synthetic oligonucleotide
agacaguccuuggaggcacagaa

SEQ ID NO: 392 - RNA, 23 nt, Artificial sequence
  Synthetic oligonucleotide
gaacagccuagacccagccaagc

SEQ ID NO: 393 - RNA, 23 nt, Artificial sequence
  Synthetic oligonucleotide
cacagaacagccuagacccagcc

SEQ ID NO: 394 - RNA, 23 nt, Artificial sequence
  Synthetic oligonucleotide
cuuggaggcacagaacagccuag

SEQ ID NO: 395 - RNA, 23 nt, Artificial sequence
  Synthetic oligonucleotide
aguccuuggaggcacagaacagc

SEQ ID NO: 396 - RNA, 23 nt, Artificial sequence
  Synthetic oligonucleotide
gaggcacagaacagccuagaccc

SEQ ID NO: 397 - RNA, 23 nt, Artificial sequence
  Synthetic oligonucleotide
agccuagacccagccaagcucua

SEQ ID NO: 398 - RNA, 23 nt, Artificial sequence
  Synthetic oligonucleotide
agcucuaggaacucacgguccca

SEQ ID NO: 399 - RNA, 23 nt, Artificial sequence
  Synthetic oligonucleotide
gccaagcucuaggaacucacggu

SEQ ID NO: 400 - RNA, 23 nt, Artificial sequence
  Synthetic oligonucleotide
cccagccaagcucuaggaacuca

SEQ ID NO: 401 - RNA, 23 nt, Artificial sequence
  Synthetic oligonucleotide
uagacccagccaagcucuaggaa

SEQ ID NO: 402 - RNA, 23 nt, Artificial sequence
  Synthetic oligonucleotide
cuaggaacucacggucccaagga

SEQ ID NO: 403 - RNA, 23 nt, Artificial sequence
  Synthetic oligonucleotide
ggucccaaggagucuagacgcuu

SEQ ID NO: 404 - RNA, 23 nt, Artificial sequence
  Synthetic oligonucleotide
ucacggucccaaggagucuagac

SEQ ID NO: 405 - RNA, 23 nt, Artificial sequence
  Synthetic oligonucleotide
gaacucacggucccaaggagucu

SEQ ID NO: 406 - RNA, 23 nt, Artificial sequence
  Synthetic oligonucleotide
ucuagacgcuuguucugaugcuc

SEQ ID NO: 407 - RNA, 23 nt, Artificial sequence
  Synthetic oligonucleotide
ggagucuagacgcuuguucugau

SEQ ID NO: 408 - RNA, 23 nt, Artificial sequence
  Synthetic oligonucleotide
ccaaggagucuagacgcuuguuc

SEQ ID NO: 409 - RNA, 23 nt, Artificial sequence
  Synthetic oligonucleotide
gacgcuuguucugaugcuccgac

SEQ ID NO: 410 - RNA, 23 nt, Artificial sequence
  Synthetic oligonucleotide
gaccguaagaaaaaugugggagu

SEQ ID NO: 411 - RNA, 23 nt, Artificial sequence
  Synthetic oligonucleotide
cuccgaccguaagaaaaaugugg

SEQ ID NO: 412 - RNA, 23 nt, Artificial sequence
  Synthetic oligonucleotide
uucugaugcuccgaccguaagaa

SEQ ID NO: 413 - RNA, 23 nt, Artificial sequence
  Synthetic oligonucleotide
gaugcuccgaccguaagaaaaau

SEQ ID NO: 414 - RNA, 23 nt, Artificial sequence
  Synthetic oligonucleotide
cuuguucugaugcuccgaccgua

SEQ ID NO: 415 - RNA, 23 nt, Artificial sequence
  Synthetic oligonucleotide
guaagaaaaaugugggagugaug

SEQ ID NO: 416 - RNA, 23 nt, Artificial sequence
  Synthetic oligonucleotide
agugaugaaggcuuuaugauuua

SEQ ID NO: 417 - RNA, 23 nt, Artificial sequence
  Synthetic oligonucleotide
ugggagugaugaaggcuuuauga

SEQ ID NO: 418 - RNA, 23 nt, Artificial sequence
  Synthetic oligonucleotide
aaugugggagugaugaaggcuuu

SEQ ID NO: 419 - RNA, 23 nt, Artificial sequence
  Synthetic oligonucleotide
gaaaaaugugggagugaugaagg

SEQ ID NO: 420 - RNA, 23 nt, Artificial sequence
  Synthetic oligonucleotide
augaaggcuuuaugauuuacuca

SEQ ID NO: 421 - RNA, 23 nt, Artificial sequence
  Synthetic oligonucleotide
uuuaugauuuacucauuauagua

SEQ ID NO: 422 - RNA, 23 nt, Artificial sequence
  Synthetic oligonucleotide
ucauuauaguaauaauagcagcc

SEQ ID NO: 423 - RNA, 23 nt, Artificial sequence
  Synthetic oligonucleotide
uauaguaauaauagcagccuagc

SEQ ID NO: 424 - RNA, 23 nt, Artificial sequence
  Synthetic oligonucleotide
ugauuuacucauuauaguaauaa

SEQ ID NO: 425 - RNA, 23 nt, Artificial sequence
  Synthetic oligonucleotide
aggcuuuaugauuuacucauuau

SEQ ID NO: 426 - RNA, 23 nt, Artificial sequence
  Synthetic oligonucleotide
uuacucauuauaguaauaauagc

SEQ ID NO: 427 - RNA, 23 nt, Artificial sequence
  Synthetic oligonucleotide
guaauaauagcagccuagcuagg

SEQ ID NO: 428 - RNA, 23 nt, Artificial sequence
  Synthetic oligonucleotide
agcuagguacaaaagcaguuaua

SEQ ID NO: 429 - RNA, 23 nt, Artificial sequence
  Synthetic oligonucleotide
gccuagcuagguacaaaagcagu

SEQ ID NO: 430 - RNA, 23 nt, Artificial sequence
  Synthetic oligonucleotide
uaauagcagccuagcuagguaca

SEQ ID NO: 431 - RNA, 23 nt, Artificial sequence
  Synthetic oligonucleotide
agcagccuagcuagguacaaaag

SEQ ID NO: 432 - RNA, 23 nt, Artificial sequence
  Synthetic oligonucleotide
agguacaaaagcaguuauaaacc

SEQ ID NO: 433 - RNA, 23 nt, Artificial sequence
  Synthetic oligonucleotide
acaaaagcaguuauaaaccauuu

SEQ ID NO: 434 - RNA, 23 nt, Artificial sequence
  Synthetic oligonucleotide
uuuauauuacacagaauuauuca

SEQ ID NO: 435 - RNA, 23 nt, Artificial sequence
  Synthetic oligonucleotide
accauuuauauuacacagaauua

SEQ ID NO: 436 - RNA, 23 nt, Artificial sequence
  Synthetic oligonucleotide
auaaaccauuuauauuacacaga

SEQ ID NO: 437 - RNA, 23 nt, Artificial sequence
  Synthetic oligonucleotide
aguuauaaaccauuuauauuaca

SEQ ID NO: 438 - RNA, 23 nt, Artificial sequence
  Synthetic oligonucleotide
aagcaguuauaaaccauuuauau

SEQ ID NO: 439 - RNA, 23 nt, Artificial sequence
  Synthetic oligonucleotide
uauuacacagaauuauucagguc

SEQ ID NO: 440 - RNA, 23 nt, Artificial sequence
  Synthetic oligonucleotide
gucuccauucuaucuuauguugu

SEQ ID NO: 441 - RNA, 23 nt, Artificial sequence
  Synthetic oligonucleotide
ucaggucuccauucuaucuuaug

SEQ ID NO: 442 - RNA, 23 nt, Artificial sequence
  Synthetic oligonucleotide
uuauucaggucuccauucuaucu

SEQ ID NO: 443 - RNA, 23 nt, Artificial sequence
  Synthetic oligonucleotide
agaauuauucaggucuccauucu

SEQ ID NO: 444 - RNA, 23 nt, Artificial sequence
  Synthetic oligonucleotide
acacagaauuauucaggucucca

SEQ ID NO: 445 - RNA, 23 nt, Artificial sequence
  Synthetic oligonucleotide
uguaaaauuguuaauuggauuuc

SEQ ID NO: 446 - RNA, 23 nt, Artificial sequence
  Synthetic oligonucleotide
ccauucuaucuuauguuguaaaa

SEQ ID NO: 447 - RNA, 23 nt, Artificial sequence
  Synthetic oligonucleotide
auguuguaaaauuguuaauugga

SEQ ID NO: 448 - RNA, 23 nt, Artificial sequence
  Synthetic oligonucleotide
ucuuauguuguaaaauuguuaau

SEQ ID NO: 449 - RNA, 23 nt, Artificial sequence
  Synthetic oligonucleotide
ucuaucuuauguuguaaaauugu

SEQ ID NO: 450 - RNA, 23 nt, Artificial sequence
  Synthetic oligonucleotide
aaauuguuaauuggauuuccagu

SEQ ID NO: 451 - RNA, 23 nt, Artificial sequence
  Synthetic oligonucleotide
uuccagugaguuguuuagaugau

SEQ ID NO: 452 - RNA, 23 nt, Artificial sequence
  Synthetic oligonucleotide
aguuguuuagaugauuagugaua

SEQ ID NO: 453 - RNA, 23 nt, Artificial sequence
  Synthetic oligonucleotide
agugaguuguuuagaugauuagu

SEQ ID NO: 454 - RNA, 23 nt, Artificial sequence
  Synthetic oligonucleotide
ggauuuccagugaguuguuuaga

SEQ ID NO: 455 - RNA, 23 nt, Artificial sequence
  Synthetic oligonucleotide
uguuaauuggauuuccagugagu

SEQ ID NO: 456 - RNA, 23 nt, Artificial sequence
  Synthetic oligonucleotide
aauuggauuuccagugaguuguu

SEQ ID NO: 457 - RNA, 23 nt, Artificial sequence
  Synthetic oligonucleotide
guuuagaugauuagugauaauaa

SEQ ID NO: 458 - RNA, 23 nt, Artificial sequence
  Synthetic oligonucleotide
auaauaaggaaugguaaauccau

SEQ ID NO: 459 - RNA, 23 nt, Artificial sequence
  Synthetic oligonucleotide
agugauaauaaggaaugguaaau

SEQ ID NO: 460 - RNA, 23 nt, Artificial sequence
  Synthetic oligonucleotide
gauuagugauaauaaggaauggu

SEQ ID NO: 461 - RNA, 23 nt, Artificial sequence
  Synthetic oligonucleotide
agaugauuagugauaauaaggaa

SEQ ID NO: 462 - RNA, 23 nt, Artificial sequence
  Synthetic oligonucleotide
uaaggaaugguaaauccauagca

SEQ ID NO: 463 - RNA, 23 nt, Artificial sequence
  Synthetic oligonucleotide
gaaugguaaauccauagcaccau

SEQ ID NO: 464 - RNA, 23 nt, Artificial sequence
  Synthetic oligonucleotide
cauuucaaagacuuguugucacu

SEQ ID NO: 465 - RNA, 23 nt, Artificial sequence
  Synthetic oligonucleotide
gcaccauuucaaagacuuguugu

SEQ ID NO: 466 - RNA, 23 nt, Artificial sequence
  Synthetic oligonucleotide
cauagcaccauuucaaagacuug

SEQ ID NO: 467 - RNA, 23 nt, Artificial sequence
  Synthetic oligonucleotide
gguaaauccauagcaccauuuca

SEQ ID NO: 468 - RNA, 23 nt, Artificial sequence
  Synthetic oligonucleotide
aauccauagcaccauuucaaaga

SEQ ID NO: 469 - RNA, 23 nt, Artificial sequence
  Synthetic oligonucleotide
ucaaagacuuguugucacugauu

SEQ ID NO: 470 - RNA, 23 nt, Artificial sequence
  Synthetic oligonucleotide
auuucucauuuagaugugugacg

SEQ ID NO: 471 - RNA, 23 nt, Artificial sequence
  Synthetic oligonucleotide
acugauuucucauuuagaugugu

SEQ ID NO: 472 - RNA, 23 nt, Artificial sequence
  Synthetic oligonucleotide
ugucacugauuucucauuuagau

SEQ ID NO: 473 - RNA, 23 nt, Artificial sequence
  Synthetic oligonucleotide
uuguugucacugauuucucauuu

SEQ ID NO: 474 - RNA, 23 nt, Artificial sequence
  Synthetic oligonucleotide
agacuuguugucacugauuucuc

SEQ ID NO: 475 - RNA, 23 nt, Artificial sequence
  Synthetic oligonucleotide
cucauuuagaugugugacgaaga

SEQ ID NO: 476 - RNA, 23 nt, Artificial sequence
  Synthetic oligonucleotide
agauacuccaccuguaagggcaa

SEQ ID NO: 477 - RNA, 23 nt, Artificial sequence
  Synthetic oligonucleotide
acgaagauacuccaccuguaagg

SEQ ID NO: 478 - RNA, 23 nt, Artificial sequence
  Synthetic oligonucleotide
ugugacgaagauacuccaccugu

SEQ ID NO: 479 - RNA, 23 nt, Artificial sequence
  Synthetic oligonucleotide
gaugugugacgaagauacuccac

SEQ ID NO: 480 - RNA, 23 nt, Artificial sequence
  Synthetic oligonucleotide
uuuagaugugugacgaagauacu

SEQ ID NO: 481 - RNA, 23 nt, Artificial sequence
  Synthetic oligonucleotide
acuccaccuguaagggcaaguau

SEQ ID NO: 482 - RNA, 23 nt, Artificial sequence
  Synthetic oligonucleotide
caaguaugccaaugccacaagcc

SEQ ID NO: 483 - RNA, 23 nt, Artificial sequence
  Synthetic oligonucleotide
uaugccaaugccacaagccgugu

SEQ ID NO: 484 - RNA, 23 nt, Artificial sequence
  Synthetic oligonucleotide
agggcaaguaugccaaugccaca

SEQ ID NO: 485 - RNA, 23 nt, Artificial sequence
  Synthetic oligonucleotide
uguaagggcaaguaugccaaugc

SEQ ID NO: 486 - RNA, 23 nt, Artificial sequence
  Synthetic oligonucleotide
caccuguaagggcaaguaugcca

SEQ ID NO: 487 - RNA, 23 nt, Artificial sequence
  Synthetic oligonucleotide
ugccacaagccguguuuuugcaa

SEQ ID NO: 488 - RNA, 23 nt, Artificial sequence
  Synthetic oligonucleotide
uguuuuugcaagggcuccaguuu

SEQ ID NO: 489 - RNA, 23 nt, Artificial sequence
  Synthetic oligonucleotide
gccguguuuuugcaagggcucca

SEQ ID NO: 490 - RNA, 23 nt, Artificial sequence
  Synthetic oligonucleotide
acaagccguguuuuugcaagggc

SEQ ID NO: 491 - RNA, 23 nt, Artificial sequence
  Synthetic oligonucleotide
ccaaugccacaagccguguuuuu

SEQ ID NO: 492 - RNA, 23 nt, Artificial sequence
  Synthetic oligonucleotide
uuugcaagggcuccaguuugggc

SEQ ID NO: 493 - RNA, 23 nt, Artificial sequence
  Synthetic oligonucleotide
caagggcuccaguuugggcauuu

SEQ ID NO: 494 - RNA, 23 nt, Artificial sequence
  Synthetic oligonucleotide
uuuuguccgugugcgcguaaagc

SEQ ID NO: 495 - RNA, 23 nt, Artificial sequence
  Synthetic oligonucleotide
uuugggcauuuuguccgugugcg

SEQ ID NO: 496 - RNA, 23 nt, Artificial sequence
  Synthetic oligonucleotide
ggcauuuuguccgugugcgcgua

SEQ ID NO: 497 - RNA, 23 nt, Artificial sequence
  Synthetic oligonucleotide
ccaguuugggcauuuuguccgug

SEQ ID NO: 498 - RNA, 23 nt, Artificial sequence
  Synthetic oligonucleotide
ggcuccaguuugggcauuuuguc

SEQ ID NO: 499 - RNA, 23 nt, Artificial sequence
  Synthetic oligonucleotide
guccgugugcgcguaaagcuuca

SEQ ID NO: 500 - RNA, 23 nt, Artificial sequence
  Synthetic oligonucleotide
ucacacagagguucgggcagcgg

SEQ ID NO: 501 - RNA, 23 nt, Artificial sequence
  Synthetic oligonucleotide
agcuucacacagagguucgggca

SEQ ID NO: 502 - RNA, 23 nt, Artificial sequence
  Synthetic oligonucleotide
guaaagcuucacacagagguucg

SEQ ID NO: 503 - RNA, 23 nt, Artificial sequence
  Synthetic oligonucleotide
gcgcguaaagcuucacacagagg

SEQ ID NO: 504 - RNA, 23 nt, Artificial sequence
  Synthetic oligonucleotide
gugugcgcguaaagcuucacaca

SEQ ID NO: 505 - RNA, 23 nt, Artificial sequence
  Synthetic oligonucleotide
agguucgggcagcggcuguuucu

SEQ ID NO: 506 - RNA, 23 nt, Artificial sequence
  Synthetic oligonucleotide
ucgggcagcggcuguuucuguug

SEQ ID NO: 507 - RNA, 23 nt, Artificial sequence
  Synthetic oligonucleotide
acagagguucgggcagcggcugu

SEQ ID NO: 508 - RNA, 23 nt, Artificial sequence
  Synthetic oligonucleotide
uguuucuguuggaugcacugggu

SEQ ID NO: 509 - RNA, 23 nt, Artificial sequence
  Synthetic oligonucleotide
cggcuguuucuguuggaugcacu

SEQ ID NO: 510 - RNA, 23 nt, Artificial sequence
  Synthetic oligonucleotide
gcagcggcuguuucuguuggaug

SEQ ID NO: 511 - RNA, 23 nt, Artificial sequence
  Synthetic oligonucleotide
ucuguuggaugcacugggucacc

SEQ ID NO: 512 - RNA, 23 nt, Artificial sequence
  Synthetic oligonucleotide
acccaccagaaaagggcuuuugg

SEQ ID NO: 513 - RNA, 23 nt, Artificial sequence
  Synthetic oligonucleotide
ggucacccaccagaaaagggcuu

SEQ ID NO: 514 - RNA, 23 nt, Artificial sequence
  Synthetic oligonucleotide
augcacugggucacccaccagaa

SEQ ID NO: 515 - RNA, 23 nt, Artificial sequence
  Synthetic oligonucleotide
uuggaugcacugggucacccacc

SEQ ID NO: 516 - RNA, 23 nt, Artificial sequence
  Synthetic oligonucleotide
acugggucacccaccagaaaagg

SEQ ID NO: 517 - RNA, 23 nt, Artificial sequence
  Synthetic oligonucleotide
accagaaaagggcuuuuggacau

SEQ ID NO: 518 - RNA, 23 nt, Artificial sequence
  Synthetic oligonucleotide
cauuugggguuucuacccacacu

SEQ ID NO: 519 - RNA, 23 nt, Artificial sequence
  Synthetic oligonucleotide
uggacauuugggguuucuaccca

SEQ ID NO: 520 - RNA, 23 nt, Artificial sequence
  Synthetic oligonucleotide
agggcuuuuggacauuugggguu

SEQ ID NO: 521 - RNA, 23 nt, Artificial sequence
  Synthetic oligonucleotide
cuuuuggacauuugggguuucua

SEQ ID NO: 522 - RNA, 23 nt, Artificial sequence
  Synthetic oligonucleotide
gaaaagggcuuuuggacauuugg

SEQ ID NO: 523 - RNA, 23 nt, Artificial sequence
  Synthetic oligonucleotide
guuucuacccacacuuugguuuu

SEQ ID NO: 524 - RNA, 23 nt, Artificial sequence
  Synthetic oligonucleotide
acuuugguuuucuaaaugaggug

SEQ ID NO: 525 - RNA, 23 nt, Artificial sequence
  Synthetic oligonucleotide
ccacacuuugguuuucuaaauga

SEQ ID NO: 526 - RNA, 23 nt, Artificial sequence
  Synthetic oligonucleotide
cuacccacacuuugguuuucuaa

SEQ ID NO: 527 - RNA, 23 nt, Artificial sequence
  Synthetic oligonucleotide
ugggguuucuacccacacuuugg

SEQ ID NO: 528 - RNA, 23 nt, Artificial sequence
  Synthetic oligonucleotide
ugguuuucuaaaugagguggacu

SEQ ID NO: 529 - RNA, 23 nt, Artificial sequence
  Synthetic oligonucleotide
uuucuaaaugagguggacuggga

SEQ ID NO: 530 - RNA, 23 nt, Artificial sequence
  Synthetic oligonucleotide
ggagggagguaucuucuuucaga

SEQ ID NO: 531 - RNA, 23 nt, Artificial sequence
  Synthetic oligonucleotide
acugggagggagguaucuucuuu

SEQ ID NO: 532 - RNA, 23 nt, Artificial sequence
  Synthetic oligonucleotide
ugagguggacugggagggaggua

SEQ ID NO: 533 - RNA, 23 nt, Artificial sequence
  Synthetic oligonucleotide
guggacugggagggagguaucuu

SEQ ID NO: 534 - RNA, 23 nt, Artificial sequence
  Synthetic oligonucleotide
uaaaugagguggacugggaggga

SEQ ID NO: 535 - RNA, 23 nt, Artificial sequence
  Synthetic oligonucleotide
ggagguaucuucuuucagaugaa

SEQ ID NO: 536 - RNA, 23 nt, Artificial sequence
  Synthetic oligonucleotide
gaaagggaaggggcgagauggag

SEQ ID NO: 537 - RNA, 23 nt, Artificial sequence
  Synthetic oligonucleotide
agaugaaagggaaggggcgagau

SEQ ID NO: 538 - RNA, 23 nt, Artificial sequence
  Synthetic oligonucleotide
cuucuuucagaugaaagggaagg

SEQ ID NO: 539 - RNA, 23 nt, Artificial sequence
  Synthetic oligonucleotide
uuucagaugaaagggaaggggcg

SEQ ID NO: 540 - RNA, 23 nt, Artificial sequence
  Synthetic oligonucleotide
guaucuucuuucagaugaaaggg

SEQ ID NO: 541 - RNA, 23 nt, Artificial sequence
  Synthetic oligonucleotide
aggggcgagauggaguuaucuua

SEQ ID NO: 542 - RNA, 23 nt, Artificial sequence
  Synthetic oligonucleotide
uaucuuauuucuggguaaaacaa

SEQ ID NO: 543 - RNA, 23 nt, Artificial sequence
  Synthetic oligonucleotide
gauggaguuaucuuauuucuggg

SEQ ID NO: 544 - RNA, 23 nt, Artificial sequence
  Synthetic oligonucleotide
gaguuaucuuauuucuggguaaa

SEQ ID NO: 545 - RNA, 23 nt, Artificial sequence
  Synthetic oligonucleotide
gcgagauggaguuaucuuauuuc

SEQ ID NO: 546 - RNA, 23 nt, Artificial sequence
  Synthetic oligonucleotide
gggaaggggcgagauggaguuau

SEQ ID NO: 547 - RNA, 23 nt, Artificial sequence
  Synthetic oligonucleotide
uucuggguaaaacaaaacaaaca

SEQ ID NO: 548 - RNA, 23 nt, Artificial sequence
  Synthetic oligonucleotide
acaaacaaaaaacaaaacaaaaa

SEQ ID NO: 549 - RNA, 23 nt, Artificial sequence
  Synthetic oligonucleotide
caaaacaaacaaacaaaaaacaa

SEQ ID NO: 550 - RNA, 23 nt, Artificial sequence
  Synthetic oligonucleotide
aaaacaaaacaaacaaacaaaaa

SEQ ID NO: 551 - RNA, 23 nt, Artificial sequence
  Synthetic oligonucleotide
ggguaaaacaaaacaaacaaaca

SEQ ID NO: 552 - RNA, 23 nt, Artificial sequence
  Synthetic oligonucleotide
uuauuucuggguaaaacaaaaca

SEQ ID NO: 553 - RNA, 23 nt, Artificial sequence
  Synthetic oligonucleotide
aaaacaaaacaaaaauacugagc

SEQ ID NO: 554 - RNA, 23 nt, Artificial sequence
  Synthetic oligonucleotide
caaaacaaaaauacugagcugga

SEQ ID NO: 555 - RNA, 23 nt, Artificial sequence
  Synthetic oligonucleotide
aaauacugagcuggauuauacug

SEQ ID NO: 556 - RNA, 23 nt, Artificial sequence
  Synthetic oligonucleotide
acaaacaaacaaaaaacaaaaca

SEQ ID NO: 557 - RNA, 23 nt, Artificial sequence
  Synthetic oligonucleotide
acaaaaaacaaaacaaaaauacu

SEQ ID NO: 558 - RNA, 23 nt, Artificial sequence
  Synthetic oligonucleotide
acaaaaauacugagcuggauuau

SEQ ID NO: 559 - RNA, 23 nt, Artificial sequence
  Synthetic oligonucleotide
acugagcuggauuauacuguuag

SEQ ID NO: 560 - RNA, 23 nt, Artificial sequence
  Synthetic oligonucleotide
uaggauguaaaguuccuuagcua

SEQ ID NO: 561 - RNA, 23 nt, Artificial sequence
  Synthetic oligonucleotide
cuguuaggauguaaaguuccuua

SEQ ID NO: 562 - RNA, 23 nt, Artificial sequence
  Synthetic oligonucleotide
uauacuguuaggauguaaaguuc

SEQ ID NO: 563 - RNA, 23 nt, Artificial sequence
  Synthetic oligonucleotide
agcuggauuauacuguuaggaug

SEQ ID NO: 564 - RNA, 23 nt, Artificial sequence
  Synthetic oligonucleotide
ggauuauacuguuaggauguaaa

SEQ ID NO: 565 - RNA, 23 nt, Artificial sequence
  Synthetic oligonucleotide
auguaaaguuccuuagcuacuuc

SEQ ID NO: 566 - RNA, 23 nt, Artificial sequence
  Synthetic oligonucleotide
uucuuuaaggcucaacacgaggc

SEQ ID NO: 567 - RNA, 23 nt, Artificial sequence
  Synthetic oligonucleotide
cuacuucuuuaaggcucaacacg

SEQ ID NO: 568 - RNA, 23 nt, Artificial sequence
  Synthetic oligonucleotide
uuagcuacuucuuuaaggcucaa

SEQ ID NO: 569 - RNA, 23 nt, Artificial sequence
  Synthetic oligonucleotide
uuccuuagcuacuucuuuaaggc

SEQ ID NO: 570 - RNA, 23 nt, Artificial sequence
  Synthetic oligonucleotide
aaaguuccuuagcuacuucuuua

SEQ ID NO: 571 - RNA, 23 nt, Artificial sequence
  Synthetic oligonucleotide
uuaaggcucaacacgaggcugau

SEQ ID NO: 572 - RNA, 23 nt, Artificial sequence
  Synthetic oligonucleotide
acgaggcugauggucaacauaaa

SEQ ID NO: 573 - RNA, 23 nt, Artificial sequence
  Synthetic oligonucleotide
gauggucaacauaaaaagcaaac

SEQ ID NO: 574 - RNA, 23 nt, Artificial sequence
  Synthetic oligonucleotide
caacacgaggcugauggucaaca

SEQ ID NO: 575 - RNA, 23 nt, Artificial sequence
  Synthetic oligonucleotide
ggcucaacacgaggcugaugguc

SEQ ID NO: 576 - RNA, 23 nt, Artificial sequence
  Synthetic oligonucleotide
ggcugauggucaacauaaaaagc

SEQ ID NO: 577 - RNA, 23 nt, Artificial sequence
  Synthetic oligonucleotide
gucaacauaaaaagcaaacaaua

SEQ ID NO: 578 - RNA, 23 nt, Artificial sequence
  Synthetic oligonucleotide
aacaauacuauguacauauaugu

SEQ ID NO: 579 - RNA, 23 nt, Artificial sequence
  Synthetic oligonucleotide
uacauauauguaaaaaguguuau

SEQ ID NO: 580 - RNA, 23 nt, Artificial sequence
  Synthetic oligonucleotide
agcaaacaauacuauguacauau

SEQ ID NO: 581 - RNA, 23 nt, Artificial sequence
  Synthetic oligonucleotide
aaaaagcaaacaauacuauguac

SEQ ID NO: 582 - RNA, 23 nt, Artificial sequence
  Synthetic oligonucleotide
acauaaaaagcaaacaauacuau

SEQ ID NO: 583 - RNA, 23 nt, Artificial sequence
  Synthetic oligonucleotide
auacuauguacauauauguaaaa

SEQ ID NO: 584 - RNA, 23 nt, Artificial sequence
  Synthetic oligonucleotide
aaaaguguuauaaauagguuuua

SEQ ID NO: 585 - RNA, 23 nt, Artificial sequence
  Synthetic oligonucleotide
uguaaaaaguguuauaaauaggu

SEQ ID NO: 586 - RNA, 23 nt, Artificial sequence
  Synthetic oligonucleotide
uauauguaaaaaguguuauaaau

SEQ ID NO: 587 - RNA, 23 nt, Artificial sequence
  Synthetic oligonucleotide
uauguacauauauguaaaaagug

SEQ ID NO: 588 - RNA, 23 nt, Artificial sequence
  Synthetic oligonucleotide
uauaaauagguuuuauaaaccgg

SEQ ID NO: 589 - RNA, 23 nt, Artificial sequence
  Synthetic oligonucleotide
auacaucuucaaucaacagcaac

SEQ ID NO: 590 - RNA, 23 nt, Artificial sequence
  Synthetic oligonucleotide
guguuauaaauagguuuuauaaa

SEQ ID NO: 591 - RNA, 23 nt, Artificial sequence
  Synthetic oligonucleotide
aaaccggagauauuauauacauc

SEQ ID NO: 592 - RNA, 23 nt, Artificial sequence
  Synthetic oligonucleotide
aauagguuuuauaaaccggagau

SEQ ID NO: 593 - RNA, 23 nt, Artificial sequence
  Synthetic oligonucleotide
uuauaaaccggagauauuauaua

SEQ ID NO: 594 - RNA, 23 nt, Artificial sequence
  Synthetic oligonucleotide
gguuuuauaaaccggagauauua

SEQ ID NO: 595 - RNA, 23 nt, Artificial sequence
  Synthetic oligonucleotide
gauauuauauacaucuucaauca

SEQ ID NO: 596 - RNA, 23 nt, Artificial sequence
  Synthetic oligonucleotide
ucaacagcaacccccaccuccac

SEQ ID NO: 597 - RNA, 23 nt, Artificial sequence
  Synthetic oligonucleotide
aucuucaaucaacagcaaccccc

SEQ ID NO: 598 - RNA, 23 nt, Artificial sequence
  Synthetic oligonucleotide
ucaaucaacagcaacccccaccu

SEQ ID NO: 599 - RNA, 23 nt, Artificial sequence
  Synthetic oligonucleotide
cggagauauuauauacaucuuca

SEQ ID NO: 600 - RNA, 23 nt, Artificial sequence
  Synthetic oligonucleotide
uuauauacaucuucaaucaacag

SEQ ID NO: 601 - RNA, 23 nt, Artificial sequence
  Synthetic oligonucleotide
cagcaacccccaccuccacugcu

SEQ ID NO: 602 - RNA, 23 nt, Artificial sequence
  Synthetic oligonucleotide
aacccccaccuccacugcuuucu

SEQ ID NO: 603 - RNA, 23 nt, Artificial sequence
  Synthetic oligonucleotide
cacugcuuucuguuugguuuggu

SEQ ID NO: 604 - RNA, 23 nt, Artificial sequence
  Synthetic oligonucleotide
cccaccuccacugcuuucuguuu

SEQ ID NO: 605 - RNA, 23 nt, Artificial sequence
  Synthetic oligonucleotide
ccuccacugcuuucuguuugguu

SEQ ID NO: 606 - RNA, 23 nt, Artificial sequence
  Synthetic oligonucleotide
gcuuucuguuugguuugguuuga

SEQ ID NO: 607 - RNA, 23 nt, Artificial sequence
  Synthetic oligonucleotide
ucuguuugguuugguuugaguuu

SEQ ID NO: 608 - RNA, 23 nt, Artificial sequence
  Synthetic oligonucleotide
ggcuagcucuuuuuucuuugucu

SEQ ID NO: 609 - RNA, 23 nt, Artificial sequence
  Synthetic oligonucleotide
ugaguuugggauuuugggcuagc

SEQ ID NO: 610 - RNA, 23 nt, Artificial sequence
  Synthetic oligonucleotide
gguuugaguuugggauuuugggc

SEQ ID NO: 611 - RNA, 23 nt, Artificial sequence
  Synthetic oligonucleotide
uuugguuugguuugaguuuggga

SEQ ID NO: 612 - RNA, 23 nt, Artificial sequence
  Synthetic oligonucleotide
guuugguuugaguuugggauuuu

SEQ ID NO: 613 - RNA, 23 nt, Artificial sequence
  Synthetic oligonucleotide
uuugggauuuugggcuagcucuu

SEQ ID NO: 614 - RNA, 23 nt, Artificial sequence
  Synthetic oligonucleotide
uuucuuugucugcuuucuguuuu

SEQ ID NO: 615 - RNA, 23 nt, Artificial sequence
  Synthetic oligonucleotide
cuuuuuucuuugucugcuuucug

SEQ ID NO: 616 - RNA, 23 nt, Artificial sequence
  Synthetic oligonucleotide
agcucuuuuuucuuugucugcuu

SEQ ID NO: 617 - RNA, 23 nt, Artificial sequence
  Synthetic oligonucleotide
uuugggcuagcucuuuuuucuuu

SEQ ID NO: 618 - RNA, 23 nt, Artificial sequence
  Synthetic oligonucleotide
ggauuuugggcuagcucuuuuuu

SEQ ID NO: 619 - RNA, 23 nt, Artificial sequence
  Synthetic oligonucleotide
uuugucugcuuucuguuuucccu

SEQ ID NO: 620 - RNA, 23 nt, Artificial sequence
  Synthetic oligonucleotide
ccucccuucccuauguacgcuca

SEQ ID NO: 621 - RNA, 23 nt, Artificial sequence
  Synthetic oligonucleotide
ccuuccucccuucccuauguacg

SEQ ID NO: 622 - RNA, 23 nt, Artificial sequence
  Synthetic oligonucleotide
uuucccuuccucccuucccuaug

SEQ ID NO: 623 - RNA, 23 nt, Artificial sequence
  Synthetic oligonucleotide
cuuucuguuuucccuuccucccu

SEQ ID NO: 624 - RNA, 23 nt, Artificial sequence
  Synthetic oligonucleotide
ucugcuuucuguuuucccuuccu

SEQ ID NO: 625 - RNA, 23 nt, Artificial sequence
  Synthetic oligonucleotide
ccuucccuauguacgcucagagc

SEQ ID NO: 626 - RNA, 23 nt, Artificial sequence
  Synthetic oligonucleotide
agccucagacagaccgucugggc

SEQ ID NO: 627 - RNA, 23 nt, Artificial sequence
  Synthetic oligonucleotide
ucagagccucagacagaccgucu

SEQ ID NO: 628 - RNA, 23 nt, Artificial sequence
  Synthetic oligonucleotide
auguacgcucagagccucagaca

SEQ ID NO: 629 - RNA, 23 nt, Artificial sequence
  Synthetic oligonucleotide
acgcucagagccucagacagacc

SEQ ID NO: 630 - RNA, 23 nt, Artificial sequence
  Synthetic oligonucleotide
cccuauguacgcucagagccuca

SEQ ID NO: 631 - RNA, 23 nt, Artificial sequence
  Synthetic oligonucleotide
cuguuuucccuuccucccuuccc

SEQ ID NO: 632 - RNA, 23 nt, Artificial sequence
  Synthetic oligonucleotide
cucagacagaccgucugggcgcc

SEQ ID NO: 633 - RNA, 21 nt, Artificial sequence
  Synthetic oligonucleotide
ggugucaucuaugugaucuug

SEQ ID NO: 634 - RNA, 21 nt, Artificial sequence
  Synthetic oligonucleotide
cuggcccuucucagcgguguc

SEQ ID NO: 635 - RNA, 21 nt, Artificial sequence
  Synthetic oligonucleotide
uggguggccuucccccuggcc

SEQ ID NO: 636 - RNA, 21 nt, Artificial sequence
  Synthetic oligonucleotide
gccuacauccuggccugggug

SEQ ID NO: 637 - RNA, 21 nt, Artificial sequence
  Synthetic oligonucleotide
aggcacccggaguggcaucuc

SEQ ID NO: 638 - RNA, 21 nt, Artificial sequence
  Synthetic oligonucleotide
aucuugcggaaacgcgaauga

SEQ ID NO: 639 - RNA, 21 nt, Artificial sequence
  Synthetic oligonucleotide
uacuccuacgguuucgccuac

SEQ ID NO: 640 - RNA, 21 nt, Artificial sequence
  Synthetic oligonucleotide
ggaaucuuccaaauucuugcu

SEQ ID NO: 641 - RNA, 21 nt, Artificial sequence
  Synthetic oligonucleotide
cuugcuggucugugcgugaug

SEQ ID NO: 642 - RNA, 21 nt, Artificial sequence
  Synthetic oligonucleotide
gugaugagugcugcggccauc

SEQ ID NO: 643 - RNA, 21 nt, Artificial sequence
  Synthetic oligonucleotide
gccaucuacacggugaggcac

SEQ ID NO: 644 - RNA, 21 nt, Artificial sequence
  Synthetic oligonucleotide
caucucaacucggauuacucc

SEQ ID NO: 645 - RNA, 21 nt, Artificial sequence
  Synthetic oligonucleotide
agguuuuacaucacuggaauc

SEQ ID NO: 646 - RNA, 21 nt, Artificial sequence
  Synthetic oligonucleotide
auccugucgaucaucuucagc

SEQ ID NO: 647 - RNA, 21 nt, Artificial sequence
  Synthetic oligonucleotide
uucagcauucugucucuguuc

SEQ ID NO: 648 - RNA, 21 nt, Artificial sequence
  Synthetic oligonucleotide
cuguuccuguucuucugccaa

SEQ ID NO: 649 - RNA, 21 nt, Artificial sequence
  Synthetic oligonucleotide
ugccaacucuucacccucacc

SEQ ID NO: 650 - RNA, 21 nt, Artificial sequence
  Synthetic oligonucleotide
cucaccaaggggggcagguuu

SEQ ID NO: 651 - RNA, 21 nt, Artificial sequence
  Synthetic oligonucleotide
guccaggccaccaugauccug

SEQ ID NO: 652 - RNA, 21 nt, Artificial sequence
  Synthetic oligonucleotide
cagaacuguagcaccucuucc

SEQ ID NO: 653 - RNA, 21 nt, Artificial sequence
  Synthetic oligonucleotide
ucuuccucaggaaauguccac

SEQ ID NO: 654 - RNA, 21 nt, Artificial sequence
  Synthetic oligonucleotide
gaauggcugcagucuguccag

SEQ ID NO: 655 - RNA, 21 nt, Artificial sequence
  Synthetic oligonucleotide
guccaccacuguuucucauca

SEQ ID NO: 656 - RNA, 21 nt, Artificial sequence
  Synthetic oligonucleotide
ucaucaucaccaaacgaaugg

SEQ ID NO: 657 - RNA, 21 nt, Artificial sequence
  Synthetic oligonucleotide
gcaacugaucucuggcagaac

SEQ ID NO: 658 - RNA, 21 nt, Artificial sequence
  Synthetic oligonucleotide
uucgucuccacgaucgucagc

SEQ ID NO: 659 - RNA, 21 nt, Artificial sequence
  Synthetic oligonucleotide
gucagccaauggaucgugggc

SEQ ID NO: 660 - RNA, 21 nt, Artificial sequence
  Synthetic oligonucleotide
gugggcaauggacacgcaacu

SEQ ID NO: 661 - RNA, 21 nt, Artificial sequence
  Synthetic oligonucleotide
guccuccacgucgcggugcug

SEQ ID NO: 662 - RNA, 21 nt, Artificial sequence
  Synthetic oligonucleotide
gugcuggugcugcuguucguc

SEQ ID NO: 663 - RNA, 21 nt, Artificial sequence
  Synthetic oligonucleotide
uugcugaguaucaucguccuc

SEQ ID NO: 664 - RNA, 21 nt, Artificial sequence
  Synthetic oligonucleotide
aagaugcauuaaaauaaacau

SEQ ID NO: 665 - RNA, 21 nt, Artificial sequence
  Synthetic oligonucleotide
ugcauuaaaauaaacaucaga

SEQ ID NO: 666 - RNA, 21 nt, Artificial sequence
  Synthetic oligonucleotide
aauaaacaucagaguaacuca

SEQ ID NO: 667 - RNA, 21 nt, Artificial sequence
  Synthetic oligonucleotide
uuaaaauaaacaucagaguaa

SEQ ID NO: 668 - RNA, 21 nt, Artificial sequence
  Synthetic oligonucleotide
gacuaagaugcauuaaaauaa

SEQ ID NO: 669 - RNA, 21 nt, Artificial sequence
  Synthetic oligonucleotide
uguggacuaagaugcauuaaa

SEQ ID NO: 670 - RNA, 21 nt, Artificial sequence
  Synthetic oligonucleotide
uuauaccaacuguguggacua

SEQ ID NO: 671 - RNA, 21 nt, Artificial sequence
  Synthetic oligonucleotide
gauuuuauaccaacugugugg

SEQ ID NO: 672 - RNA, 21 nt, Artificial sequence
  Synthetic oligonucleotide
accaacuguguggacuaagau

SEQ ID NO: 673 - RNA, 21 nt, Artificial sequence
  Synthetic oligonucleotide
acuguguggacuaagaugcau

SEQ ID NO: 674 - RNA, 21 nt, Artificial sequence
  Synthetic oligonucleotide
uucugauuuuauaccaacugu

SEQ ID NO: 675 - RNA, 21 nt, Artificial sequence
  Synthetic oligonucleotide
cauuuucugauuuuauaccaa

SEQ ID NO: 676 - RNA, 21 nt, Artificial sequence
  Synthetic oligonucleotide
uuuuguuuuugcuuugcauuu

SEQ ID NO: 677 - RNA, 21 nt, Artificial sequence
  Synthetic oligonucleotide
gaccuuuuguuuuugcuuugc

SEQ ID NO: 678 - RNA, 21 nt, Artificial sequence
  Synthetic oligonucleotide
guuuuugcuuugcauuuucug

SEQ ID NO: 679 - RNA, 21 nt, Artificial sequence
  Synthetic oligonucleotide
uugcuuugcauuuucugauuu

SEQ ID NO: 680 - RNA, 21 nt, Artificial sequence
  Synthetic oligonucleotide
uuugcauuuucugauuuuaua

SEQ ID NO: 681 - RNA, 21 nt, Artificial sequence
  Synthetic oligonucleotide
uccagaccuuuuguuuuugcu

SEQ ID NO: 682 - RNA, 21 nt, Artificial sequence
  Synthetic oligonucleotide
agacuccagaccuuuuguuuu

SEQ ID NO: 683 - RNA, 21 nt, Artificial sequence
  Synthetic oligonucleotide
ugaugcuaagacuccagaccu

SEQ ID NO: 684 - RNA, 21 nt, Artificial sequence
  Synthetic oligonucleotide
gcuaagacuccagaccuuuug

SEQ ID NO: 685 - RNA, 21 nt, Artificial sequence
  Synthetic oligonucleotide
ugcccuucugaugcuaagacu

SEQ ID NO: 686 - RNA, 21 nt, Artificial sequence
  Synthetic oligonucleotide
cuucugaugcuaagacuccag

SEQ ID NO: 687 - RNA, 21 nt, Artificial sequence
  Synthetic oligonucleotide
auggugcccuucugaugcuaa

SEQ ID NO: 688 - RNA, 21 nt, Artificial sequence
  Synthetic oligonucleotide
auguauauauggugcccuucu

SEQ ID NO: 689 - RNA, 21 nt, Artificial sequence
  Synthetic oligonucleotide
auauauggugcccuucugaug

SEQ ID NO: 690 - RNA, 21 nt, Artificial sequence
  Synthetic oligonucleotide
caccaacuguagauguauaua

SEQ ID NO: 691 - RNA, 21 nt, Artificial sequence
  Synthetic oligonucleotide
aacuguagauguauauauggu

SEQ ID NO: 692 - RNA, 21 nt, Artificial sequence
  Synthetic oligonucleotide
guagauguauauauggugccc

SEQ ID NO: 693 - RNA, 21 nt, Artificial sequence
  Synthetic oligonucleotide
uggccaccaacuguagaugua

SEQ ID NO: 694 - RNA, 21 nt, Artificial sequence
  Synthetic oligonucleotide
acuuguauuggccaccaacug

SEQ ID NO: 695 - RNA, 21 nt, Artificial sequence
  Synthetic oligonucleotide
guauuggccaccaacuguaga

SEQ ID NO: 696 - RNA, 21 nt, Artificial sequence
  Synthetic oligonucleotide
aaugacuuguauuggccacca

SEQ ID NO: 697 - RNA, 21 nt, Artificial sequence
  Synthetic oligonucleotide
ugucuggcaaugacuuguauu

SEQ ID NO: 698 - RNA, 21 nt, Artificial sequence
  Synthetic oligonucleotide
gccuccaaggacugucuggca

SEQ ID NO: 699 - RNA, 21 nt, Artificial sequence
  Synthetic oligonucleotide
uggcaaugacuuguauuggcc

SEQ ID NO: 700 - RNA, 21 nt, Artificial sequence
  Synthetic oligonucleotide
ggacugucuggcaaugacuug

SEQ ID NO: 701 - RNA, 21 nt, Artificial sequence
  Synthetic oligonucleotide
ccaaggacugucuggcaauga

SEQ ID NO: 702 - RNA, 21 nt, Artificial sequence
  Synthetic oligonucleotide
cugugccuccaaggacugucu

SEQ ID NO: 703 - RNA, 21 nt, Artificial sequence
  Synthetic oligonucleotide
uuggcugggucuaggcuguuc

SEQ ID NO: 704 - RNA, 21 nt, Artificial sequence
  Synthetic oligonucleotide
cugggucuaggcuguucugug

SEQ ID NO: 705 - RNA, 21 nt, Artificial sequence
  Synthetic oligonucleotide
aggcuguucugugccuccaag

SEQ ID NO: 706 - RNA, 21 nt, Artificial sequence
  Synthetic oligonucleotide
uguucugugccuccaaggacu

SEQ ID NO: 707 - RNA, 21 nt, Artificial sequence
  Synthetic oligonucleotide
gucuaggcuguucugugccuc

SEQ ID NO: 708 - RNA, 21 nt, Artificial sequence
  Synthetic oligonucleotide
gagcuuggcugggucuaggcu

SEQ ID NO: 709 - RNA, 21 nt, Artificial sequence
  Synthetic oligonucleotide
ggaccgugaguuccuagagcu

SEQ ID NO: 710 - RNA, 21 nt, Artificial sequence
  Synthetic oligonucleotide
cgugaguuccuagagcuuggc

SEQ ID NO: 711 - RNA, 21 nt, Artificial sequence
  Synthetic oligonucleotide
aguuccuagagcuuggcuggg

SEQ ID NO: 712 - RNA, 21 nt, Artificial sequence
  Synthetic oligonucleotide
ccuagagcuuggcugggucua

SEQ ID NO: 713 - RNA, 21 nt, Artificial sequence
  Synthetic oligonucleotide
cuugggaccgugaguuccuag

SEQ ID NO: 714 - RNA, 21 nt, Artificial sequence
  Synthetic oligonucleotide
gcgucuagacuccuugggacc

SEQ ID NO: 715 - RNA, 21 nt, Artificial sequence
  Synthetic oligonucleotide
cuagacuccuugggaccguga

SEQ ID NO: 716 - RNA, 21 nt, Artificial sequence
  Synthetic oligonucleotide
acuccuugggaccgugaguuc

SEQ ID NO: 717 - RNA, 21 nt, Artificial sequence
  Synthetic oligonucleotide
gcaucagaacaagcgucuaga

SEQ ID NO: 718 - RNA, 21 nt, Artificial sequence
  Synthetic oligonucleotide
cagaacaagcgucuagacucc

SEQ ID NO: 719 - RNA, 21 nt, Artificial sequence
  Synthetic oligonucleotide
acaagcgucuagacuccuugg

SEQ ID NO: 720 - RNA, 21 nt, Artificial sequence
  Synthetic oligonucleotide
cggagcaucagaacaagcguc

SEQ ID NO: 721 - RNA, 21 nt, Artificial sequence
  Synthetic oligonucleotide
ucccacauuuuucuuacgguc

SEQ ID NO: 722 - RNA, 21 nt, Artificial sequence
  Synthetic oligonucleotide
acauuuuucuuacggucggag

SEQ ID NO: 723 - RNA, 21 nt, Artificial sequence
  Synthetic oligonucleotide
cuuacggucggagcaucagaa

SEQ ID NO: 724 - RNA, 21 nt, Artificial sequence
  Synthetic oligonucleotide
uuuucuuacggucggagcauc

SEQ ID NO: 725 - RNA, 21 nt, Artificial sequence
  Synthetic oligonucleotide
cggucggagcaucagaacaag

SEQ ID NO: 726 - RNA, 21 nt, Artificial sequence
  Synthetic oligonucleotide
ucacucccacauuuuucuuac

SEQ ID NO: 727 - RNA, 21 nt, Artificial sequence
  Synthetic oligonucleotide
aaucauaaagccuucaucacu

SEQ ID NO: 728 - RNA, 21 nt, Artificial sequence
  Synthetic oligonucleotide
auaaagccuucaucacuccca

SEQ ID NO: 729 - RNA, 21 nt, Artificial sequence
  Synthetic oligonucleotide
agccuucaucacucccacauu

SEQ ID NO: 730 - RNA, 21 nt, Artificial sequence
  Synthetic oligonucleotide
uucaucacucccacauuuuuc

SEQ ID NO: 731 - RNA, 21 nt, Artificial sequence
  Synthetic oligonucleotide
aguaaaucauaaagccuucau

SEQ ID NO: 732 - RNA, 21 nt, Artificial sequence
  Synthetic oligonucleotide
cuauaaugaguaaaucauaaa

SEQ ID NO: 733 - RNA, 21 nt, Artificial sequence
  Synthetic oligonucleotide
cugcuauuauuacuauaauga

SEQ ID NO: 734 - RNA, 21 nt, Artificial sequence
  Synthetic oligonucleotide
uaggcugcuauuauuacuaua

SEQ ID NO: 735 - RNA, 21 nt, Artificial sequence
  Synthetic oligonucleotide
auuacuauaaugaguaaauca

SEQ ID NO: 736 - RNA, 21 nt, Artificial sequence
  Synthetic oligonucleotide
aaugaguaaaucauaaagccu

SEQ ID NO: 737 - RNA, 21 nt, Artificial sequence
  Synthetic oligonucleotide
uauuauuacuauaaugaguaa

SEQ ID NO: 738 - RNA, 21 nt, Artificial sequence
  Synthetic oligonucleotide
uagcuaggcugcuauuauuac

SEQ ID NO: 739 - RNA, 21 nt, Artificial sequence
  Synthetic oligonucleotide
uaacugcuuuuguaccuagcu

SEQ ID NO: 740 - RNA, 21 nt, Artificial sequence
  Synthetic oligonucleotide
ugcuuuuguaccuagcuaggc

SEQ ID NO: 741 - RNA, 21 nt, Artificial sequence
  Synthetic oligonucleotide
uaccuagcuaggcugcuauua

SEQ ID NO: 742 - RNA, 21 nt, Artificial sequence
  Synthetic oligonucleotide
uuuguaccuagcuaggcugcu

SEQ ID NO: 743 - RNA, 21 nt, Artificial sequence
  Synthetic oligonucleotide
uuuauaacugcuuuuguaccu

SEQ ID NO: 744 - RNA, 21 nt, Artificial sequence
  Synthetic oligonucleotide
augguuuauaacugcuuuugu

SEQ ID NO: 745 - RNA, 21 nt, Artificial sequence
  Synthetic oligonucleotide
aauaauucuguguaauauaaa

SEQ ID NO: 746 - RNA, 21 nt, Artificial sequence
  Synthetic oligonucleotide
auucuguguaauauaaauggu

SEQ ID NO: 747 - RNA, 21 nt, Artificial sequence
  Synthetic oligonucleotide
uguguaauauaaaugguuuau

SEQ ID NO: 748 - RNA, 21 nt, Artificial sequence
  Synthetic oligonucleotide
uaauauaaaugguuuauaacu

SEQ ID NO: 749 - RNA, 21 nt, Artificial sequence
  Synthetic oligonucleotide
auaaaugguuuauaacugcuu

SEQ ID NO: 750 - RNA, 21 nt, Artificial sequence
  Synthetic oligonucleotide
ccugaauaauucuguguaaua

SEQ ID NO: 751 - RNA, 21 nt, Artificial sequence
  Synthetic oligonucleotide
aacauaagauagaauggagac

SEQ ID NO: 752 - RNA, 21 nt, Artificial sequence
  Synthetic oligonucleotide
uaagauagaauggagaccuga

SEQ ID NO: 753 - RNA, 21 nt, Artificial sequence
  Synthetic oligonucleotide
auagaauggagaccugaauaa

SEQ ID NO: 754 - RNA, 21 nt, Artificial sequence
  Synthetic oligonucleotide
aauggagaccugaauaauucu

SEQ ID NO: 755 - RNA, 21 nt, Artificial sequence
  Synthetic oligonucleotide
gagaccugaauaauucugugu

SEQ ID NO: 756 - RNA, 21 nt, Artificial sequence
  Synthetic oligonucleotide
aauccaauuaacaauuuuaca

SEQ ID NO: 757 - RNA, 21 nt, Artificial sequence
  Synthetic oligonucleotide
uuacaacauaagauagaaugg

SEQ ID NO: 758 - RNA, 21 nt, Artificial sequence
  Synthetic oligonucleotide
caauuaacaauuuuacaacau

SEQ ID NO: 759 - RNA, 21 nt, Artificial sequence
  Synthetic oligonucleotide
uaacaauuuuacaacauaaga

SEQ ID NO: 760 - RNA, 21 nt, Artificial sequence
  Synthetic oligonucleotide
aauuuuacaacauaagauaga

SEQ ID NO: 761 - RNA, 21 nt, Artificial sequence
  Synthetic oligonucleotide
uggaaauccaauuaacaauuu

SEQ ID NO: 762 - RNA, 21 nt, Artificial sequence
  Synthetic oligonucleotide
caucuaaacaacucacuggaa

SEQ ID NO: 763 - RNA, 21 nt, Artificial sequence
  Synthetic oligonucleotide
ucacuaaucaucuaaacaacu

SEQ ID NO: 764 - RNA, 21 nt, Artificial sequence
  Synthetic oligonucleotide
uaaucaucuaaacaacucacu

SEQ ID NO: 765 - RNA, 21 nt, Artificial sequence
  Synthetic oligonucleotide
uaaacaacucacuggaaaucc

SEQ ID NO: 766 - RNA, 21 nt, Artificial sequence
  Synthetic oligonucleotide
ucacuggaaauccaauuaaca

SEQ ID NO: 767 - RNA, 21 nt, Artificial sequence
  Synthetic oligonucleotide
caacucacuggaaauccaauu

SEQ ID NO: 768 - RNA, 21 nt, Artificial sequence
  Synthetic oligonucleotide
auuaucacuaaucaucuaaac

SEQ ID NO: 769 - RNA, 21 nt, Artificial sequence
  Synthetic oligonucleotide
ggauuuaccauuccuuauuau

SEQ ID NO: 770 - RNA, 21 nt, Artificial sequence
  Synthetic oligonucleotide
uuaccauuccuuauuaucacu

SEQ ID NO: 771 - RNA, 21 nt, Artificial sequence
  Synthetic oligonucleotide
cauuccuuauuaucacuaauc

SEQ ID NO: 772 - RNA, 21 nt, Artificial sequence
  Synthetic oligonucleotide
ccuuauuaucacuaaucaucu

SEQ ID NO: 773 - RNA, 21 nt, Artificial sequence
  Synthetic oligonucleotide
cuauggauuuaccauuccuua

SEQ ID NO: 774 - RNA, 21 nt, Artificial sequence
  Synthetic oligonucleotide
ggugcuauggauuuaccauuc

SEQ ID NO: 775 - RNA, 21 nt, Artificial sequence
  Synthetic oligonucleotide
ugacaacaagucuuugaaaug

SEQ ID NO: 776 - RNA, 21 nt, Artificial sequence
  Synthetic oligonucleotide
aacaagucuuugaaauggugc

SEQ ID NO: 777 - RNA, 21 nt, Artificial sequence
  Synthetic oligonucleotide
agucuuugaaauggugcuaug

SEQ ID NO: 778 - RNA, 21 nt, Artificial sequence
  Synthetic oligonucleotide
aaauggugcuauggauuuacc

SEQ ID NO: 779 - RNA, 21 nt, Artificial sequence
  Synthetic oligonucleotide
uuugaaauggugcuauggauu

SEQ ID NO: 780 - RNA, 21 nt, Artificial sequence
  Synthetic oligonucleotide
ucagugacaacaagucuuuga

SEQ ID NO: 781 - RNA, 21 nt, Artificial sequence
  Synthetic oligonucleotide
ucacacaucuaaaugagaaau

SEQ ID NO: 782 - RNA, 21 nt, Artificial sequence
  Synthetic oligonucleotide
acaucuaaaugagaaaucagu

SEQ ID NO: 783 - RNA, 21 nt, Artificial sequence
  Synthetic oligonucleotide
cuaaaugagaaaucagugaca

SEQ ID NO: 784 - RNA, 21 nt, Artificial sequence
  Synthetic oligonucleotide
augagaaaucagugacaacaa

SEQ ID NO: 785 - RNA, 21 nt, Artificial sequence
  Synthetic oligonucleotide
gaaaucagugacaacaagucu

SEQ ID NO: 786 - RNA, 21 nt, Artificial sequence
  Synthetic oligonucleotide
uucgucacacaucuaaaugag

SEQ ID NO: 787 - RNA, 21 nt, Artificial sequence
  Synthetic oligonucleotide
gcccuuacagguggaguaucu

SEQ ID NO: 788 - RNA, 21 nt, Artificial sequence
  Synthetic oligonucleotide
uuacagguggaguaucuucgu

SEQ ID NO: 789 - RNA, 21 nt, Artificial sequence
  Synthetic oligonucleotide
agguggaguaucuucgucaca

SEQ ID NO: 790 - RNA, 21 nt, Artificial sequence
  Synthetic oligonucleotide
ggaguaucuucgucacacauc

SEQ ID NO: 791 - RNA, 21 nt, Artificial sequence
  Synthetic oligonucleotide
uaucuucgucacacaucuaaa

SEQ ID NO: 792 - RNA, 21 nt, Artificial sequence
  Synthetic oligonucleotide
acuugcccuuacagguggagu

SEQ ID NO: 793 - RNA, 21 nt, Artificial sequence
  Synthetic oligonucleotide
cuuguggcauuggcauacuug

SEQ ID NO: 794 - RNA, 21 nt, Artificial sequence
  Synthetic oligonucleotide
acggcuuguggcauuggcaua

SEQ ID NO: 795 - RNA, 21 nt, Artificial sequence
  Synthetic oligonucleotide
uggcauuggcauacuugcccu

SEQ ID NO: 796 - RNA, 21 nt, Artificial sequence
  Synthetic oligonucleotide
auuggcauacuugcccuuaca

SEQ ID NO: 797 - RNA, 21 nt, Artificial sequence
  Synthetic oligonucleotide
gcauacuugcccuuacaggug

SEQ ID NO: 798 - RNA, 21 nt, Artificial sequence
  Synthetic oligonucleotide
gcaaaaacacggcuuguggca

SEQ ID NO: 799 - RNA, 21 nt, Artificial sequence
  Synthetic oligonucleotide
acuggagcccuugcaaaaaca

SEQ ID NO: 800 - RNA, 21 nt, Artificial sequence
  Synthetic oligonucleotide
gagcccuugcaaaaacacggc

SEQ ID NO: 801 - RNA, 21 nt, Artificial sequence
  Synthetic oligonucleotide
ccuugcaaaaacacggcuugu

SEQ ID NO: 802 - RNA, 21 nt, Artificial sequence
  Synthetic oligonucleotide
aaacacggcuuguggcauugg

SEQ ID NO: 803 - RNA, 21 nt, Artificial sequence
  Synthetic oligonucleotide
ccaaacuggagcccuugcaaa

SEQ ID NO: 804 - RNA, 21 nt, Artificial sequence
  Synthetic oligonucleotide
augcccaaacuggagcccuug

SEQ ID NO: 805 - RNA, 21 nt, Artificial sequence
  Synthetic oligonucleotide
uuuacgcgcacacggacaaaa

SEQ ID NO: 806 - RNA, 21 nt, Artificial sequence
  Synthetic oligonucleotide
cacacggacaaaaugcccaaa

SEQ ID NO: 807 - RNA, 21 nt, Artificial sequence
  Synthetic oligonucleotide
cgcgcacacggacaaaaugcc

SEQ ID NO: 808 - RNA, 21 nt, Artificial sequence
  Synthetic oligonucleotide
cggacaaaaugcccaaacugg

SEQ ID NO: 809 - RNA, 21 nt, Artificial sequence
  Synthetic oligonucleotide
caaaaugcccaaacuggagcc

SEQ ID NO: 810 - RNA, 21 nt, Artificial sequence
  Synthetic oligonucleotide
aagcuuuacgcgcacacggac

SEQ ID NO: 811 - RNA, 21 nt, Artificial sequence
  Synthetic oligonucleotide
gcugcccgaaccucuguguga

SEQ ID NO: 812 - RNA, 21 nt, Artificial sequence
  Synthetic oligonucleotide
cccgaaccucugugugaagcu

SEQ ID NO: 813 - RNA, 21 nt, Artificial sequence
  Synthetic oligonucleotide
aaccucugugugaagcuuuac

SEQ ID NO: 814 - RNA, 21 nt, Artificial sequence
  Synthetic oligonucleotide
ucugugugaagcuuuacgcgc

SEQ ID NO: 815 - RNA, 21 nt, Artificial sequence
  Synthetic oligonucleotide
ugugaagcuuuacgcgcacac

SEQ ID NO: 816 - RNA, 21 nt, Artificial sequence
  Synthetic oligonucleotide
aaacagccgcugcccgaaccu

SEQ ID NO: 817 - RNA, 21 nt, Artificial sequence
  Synthetic oligonucleotide
acagaaacagccgcugcccga

SEQ ID NO: 818 - RNA, 21 nt, Artificial sequence
  Synthetic oligonucleotide
agccgcugcccgaaccucugu

SEQ ID NO: 819 - RNA, 21 nt, Artificial sequence
  Synthetic oligonucleotide
ccagugcauccaacagaaaca

SEQ ID NO: 820 - RNA, 21 nt, Artificial sequence
  Synthetic oligonucleotide
ugcauccaacagaaacagccg

SEQ ID NO: 821 - RNA, 21 nt, Artificial sequence
  Synthetic oligonucleotide
uccaacagaaacagccgcugc

SEQ ID NO: 822 - RNA, 21 nt, Artificial sequence
  Synthetic oligonucleotide
ugacccagugcauccaacaga

SEQ ID NO: 823 - RNA, 21 nt, Artificial sequence
  Synthetic oligonucleotide
aaaagcccuuuucuggugggu

SEQ ID NO: 824 - RNA, 21 nt, Artificial sequence
  Synthetic oligonucleotide
gcccuuuucuggugggugacc

SEQ ID NO: 825 - RNA, 21 nt, Artificial sequence
  Synthetic oligonucleotide
cuggugggugacccagugcau

SEQ ID NO: 826 - RNA, 21 nt, Artificial sequence
  Synthetic oligonucleotide
ugggugacccagugcauccaa

SEQ ID NO: 827 - RNA, 21 nt, Artificial sequence
  Synthetic oligonucleotide
uuuucuggugggugacccagu

SEQ ID NO: 828 - RNA, 21 nt, Artificial sequence
  Synthetic oligonucleotide
guccaaaagcccuuuucuggu

SEQ ID NO: 829 - RNA, 21 nt, Artificial sequence
  Synthetic oligonucleotide
uguggguagaaaccccaaaug

SEQ ID NO: 830 - RNA, 21 nt, Artificial sequence
  Synthetic oligonucleotide
gguagaaaccccaaaugucca

SEQ ID NO: 831 - RNA, 21 nt, Artificial sequence
  Synthetic oligonucleotide
ccccaaauguccaaaagcccu

SEQ ID NO: 832 - RNA, 21 nt, Artificial sequence
  Synthetic oligonucleotide
gaaaccccaaauguccaaaag

SEQ ID NO: 833 - RNA, 21 nt, Artificial sequence
  Synthetic oligonucleotide
aaauguccaaaagcccuuuuc

SEQ ID NO: 834 - RNA, 21 nt, Artificial sequence
  Synthetic oligonucleotide
aaccaaaguguggguagaaac

SEQ ID NO: 835 - RNA, 21 nt, Artificial sequence
  Synthetic oligonucleotide
ccucauuuagaaaaccaaagu

SEQ ID NO: 836 - RNA, 21 nt, Artificial sequence
  Synthetic oligonucleotide
auuuagaaaaccaaagugugg

SEQ ID NO: 837 - RNA, 21 nt, Artificial sequence
  Synthetic oligonucleotide
agaaaaccaaaguguggguag

SEQ ID NO: 838 - RNA, 21 nt, Artificial sequence
  Synthetic oligonucleotide
aaaguguggguagaaacccca

SEQ ID NO: 839 - RNA, 21 nt, Artificial sequence
  Synthetic oligonucleotide
uccaccucauuuagaaaacca

SEQ ID NO: 840 - RNA, 21 nt, Artificial sequence
  Synthetic oligonucleotide
ccaguccaccucauuuagaaa

SEQ ID NO: 841 - RNA, 21 nt, Artificial sequence
  Synthetic oligonucleotide
ugaaagaagauaccucccucc

SEQ ID NO: 842 - RNA, 21 nt, Artificial sequence
  Synthetic oligonucleotide
agaagauaccucccucccagu

SEQ ID NO: 843 - RNA, 21 nt, Artificial sequence
  Synthetic oligonucleotide
ccucccucccaguccaccuca

SEQ ID NO: 844 - RNA, 21 nt, Artificial sequence
  Synthetic oligonucleotide
gauaccucccucccaguccac

SEQ ID NO: 845 - RNA, 21 nt, Artificial sequence
  Synthetic oligonucleotide
ccucccaguccaccucauuua

SEQ ID NO: 846 - RNA, 21 nt, Artificial sequence
  Synthetic oligonucleotide
caucugaaagaagauaccucc

SEQ ID NO: 847 - RNA, 21 nt, Artificial sequence
  Synthetic oligonucleotide
ccaucucgccccuucccuuuc

SEQ ID NO: 848 - RNA, 21 nt, Artificial sequence
  Synthetic oligonucleotide
cucgccccuucccuuucaucu

SEQ ID NO: 849 - RNA, 21 nt, Artificial sequence
  Synthetic oligonucleotide
uucccuuucaucugaaagaag

SEQ ID NO: 850 - RNA, 21 nt, Artificial sequence
  Synthetic oligonucleotide
ccccuucccuuucaucugaaa

SEQ ID NO: 851 - RNA, 21 nt, Artificial sequence
  Synthetic oligonucleotide
cuuucaucugaaagaagauac

SEQ ID NO: 852 - RNA, 21 nt, Artificial sequence
  Synthetic oligonucleotide
agauaacuccaucucgccccu

SEQ ID NO: 853 - RNA, 21 nt, Artificial sequence
  Synthetic oligonucleotide
guuuuacccagaaauaagaua

SEQ ID NO: 854 - RNA, 21 nt, Artificial sequence
  Synthetic oligonucleotide
cagaaauaagauaacuccauc

SEQ ID NO: 855 - RNA, 21 nt, Artificial sequence
  Synthetic oligonucleotide
uacccagaaauaagauaacuc

SEQ ID NO: 856 - RNA, 21 nt, Artificial sequence
  Synthetic oligonucleotide
aauaagauaacuccaucucgc

SEQ ID NO: 857 - RNA, 21 nt, Artificial sequence
  Synthetic oligonucleotide
aacuccaucucgccccuuccc

SEQ ID NO: 858 - RNA, 21 nt, Artificial sequence
  Synthetic oligonucleotide
uuuguuuuguuuuacccagaa

SEQ ID NO: 859 - RNA, 21 nt, Artificial sequence
  Synthetic oligonucleotide
uuuguuuuguuuuuuguuugu

SEQ ID NO: 860 - RNA, 21 nt, Artificial sequence
  Synthetic oligonucleotide
guuuuuuguuuguuuguuuug

SEQ ID NO: 861 - RNA, 21 nt, Artificial sequence
  Synthetic oligonucleotide
uuuguuuguuuguuuuguuuu

SEQ ID NO: 862 - RNA, 21 nt, Artificial sequence
  Synthetic oligonucleotide
uuuguuuguuuuguuuuaccc

SEQ ID NO: 863 - RNA, 21 nt, Artificial sequence
  Synthetic oligonucleotide
uuuuguuuuacccagaaauaa

SEQ ID NO: 864 - RNA, 21 nt, Artificial sequence
  Synthetic oligonucleotide
ucaguauuuuuguuuuguuuu

SEQ ID NO: 865 - RNA, 21 nt, Artificial sequence
  Synthetic oligonucleotide
cagcucaguauuuuuguuuug

SEQ ID NO: 866 - RNA, 21 nt, Artificial sequence
  Synthetic oligonucleotide
guauaauccagcucaguauuu

SEQ ID NO: 867 - RNA, 21 nt, Artificial sequence
  Synthetic oligonucleotide
uuuuguuuuuuguuuguuugu

SEQ ID NO: 868 - RNA, 21 nt, Artificial sequence
  Synthetic oligonucleotide
uauuuuuguuuuguuuuuugu

SEQ ID NO: 869 - RNA, 21 nt, Artificial sequence
  Synthetic oligonucleotide
aauccagcucaguauuuuugu

SEQ ID NO: 870 - RNA, 21 nt, Artificial sequence
  Synthetic oligonucleotide
aacaguauaauccagcucagu

SEQ ID NO: 871 - RNA, 21 nt, Artificial sequence
  Synthetic oligonucleotide
gcuaaggaacuuuacauccua

SEQ ID NO: 872 - RNA, 21 nt, Artificial sequence
  Synthetic oligonucleotide
aggaacuuuacauccuaacag

SEQ ID NO: 873 - RNA, 21 nt, Artificial sequence
  Synthetic oligonucleotide
acuuuacauccuaacaguaua

SEQ ID NO: 874 - RNA, 21 nt, Artificial sequence
  Synthetic oligonucleotide
uccuaacaguauaauccagcu

SEQ ID NO: 875 - RNA, 21 nt, Artificial sequence
  Synthetic oligonucleotide
uacauccuaacaguauaaucc

SEQ ID NO: 876 - RNA, 21 nt, Artificial sequence
  Synthetic oligonucleotide
aguagcuaaggaacuuuacau

SEQ ID NO: 877 - RNA, 21 nt, Artificial sequence
  Synthetic oligonucleotide
cucguguugagccuuaaagaa

SEQ ID NO: 878 - RNA, 21 nt, Artificial sequence
  Synthetic oligonucleotide
uguugagccuuaaagaaguag

SEQ ID NO: 879 - RNA, 21 nt, Artificial sequence
  Synthetic oligonucleotide
gagccuuaaagaaguagcuaa

SEQ ID NO: 880 - RNA, 21 nt, Artificial sequence
  Synthetic oligonucleotide
cuuaaagaaguagcuaaggaa

SEQ ID NO: 881 - RNA, 21 nt, Artificial sequence
  Synthetic oligonucleotide
aagaaguagcuaaggaacuuu

SEQ ID NO: 882 - RNA, 21 nt, Artificial sequence
  Synthetic oligonucleotide
cagccucguguugagccuuaa

SEQ ID NO: 883 - RNA, 21 nt, Artificial sequence
  Synthetic oligonucleotide
uauguugaccaucagccucgu

SEQ ID NO: 884 - RNA, 21 nt, Artificial sequence
  Synthetic oligonucleotide
uugcuuuuuauguugaccauc

SEQ ID NO: 885 - RNA, 21 nt, Artificial sequence
  Synthetic oligonucleotide
uugaccaucagccucguguug

SEQ ID NO: 886 - RNA, 21 nt, Artificial sequence
  Synthetic oligonucleotide
ccaucagccucguguugagcc

SEQ ID NO: 887 - RNA, 21 nt, Artificial sequence
  Synthetic oligonucleotide
uuuuuauguugaccaucagcc

SEQ ID NO: 888 - RNA, 21 nt, Artificial sequence
  Synthetic oligonucleotide
uuguuugcuuuuuauguugac

SEQ ID NO: 889 - RNA, 21 nt, Artificial sequence
  Synthetic oligonucleotide
auauauguacauaguauuguu

SEQ ID NO: 890 - RNA, 21 nt, Artificial sequence
  Synthetic oligonucleotide
aacacuuuuuacauauaugua

SEQ ID NO: 891 - RNA, 21 nt, Artificial sequence
  Synthetic oligonucleotide
auguacauaguauuguuugcu

SEQ ID NO: 892 - RNA, 21 nt, Artificial sequence
  Synthetic oligonucleotide
acauaguauuguuugcuuuuu

SEQ ID NO: 893 - RNA, 21 nt, Artificial sequence
  Synthetic oligonucleotide
aguauuguuugcuuuuuaugu

SEQ ID NO: 894 - RNA, 21 nt, Artificial sequence
  Synthetic oligonucleotide
uuacauauauguacauaguau

SEQ ID NO: 895 - RNA, 21 nt, Artificial sequence
  Synthetic oligonucleotide
aaaccuauuuauaacacuuuu

SEQ ID NO: 896 - RNA, 21 nt, Artificial sequence
  Synthetic oligonucleotide
cuauuuauaacacuuuuuaca

SEQ ID NO: 897 - RNA, 21 nt, Artificial sequence
  Synthetic oligonucleotide
uuauaacacuuuuuacauaua

SEQ ID NO: 898 - RNA, 21 nt, Artificial sequence
  Synthetic oligonucleotide
cuuuuuacauauauguacaua

SEQ ID NO: 899 - RNA, 21 nt, Artificial sequence
  Synthetic oligonucleotide
gguuuauaaaaccuauuuaua

SEQ ID NO: 900 - RNA, 21 nt, Artificial sequence
  Synthetic oligonucleotide
ugcuguugauugaagauguau

SEQ ID NO: 901 - RNA, 21 nt, Artificial sequence
  Synthetic oligonucleotide
uauaaaaccuauuuauaacac

SEQ ID NO: 902 - RNA, 21 nt, Artificial sequence
  Synthetic oligonucleotide
uguauauaauaucuccgguuu

SEQ ID NO: 903 - RNA, 21 nt, Artificial sequence
  Synthetic oligonucleotide
cuccgguuuauaaaaccuauu

SEQ ID NO: 904 - RNA, 21 nt, Artificial sequence
  Synthetic oligonucleotide
uauaauaucuccgguuuauaa

SEQ ID NO: 905 - RNA, 21 nt, Artificial sequence
  Synthetic oligonucleotide
auaucuccgguuuauaaaacc

SEQ ID NO: 906 - RNA, 21 nt, Artificial sequence
  Synthetic oligonucleotide
auugaagauguauauaauauc

SEQ ID NO: 907 - RNA, 21 nt, Artificial sequence
  Synthetic oligonucleotide
ggagguggggguugcuguuga

SEQ ID NO: 908 - RNA, 21 nt, Artificial sequence
  Synthetic oligonucleotide
ggguugcuguugauugaagau

SEQ ID NO: 909 - RNA, 21 nt, Artificial sequence
  Synthetic oligonucleotide
guggggguugcuguugauuga

SEQ ID NO: 910 - RNA, 21 nt, Artificial sequence
  Synthetic oligonucleotide
aagauguauauaauaucuccg

SEQ ID NO: 911 - RNA, 21 nt, Artificial sequence
  Synthetic oligonucleotide
guugauugaagauguauauaa

SEQ ID NO: 912 - RNA, 21 nt, Artificial sequence
  Synthetic oligonucleotide
caguggagguggggguugcug

SEQ ID NO: 913 - RNA, 21 nt, Artificial sequence
  Synthetic oligonucleotide
aaagcaguggagguggggguu

SEQ ID NO: 914 - RNA, 21 nt, Artificial sequence
  Synthetic oligonucleotide
caaaccaaacagaaagcagug

SEQ ID NO: 915 - RNA, 21 nt, Artificial sequence
  Synthetic oligonucleotide
acagaaagcaguggagguggg

SEQ ID NO: 916 - RNA, 21 nt, Artificial sequence
  Synthetic oligonucleotide
ccaaacagaaagcaguggagg

SEQ ID NO: 917 - RNA, 21 nt, Artificial sequence
  Synthetic oligonucleotide
aaaccaaaccaaacagaaagc

SEQ ID NO: 918 - RNA, 21 nt, Artificial sequence
  Synthetic oligonucleotide
acucaaaccaaaccaaacaga

SEQ ID NO: 919 - RNA, 21 nt, Artificial sequence
  Synthetic oligonucleotide
acaaagaaaaaagagcuagcc

SEQ ID NO: 920 - RNA, 21 nt, Artificial sequence
  Synthetic oligonucleotide
uagcccaaaaucccaaacuca

SEQ ID NO: 921 - RNA, 21 nt, Artificial sequence
  Synthetic oligonucleotide
ccaaaaucccaaacucaaacc

SEQ ID NO: 922 - RNA, 21 nt, Artificial sequence
  Synthetic oligonucleotide
ccaaacucaaaccaaaccaaa

SEQ ID NO: 923 - RNA, 21 nt, Artificial sequence
  Synthetic oligonucleotide
aaucccaaacucaaaccaaac

SEQ ID NO: 924 - RNA, 21 nt, Artificial sequence
  Synthetic oligonucleotide
gagcuagcccaaaaucccaaa

SEQ ID NO: 925 - RNA, 21 nt, Artificial sequence
  Synthetic oligonucleotide
aacagaaagcagacaaagaaa

SEQ ID NO: 926 - RNA, 21 nt, Artificial sequence
  Synthetic oligonucleotide
gaaagcagacaaagaaaaaag

SEQ ID NO: 927 - RNA, 21 nt, Artificial sequence
  Synthetic oligonucleotide
gcagacaaagaaaaaagagcu

SEQ ID NO: 928 - RNA, 21 nt, Artificial sequence
  Synthetic oligonucleotide
agaaaaaagagcuagcccaaa

SEQ ID NO: 929 - RNA, 21 nt, Artificial sequence
  Synthetic oligonucleotide
aaaagagcuagcccaaaaucc

SEQ ID NO: 930 - RNA, 21 nt, Artificial sequence
  Synthetic oligonucleotide
ggaaaacagaaagcagacaaa

SEQ ID NO: 931 - RNA, 21 nt, Artificial sequence
  Synthetic oligonucleotide
agcguacauagggaagggagg

SEQ ID NO: 932 - RNA, 21 nt, Artificial sequence
  Synthetic oligonucleotide
uacauagggaagggaggaagg

SEQ ID NO: 933 - RNA, 21 nt, Artificial sequence
  Synthetic oligonucleotide
uagggaagggaggaagggaaa

SEQ ID NO: 934 - RNA, 21 nt, Artificial sequence
  Synthetic oligonucleotide
ggaggaagggaaaacagaaag

SEQ ID NO: 935 - RNA, 21 nt, Artificial sequence
  Synthetic oligonucleotide
gaagggaaaacagaaagcaga

SEQ ID NO: 936 - RNA, 21 nt, Artificial sequence
  Synthetic oligonucleotide
ucugagcguacauagggaagg

SEQ ID NO: 937 - RNA, 21 nt, Artificial sequence
  Synthetic oligonucleotide
ccagacggucugucugaggcu

SEQ ID NO: 938 - RNA, 21 nt, Artificial sequence
  Synthetic oligonucleotide
acggucugucugaggcucuga

SEQ ID NO: 939 - RNA, 21 nt, Artificial sequence
  Synthetic oligonucleotide
ucugaggcucugagcguacau

SEQ ID NO: 940 - RNA, 21 nt, Artificial sequence
  Synthetic oligonucleotide
ucugucugaggcucugagcgu

SEQ ID NO: 941 - RNA, 21 nt, Artificial sequence
  Synthetic oligonucleotide
aggcucugagcguacauaggg

SEQ ID NO: 942 - RNA, 21 nt, Artificial sequence
  Synthetic oligonucleotide
gaagggaggaagggaaaacag

SEQ ID NO: 943 - RNA, 21 nt, Artificial sequence
  Synthetic oligonucleotide
cgcccagacggucugucugag

SEQ ID NO: 944 - RNA, 22 nt, Artificial sequence
  Synthetic oligonucleotide
ucagacagaccgucugggcgcc

SEQ ID NO: 945 - RNA, 20 nt, Artificial sequence
  Synthetic oligonucleotide
cgcccagacggucugucuga

SEQ ID NO: 946 - DNA, 19 bp, Artificial sequence
  Primer
gaaggtgaaggtcggagtc

SEQ ID NO: 947 - DNA, 20 bp, Artificial sequence
  Primer
gaagatggtgatgggatttc

SEQ ID NO: 948 - DNA, 20 bp, Artificial sequence
  Probe
caagcttcccgttctcagcc